(19) United States
(12) Patent Application Publication
(10) Pub. No.: US 2026/0152735 A1
(43) Pub. Date: Jun. 4, 2026

(54) CHIMERIC GENOME ORGANISMS AND METHODS SYNTHESIZING THEREOF

(71) Applicant: California Institute of Technology, Pasadena, CA (US)

(72) Inventors: Charles J. Sanfiorenzo, Pasadena, CA (US); Raymond J. Zhang, Pasadena, CA (US); Kaihang Wang, Pasadena, CA (US)

(21) Appl. No.: 19/405,152

(22) Filed: Dec. 1, 2025

Related U.S. Application Data
(60) Provisional application No. 63/726,824, filed on Dec. 2, 2024.

(51) Int. Cl. C12N 15/03; C12N 1/20; C12N 5/12; C12N 9/22; C12N 15/113; C12N 15/74; C12N 15/79; C12R 1/63

(52) U.S. Cl. CPC C12N 15/03; C12N 1/20; C12N 5/12; C12N 9/222; C12N 15/113; C12N 15/74; C12N 15/79; C12N 2310/20; C12R 2001/63

(57) ABSTRACT

Disclosed herein include methods, compositions, and kits suitable for use in generation of genome-expanded organisms comprising, e.g., a chimeric genome. In some embodiments, the methods, systems and nucleic acid compositions include systems comprising a donor cell comprising one or more components of a delivery module and a recipient cell comprising one or more components of an integrator module. In some embodiments, the methods, systems and nucleic acid compositions comprise polynucleotides encoding one or more components of a delivery module, and one or more components of an integrator module.

### RELATED APPLICATIONS

The present application claims priority to U.S. Provisional Application No. 63/726,824, filed Dec. 2, 2024. The content of this related application is incorporated herein by reference in its entirety for all purposes.

### STATEMENT REGARDING FEDERALLY SPONSORED R&D

This invention was made with government support under Grant No. GM140937 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810017-US_SequenceListing, created Nov. 27, 2025, which is 1.21 MB in size. The information in the electronic format of the Sequence Listing is incorporated herein by reference in its entirety.

### BACKGROUND

### Field

The present disclosure relates generally to the field of genetic engineering.

### Description of the Related Art

The design and construction of custom genomes furthers our ability to understand and engineer biology. Organisms with designer genomes allow us to disentangle the complexities of gene functionality and essentiality, transcriptional and translational control, and genome fold and architecture. Moreover, designed genomes enable us to engineer novel properties and encode new functions into organisms, enhancing the potential of living systems to produce biological products and perform programmed tasks for health, environmental and manufacturing needs.

Landmark genome construction projects include the assembly of minimized Mycoplasma genomes, the recoding of Escherichia coli Syn61 genome, and the recent consolidation of multiple synthetic chromosomes in the “S. cerevisiae 2.0” synthetic yeast genome project. On-going efforts include recoding the E. coli Syn57 genome and the Salmonella typhimurium genome. These custom genomes can be typified as being minimized, recoded, reorganized, refactored or a combination of all the above.

One notable class of designer genomes consists of those harboring large additions or expansions of heterologous DNA. Rebooting large genome-scale fragments of DNA, from both natural and synthetic sources has implications for studying and engineering biology. However, prior studies have largely been constrained to investigating the factors determining heterologous expression of single genes. One notable study reported the assembly of a fragmented Synechocystis genome across the Bacillus subtilis genome in multiple non-continuous segments using SSRs, demonstrating the possibility of integrating large genome segments from phylogenetically distant sources into a recipient bacteria. However, the genome construction workflow used to install the entire 3.5 megabase (mb) genome is painstaking and time-consuming. Moreover, the inserted DNA was characterized to be transcriptionally silent. An important component for rebooting genomic fragments is the evaluation of the transcription and translation and ultimately physiological outcome resulting transferred DNA incorporated in the recipient genome. An open question is testing how large genomic fragments behave in expression as a function of increasing phylogenetic distance.

The many tools for transferring segments of DNA in microbes have their benefits and drawbacks. Existing reports involving interbacterial transfer of large fragments of DNA include the dual origin of transfer (oriT) cloning or site-specific integration of DNA into Agrobacterium tumefaciens and E. coli, which have been demonstrated for up to 240-kb of DNA. Recently, dual oriT combined with site-specific recombination into engineered bacterial artificial chromosomes have enabled expansion of this technique for megabase sized transfer and rearrangements in E. coli. However, these techniques rely on site-specific recombinases, which require an attachment site pre-engineered in the recipient genome and are thus limited in target versatility. Random, natural Hfr conjugative mating, conjugation coupled homologous recombination (CAGE) and genome fission and fusion have been used to hybridize natural and synthetic enterobacterial strains but are also at times limited in targeting and are dependent on homologous recombination and have only been demonstrated in enterobacteria. Cas9-coupled homologous recombination methods, used to synthesize the recoded E. coli Syn61 genome, have also been shown to enable iterative construction of heterologous human DNA in E. coli, but are restricted to the addition of 100-kb segments in a single step. Finally, in vitro methods to isolate large DNA for transformation of bacterial cells have been used to manipulate large 1-mb episomes in E. coli or to transplant fully synthetic genome into Mycoplasma. However, these methods have not been shown to be broadly generalizable to other organisms and are technically challenging. In sum, new technologies specifically enabling programmable megabase scale genomic recombination-independent integration in a single step are needed and would introduce new versatility and power to the toolbox of custom genome construction and large genomic fragment rebooting.

### SUMMARY

Disclosed herein include systems for producing a chimeric or trimeric genome. In some embodiments, the system comprises: a) a donor cell comprising: (i) a first start cassette (Cassette^{START}) comprising, from 5′ to 3′, at least one cis-acting component of a first delivery module and a first integration sequence, and (ii) a first stop cassette (Cassette^{STOP}) comprising, from 5′ to 3′, a second integration sequence and at least one cis-acting component of the first delivery module, wherein the first Cassette^{START} is integrated at a first site in the genome of the donor cell and the first Cassette^{STOP} is integrated at a second site in the genome of the donor cell, wherein intervening sequence between the first Cassette^{START} and the first Cassette^{STOP} defines a first genomic transfer window; and (iii) one or more trans-acting components of the first delivery module or one or more first helper polynucleotides each comprising a sequence encoding a trans-acting component of the first delivery module, wherein the first delivery module comprises the one or more trans-acting components; and b) a recipient cell comprising one or more components of an integrator module or one or more second helper polynucleotides each comprising a sequence encoding a component of an integrator module, wherein the integrator module comprises the one or more components.

In some embodiments: (1) a sequence defined by the first genomic transfer window is capable of being transferred from the donor cell to the recipient cell upon expression of the one or more trans-acting components of the first delivery module in the donor cell, thereby generating a first donor genomic segment in the recipient cell flanked by the first integration sequence and the second integration sequence of the first Cassette^{START} and the first Cassette^{STOP}; and (2) the first donor genomic segment is capable of being inserted into a first double-stranded target sequence in the genome of the recipient cell upon expression of the one or more components of the integrator module, thereby generating a chimeric genome in the recipient cell.

The system can comprise: c) a second donor cell comprising: (i) a second start cassette (Cassette^{START}) comprising, from 5′ to 3′, at least one cis-acting component of a second delivery module and a first integration sequence, and (ii) a second stop cassette (Cassette^{STOP}) comprising, from 5′ to 3′, a second integration sequence and at least one cis-acting component of the second delivery module, wherein the second Cassette^{START} is integrated at a first site in the genome of the second donor cell and the second Cassette^{STOP} is integrated at a second site in the genome of the second donor cell, thereby defining a second genomic transfer window; and (iii) one or more trans-acting components of the second delivery module or one or more first helper polynucleotides each comprising a sequence encoding a trans-acting component of the second delivery module, wherein the second delivery module comprises the one or more trans-acting components.

In some embodiments: (3) a sequence defined by the second genomic transfer window is capable of being transferred from the second donor cell to the recipient cell comprising the chimeric genome upon expression of the one or more trans-acting components of the second delivery module in the second donor cell, thereby generating a second donor genomic segment flanked by the first integration sequence and the second integration sequence of the second Cassette^{START} and the second Cassette^{STOP} in the recipient cell comprising the chimeric genome; and (4) the second donor genomic segment is capable of being inserted into a second double-stranded target sequence in the genome of the recipient cell comprising the chimeric genome upon expression of the one or more components of the integrator module, thereby generating a trimeric genome in the recipient cell comprising the chimeric genome. In some embodiments, the second double-stranded target sequence is comprised within the first donor genomic segment.

In some embodiments, the first and/or second genomic transfer window is about 10 kb to at least 2 MB in length. In some embodiments, the first and/or second donor genomic segment is about 10 kb to at least 2 MB in length. In some embodiments, any transcription termination sequence (ter) comprised within the first and/or second donor genomic segment is in the same orientation as any ter in or near: the first double-stranded target sequence in the genome of the recipient cell; and/or the second double-stranded target sequence in the genome of the recipient cell comprising the chimeric genome. In some embodiments, the chimeric genome and/or the trimeric genome is at least about 4 MB in length. In some embodiments, the chimeric genome and/or the trimeric genome is at least about 4 MB, 4.5 MB, 5 MB, 6 MB, 6.5 MB, 7 MB, 7.5 MB, 8 MB, 8.5 MB, 9 MB, 9.5 MB, or 10 MB in length.

In some embodiments, the donor cell, the second donor cell, the recipient cell, or any combination thereof is a eukaryotic cell. In some embodiments, the eukaryotic cell is a fungal cell. In some embodiments, the eukaryotic cell is a plant cell. In some embodiments, the donor cell, the second donor cell, the recipient cell, or any combination thereof is a prokaryotic cell. In some embodiments, the prokaryotic cell is or is derived from a bacterial species selected from the group comprising: an Acinetobacter species, an Actinobacillus species, an Actinomycetes species, an Actinomyces species, an Aerococcus species, an Aeromonas species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Chlamydia species, a Citrobacter species, a Coxiella species, a Corynebacterium species, a Clostridium species, an Eikenella species, an Enterobacter species, an Escherichia species, an Enterococcus species, an Ehrlichia species, an Epidermophyton species, an Erysipelothrix species, a Eubacterium species, a Francisella species, a Fusobacterium species, a Gardnerella species, a Gemella species, a Haemophilus species, a Helicobacter species, a Kingella species, a Klebsiella species, a Lactobacillus species, a Lactococcus species, a Listeria species, a Leptospira species, a Legionella species, a Leptospira species, Leuconostoc species, a Mannheimia species, a Microsporum species, a Micrococcus species, a Moraxella species, a Morganella species, a Mobiluncus species, a Micrococcus species, Mycobacterium species, a Mycoplasma species, a Nocardia species, a Neisseria species, a Pasteurella species, a Pediococcus species, a Peptostreptococcus species, a Pityrosporum species, a Plesiomonas species, a Prevotella species, a Porphyromonas species, a Proteus species, a Providencia species, a Pseudomonas species, a Propionibacterium species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, or a Xanthomonas species. In some embodiments, the prokaryotic cell is or is derived from an archaeal species. In some embodiments, the genera and/or species of the donor cell and/or the second donor cell and the genera and/or the species of the recipient cell are the same or different.

Disclosed herein include nucleic acid compositions for producing a chimeric or trimeric genome. In some embodiments, the nucleic acid composition comprises: i) a first and/or second start cassette (Cassette^{START}) each comprising, from 5′ to 3′: at least one cis-acting component of a first and/or second delivery module and a first integration sequence; ii) a first and/or second stop cassette (Cassette^{STOP}) each comprising, from 5′ to 3′: a second integration sequence and at least one cis-acting component of the first and/or second delivery module; iii) one or more first helper polynucleotides each comprising a sequence encoding a trans-acting component of the first and/or second delivery module, wherein the first and/or second delivery module comprise one or more trans-acting components; and optionally iv) one or more second helper polynucleotides each comprising a sequence encoding a component of an integrator module, wherein the integrator module comprises one or more components.

In some embodiments: the first and/or second Cassette^{START} is comprised within a first orthogonal integration vector further comprising one or more third helper polynucleotides each comprising a sequence encoding a component of a first orthogonal integrator module, wherein the first orthogonal integrator module comprises one or more components; and the first and/or second Cassette^{STOP} is comprised within a second orthogonal integration vector further comprising one or more fourth helper polynucleotides each comprising a sequence encoding a component of a second orthogonal integrator module, wherein the second orthogonal integrator module comprises one or more components. In some embodiments, the first and/or second Cassette^{START} is flanked by a 5′ RE and a 3′ LE and the first and/or second Cassette^{STOP} flanked by a 5′ RE and a 3′ LE, and the integrator module, the first orthogonal integrator module, and the second orthogonal integrator module are orthogonal to each other.

In some embodiments, the first orthogonal integrator module and/or the second orthogonal integrator module are each derived from a Type I-B, Type I-D, Type I-F, or Type V-K CRISPR-associated transposase system of a bacteria. In some embodiments, the bacteria comprise Vibrio cholera (Vch), Pseudoalteromonas (Pse), or Scytonema hofmanni (Sho). In some embodiments, the first orthogonal integrator module and/or the second orthogonal integrator module are each derived from the Type I-F CRISPR-associated transposase system, wherein the Type I-F CRISPR-associated transposase system is Tn6677, Tn7000, Tn7001, Tn7002, Tn7003, Tn7004, Tn7005, Tn7006, Tn7007, Tn7008, Tn7009, Tn6900, Tn7010, Tn7011, Tn7012, Tn7013, Tn7014, Tn7015, Tn7016, or Tn7017.

In some embodiments: the one or more components of the first orthogonal integrator module and/or the one or more components of the second orthogonal integrator module comprise: i) an RNA-guided DNA binding complex, wherein the RNA-guided DNA binding complex comprises a Cas protein, a transposase, one or more crRNAs, or any combination thereof, and ii) a transposition complex comprising one or more transposases.

In some embodiments, the RE comprises the sequence of any one of SEQ ID NOs: 343-344 or a sequence having one, two or three mismatches relative to the sequence of any one of SEQ ID NOs: 343-344 and wherein the LE comprises the sequence of any one of SEQ ID NOs: 345-346 or a sequence having one, two or three mismatches relative to the sequence of any one of SEQ ID NOs: 345-346. In some embodiments, the one or more Cas proteins comprise a Cas6 protein, a Cas7 protein, and a Cas8 protein. In some embodiments: the Cas6 protein comprises an amino acid sequence that is at least 80%, 85%, 90%, 95%, 98%, 99%, or 100% identical to SEQ ID NO: 347 or SEQ ID NO: 348; the Cas7 protein comprises an amino acid sequence that is at least 80%, 85%, 90%, 95%, 98%, 99%, or 100% identical to SEQ ID NO: 349 or SEQ ID NO: 350; and/or the Cas8 protein comprises an amino acid sequence that is at least 80%, 85%, 90%, 95%, 98%, 99%, or 100% identical to SEQ ID NO: 351 or SEQ ID NO: 352. In some embodiments, the transposase of the RNA-guided DNA binding complex comprises a TniQ protein. In some embodiments, the TniQ protein comprises an amino acid sequence that is at least 80%, 85%, 90%, 95%, 98%, 99%, or 100% identical to SEQ ID NO: 353 or SEQ ID NO: 354. In some embodiments, the one or more transposases of the transposition complex comprise a TnsA protein, a TnsB protein, and a TnsC protein. In some embodiments, the one or more transposases of the transposition complex comprise a TnsAB fusion protein and a TnsC protein. In some embodiments: the TnsA protein comprises an amino acid sequence that is at least 80%, 85%, 90%, 95%, 98%, 99%, or 100% identical to SEQ ID NO: 355 or SEQ ID NO: 356; the TnsB protein comprises an amino acid sequences that is at least 80%, 85%, 90%, 95%, 98%, 99%, or 100% identical to SEQ ID NO: 357 or SEQ ID NO: 358; and/or the TnsC protein comprises an amino acid sequence that is at least 80%, 85%, 90%, 95%, 98%, 99%, or 100% identical to SEQ ID NO: 359 or SEQ ID NO: 360. In some embodiments, the RNA-guided DNA binding complex comprises one or more helper accessory proteins comprising ClpX and/or ClpP.

In some embodiments: the one or more crRNAs of the first orthogonal integrator module comprise a scaffold comprising the sequence of SEQ ID NO: 389 and/or wherein the nucleic acid sequence encoding the scaffold of the one or more crRNAs of the first orthogonal integrator module comprises the sequence of SEQ ID NO: 386; and or the one or more crRNAs of the second orthogonal integrator module comprise a scaffold comprising the sequence of SEQ ID NO: 390 and/or wherein the nucleic acid sequence encoding the scaffold of the one or more crRNAs of the second orthogonal integrator module comprises the sequence of SEQ ID NO: 387. In some embodiments, one of the one or more crRNAs of the first orthogonal integrator module comprises a spacer that is complementary to a search target sequence on a first strand of an upstream double stranded target sequence of a cell and/or one of the one or more the crRNAs of the second orthogonal integrator module comprises a spacer that is complementary to a search target sequence on a first strand of a downstream double stranded target sequence of the cell, wherein upon expression of the one or more components of the first orthogonal integrator module and/or the one or more components of the second orthogonal integrator module in the cell, the first and/or second Cassette^{START} is capable of being inserted into the upstream double-stranded target sequence in the genome of the cell and the first and/or second Cassette^{STOP} is capable of being inserted into the downstream double stranded target sequence in the genome of the cell, thereby generating a donor cell.

In some embodiments, the first and/or second delivery module comprises or is derived from a bacterial conjugation system. In some embodiments, the bacterial conjugation system is an RP4 (IncP), pKM101 (IncN), R388 (IncW), F, or Tumor-inducing (Ti) system. In some embodiments, the bacterial conjugation system is an RP4 (IncP) plasmid system, and wherein: the at least one cis-acting component comprises an RP4 origin of transfer (oriT) sequence. In some embodiments, the oriT sequence comprises a sequence of SEQ ID NO: 384 or a sequence that is at least 80%, 85%, 90%, 95%, 98%, 99%, or 100% identical to SEQ ID NO: 384; and the one or more trans-acting components comprise or are derived from gene products of the RP4 plasmid.

In some embodiments, the bacterial conjugation system is a Ti plasmid system, and wherein: the at least one cis-acting component comprises a Ti origin of transfer (oriT) sequence; and the one or more trans-acting components comprise or are derived from gene products of the Ti plasmid. In some embodiments, the bacterial conjugation system is a pKM101 (IncN) plasmid system, and wherein: the at least one cis-acting component comprises a pKM101 (IncN) origin of transfer (oriT) sequence; and the one or more trans-acting components comprise or are derived from gene products of the pKM101 (IncN) plasmid. In some embodiments, the bacterial conjugation system is an R388 (IncW) plasmid system, and wherein: the at least one cis-acting component comprises an R388 (IncW) origin of transfer (oriT) sequence; and the one or more trans-acting components comprise or are derived from gene products of the R388 (IncW) plasmid. In some embodiments, the bacterial conjugation system is an F plasmid system, and wherein: the at least one cis-acting component comprises an F plasmid origin of transfer (oriT) sequence; and the one or more trans-acting components comprise or are derived from gene products of the F plasmid.

In some embodiments, the integrator module is derived from a transposase system or a site-directed DNA recombinase system. In some embodiments, the site-directed DNA recombinase system comprises a site-directed integrase system. In some embodiments, the integrator module is derived from a Type I-B, Type I-D, Type I-F, or Type V-K CRISPR-associated transposase system of a bacteria. In some embodiments, the bacteria comprises Vibrio cholera (Vch), Pseudoalteromonas (Pse), or Scytonema hofmanni (Sho). In some embodiments, the integrator module is derived from the Type I-F CRISPR-associated transposase system. In some embodiments, the Type I-F CRISPR-associated transposase system is Tn6677, Tn7000, Tn7001, Tn7002, Tn7003, Tn7004, Tn7005, Tn7006, Tn7007, Tn7008, Tn7009, Tn6900, Tn7010, Tn7011, Tn7012, Tn7013, Tn7014, Tn7015, Tn7016, or Tn7017. In some embodiments, the Type I-F CRISPR-associated transposase system is Tn6677.

In some embodiments: the one or more components of the integrator module comprise: i) an RNA-guided DNA binding complex, wherein the RNA-guided DNA binding complex comprises one or more Cas proteins, a transposase, one or more crRNAs, or any combination thereof, and ii) a transposition complex comprising one or more transposases; and the first integration sequence of the first and/or second Cassette^{START} comprises an R-TE (RE) and the second integration sequence of the first and/or second Cassette^{STOP} comprises an L-TE (LE).

In some embodiments, the RE comprises the sequence of any one of SEQ ID NOs: 361 or a sequence having one, two or three mismatches relative to the sequence of any one of SEQ ID NOs: 361 and wherein the LE comprises the sequence of any one of SEQ ID NOs: 362 or a sequence having one, two or three mismatches relative to the sequence of any one of SEQ ID NOs: 362. In some embodiments, the one or more Cas proteins comprise a Cas6 protein, a Cas7 protein, and a Cas8 protein. In some embodiments: the Cas6 protein comprises an amino acid sequence that is at least 80%, 85%, 90%, 95%, 98%, 99%, or 100% identical to SEQ ID NO: 363; the Cas7 protein comprises an amino acid sequence that is at least 80%, 85%, 90%, 95%, 98%, 99%, or 100% identical to SEQ ID NO: 364; and/or the Cas8 protein comprises an amino acid sequence that is at least 80%, 85%, 90%, 95%, 98%, 99%, or 100% identical to SEQ ID NO: 365. In some embodiments, the transposase of the RNA-guided DNA binding complex comprises a TniQ protein. In some embodiments, the TniQ protein comprises an amino acid sequence that is at least 80%, 85%, 90%, 95%, 98%, 99%, or 100% identical to SEQ ID NO: 366. In some embodiments, the one or more transposases of the transposition complex comprise a TnsA protein, a TnsB protein, and a TnsC protein. In some embodiments, the one or more transposases of the transposition complex comprise a TnsAB fusion protein and a TnsC protein. In some embodiments: the TnsA protein comprises an amino acid sequence that is at least 80%, 85%, 90%, 95%, 98%, 99%, or 100% identical to SEQ ID NO: 367; the TnsB protein comprises an amino acid sequences that is at least 80%, 85%, 90%, 95%, 98%, 99%, or 100% identical to SEQ ID NO: 368; and/or the TnsC protein comprises an amino acid sequence that is at least 80%, 85%, 90%, 95%, 98%, 99%, or 100% identical to any one of SEQ ID NOs: 369. In some embodiments, the RNA-guided DNA binding complex comprises one or more helper accessory proteins comprising ClpX and/or ClpP.

In some embodiments, the one or more crRNAs of the integrator module comprise a scaffold comprising the sequence of SEQ ID NO: 385 and/or wherein the nucleic acid sequence encoding the scaffold of the one or more crRNAs of the integrator module comprises the sequence of SEQ ID NO: 388. In some embodiments, at least one of the one or more crRNAs comprise a spacer that is complementary to a search target sequence on a first strand of the first double stranded target sequence. In some embodiments, at least one of the one or more crRNAs comprise a spacer that is complementary to a search target sequence on a first strand of the second double-stranded target sequence.

In some embodiments, the integrator module is derived from a Type V-K CRISPR-associated transposase system. In some embodiments: the one or more components of the integrator module comprise: i) an RNA-guided DNA binding complex, wherein the RNA-guided DNA binding complex comprises a Cas protein and one or more crRNAs, or any combination thereof, and ii) a transposition complex comprising one or more transposases; and wherein the first integration sequence of the first and/or second Cassette^{START} comprises an R-TE (RE) and the second integration sequence of the first and/or second Cassette^{STOP} comprises an L-TE (RE). In some embodiments, the Cas protein comprises Cas12k. In some embodiments, the one or more transposases of the transposition complex comprise TniQ, TnsB, TnsC, or any combination thereof. In some embodiments, at least one of the one or more crRNAs comprise a spacer that is complementary to a search target sequence on a first strand of the first double stranded target sequence. In some embodiments, at least one of the one or more crRNAs comprise a spacer that is complementary to a search target sequence on a first strand of the second double-stranded target sequence.

In some embodiments: the one or more components of the integrator module comprise an integrase. In some embodiments, the integrase is Bxb1 and the first and/or second integration sequences comprise an attB site. In some embodiments: the genome of the recipient cell comprises at least one attP site. In some embodiments: the one or more components of the integrator module comprise an integrase. In some embodiments, the integrase is phiC31 and the first and/or second integration sequences comprise an attB site. In some embodiments: the genome of the recipient cell comprises at least one attP site. In some embodiments: the one or more components of the integrator module comprise a recombinase. In some embodiments, the recombinase is Cre or FLP and the first and/or second integration sequences comprise a loxP site or an FRT site. In some embodiments, wherein: the genome of the recipient cell comprises at least one loxP site or FRT site.

In some embodiments, the first and/or second Cassette^{START}, the first and/or second Cassette^{STOP} or any combination thereof comprise: one or more marker polynucleotides each comprising a sequence encoding a positive or negative screening and/or selection marker. In some embodiments, the one or more marker polynucleotides are situated 5′ of the at least one cis-acting component of the first and/or second delivery module in the first and/or second Cassette^{START} and/or the one or more marker polynucleotides are situated 5′ of the second integration sequence in the first and/or second Cassette^{STOP}. In some embodiments: the positive screening and/or selection marker is selected from the group comprising: a fluorescent protein, an antibiotic resistance cassette, an enzyme, or any combination thereof. In some embodiments, the enzyme is β-galactosidase. In some embodiments, the negative screening and/or selection marker is selected from the group comprising: a fluorescent protein, an enzyme, or a combination thereof. In some embodiments, the enzyme is encoded by the sacB gene and/or the pheS^{mut} gene. In some embodiments, the antibiotic resistance cassette confers resistance to an antibiotic comprising phleomycin D1 (ZEOCIN™), kanamycin, spectinomycin, streptomycin, ampicillin, carbenicillin, bleomycin, erythromycin, polymyxin B, tetracycline, chloramphenicol, or any combination thereof. In some embodiments, the fluorescent protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof. In some embodiments, at least one of the one or more marker polynucleotides is flanked by recombinase sites. In some embodiments, the one or more marker polynucleotides of the first Cassette^{START} and/or the first Cassette^{STOP} are flanked by recombinase sites. In some embodiments, the at least one of the one or more marker polynucleotides of the first Cassette^{START} and/or the first Cassette^{STOP} flanked by recombinase sites are capable of being removed from the genome of the recipient cell comprising the chimeric genome upon expression of at least one recombinase or integrase in the recipient cell comprising the chimeric genome. In some embodiments, the at least one recombinase or integrase comprises phiC31, Bxb1, Cre, and/or FLP.

In some embodiments: one or more of the one or more first helper polynucleotides comprise a first promoter operably linked to the sequence encoding a trans-acting component of the first and/or second delivery module; one or more of the one or more second helper polynucleotides comprise a second promoter operably linked to the sequence encoding a component of an integrator module; one or more of the one or more third helper polynucleotides comprise a third promoter operably linked to the sequence encoding a component of a first orthogonal integrator module; and/or one or more of the one or more fourth helper polynucleotides comprise a fourth promoter operably linked to the sequence encoding a component of a second orthogonal integrator module. In some embodiments, the first, second, third, and/or fourth promoters are the same or different. In some embodiments, the first, second, third, and/or fourth promoters comprise a constitutive promoter, an inducible promoter, or a combination thereof.

In some embodiments, the first, second, third, and/or fourth promoters comprises the TlpA operator/promoter, lambda phage pL, lambda phage pR, lambda phage pRM, or any combination thereof. In some embodiments, the first, second, third, and/or fourth promoters comprise a promoter selected from the group comprising: a bacteriophage promoter, (e.g., Pls1con, T3, T7, SP6, or PL); a bacterial promoter, (e.g., Pbad, PmgrB, Ptrc2, Plac/ara, Ptac, or Pm); and/or a bacterial-bacteriophage hybrid promoter, (e.g., PLlacO or PLtetO). In some embodiments, the first, second, third, and/or fourth promoters comprise a positively regulated E. coli promoter selected from the group comprising: a σ⁷⁰ promoter, (e.g., inducible pBad/araC promoter, Lux cassette right promoter, modified lambda Prm promoter, plac Or2-62 (positive), pBad/AraC with extra REN sites, pBad, P(Las) TetO, P(Las) CIO, P(Rhl), Pu, FecA, pRE, cadC, hns, pLas, or pLux); a σ^{S} promoter, (e.g., Pdps); a σ³² promoter, (e.g., heat shock); and/or a σ⁵⁴ promoter, (e.g., glnAp2). In some embodiments, the first, second, third, and/or fourth promoters comprise a negatively regulated E. coli promoter selected from the group comprising: a σ⁷⁰ promoter, (e.g., Promoter (PRM+), modified lambda Prm promoter, TetR-TetR-4C P(Las) TetO, P(Las) CIO, P(Lac) IQ, RecA_DlexO_DLacOl, dapAp, FecA, Pspac-hy, pcI, plux-cI, plux-lac, CinR, CinL, glucose controlled, modifed Pr, modifed Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLaclq, rrnB P1, cadC, hns, PfhuA, pBad/araC, nhaA, OmpF, or RcnR); a σ^{S} promoter, (e.g., Lutz-Bujard LacO with alternative sigma factor σ³⁸); a σ³² promoter, (e.g., Lutz-Bujard LacO with alternative sigma factor σ³²); and/or a σ⁵⁴ promoter, (e.g., glnAp2). In some embodiments, the first, second, third, and/or fourth promoters comprise a P7 promoter. In some embodiments, the first, second, third, and/or fourth promoters comprise a heat-shock promoter, (e.g., pTSR, pR-pL, GrpE, HtpG, Lon, RpoH, Clp, and/or DnaK). In some embodiments, the first, second, third, and/or fourth promoters comprise a constitutive Escherichia coli as promoter, (e.g., osmY promoter (BBa_J45993)); a constitutive Escherichia coli σ³² promoter (e.g., htpG heat shock promoter (BBa_J45504)); a constitutive Escherichia coli σ⁷⁰ promoter (e.g., lacq promoter (BBa_J54200 or BBa_J56015), E. coli CreABCD phosphate sensing operon promoter (BBa_J64951), GlnRS promoter (BBa_K088007), lacZ promoter (BBa_K119000 or flfa_K119001), M13K07 gene I promoter (BBa_M13101), M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), or M13110 (BBa_M13110)); a constitutive Bacillus subtilis σ^{A} promoter (e.g., promoter veg (BBa_K143013), promoter 43 (BBa_K143013), P_{liaG} (BBa_K823000), P_{lepA} (BBa_K823002), or P_{veg}(BBa_K823003)); a constitutive Bacillus subtilis σ^{B} promoter, (e.g., promoter ctc (BBa_K143010) or promoter gsiB (BBa_K143011)); a Salmonella promoter (e.g., Pspv2 from Salmonella (BBa_K112706) or Pspv from Salmonella (BBa_K112707)); a bacteriophage T7 promoter (e.g., BBa_I712074, BBa_1719005, BBa_J34814, BBa_J64997, BBa_K113010, BBa_K113011, BBa_K113012, BBa_R0085, BBa_R0180, BBa_R0181, BBa_R0182, BBa_R0183, BBa_Z0251, BBa_Z0252, or BBa_Z0253); and/or a bacteriophage SP6 promoter, (e.g., SP6 promoter (BBa_J64998)). In some embodiments, the first, second, third, and/or fourth promoters comprise an Arabinose inducible promoter (P^{Ara}).

In some embodiments: the one or more first helper polynucleotides are situated on the same nucleic acid or different nucleic acids; and/or the one or more second helper polynucleotides are situated on the same nucleic acid or different nucleic acids. In some embodiments: two or more of the one or more first helper polynucleotides situated on the same nucleic acid, two or more of the one or more second helper polynucleotides situated on the same nucleic acid, two or more of the one or more third helper polynucleotides comprised within the first orthogonal integration vector, and/or two or more of the one or more fourth helper polynucleotides comprised within the second orthogonal integration vector, are comprised within an operon or operably linked via a tandem expression element. In some embodiments, the tandem expression element is selected from the group comprising a bacterial ribosome binding site (RBS), an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof.

In some embodiments, any of the one or more first helper polynucleotides, the one or more second helper polynucleotides, the one or more third helper polynucleotides, the one or more fourth helper polynucleotides, and/or the bacterial operon comprise a 5′UTR and/or a 3′ UTR. In some embodiments, the one or more first helper polynucleotides, the one or more second helper polynucleotides, the one or more third helper polynucleotides, and/or the one or more fourth helper polynucleotides comprise a transcript stabilization element.

In some embodiments, the one or more first helper polynucleotides or the one or more second helper polynucleotides are comprised within one or more vectors. In some embodiments, the one or more vectors, the first orthogonal vector, and/or the second orthogonal vector comprise an RNA viral vector, a DNA viral vector, a plasmid vector, an artificial chromosome, or any combination thereof.

In some embodiments: i) the first and/or second Cassette^{START} comprises the sequence of SEQ ID NO: 370 or a sequence that is at least 80%, 85%, 90%, 95%, 98%, 99%, or 100% identical to SEQ ID NO: 370; ii) the first and/or second Cassette^{STOP} comprises the sequence of SEQ ID NO: 371 or a sequence that is at least 80%, 85%, 90%, 95%, 98%, 99%, or 100% identical to SEQ ID NO: 371; iii) the first and/or second Cassette^{START} flanked by a 5′ RE and a 3′ LE and comprises the sequence of any one of SEQ ID NOs: 372-373 or a sequence that is at least 80%, 85%, 90%, 95%, 98%, 99%, or 100% identical to any one of SEQ ID NOs: 372-373; and/or iv) the first and/or second Cassette^{STOP} flanked by a 5′ RE and a 3′ LE comprises sequence of any one of SEQ ID NOs: 374-375 or a sequence that is at least 80%, 85%, 90%, 95%, 98%, 99%, or 100% identical to any one of SEQ ID NOs: 374-375.

In some embodiments: the two or more first helper polynucleotides comprised within an operon or operably linked via a tandem expression element comprise the sequence of SEQ ID NO: 376 or a sequence that is at least 80%, 85%, 90%, 95%, 98%, 99%, or 100% identical to any one of SEQ ID NO: 376; the two more second helper polynucleotides comprised within an operon or operably linked via a tandem expression element comprise the sequence of SEQ ID NO: 377 or a sequence that is at least 80%, 85%, 90%, 95%, 98%, 99%, or 100% identical to any one of SEQ ID NO: 377; the two or more third helper polynucleotides comprised within an operon or operably linked via a tandem expression element comprise the sequence of SEQ ID NO: 378 or a sequence that is at least 80%, 85%, 90%, 95%, 98%, 99%, or 100% identical to any one of SEQ ID NO: 378; and/or the two or more fourth helper polynucleotides comprised within an operon or operably linked via a tandem expression element comprise the sequence of SEQ ID NO: 379 or a sequence that is at least 80%, 85%, 90%, 95%, 98%, 99%, or 100% identical to any one of SEQ ID NO: 379.

In some embodiments: i) the two or more first helper polynucleotides are comprised within a plasmid, and wherein the plasmid comprises the sequence of SEQ ID NO: 380; ii) the two more second helper polynucleotides are comprised within a plasmid, and wherein the plasmid comprises the sequence of SEQ ID NO: 381; iii) the first orthogonal integration vector comprises the sequence of SEQ ID NO: 382; and/or iv) the second orthogonal integration vector comprises the sequence of SEQ ID NO: 383.

Provided herein are kits comprising any of the systems or nucleic acid compositions of the disclosure, and set of instructions for use.

Disclosed herein are methods for producing a chimeric genome. In some embodiments, the method comprises: contacting a recipient cell disclosed herein with a donor cell disclosed herein, and: expressing the one or more trans-acting components of the first delivery module in the donor cell, thereby the sequence defined by the genomic transfer window is transferred from the donor cell to the recipient cell and thereby the first donor genomic segment comprising the first donor sequence flanked by the first integration sequence and the second integration sequence of the first Cassette^{START} and the first Cassette^{STOP} is generated in the recipient cell; and expressing the one or more components of the integrator module in the recipient cell, thereby the first donor genomic segment is inserted into the first double-stranded target sequence in the genome of the recipient cell, thereby generating the chimeric genome in the recipient cell.

The method can comprise repeating the contacting step one or more times, wherein each contacting step comprises contacting an nth-donor cell with the recipient cell, thereby generating a recipient cell comprising an n-meric genome, wherein each nth-donor cell comprises: (i) an nth start cassette (Cassette^{START}) comprising, from 5′ to 3′, at least one cis-acting component of an nth delivery module and an nth integration sequence, and (ii) an nth stop cassette (Cassette^{STOP}) comprising, from 5′ to 3′, an nth integration sequence and at least one cis-acting component of the nth delivery module, wherein the nth Cassette^{START} is integrated at a first site in the genome of the nth donor cell and the nth Cassette^{STOP} is integrated at a second site in the genome of the nth donor cell, wherein intervening sequence between the nth Cassette^{START} and the nth Cassette^{STOP} defines an nth genomic transfer window; and (iii) one or more trans-acting components of the nth delivery module or one or more first helper polynucleotides each comprising a sequence encoding a trans-acting component of the nth delivery module, wherein the nth delivery module comprises the one or more trans-acting components, and optionally wherein the sequence of each nth genomic transfer window is different from each other.

Disclosed herein are methods for producing a trimeric genome. In some embodiments, the method comprises: (a) contacting a recipient cell of the disclosure with the donor cell of the disclosure, and: expressing the one or more trans-acting components of the first delivery module in the donor cell, thereby the sequence defined by the genomic transfer window is transferred from the donor cell to the recipient cell and thereby the first donor genomic segment comprising the first donor sequence flanked by the first integration sequence and the second integration sequence of the first Cassette^{START} and the first Cassette^{STOP} is generated in the recipient cell; and expressing the one or more components of the integrator module in the recipient cell, thereby the first donor genomic segment is inserted into the first double-stranded target sequence in the genome of the recipient cell, thereby generating the chimeric genome in the recipient cell, and (b) contacting the recipient cell comprising the chimeric genome with a second donor cell of the disclosure, and: expressing the one or more trans-acting components of the second delivery module in the second donor cell, thereby the sequence defined by the second genomic transfer window is transferred from the second donor cell to the recipient cell comprising the chimeric genome and thereby the second donor genomic segment comprising the second donor sequence flanked by the first integration sequence and the second integration sequence of the second Cassette^{START} and the second Cassette^{STOP} is generated in the recipient cell comprising the chimeric genome; and expressing the one or more components of the integrator module in the recipient cell comprising the chimeric genome, thereby the second donor genomic segment is inserted into the second double-stranded target site in the genome of the recipient cell comprising the chimeric genome, thereby generating the trimeric genome in the recipient cell comprising the chimeric genome, optionally wherein the second double-stranded target site is comprised within the first donor genomic segment.

In some embodiments, prior to the contacting of step (b), the method comprises expressing at least one recombinase or integrase in the recipient cell comprising the chimeric genome, thereby the at least one of the one or more marker polynucleotides of the first Cassette^{START} and/or the first Cassette^{STOP} flanked by recombinase sites are removed from the genome of the recipient cell comprising the chimeric genome. In some embodiments, the ratio of the donor cell to the recipient cell is approximately 4:1 (v/v) and/or the ratio of the second donor cell to the recipient cell comprising the chimeric genome is approximately 4:1 (v/v). In some embodiments, the first and/or second donor genomic segment is inserted into the first and/or second double-stranded target sequence in the genome of the recipient cell and/or the genome of the recipient cell comprising the chimeric genome at an integration efficiency of at least 10⁴ CFU. In some embodiments, the method has an on-target insertion rate of at least 90%.

Disclosed herein are genome-expanded organisms. In some embodiments, the genome-expanded organism comprises a chimeric genome, trimeric genome, or n-meric genome generated by a method provided herein. In some embodiments, the chimeric, trimeric genome, or n-meric genome is stable following at least 100 generations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1B are related to the design and overview of ACE. FIG. 1A displays a schematic representation of the ACE system, including the Cassette^{START}, Cassette^{STOP} pAra-Trans, and pAra-CAST components. Cassette^{START} contains genes conferring sucrose susceptibility (sacB/−1) and chloramphenicol resistance (cm^{R}/+1), a RK24 origin of transfer (oriT), and the right end (RE) of Tn6677. Cassette^{STOP} contains genes for p-chlorophenylalanine susceptibility (pheS/−2) and hygromycin resistance (hygroR/+2), a green fluorescent protein (gfp) gene, a RK24 oriT, and the left end (LE) of Tn6677. Cassette^{START} and Cassette^{STOP} can be contained in pOrthoCAST-1 and pOrthoCAST-2 plasmids, respectively. pAra-Trans encodes RK24 conjugation machinery under an arabinose-inducible (P^{Ara}) promoter on a BAC vector conferring apramycin resistance (apraR/+3). pAra-CAST encodes the RNA-guided Tn6677 transposase under p^{Ara} and a constitutively expressed Tn6677 crRNA on a spec^{R}/+4 CDF replication backbone. FIG. 1B depicts an overview of the ACE process: (i) Cassette^{START} and Cassette^{STOP}, either in the form of PCR products or contained in plasmids are integrated into the donor genome to demarcate the transfer window; (ii) pAra-Trans is introduced into the donor strain to prime for conjugation; (iii) pAra-CAST is introduced into the recipient strain to prime for integration; (iv) Donor and recipient strains are mixed and pAra-Trans/pAra-CAST are induced with arabinose; (v) Single-stranded DNA (ssDNA) is transferred from the donor to the recipient, initiating at the oriT in Cassette^{START} and terminating at the oriT in Cassette^{STOP}; (vi) The ssDNA circularizes and is converted to double-stranded DNA (dsDNA) in the recipient. The CAST complex then integrates the dsDNA into the recipient genome in a crRNA-dependent manner. Two integration outcomes (RE-LE and LE-RE integrants) in cell Y are possible due to the bidirectional transposition properties of CASTs; (vii) Successful ACE events are identified through antibiotic selection and gfp marker screening.

FIG. 2A-FIG. 2G display non-limiting exemplary data related to the validation and characterization of ACE. As shown in FIG. 2A, Cassette^{START} and Cassette^{STOP} are integrated into different locations flanking a synthetic DNA segment (Syn100) on the E. coli DH10B genome. Cassette^{START} insertion occurs at a fixed site (locus¹). Cassette^{STOP} can be inserted at three distinct locations (locus^{2a}, locus^{2b}, or locus^{2c}) to create donor strains for variable transfer lengths: 10, 50, or 100-kb. As shown in FIG. 2B, recipient E. coli DH10B^{Δdif::lux} strains are engineered with plasmids encoding diverse crRNAs (colored squares), each targeting specific integration sites (colored triangles). Color association (TS1: purple, TS2: blue, and TS3: pink) distinguishes between crRNAs and target sites. FIG. 2C displays exemplary data related to ACE efficiency, measured by GFP+ colony forming units (c.f.u.) per experiment as a function of Syn100 transfer lengths up to 100-kb. FIG. 2D displays data related to ACE specificity, calculated as the percentage of lux-/GFP+c.f.u. resulting from on-target integration into different positions within the lux operon (crRNA^{TS2}, crRNA^{luxA*}, crRNA^{luxB1}, crRNA^{luxB2}) Precision of 10 and 50-kb transfers were assessed only with crRNA^{TS2} and ΔcrRNA while 100-kb was also assessed with the three variant crRNAs. FIG. 2E displays results of colony PCR interrogating the orientation and integrity of a Syn100 integration (100-kb) into the recipient genome at the TS2 site. Two primer sets verify junctions between (i) transferred DNA and the recipient genome and (ii) internal regions within the Syn100 segment. FIG. 2F displays exemplary results from Tn-seq analysis revealing on-target RE-LE/LE-RE ACE integration frequency at TS1, TS2, and TS3 across all transfer lengths (10, 50, and 100-kb). Off-target events are represented by colored circles corresponding to target sites. FIG. 2G shows heat maps of integration site distributions (x-axis), measured in percent reads mapped, for 10, 50 and 100-kb Syn100 donor cargo integrations at TS1 (purple), TS2 (blue) and TS3 (pink) as determined by Tn-seq of transfer populations. Note: Efficiency and specificity are presented as the mean±standard deviation of n=3 independent experiments, with individual values displayed as white circles.

FIG. 3A-FIG. 3O display non-limiting exemplary data showing ACE enables modular and scalable megabase-scale additions between synthetic and natural strains to create transcriptionally active genomic chimeras. FIG. 3A displays a diagram of seg. 5 transfer from Syn61 to DH10B^{Δdif::lux} at TS3 (pink rectangle-triangle). Chimeric genome represented as the TE-flanked Syn61 segment (red) integrated into the DH10B genome (gray). FIG. 3B shows genome-wide read coverages (RPGC) for the seg. 5 chimeric strain. Syn61 genomic segments shaded in pink, and homologous DH10B genomic segments shaded in dark-gray. Non-redundant sections of the E. coli genome shaded in light-gray; TS3 target site depicted by the pink triangle. Read fraction mapping to the Syn61 and homologous DH10B segments set above the coverage plot.

FIG. 3C-FIG. 3D show diagrams and charts as in FIG. 3A-FIG. 3B, but depicting seg. 4 transfer at TS3 (pink). FIG. 3E-FIG. 3F show diagrams and charts as in FIG. 3A-FIG. 3B, but depicting seg. 3 transfer at TS3 (pink), which is centered around the oriC of Syn61. The genomic chimera produced by this transfer has an ectopic oriC inserted at the TS3 region, which forms an additional peak in read coverage alongside the wild type oriC. Dark gray and pink arrows illustrate the two sets of replication forks emanating from the WT and ectopic oriCs, respectively. FIG. 3G-FIG. 3H show diagrams as in FIG. 3A-FIG. 3B, but depicting seg. 2 transfer at TS1 (purple). FIG. 3I-FIG. 3J show diagrams and charts as in FIG. 3A-FIG. 3B, but depicting seg. 1 transfer at TS2 (blue). FIG. 3K displays genome-wide mapping of Syn61 genes (pink) to corresponding DH10B homologs (dark-gray) in the seg. 1 Chimeric genome. DH10B and Syn61 homolog-pairs connect by pink/yellow lines. FIG. 3L displays hierarchical clustering of transcript counts (log(TPM+1)) of Syn61 and DH10B homologs in DH10B, Chimeric and Syn61 strains. FIG. 3M shows genome-wide scatterplot (top) and heat-map (bottom) of positional expression (log(TPM+1)) of each Syn61 (pink) and DH10B (dark-gray) homolog within the Chimeric strain. FIG. 3N shows expression distribution (log(TPM+1)) of Syn61 (pink) and DH10B (dark-gray) homologs in DH10B, Chimeric and Syn61 strains. Asterisks represent statistical outliers. FIG. 3O displays differential expression of redundant (red) and non-redundant (pink) genes between Chimeric and DH10B background strains. The log 2 of the fold-change of redundant (red) and non-redundant (pink) genes are plotted alongside their respective −log₁₀ adjusted p-value. P-value cutoff set at 10-3 and log 2 fold-change cutoff set to 1; points below the cutoff are in gray. Note: Transcript counts and differential expression are derived from up to n=3 independent samples.

FIG. 4A-FIG. 4K display non-limiting exemplary data related to the creation of cross-genus genomic chimeras and trimeras. FIG. 4A depicts a diagram of ˜1-Mb S. flexneri to E. coli DH10B and MDS42 at the dif site (TS: gray rectangle-triangle). Chimeric genomes represented as the TE-flanked S. flexneri segment (dark-blue) integrated into DH10B and MDS42 genomes (light-/dark-gray). FIG. 4B shows genome-wide read coverages (RPGC) for E. coli DH10B+1-Mb S. flexneri (top) and E. coli MDS42+1-Mb S. flexneri (bottom) chimeric strains as in FIG. 3B, but with a dif target (TS: gray triangle). Shown in FIG. 4C is genome-wide mapping of S. flexneri gene variants given in blue to their corresponding E. coli wild-type homologs given in dark gray in the E. coli+1-Mb S. flexneri chimeric genome with a R-L integration orientation. Pairs of DH10B and Shigella homologs are connected by either blue or light gray lines. FIG. 4D shows expression distribution of bulk S. flexneri transcripts (blue) compared to bulk homologous E. coli transcripts (gray) in DH10B, MDS42, S. flexneri, and Chimeric strains measured in log(TPM+1). Distributions are based on the data from n 3 biologically independent samples. Edges of boxes close to zero represent outliers that are the result of bioinformatic and sequencing artifacts with no biological significance. FIG. 4E shows hierarchical clustering of transcript counts, measured in log(TPM+1) in the S. flexneri, Chimeric and MDS42 strains, of S. flexneri gene homologs originally deleted in the MDS42 strain background that are reconstituted via ACE chimerization with S. flexneri. Three individual replicates are shown for each strain. Genes are clustered based on expression level within each genomic background. Shown in FIG. 4F is positional heat map of expression in log(TPM+1) of two unique gene-rich S. flexneri loci in DH10B, MDS42, S. flexneri, and their respective E. coli+1-Mb S. flexneri chimeras. FIG. 4G displays a diagram of the stepwise ACE synthesis of a tripartite-genome-chimera (trimera) between E. coli DH10B, E. coli Syn61 and S. flexneri. In a two-step process, S. flexneri segment (dark-blue; step 2) integration into the DH10B+2-Mb Syn61 dif site (TS′: pink rectangle; generated in step 1) results in a 7.4-Mb trimeric genome. FIG. 4H displays phase-contrast confocal microscopy of synthesized chimeras. Scale bar is provided in the S. flexneri image. Shown in FIG. 4I is genome-wide read coverage (RPGC) for the DH10B+2-Mb Syn61 chimeric and the +1-Mb S. flexneri trimeric strains as in FIG. 3B. S. flexneri genomic segments shaded in blue; Syn61 and DH10B segments are respectively shaded in pink and gray, with TS/TS' given by gray/pink triangles. FIG. 4J depicts data of the expression distribution of bulk Syn61 transcripts (pink), S. flexneri transcripts (blue) compared to bulk homologous DH10B transcripts (gray) in DH10B, S. flexneri, Syn61, Chimeric and Trimeric strains measured in log(TPM+1). Distributions are based on the data from n 3 biologically independent samples. Edges of boxes close to zero represent outliers that are the result of bioinformatic and sequencing artifacts with no biological significance. Shown in FIG. 4K is differential expression of 3-fold (blue), 2-fold redundant genes (red) and non-redundant genes (black) between the trimeric strain and the DH10B background strain. The log 2 of the ratio of the expression level of the 3-fold and 2-fold redundant genes in the trimeric strain to the expression level of their homolog in the DH10B strain (blue and red) is plotted alongside log 2 of the ratio of the expression level of non-redundant genes in the trimeric strain to the expression level of its homolog in the DH10B strain (black). Points are also plotted based on their −log₁₀ adjusted p-value. P-value cutoff was set at 0.001 and the log 2 fold-change cutoff was set to ±1 so that points below the cutoff are in gray.

FIG. 5A-FIG. 5I display non-limiting exemplary data related to the creation of cross-order and cross-class genome chimeras. FIG. 5A shows a simplified phylogenetic tree of Alphaproteobacteria and Gammaproteobacteria illustrating relationships between tested ACE donors and recipients. Cross-genus and cross-order transfers from Shigella to Escherichia, Escherichia to Vibrio and Pseudomonas to Escherichia are respectively shown by dark blue, purple and light blue arrows. The tree shows cross-class transfer from Escherichia to Agrobacterium through a dark pink arrow. The direction of the arrow denotes the direction of transfer. FIG. 5B displays a diagram of 100-kb E. coli Syn61 transfer to A. tumefaciens and corresponding genome-wide read coverage for the outcome chimeric genome with integration 100-kb Syn61 into the A. tumefaciens genome at the genomic dif site. The Syn61 genome is depicted by the red circle and the A. tumefaciens genomes are depicted with chromosome 1 as a yellow circle and chromosome 2 as an orange segment. Shown in FIG. 5C is a diagram of TE-flanked 1-Mb E. coli MDS42 segment (dark-gray) transfer to V natriegens (Chr.1 dark-purple; Chr.2: light-purple) genome at either Chr.1 TS^{C1} or Chr.2 TS^{C2} chromosomal dif-targets (dark-/light-purple triangles). FIG. 5D shows the genome-wide read coverage (RPGC) for the V. natriegens chr.1+1-Mb MDS42 and V. natriegens chr.2+1-Mb MDS42 chimeric strains as in FIG. 3B, but with TS^{C1}/TS^{C2} respectively given by dark/light purple triangles. FIG. 5E shows expression distribution of bulk V. natriegens transcripts (purple) compared to bulk E. coli transcripts (gray) in MDS42, V. natriegens, and 1-Mb MDS42 Chimeric Vibrio strains measured in log(TPM+1). Distributions are based on the data from n 3 biologically independent samples. Edges of boxes close to zero represent outliers that are the result of bioinformatic and sequencing artifacts with no biological significance. Shown in FIG. 5F is a diagram of 522-kb P. protegens pf-5 transfer into E. coli DH10B. The P. protegens genome is depicted by the blue circle and the E. coli genomes are depicted with the gray circle. Genome integration sites TS4, dif and TS3 are indicated by their respective colored rectangle triangles. FIG. 5G shows a diagram as in FIG. 5F, but for the 415-kb P. protegens pf-5 transfer to E. coli DH10B into genomic sites TS2 and dif.

FIG. 5H shows the genome-wide read coverages for the E. coli DH10B chimeric genomes with integration of 522-kb P. protegens at TS4, dif and TS3 genomic loci, respectively. Transferred DNA is highlighted in blue and integration sites indicated with colored triangles. FIG. 5I shows data as in FIG. 5H, but genome-wide read coverages for 415-kb P. protegens transfer into E. coli DH10B at the dif and TS2 genomic loci, respectively.

FIG. 6A-FIG. 6H display non-limiting exemplary data showing functional interaction and multi-omics of megabase-scale P. protegens-E. coli chimeras uncover emergent metabolic landscape. FIG. 6A displays results of a hemolysis phenotype assay of 522-kb P. protegens-E. coli chimeras on LB agar and Columbia agar supplemented with 5% v/v defibrinated sheep blood. Shown in FIG. 6B is a 3D MS1 heatmap of P. protegens and E. coli DH10B showing m/z vs. retention time vs. intensity landscape. FIG. 6C displays a 3D MS1 heatmap of 415-kb P. protegens TS2 and dif E. coli DH10B chimeras showing m/z vs. retention time vs. intensity landscape. FIG. 6D depicts m/z peaks at representative retention times for the 415-kb P. protegens dif E. coli DH10B chimera compared to the control donor and recipient bacteria. Shown in FIG. 6E is a Venn diagram of distinct m/z peaks in E. coli DH10B, P. protegens pf-5 and 415-kb P. protegens-E. coli TS2 and dif chimera highlighting the set of metabolites unique to and shared with between the bacteria. FIG. 6F shows the distribution of unique m/z counts shared between experimental replicates of 415-kb P. protegens chimera. FIG. 6G shows a diagram as in FIG. 5F, but for the 522-kb P. protegens pf-5 transfer to 415-kb P. protegens dif E. coli DH10B into genomic TS3 site. FIG. 6H shows data as in FIG. 5H, but genome-wide read coverages for the 937-kb P. protegens-E. coli genome chimera.

FIG. 7A-FIG. 7H display non-limited exemplary data related to the detailed characterization of the fidelity and specificity of Syn100 transfers with ACE. FIG. 7A shows representative plates of 100-kb Syn100 ACE transfers into TS1-3 in E. coli DH10B Δdif::lux. The top panel shows colony luminescence while the bottom panel shows colony fluorescence under blue light. The 10⁻⁴ dilution plate was shown for each experiment. FIG. 7B shows genotyping colony PCR gels for six integrant colonies of the 100-kb Syn100 transfer into the DH10B Δdif::lux TS1 site. In the schematic above the gel, the red rectangle represents the Syn100 insert, the yellow boxes represent the LE and RE, and the purple rectangles represent the TS1 site. The integration site in TS1 targeted by crRNA^{TS1} is shown with a purple triangle. The right and left junctions for a RL integration are checked with primers specific to TS1 and the Syn100 segment, shown as blue and red arrows, respectively. D and R denote donor and recipient controls in each assay. Lengths of each PCR product are shown above each set of arrows in the schematic. FIG. 7C displays sanger sequencing traces for the newly formed transposition junctions detected by genotyping colony PCR of clone 1 in b. for the 100-kb Syn100 transfer into DH10B Δdif::lux. FIG. 7D shows genotyping colony PCR gels as in FIG. 7B, but genotyping the same six integrant colonies for the right and left junctions for a LR integration, represented with the blue and red arrow pairs. FIG. 7E shows genotyping colony PCR gels as in FIG. 7B, but genotyping the same six integrant colonies for two internal stretches of the Syn100 transfer with primer pairs denoted by the blue and red arrow pairs priming within the Syn100 region. FIG. 7F shows genotyping colony PCR gels as in FIG. 7B, but genotyping colony PCR for six integrant colonies of the 100-kb Syn100 transfer into DH10B Δdif::lux TS3 site for the right and left junctions for a RL integration, represented with the blue and red arrow pairs. FIG. 7G shows genotyping colony PCR gels as in FIG. 7E, but genotyping the six integrant colonies of the 100-kb Syn100 transfer into DH10B Δdif::lux TS3 site for two internal stretches of the Syn100 transfer with primer pairs denoted by the blue and red arrow pairs priming within the Syn100 region. Shown in FIG. 7H are growth and fluorescence of Syn100 donor (D), DH01B recipient (R) and the chimeric outcome strains (1-6) for each of the three integration positions (TS1-3) on different solid growth media conditions. Cells were spotted LB agar supplemented with one of the following in order from top to bottom (100 μg/ml streptomycin, 200 μg/ml hygromycin, 2.5 mM 4-chlorophenylalanine, 25 μg/ml chloramphenicol, 7.5% w/v sucrose, 50 μg/ml spectinomycin, 50 μg/ml apramycin). Chemiluminescence and fluorescence of the spotted colonies was assessed on the streptomycin condition.

FIG. 8A-FIG. 8I show non-limiting exemplary data related to the description and demonstration of Tn-seq methodology and characterization of mutant CAST insertion behaviors with the Syn100 donor. Shown in FIG. 8A is the Tn-seq workflow; (1) Bacterial colonies formed by ACE transfer on selective agar plates are (2) scraped and collected for genomic DNA extraction. A subpopulation of the extracted DNA contains fragments carrying transposon ends (TEs), denoted in beige, as a hallmark of a conjugative CAST event. (3) DNA fragments are randomly tagged with adapters and fragmented (tagmented) using bead-linked transposomes using routine library preparation protocols to append 5′ and 3′ sequences for standard sequencing primer annealing (blue and green). (4) Indices, P5 and P7 adapters are then added using a custom PCR-based library amplification step that allows for enrichment of sequences containing TEs from the total population of tagmented DNA. Briefly, custom primers containing the P5 adapter sequence (pink) as well as an index sequence (purple) were designed to anneal to the RE of the transposon (beige) were paired with a standard primer containing a P7 adapter (orange) and an index sequence (dark purple) which anneals to the standard sequencing primer binding site (blue) in a low cycle PCR. (5) This TE-enriched sequencing library is then sequenced on a MiSeq using a custom sequencing primer that anneals to the RE. FIG. 8B shows genome-wide distribution of Tn-seq reads, measured in percent reads, from ACE transfer experiments of 10-kb, 50-kb and 100-kb Syn100 cargoes into TS1 (purple bars), TS2 (blue bars) and TS3 (pink bars). Locations of each target site are given by triangles of the appropriate color. Genome coordinates are plotted with the origin of replication (oriC) as the zeroth coordinate. FIG. 8C shows integration orientation for ACE transfer experiments of 10-kb, 50-kb and 100-kb Syn100 cargoes into TS1 (purple, left bars for each length), TS2 (blue, middle bars for each length) and TS3 (pink, right bars for each length) given in percent Tn-seq reads. RL integration is given in blue and LR integration is given in light blue. Shown in FIG. 8D is the genome-wide distribution of off-target Tn-seq reads for ACE transfer experiments of 100-kb Syn100 cargo into TS1 (purple circles), TS2 (blue circles) and TS3 (pink circles). Frequency of off-target integration is given as a read fraction normalized to the most frequent off-target integration position. The most frequent off-target integration position for each of the TS1-3 experiments is additionally labeled with the percent reads in the Tn-seq mapping to said position. Genome coordinates are plotted with the origin of replication (oriC) as the zeroth coordinate. FIG. 8E displays a diagram illustrating the variants of the pAra-CAST plasmids used in 100-kb Syn100 cargo transfers and corresponding efficiency of ACE transfer with each variant, measured in GFP+ c.f.u. Data is shown as the mean±s.d. for n=3 biologically independent samples. Shown in FIG. 8F is the genome-wide distribution of Tn-seq reads for ACE transfer of 100-kb Syn100 cargo with pAra-CAST-crRNA^{NT}. Frequency of integration is given as a read fraction normalized to the most frequent integration position. The most frequent integration position is additionally labeled with the percent reads in the Tn-seq mapping to said position. Genome coordinates are plotted with the origin of replication (oriC) as the zeroth coordinate. FIG. 8G is similar to FIG. 8F but shows genome-wide distribution of Tn-seq reads for ACE transfer of 100-kb Syn100 cargo with pAra-CAST-ΔcrRNA. FIG. 8H is similar to FIG. 8F, but shows genome-wide distribution of Tn-seq reads for ACE transfer of 100-kb Syn100 cargo with pAra-CAST-Δcascade. FIG. 8I is similar to FIG. 8F but shows genome-wide distribution of Tn-seq reads for ACE transfer of 100-kb Syn100 cargo with pAra-CAST-ΔQcascade.

FIG. 9A-FIG. 9G display non-limiting exemplary data showing ACE enables megabase-scale additions between synthetic and natural strains. FIG. 9A shows a depiction of the synthetic E. coli Syn6l donor genome (pink ring), recoded across 18,000 codons in the MDS42 genome (gray ring). Three transfer windows (red segments) are defined for 100-kb, 600-kb and 1-Mb centered around the dif site of Syn61. FIG. 9B displays diagrams representing 100-kb, 600-kb and 1-Mb Syn6l insertions at TS1, TS2 and TS3 E. coli DH10B recipients. Unstable chimeric genomes are grayed out and set behind their stable counterparts for 600-kb and 1-Mb Syn6l transfers. FIG. 9C shows a graph of ACE transfer efficiency, measured in GFP+c.f.u., of 100-kb, 600-kb and 1-Mb Syn6l transfers in recipient strains (TS1: purple, TS2: blue, TS3: pink, crRNA^{NT}: light-gray, ΔcrRNA: gray, and ΔCAST: dark-gray). FIG. 9D shows schematics and data related ACE specificity at TS2 target site for 100-kb, 600-kb and 1-Mb Syn6l transfers. On the left, representation of the TS2 locus with 100-kb, 600-kb and 1-Mb insertions (red). ACE precision calculated as the percentage of lux-GFP+c.f.u. out of total GFP+c.f.u. Data shown as the mean±s.d. for n=3 biologically independent samples; sample values are reported as white circles. FIG. 9E shows the genome-wide distribution of Tn-seq reads, measured in percent reads, from ACE transfer experiments of 100-kb, 600-kb and 1-Mb integrations into TS1 (purple), TS2 (blue) and TS3 (pink). Data represented as in FIG. 2F. FIG. 9F shows heat maps of integration site distributions, measured in percent reads mapped, for 100-kb, 600-kb and 1-Mb integrations at TS1 (purple), TS2 (blue) and TS3 (pink) as determined by Tn-seq. Shown in FIG. 9G are genome-wide read coverages (in RPGC) for the DH10B+1-Mb-Syn61 genomic chimeric strain. Syn6l genomic segments shaded in pink, and homologous DH10B genomic segments shaded in dark-gray. Non-redundant sections of the E. coli genome shaded in light-gray; TS2 target site depicted by the blue triangle. Read fraction mapping to the Syn6l and homologous DH10B segments set above the coverage plot.

FIG. 10A-FIG. 10E display non-limiting exemplary data related to the efficiency and specificity of ACE relative to comparable genome-scale integration techniques and orientation preference for 100-kb Syn6l transfers into E. coli DH10B Δdif: lux. Shown in FIG. 10A is a transfer diagram of 100-kb, 1-Mb and 2-Mb fragments of Syn6l into E. coli DH10B. Insertion of the cargo DNA into the TS2 site is accomplished with programmable integration techniques such as ACE, λ-red recombination or an adapted REXER 2 (Cas9 excision in tandem with λ-red recombination). FIG. 10B shows ACE efficiency compared to λ-red recombination and REXER 2, measured by GFP+ colony forming units (c.f.u.) per experiment as a function of Syn61 transfer lengths up to 2-Mb. Data for ACE, λ-red recombination and REXER are shown from left to right for each size. FIG. 10C shows ACE specificity compared to λ-red recombination and REXER 2, calculated as the percentage of lux-/GFP+c.f.u. resulting from on-target integration into TS2 within the lux operon as a function of Syn61 transfer lengths up to 2-Mb. Data for ACE, λ-red recombination and REXER are shown from left to right for each size. Shown in FIG. 10D is a diagram of the E. coli Syn61 (red) and E. coli DH10B Δdif::lux (gray) genomes showing the positions of ter sites, dif site and origin of replication (oriC). Placements of the TEs are marked with yellow bars on the Syn61 genome corresponding to the 100-kb, 600-kb and 1-Mb Syn61 donor. Locations of the target sites 1-3 (TS1-3) in DH10B Δdif::lux are represented with purple, blue and pink bars. FIG. 10E shows an integration diagram of DH10B Δdif::lux genome with transfer of 100-kb Syn61 donor into either TS1, TS2 or TS3 via ACE. The transferred DNA is represented by the red ring segment flanked by yellow bars denoting the TEs labeled with either R or L to distinguish between RE and LE. Key sequences in the donor such as ter sites and the dif site are presented as red arrows and red bars, respectively. At each integration site (TS1-3), the two possible transposition orientations of the donor are depicted. (+) represents RL integration while (−) represents LR integration. At the TS1 and TS3 sites, one integration orientation and not the other would result in a chimeric genome that experiences collision (labeled Col. in the diagram) between replisome and the non-permissive side of a tus-bound ter site during chromosome synthesis, leading to termination of DNA replication (marked with a red X). The other orientation will lead to no collision (labeled N. Col.) as the replication fork will pass through the permissive end of the tus-ter complex (marked with a green checkmark). Adjacent to each integration is a corresponding genome-wide coverage map (in RPGC) for each E. coli DH10B+100-kb Syn61 genomic chimera strain. Syn61 genomic segments are shaded in pink, and homologous E. coli DH10B genomic segments are shaded in dark gray. Non-redundant sections of the E. coli genome are in lighter grays. Locations of the TS is given by the triangle on the X-axis, triangles pointing down indicate the target is the sense strand, while triangles pointing up indicate the target is the antisense strand. Read fraction mapping to the Syn61 segment and the homologous DH10B segment is set above the coverage plot. Read fractions share the same color coding as the coverage plot.

FIG. 11A-FIG. 11B display non-limiting exemplary schematics and data related to the architecture and stability of massively expanded E. coli synthetic genomes. FIG. 11A, as in FIG. 10 (e.g., FIGS. 10B, 10D), except integration diagrams of DH10B Δdif::lux genome with transfer of 600-kb Syn61 donor into either TS1, TS2 or TS3 via ACE. At the TS1 and TS3 sites, both orientations of integration would result in replication fork collision with the non-permissive side of tus-bound ter site (Col., marked with a red X). Labels of TS1 and TS3 genome-wide coverage plots are grayed out to show instability and loss of the transferred segment following transposition. At the TS2 site, both integration orientations would lead to chimeric genomes that do not experience replication fork collision with the non-permissive side of a ter-tus complex. The corresponding genome coverage plot indicates the stability of the transferred segment. Similar to FIG. 11A, FIG. 11B shows integration diagrams of DH10B Δdif::lux genome with transfer of 1-Mb Syn61 donor into either TS1, TS2 or TS3 via ACE.

FIG. 12A-FIG. 12D display non-limiting exemplary schematics and data related to the stability and fidelity of Syn-segment transfers throughout the E. coli genome. Shown in FIG. 12A are diagrams representing the transfer of 1-Mb seg. 2, 500-kb seg. 4 and 500-Kb seg. 5 Syn61 donors for insertion at TS1, TS2 and TS3 in recipient E. coli DH10B Δdif::lux strains leading to nine possible chimeric genomes. The size of each outcome genome is labeled in bold in the chimeric strain. Note that integration orientation of the cargo is not explicitly depicted in the final product; each chimeric genome can adopt either an RL or an LR configuration. FIG. 12B shows genome-wide read coverages (in RPGC) for the E. coli DH10B+1-Mb seg. 2 genomic chimeric strain integrated at TS2 and TS3. Syn61 genomic segments are shaded in pink, and homologous E. coli DH10B genomic segments are shaded in dark gray. Non-redundant sections of the E. coli genome are in lighter grays. Location of the TS2 and TS3 targets is given by the blue and pink triangles, respectively. Read fraction mapping to the Syn61 segment and the homologous DH10B segment is set above the coverage plot. Read fractions share the same color coding as the coverage plot. FIG. 12C shows genome-wide read coverages as in FIG. 12B, but genome-wide read coverage plot and read fraction mapping of 500-kb seg. 4 donor for insertion at TS1 (purple) and TS2 (blue) in recipient E. coli DH10B Δdif::lux strains. FIG. 12D shows genome wide read coverages in FIG. 12B, but genome-wide read coverage plot and read fraction mapping of 500-kb seg. 5 donor for insertion at TS1 (purple) and TS2 (blue) in recipient E. coli DH10B Δdif lux strains.

FIG. 13A-FIG. 13G display non-limiting exemplary schematics and data related to the construction, validation, and characterization of an organism with a trimeric genome in addition to extended stability of chimeric genomes. Shown in FIG. 13A is a schematic related to using PhiC31 recombinase to excise +2/2 selection and GFP markers from Cassette^{STOP} of a dif integrated E. coli DH10B+2-Mb Syn-seg. 1 genome chimera strain. The expression of PhiC31 in this strain triggers a crossing-over event between the attP and attB sequences flanking the selection and screening markers in Cassette^{STOP}, leading to marker loss, and enabling subsequent ACE events in this chimeric strain as shown in FIG. 5E. FIG. 13B displays a gel of results for assaying for Cassette^{STOP} marker excision via genotyping colony PCR of dif integrated E. coli DH10B+2-Mb seg. 1 cells before (+) and after (−) expression of PhiC31 recombinase. Chimeric cells before (+) and after (−) PhiC31 induction were also stamped onto selective LB agar containing either 200 μg/ml hygromycin (Hygro) or 2.5 mM 4-chlorophenylalanine (Cl-Phe). Shown in FIG. 13C are genotyping colony PCR gels for eight integrant colonies of the 1-Mb S. flexneri transfer into the dif integrated E. coli DH10B+2-Mb seg. 1. In the schematic above the gel, the blue rectangle represents the 1 Mb Shigella insert, the yellow boxes represent the LE and RE, and the red duplex DNA represents 2 Mb seg. 1 dif site. The integration site in dif targeted by crRNA^{TS(d(f)} is shown with a red triangle. The right and left junctions for a RL integration are checked with primers specific to dif and the Shigella segment, shown as blue and red arrows, respectively. D and R denote donor and recipient controls in each assay. Lengths of each PCR product are shown above each set of arrows in the schematic. Shown in FIG. 13D are genotyping colony PCR gels for eight integrant colonies of the 1-Mb S. flexneri transfer into the dif integrated E. coli DH10B+2-Mb Syn-seg. 1. In the schematic above the gel, the blue rectangle represents the 1 Mb Shigella insert, the yellow boxes represent the LE and RE, the red rectangles represent 2-Mb seg. 1 genomic stretches and the gray duplex DNA represents the DH10B dif site. The integration site in dif targeted by crRNA^{TS1(d(f)} is shown with a gray triangle. The right and left junctions for a RL integration of 2-Mb seg. 1 at the DH10B dif are checked with primers specific to dif and the Syn61 segment, shown as blue and red arrows, respectively. D and R denote donor and recipient controls in each assay. Lengths of each PCR product are shown above each set of arrows in the schematic. FIG. 13E displays a semi-log plot of growth curves (optical density as a function of hours elapsed) for chimeric and trimeric genome bacteria as well as their wild-type precursor strains. E. coli DH10B is given by the blue curve, S. flexneri is given by the orange curve, E. coli Syn61 is given by the green curve, DH10B+1-Mb Shigella is given by the red curve, DH10B+1-Mb Syn61 is given by the purple curve, DH10B+2-Mb Syn61 is given by the brown curve and DH10B+2-Mb Syn61+1-Mb Shigella is given by the pink curve. Reported curves are the average of four independently grown biological replicates of each strain, with the solid curve representing the mean OD at each time point and the colored halo surrounding each curve representing +/−s.d. Note that optical density is plotted on a logarithmic scale, hence the semi-log plot. FIG. 13F shows a graph of doubling times for chimeric and trimeric genome bacteria as well as their wild-type precursor strains. Reported doubling time, shown as mean+/−s.d., are the average of four independently grown biological replicates of each strain. The doubling time reported for each individual experiment is represented by dots. Shown in FIG. 13G are data related to the percentage of a panel of chimeric genomes stable (blue) and unstable (red) after prolonged growth exceeding 100 generations in select growth mediums (LB, TB and M9 media) and temperatures (30° C. or 37° C.). Stability is characterized by the retention of the external megabase-scale DNA at the end of extended growth as evidenced by genotyping colony PCR and genome read coverage from short-read sequencing.

FIG. 14A-FIG. 14J display non-limiting exemplary data related to extended transcriptomic characterization of E. coli Shigella and E. coli Vibrio genomic chimeras. Shown in FIG. 14A is hierarchical clustering of transcript counts, measured in log of transcripts per million+1 (log(TPM+1)), of S. flexneri gene homologs and E. coli gene homologs in the DH10B, MDS42 DH10B-Shigella, MDS42-Shigella and Shigella strains. Three individual replicates are shown for each strain. Genes are clustered based on expression level within each genomic background. FIG. 14B displays a graph of differential expression of redundant genes (dark blue) and non-redundant genes (light blue) between the chimeric strain and the E. coli background strain for both E. coli DH10B+1 Mb S. flexneri and E. coli MDS42+1 Mb S. flexneri. The log 2 of the ratio of the expression level of the redundant genes in the chimeric strain to the expression level of their homolog in the E. coli strain (dark blue) is plotted alongside log 2 of the ratio of the expression level of non-redundant genes in the chimeric strain to the expression level of its homolog in the E. coli strain (light blue). Points are also plotted based on their −log₁₀ adjusted p-value. P-value cutoff was set at 0.001 and the log 2 fold-change cutoff was set to 1 so that points below the cutoff are in gray. Shown in FIG. 14C is a Venn diagram of the redundant genes overexpressed in the E. coli DH10B+1-Mb S. flexneri chimera and the E. coli MDS42+1-Mb S. flexneri chimera and highlighting the set of redundant genes present and overexpressed in both chimeric strains. FIG. 14D displays results of analysis of the set of redundant genes overexpressed in both chimeric strains, grouped by GO (gene ontology) terms. Functional groupings are arranged by fold-enrichment of overexpression as well as the −log₁₀ false discovery rate (FDR). Shown in FIG. 14E is hierarchical clustering of transcript counts, measured in log of transcripts per million+1 (log(TPM+1)), of imported E. coli genes and V. natriegens genes in the MDS42, V. natriegens, Vibrio-MDS42 (Chr.1) chimera and Vibrio-MDS42 (Chr.2) chimera strains. Three individual replicates are shown for each strain. Genes are clustered based on expression level within each genomic background. FIG. 14F-FIG. 14G show graphs of differential expression of Vibrio genes (purple) between the chimeric strain and the V. natriegens background strain for both Vibrio+1 Mb MDS42 (Chr.1) and Vibrio+1 Mb MDS42 (Chr.2) chimeras. The log 2 of the ratio of the expression level of Vibrio genes in the chimeric strain to the expression level in the background strain is plotted. Points are also plotted based on their −log₁₀ adjusted p-value. P-value cutoff was set at 0.001 and the log 2 fold-change cutoff was set to 1 so that points below the cutoff are in gray. Shown in FIG. 14H is a Venn diagram of the V. natriegens upregulated and downregulated genes for both Vibrio+1 Mb MDS42 (Chr.1) and Vibrio+1 Mb MDS42 (Chr.2) chimeras highlighting the set of genes present and up- and down-regulated in both chimeric strains. FIG. 14I-FIG. 14J show results of analysis of the set of up- and down-regulated genes in both chimeric strains and in Vibrio+1 Mb MDS42 (Chr.1) chimera, grouped by GO (gene ontology) terms. Functional groupings are arranged by fold-enrichment of overexpression as well as the −log₁₀ false discovery rate (FDR).

FIG. 15A-FIG. E display non-limiting exemplary schematics and data related to genomic transfers from E. coli to Agrobacterium tumefaciens for functional agroinfiltration of large T-DNA and Pseudomonas putida. Shown in FIG. 15A is a diagram of 100-kb E. coli Syn61 transfer to A. tumefaciens. The Syn61 genome is depicted by the red circle and the A. tumefaciens genomes are depicted with chromosome 1 as a yellow circle and chromosome 2 as an orange segment. Sizes of transferred region, recipient genome and chimeric genome are given in the schematic. The transferred region in Syn61 is represented by the red portion of the circle flanked by TEs, shown as yellow rectangles. Transfer direction is shown by the arrow. The flaA, dif tetA and agpI genes of A. tumefaciens are indicated by green, red, orange and blue triangle rectangles, respectively. The outcome chimeric genome is represented as an integration of the red segment of the Syn61 genome into the A. tumefaciens genome at any of the four loci. Importantly, the 100-kb Syn61 contains a YFP T-DNA cargo. Shown in FIG. 15B is a schematic depicting Agroinfiltration of Nicotiana benthamiana leaves with chimeric Agrobacterium engineered with genomic YFP T-DNA. FIG. 15C displays exemplary confocal microscope images of YFP signal from agroinfiltrated Nicotiana leaf tissue of chimeric genome T-DNA expressing YFP alongside plasmid and genome YFP T-DNA controls. Images are captured with 20× magnification. Shown in FIG. 15D is a diagram of 100-kb Syn100 transfer to Pseudomonas putida. The E. coli Syn100 genome is depicted by the gray circle and the P. putida genome is depicted by the green circle. Sizes of transferred region, recipient genome and chimeric genome are given in the schematic. The transferred region Syn100 is represented by the red portion of the circle flanked by TEs, shown as yellow rectangles. Transfer direction is shown by the arrow. The glmS gene of P. putida is given by the orange rectangle, which is targeted by the CAST by the TS^{glmS} crRNA, shown as an orange triangle. The outcome chimeric genome is represented as an integration of the red Syn100 segment into the P. putida genome at glmS. FIG. 15E shows genome-wide read coverages (in RPGC) for the P. putida KT2440+100-kb Syn100 genomic chimeric strain. The Syn100 segment is shaded in red, and P. putida genome is shaded in light green. Location of the TS glmS target is given by the orange triangle.

FIG. 16A-FIG. 16H display non-limiting exemplary data related to extended P. protegens-E. coli chimera genome generation and metabolomic characterization. Shown in FIG. 16A is a diagram of 140-kb P. protegens pf-5 transfer into E. coli DH10B and corresponding genome-wide read coverages for the resultant chimeric genome. The P. protegens genome is depicted by the blue circle and the E. coli genomes are depicted with the gray circle. Genome integration sites TS2 and TS3 are indicated by their respective colored rectangle triangles. In the coverage plot, transferred DNA is highlighted in blue and integration sites indicated with colored triangles. Shown in FIG. 16B is a Venn diagram of distinct m/z peaks in E. coli DH10B, P. protegens pf-5 and 140-kb P. protegens-E. coli chimera highlighting the set of metabolites unique to and shared with between the three bacteria. FIG. 16C displays a 3D MS1 heatmap of 140-kb P. protegens TS2 and TS3 E. coli DH10B chimeras showing m/z vs. retention time vs. intensity landscape. FIG. 16D shows a graph of the distribution of unique m/z counts shared between experimental replicates of 140-kb P. protegens chimera. Shown in FIG. 16E is a Venn diagram of distinct m/z peaks in 415-kb P. protegens-E. coli TS2 and 140-kb P. protegens-E. coli TS2 chimera highlighting the set of metabolites unique to and shared with between the two chimeras. FIG. 16F displays results of principal component analysis of the 140-kb and 415-kb chimeras alongside E. coli DH10B and P. protegens pf-5. Shown in FIG. 16G is a schematic and data as in FIG. 16A, but diagrams and associated genome-wide read coverage plots for the 246-kb P. protegens pf-5 transfer to E. coli DH10B into genomic sites TS3 and TS5. FIG. 16H shows a schematic and data as in FIG. 16A, but diagrams and associated genome-wide read coverage plots for the 395-kb P. protegens pf-5 transfer of E. coli DH10B into genomic sites TS4, dif and TS3.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include systems for producing a chimeric or trimeric genome. In some embodiments, the system comprises: a) a donor cell comprising: (i) a first start cassette (Cassette^{START}) comprising, from 5′ to 3′, at least one cis-acting component of a first delivery module and a first integration sequence, and (ii) a first stop cassette (Cassette^{STOP}) comprising, from 5′ to 3′, a second integration sequence and at least one cis-acting component of the first delivery module, wherein the first Cassette^{START} is integrated at a first site in the genome of the donor cell and the first Cassette^{STOP} is integrated at a second site in the genome of the donor cell, wherein intervening sequence between the first Cassette^{START} and the first Cassette^{STOP} defines a first genomic transfer window; and (iii) one or more trans-acting components of the first delivery module or one or more first helper polynucleotides each comprising a sequence encoding a trans-acting component of the first delivery module, wherein the first delivery module comprises the one or more trans-acting components; and b) a recipient cell comprising one or more components of an integrator module or one or more second helper polynucleotides each comprising a sequence encoding a component of an integrator module, wherein the integrator module comprises the one or more components.

Disclosed herein include nucleic acid compositions for producing a chimeric or trimeric genome. In some embodiments, the nucleic acid composition comprises: i) a first and/or second start cassette (Cassette^{START}) each comprising, from 5′ to 3′: at least one cis-acting component of a first and/or second delivery module and a first integration sequence; ii) a first and/or second stop cassette (Cassette^{STOP}) each comprising, from 5′ to 3′: a second integration sequence and at least one cis-acting component of the first and/or second delivery module; iii) one or more first helper polynucleotides each comprising a sequence encoding a trans-acting component of the first and/or second delivery module, wherein the first and/or second delivery module comprise one or more trans-acting components; and optionally iv) one or more second helper polynucleotides each comprising a sequence encoding a component of an integrator module, wherein the integrator module comprises one or more components.

Provided herein are kits comprising any of the systems or nucleic acid compositions of the disclosure, and set of instructions for use.

Disclosed herein are methods for producing a chimeric genome. In some embodiments, the method comprises: contacting a recipient cell disclosed herein with a donor cell disclosed herein, and: expressing the one or more trans-acting components of the first delivery module in the donor cell, thereby the sequence defined by the genomic transfer window is transferred from the donor cell to the recipient cell and thereby the first donor genomic segment comprising the first donor sequence flanked by the first integration sequence and the second integration sequence of the first Cassette^{START} and the first Cassette^{STOP} is generated in the recipient cell; and expressing the one or more components of the integrator module in the recipient cell, thereby the first donor genomic segment is inserted into the first double-stranded target sequence in the genome of the recipient cell, thereby generating the chimeric genome in the recipient cell.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” means plus or minus 5% of the provided value.

As used herein, the term “double-stranded target DNA” refers to a DNA that includes a “target site” or “target sequence.” The term “target sequence” is used herein to refer to a nucleic acid sequence present in a double-stranded target DNA to which a DNA-targeting sequence or segment (also referred to herein as a “spacer”) of a crRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a crRNA and the target sequence can, for example, be based on Watson-Crick base pairing rules, which enables programmability in the DNA-targeting sequence or segment. The DNA-targeting sequence or segment of a crRNA can be designed, for instance, to hybridize with any target sequence.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases. In some embodiments, a polynucleotide comprises a nucleotide sequence encoding a gene product operably linked to one or more expression control elements (e.g., a promoter), as an expression cassette. Any of the RNA sequences disclosed herein may also be DNA (either single-stranded or double-stranded), e.g., wherein “U” is converted to “T.” Any of the DNA sequences disclosed herein may also be RNA, e.g., wherein “T” is converted to “U.”

As used herein, the term “binding” refers to a non-covalent interaction between macromolecules (e.g., between a protein and a nucleic acid). While in a state of non-covalent interaction, the macromolecules are said to be “associated” or “interacting” or “binding” (e g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸M, 10⁻⁹ M, 10⁻⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g. complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e. 100%. For example, the complementary candidate sequence segment is perfectly complementary to the candidate sequence segment, whose sequence can be deducted from the candidate sequence segment using the Watson-Crick base pairing rules.

The term “vector” as used herein, can refer to a vehicle for carrying or transferring a nucleic acid. Non-limiting examples of vectors include plasmids, bacteria, and viruses (for example, Agrobacterium tumefaciens Ti vectors).

The term “construct,” as used herein, can refer to a recombinant nucleic acid that has been generated for the purpose of the expression of a specific nucleotide sequence(s), or that is to be used in the construction of other recombinant nucleotide sequences. As used herein, the term “plasmid” can refer to a nucleic acid that can be used to replicate recombinant DNA sequences within a host organism. The sequence can be a double stranded DNA.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein in the term “derived from”, in the context of an amino acid sequence or polynucleotide sequence (e.g., an amino acid sequence “derived from” a conjugation system or a transposase system), is meant to indicate that the polypeptide or nucleic acid has a sequence that is based on that of a reference polypeptide or nucleic acid, and is not meant to be limiting as to the source or method in which the protein or nucleic acid is made. By way of example, the term “derived from” includes homologs or variants of reference amino acid or DNA sequences.

As used herein, the term “derived from” can also refer to a specified nucleotide sequence that may be obtained from a particular specified source or species, albeit not necessarily directly from that specified source or species.

Standard techniques can be used for recombinant DNA, oligonucleotide synthesis, and cell culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

There are provided, in some embodiments, synthetic chimeric organisms and novel methods for their generation. The methods disclosed herein can enable the precise insertion of genetic material from multiple organisms into a recipient organism, resulting in the generation of entirely new life forms with unique properties. As demonstrated in Example 1, these synthetic lifeforms were shown to express the inserted genetic material, manifesting genotype to phenotype and thereby enabling functional applications. Provided herein include compositions, methods, systems, and kits for the designation and modular construction of “donor” and “recipient” organisms, as well as applications and methods of using.

Currently available approaches in the field of synthetic genomes have been limited to recoding, rearranging (refactoring) and minimizing existing genomes. The methods used for these purposes are not suitable for creating synthetic chimeric genomes requiring large DNA integrations. Efforts to expand the size of bacteria genomes with one-step insertions of multiple megabases of DNA have not been reported previously. Existing efforts to expand the gene content of a bacteria organism have been relegated to low-throughput, inflexible large insertions on specially prepared bacteria episomes, or through many small, fragmented insertions scattered throughout the bacterial genome. In both cases, the methods used to attain these outcomes are not able to perform genome-scale insertions directly on a native bacterial chromosome.

The methods and compositions provided herein can overcome the limitations of prior methods by providing highly efficient and versatile processes for creating synthetic, chimeric organisms. The method can comprise the use of novel artificial conjugative elements, comprising naturally occurring or engineered CRISPR-associated transposases (CASTs) and/or site-specific DNA recombinase/integrases as an integrator module, coupled to synthetic microbial DNA transfer systems as the delivery module. As shown in Example 1, the disclosed compositions and methods can universally transfer and stably integrate DNA into genomes or episomes at the whole-genome level, with demonstrations performed beyond 2-Mb (at least half of the genome of a typical bacterial organism) using several distinct donor species combinatorially across several distinct recipient species. Using methods and compositions provided herein, the entirety of a microbial genome was shown to be modularized and ported over to designated recipient strains, across distinct genomic loci in a programmable manner. Also provided herein include methods and novel genetic components enabling the transfer, selection, and rapid screening of imported genetic material, such as the use of selection markers and optimized fluorescence expression systems. Altogether, the disclosed methods and compositions provided allow for stable and robust expression of newly-gained foreign genes alongside previously present native genes, enabling the first-in-class streamlined creation of organisms with desirable phenotypic traits.

In some embodiments, alternatives to using Type I-F Tn6677 CAST and RP4-family conjugation described herein include using either other CRISPR-associated transposons (such as other Type I-F, Type I-B, Type I-D or Type V-K CASTs, among others) or site-specific DNA recombinases and integrases (e.g., attP-attB recombination with Bxb1, PhiC31 or Cre-lox recombinases) as the integrator module and other conjugative plasmids (e.g., incompatibility (Inc) P, F, N family plasmids and Tumor inducing (Ti) plasmids) as the delivery module.

As described herein, designation and modular construction of “donor” and “recipient” organisms can also be accomplished with multiple systems. In some embodiments, the “donor” organism is constructed using λ red mediated homologous recombination. In other embodiments, “donor” organisms can be constructed using CAST systems (such as related but mutually non-interacting Type I-F systems). “Recipient” organisms and their target sites can be established by the use of Prime editing or conventional recombination proteins (e.g. Lambda-Red, recT systems) to write in attP and attB sequences on their genomes when site-specific recombinases and integrases are employed as the integrator module.

Chimeric genome bacteria synthesized with the methods and compositions provided herein can enable applications where these large insertions confer novel functional phenotypes to the engineered organisms. In some embodiments, and without being bound by any particular theory, these chimeric DNA are sourced for genomic islands (GI) and the microbial mobilome, and transferring megabase sized segments of diverse bacterial genomes with different environmental niches can create organisms suited for growth in harsh environments, production of economically valuable secondary metabolites and biomolecules and as a means to study genome fragments from a variety of sources for biological discovery and innovation.

The methods and compositions disclosed herein can revolutionize and drive actionable field applications in a number of areas, including, for example, one or more of the following: (i) Agriculture (e.g., genetically modified agro-biologicals for the treatment, protection, and enhancement of crop yield, tolerance to pests, diseases, and weather conditions; (ii) Biological nitrogen fixation (e.g., synthetic chimeric bacteria can be applied to associate with agriculturally relevant crops for nitrogen fixation); (iii) Pharmaceuticals (e.g., new antibiotics, cancer treatments, or vaccines, as well as Novel and genetically controlled delivery vehicles for DNA, RNA, and/or protein); (iv) Industrial biotechnology (e.g., synthetic chimeric bacteria can be used to produce enzymes, drugs, and other valuable products currently inaccessible via natural, wild bacteria); (v) Biofuels (e.g., production of fuel from renewable sources); (vi) Bioremediation (e.g., cleaning up polluted soil and water, and synthetic chimeric bacteria can be created to be resistant to heavy metals, chemicals and other extreme stressors of temperature, pressure and radiation); (vii) Biocontainment (e.g., genetically controlled containment of microbial agents and technologies); (viii) Bioplastics (e.g., development of environmentally friendly plastics); and (ix) DNA Storage/Cryptography (e.g., stable encryption and decryption of genetically encoded computational information into microbial genomes).

Disclosed herein include systems, compositions, and kits for producing a chimeric, a trimeric genome, and/or an n-meric genome. An organism's physiology is heavily influenced by its genomic content. Building genomic chimeras can enable melding of the diverse functions and properties of life. However, prior arts in genome synthesis are limited to reconstituting pre-existing functions within a singular genome rather than combining and extending diverse genomic functions across multiple distinct genomes. Existing methods are also prohibitively expensive, labor intensive, and time consuming, and thus fundamentally not scalable for creating large synthetic chimeric genomes. To address all these limitations, there is disclosed and described herein Additive Conjugative-CAST Engineering (ACE) combining conjugation with CRISPR-associated transposition (CAST) to deliver and integrate up to half a genome per step from a donor cell into a precisely defined position in the recipient's genome. Demonstrated herein is ACE's capability to integrate at least a 2-megabase donor genomic segment in a single step and at least 3 megabase in two consecutive steps. Importantly, ACE, when coupled to Orthogonal CAST sequential integration system (OASIS), allows for the generalizable creation of multiple genomic chimeras across species, genus, order and class barriers. It is further demonstrated herein that such chimeric organisms, also referred to herein as genome expanded organisms (GEOs), can be stably forged from at least three starting bacterial strains, can be stably maintained to express all their acquired genomic parts in a single chimera, and can demonstrate emergent metabolic signatures as evidenced by multi-omics exploration. ACE offers a new paradigm of creating artificial lifeforms to combine desired properties, generate novel functions and offers a unique perspective to probe unexplored plasticity in genome architecture and expression patterns of novel, massively expanded genomes.

Disclosed herein include systems for producing a chimeric or trimeric genome. In some embodiments, the system comprises: a) a donor cell comprising: (i) a first start cassette (Cassette^{START}) comprising, from 5′ to 3′, at least one cis-acting component of a first delivery module and a first integration sequence, and (ii) a first stop cassette (Cassette^{STOP}) comprising, from 5′ to 3′, a second integration sequence and at least one cis-acting component of the first delivery module, wherein the first Cassette^{START} is integrated at a first site in the genome of the donor cell and the first Cassette^{STOP} is integrated at a second site in the genome of the donor cell, wherein intervening sequence between the first Cassette^{START} and the first Cassette^{STOP} defines a first genomic transfer window; and (iii) one or more trans-acting components of the first delivery module or one or more first helper polynucleotides each comprising a sequence encoding a trans-acting component of the first delivery module, wherein the first delivery module comprises the one or more trans-acting components; and b) a recipient cell comprising one or more components of an integrator module or one or more second helper polynucleotides each comprising a sequence encoding a component of an integrator module, wherein the integrator module comprises the one or more components.

In some embodiments: (1) a sequence defined by the first genomic transfer window is capable of being transferred from the donor cell to the recipient cell upon expression of the one or more trans-acting components of the first delivery module in the donor cell, thereby generating a first donor genomic segment in the recipient cell flanked by the first integration sequence and the second integration sequence of the first Cassette^{START} and the first Cassette^{STOP}. and (2) the first donor genomic segment is capable of being inserted into a first double-stranded target sequence in the genome of the recipient cell upon expression of the one or more components of the integrator module, thereby generating a chimeric genome in the recipient cell.

The system can comprise: c) a second donor cell comprising: (i) a second start cassette (Cassette^{START}) comprising, from 5′ to 3′, at least one cis-acting component of a second delivery module and a first integration sequence, and (ii) a second stop cassette (Cassette^{STOP}) comprising, from 5′ to 3′, a second integration sequence and at least one cis-acting component of the second delivery module, wherein the second Cassette^{START} is integrated at a first site in the genome of the second donor cell and the second Cassette^{STOP} is integrated at a second site in the genome of the second donor cell, thereby defining a second genomic transfer window; and (iii) one or more trans-acting components of the second delivery module or one or more first helper polynucleotides each comprising a sequence encoding a trans-acting component of the second delivery module, wherein the second delivery module comprises the one or more trans-acting components.

In some embodiments: (3) a sequence defined by the second genomic transfer window is capable of being transferred from the second donor cell to the recipient cell comprising the chimeric genome upon expression of the one or more trans-acting components of the second delivery module in the second donor cell, thereby generating a second donor genomic segment flanked by the first integration sequence and the second integration sequence of the second Cassette^{START} and the second Cassette^{STOP} in the recipient cell comprising the chimeric genome; and (4) the second donor genomic segment is capable of being inserted into a second double-stranded target sequence in the genome of the recipient cell comprising the chimeric genome upon expression of the one or more components of the integrator module, thereby generating a trimeric genome in the recipient cell comprising the chimeric genome. The second double-stranded target sequence can be comprised within the first donor genomic segment.

Disclosed herein include nucleic acid compositions for producing a chimeric or trimeric genome. In some embodiments, the nucleic acid composition comprises: i) a first and/or second start cassette (Cassette^{START}) each comprising, from 5′ to 3′: at least one cis-acting component of a first and/or second delivery module and a first integration sequence; ii) a first and/or second stop cassette (Cassette^{STOP}) each comprising, from 5′ to 3′: a second integration sequence and at least one cis-acting component of the first and/or second delivery module; iii) one or more first helper polynucleotides each comprising a sequence encoding a trans-acting component of the first and/or second delivery module, wherein the first and/or second delivery module comprise one or more trans-acting components; and optionally iv) one or more second helper polynucleotides each comprising a sequence encoding a component of an integrator module, wherein the integrator module comprises one or more components.

The length of a genomic transfer window (e.g., a first and/or second genomic transfer window) can vary. The first and/or second genomic transfer window can be about 10 kb to at least 2 MB in length. In some embodiments, a genomic transfer window (e.g., first and/or second genomic transfer window) can be less than 10 kb or more than 2 MB (e.g., 2000 kb) in length. In some embodiments, the first and/or second genomic transfer window can be, can be at least, or can be at least about, 10 kb, 20 kb, 30 kb, 40 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 210 kb, 220 kb, 230 kb, 240 kb, 250 kb, 260 kb, 270 kb, 280 kb, 290 kb, 300 kb, 310 kb, 320 kb, 330 kb, 340 kb, 350 kb, 360 kb, 370 kb, 380 kb, 390 kb, 400 kb, 410 kb, 420 kb, 430 kb, 440 kb, 450 kb, 460 kb, 470 kb, 480 kb, 490 kb, 500 kb, 510 kb, 520 kb, 530 kb, 540 kb, 550 kb, 560 kb, 570 kb, 580 kb, 590 kb, 600 kb, 610 kb, 620 kb, 630 kb, 640 kb, 650 kb, 660 kb, 670 kb, 680 kb, 690 kb, 700 kb, 710 kb, 720 kb, 730 kb, 740 kb, 750 kb, 760 kb, 770 kb, 780 kb, 790 kb, 800 kb, 810 kb, 820 kb, 830 kb, 840 kb, 850 kb, 860 kb, 870 kb, 880 kb, 890 kb, 900 kb, 910 kb, 920 kb, 930 kb, 940 kb, 950 kb, 960 kb, 970 kb, 980 kb, 990 kb, 1000 kb, 1010 kb, 1020 kb, 1030 kb, 1040 kb, 1050 kb, 1060 kb, 1070 kb, 1080 kb, 1090 kb, 1100 kb, 1110 kb, 1120 kb, 1130 kb, 1140 kb, 1150 kb, 1160 kb, 1170 kb, 1180 kb, 1190 kb, 1200 kb, 1210 kb, 1220 kb, 1230 kb, 1240 kb, 1250 kb, 1260 kb, 1270 kb, 1280 kb, 1290 kb, 1300 kb, 1310 kb, 1320 kb, 1330 kb, 1340 kb, 1350 kb, 1360 kb, 1370 kb, 1380 kb, 1390 kb, 1400 kb, 1410 kb, 1420 kb, 1430 kb, 1440 kb, 1450 kb, 1460 kb, 1470 kb, 1480 kb, 1490 kb, 1500 kb, 1510 kb, 1520 kb, 1530 kb, 1540 kb, 1550 kb, 1560 kb, 1570 kb, 1580 kb, 1590 kb, 1600 kb, 1610 kb, 1620 kb, 1630 kb, 1640 kb, 1650 kb, 1660 kb, 1670 kb, 1680 kb, 1690 kb, 1700 kb, 1710 kb, 1720 kb, 1730 kb, 1740 kb, 1750 kb, 1760 kb, 1770 kb, 1780 kb, 1790 kb, 1800 kb, 1810 kb, 1820 kb, 1830 kb, 1840 kb, 1850 kb, 1860 kb, 1870 kb, 1880 kb, 1890 kb, 1900 kb, 1910 kb, 1920 kb, 1930 kb, 1940 kb, 1950 kb, 1960 kb, 1970 kb, 1980 kb, 1990 kb, 2000 kb, in length, or a number or range between any two of these values.

The length of a donor genomic segment (e.g., first and/or second donor genomic segment) can vary. The first and/or second donor genomic segment can be about 10 kb to at least 2 MB in length. The donor genomic segment, e.g., the first and/or second donor genomic segment can be less than 10 kb or more than 2 MB in length. The donor segment, e.g., the first and/or second donor genomic segment, can be, can be at least, or can be at least about 10 kb, 20 kb, 30 kb, 40 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 210 kb, 220 kb, 230 kb, 240 kb, 250 kb, 260 kb, 270 kb, 280 kb, 290 kb, 300 kb, 310 kb, 320 kb, 330 kb, 340 kb, 350 kb, 360 kb, 370 kb, 380 kb, 390 kb, 400 kb, 410 kb, 420 kb, 430 kb, 440 kb, 450 kb, 460 kb, 470 kb, 480 kb, 490 kb, 500 kb, 510 kb, 520 kb, 530 kb, 540 kb, 550 kb, 560 kb, 570 kb, 580 kb, 590 kb, 600 kb, 610 kb, 620 kb, 630 kb, 640 kb, 650 kb, 660 kb, 670 kb, 680 kb, 690 kb, 700 kb, 710 kb, 720 kb, 730 kb, 740 kb, 750 kb, 760 kb, 770 kb, 780 kb, 790 kb, 800 kb, 810 kb, 820 kb, 830 kb, 840 kb, 850 kb, 860 kb, 870 kb, 880 kb, 890 kb, 900 kb, 910 kb, 920 kb, 930 kb, 940 kb, 950 kb, 960 kb, 970 kb, 980 kb, 990 kb, 1000 kb, 1010 kb, 1020 kb, 1030 kb, 1040 kb, 1050 kb, 1060 kb, 1070 kb, 1080 kb, 1090 kb, 1100 kb, 1110 kb, 1120 kb, 1130 kb, 1140 kb, 1150 kb, 1160 kb, 1170 kb, 1180 kb, 1190 kb, 1200 kb, 1210 kb, 1220 kb, 1230 kb, 1240 kb, 1250 kb, 1260 kb, 1270 kb, 1280 kb, 1290 kb, 1300 kb, 1310 kb, 1320 kb, 1330 kb, 1340 kb, 1350 kb, 1360 kb, 1370 kb, 1380 kb, 1390 kb, 1400 kb, 1410 kb, 1420 kb, 1430 kb, 1440 kb, 1450 kb, 1460 kb, 1470 kb, 1480 kb, 1490 kb, 1500 kb, 1510 kb, 1520 kb, 1530 kb, 1540 kb, 1550 kb, 1560 kb, 1570 kb, 1580 kb, 1590 kb, 1600 kb, 1610 kb, 1620 kb, 1630 kb, 1640 kb, 1650 kb, 1660 kb, 1670 kb, 1680 kb, 1690 kb, 1700 kb, 1710 kb, 1720 kb, 1730 kb, 1740 kb, 1750 kb, 1760 kb, 1770 kb, 1780 kb, 1790 kb, 1800 kb, 1810 kb, 1820 kb, 1830 kb, 1840 kb, 1850 kb, 1860 kb, 1870 kb, 1880 kb, 1890 kb, 1900 kb, 1910 kb, 1920 kb, 1930 kb, 1940 kb, 1950 kb, 1960 kb, 1970 kb, 1980 kb, 1990 kb, 2000 kb, in length, or a number or range between any two of these values.

In some embodiments, any transcription termination sequence (ter) comprised within the first and/or second donor genomic segment is in the same orientation as any ter in or near: the first double-stranded target sequence in the genome of the recipient cell; and/or the second double-stranded target sequence in the genome of the recipient cell comprising the chimeric genome.

The length of a chimeric genome, trimeric genome, or n-meric genome can vary. A chimeric genome, trimeric genome, or n-meric genome can be at least about 4 MB in length. A chimeric genome, trimeric genome, or n-meric genome can be at least about 4 MB, 4.5 MB, 5 MB, 6 MB, 6.5 MB, 7 MB, 7.5 MB, 8 MB, 8.5 MB, 9 MB, 9.5 MB, or 10 MB in length. A chimeric genome, trimeric genome, or n-meric genome can be, can be at least, or can be at least about 4.0 MB, 4.1 MB, 4.2 MB, 4.3 MB, 4.4 MB, 4.5 MB, 4.6 MB, 4.7 MB, 4.8 MB, 4.9 MB, 5.0 MB, 5.1 MB, 5.2 MB, 5.3 MB, 5.4 MB, 5.5 MB, 5.6 MB, 5.7 MB, 5.8 MB, 5.9 MB, 6.0 MB, 6.1 MB, 6.2 MB, 6.3 MB, 6.4 MB, 6.5 MB, 6.6 MB, 6.7 MB, 6.8 MB, 6.9 MB, 7.0 MB, 7.1 MB, 7.2 MB, 7.3 MB, 7.4 MB, 7.5 MB, 7.6 MB, 7.7 MB, 7.8 MB, 7.9 MB, 8.0 MB, 8.1 MB, 8.2 MB, 8.3 MB, 8.4 MB, 8.5 MB, 8.6 MB, 8.7 MB, 8.8 MB, 8.9 MB, 9.0 MB, 9.1 MB, 9.2 MB, 9.3 MB, 9.4 MB, 9.5 MB, 9.6 MB, 9.7 MB, 9.8 MB, 9.9 MB, 10.0 MB, or longer, in length, or a number or range between any two of these values. A chimeric genome, trimeric genome, or n-meric genome at least about 3 MB (e.g., megabases) in length. A chimeric genome, trimeric genome, or n-meric genome can be, can be at least, or can be at least about 3.0 MB, 3.1 MB, 3.2 MB, 3.3 MB, 3.4 MB, 3.5 MB, 3.6 MB, 3.7 MB, 3.8 MB, 3.9 MB, 4.0 MB, 4.1 MB, 4.2 MB, 4.3 MB, 4.4 MB, 4.5 MB, 4.6 MB, 4.7 MB, 4.8 MB, 4.9 MB, 5.0 MB, 5.1 MB, 5.2 MB, 5.3 MB, 5.4 MB, 5.5 MB, 5.6 MB, 5.7 MB, 5.8 MB, 5.9 MB, 6.0 MB, 6.1 MB, 6.2 MB, 6.3 MB, 6.4 MB, 6.5 MB, 6.6 MB, 6.7 MB, 6.8 MB, 6.9 MB, 7.0 MB, 7.1 MB, 7.2 MB, 7.3 MB, 7.4 MB, 7.5 MB, 7.6 MB, 7.7 MB, 7.8 MB, 7.9 MB, 8.0 MB, 8.1 MB, 8.2 MB, 8.3 MB, 8.4 MB, 8.5 MB, 8.6 MB, 8.7 MB, 8.8 MB, 8.9 MB, 9.0 MB, 9.1 MB, 9.2 MB, 9.3 MB, 9.4 MB, 9.5 MB, 9.6 MB, 9.7 MB, 9.8 MB, 9.9 MB, 10.0 MB, or longer, in length, or a number or range between any two of these values.

The cell type (e.g., organism) of a donor sell, a second donor cell, and/or a recipient cell can vary. The donor cell, the second donor cell, the recipient cell, or any combination thereof can be a eukaryotic cell. The eukaryotic cell can be a fungal cell. The eukaryotic cell can be a plant cell. The donor cell, the second donor cell, the recipient cell, or any combination thereof can be a prokaryotic cell.

In some embodiments, the prokaryotic cell is or is derived from a bacterial species selected from the group comprising: Acinetobacter species, Actinobacillus species, Actinomycetes species, an Actinomyces species, Aerococcus species an Aeromonas species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Chlamydia species, a Citrobacter species, a Coxiella species, a Corynebacterium species, a Clostridium species, an Eikenella species, an Enterobacter species, an Escherichia species, an Enterococcus species, an Ehrlichia species, an Epidermophyton species, an Erysipelothrix species, a Eubacterium species, a Francisella species, a Fusobacterium species, a Gardnerella species, a Gemella species, a Haemophilus species, a Helicobacter species, a Kingella species, a Klebsiella species, a Lactobacillus species, a Lactococcus species, a Listeria species, a Leptospira species, a Legionella species, a Leptospira species, Leuconostoc species, a Mannheimia species, a Microsporum species, a Micrococcus species, a Moraxella species, a Morganella species, a Mobiluncus species, a Micrococcus species, Mycobacterium species, a Mycoplasma species, a Nocardia species, a Neisseria species, a Pasteurella species, a Pediococcus species, a Peptostreptococcus species, a Pityrosporum species, a Plesiomonas species, a Prevotella species, a Porphyromonas species, a Proteus species, a Providencia species, a Pseudomonas species, a Propionibacterium species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, or a Xanthomonas species. In some embodiments, the prokaryotic cell is or is derived from an archaeal species.

In some embodiments, the genera and/or species of the donor cell and/or the second donor cell and the genera and/or the species of the recipient cell are the same or different. For example, any two of the donor cell, the second donor cell, and the recipient cell can be from the same species and/or the same genus. In some embodiments, any two of the donor cell, the second donor cell, and the recipient cell are from a different genus and/or species.

### Delivery Module

Provided herein are delivery modules. A delivery module can comprise one or more components encoded by or derived from a bacterial conjugation system.

Conjugation was first discovered in 1946 by Edward Tatum and Joshua Lederberg, who showed that bacteria could exchange genetic information through the unidirectional transfer of DNA, mediated by a so-called F (Fertility) factor. It was later realized that the F factor is a replicative extra-chromosomal genetic element, e.g., plasmid, which can be transferred across the cell membranes of the parental strains. Since this seminal discovery, the identification of a plethora of conjugative elements, including plasmids, conjugative transposons, and integrative conjugative elements (ICEs), has revealed that conjugation is a universally conserved DNA transfer mechanism among Gram-negative and Gram-positive bacteria.

Conjugative plasmids generally carry all the genes required for their maintenance during the vertical transfer from the mother to the daughter cells, as well as the genes necessary for horizontal transfer during conjugation from the donor to the recipient cell. These functions are encoded by different regions of the plasmid. Isolation and sequence analysis of an increasing number of conjugative plasmids has revealed considerable diversity in terms of genetic properties and organization. This diversity also indicates that different plasmids might use various regulations, molecular reactions, and strategies to achieve productive conjugational transfer and maintenance.

Described below is the life cycle of the F plasmid during conjugational transfer from the donor to the recipient cell, as an exemplar of conjugation. The proteins and mechanism of conjugation are highly conserved. Table 1 list proteins of the F plasmid conjugation system, with homologs (e.g., for RP4, Ti plasmid, etc.) shown. The F plasmid backbone is composed of the tra regions encoding all genes involved in conjugational transfer; the origin of transfer oriT; the leading region, which is the first to be transferred into the recipient cell; and the maintenance region involved in plasmid replication and partition. The initiation of conjugation requires the expression of the tra genes. Some of the produced Tra proteins form the T4SS and the conjugative pilus that will recruit the recipient cell and mediate mating pair stabilization. Other Tra proteins constitute the relaxosome (TraI, TraM, and TraY), which, in combination with the integration host factor (IHF), bind to the oriT and prepare the plasmid for transfer by inducing the nicking reaction by the TraI relaxase. Interaction between the relaxosome and the Type IV Coupling Protein (T4CP) initiates the transfer of the T-strand by the T4SS. Transfer of the TraI-bound T-strand in the recipient is concomitant with the conversion of the ssDNA into dsDNA by Rolling Circle Replication (RCR) in the donor. Upon entry into the recipient, the ssDNA T-strand is coated by the host chromosomal SSB, and the single-stranded promotor Frpo adopts a stem-loop structure recognized by the host RNA polymerase to initiate the synthesis of RNA primers. TraI performs the circularization of the fully internalized T-strand. The RNA-DNA duplex is recognized by the host DNA polymerase to initiate the complementary strand synthesis reaction.

TABLE 1
Conjugation Proteins

| Protein (F plasmid) | Proposed Function | Homolog |
|---|---|---|
| TraM | Relaxosome | |
| TraJ | Regulation | |
| TraY | Relaxosome Regulation | |
| TraA | Pilin | VirB2 (pTi); TrbC (RP4) |
| TraL | Pilus assembly | VirB3 (pTi); TrbD (RP4) |
| TraE | Pilus assembly | VirB5 (pTi) |
| TraK | Pilus assembly | VirB9 (pTi) |
| TraB | Pilus extension | VirB10 (pTi); TrbI (RP4) |
| TraP | Pilus extension | |
| TraG | Pilus assembly; Mating pair stabilization; | VirB6/VirB8 (pTi) |
| | Exclusion | |
| TraV | Pilus extension | VirB7 (pTi) |
| TraR | Regulation | |
| TraC | Pilus assembly | VirB4 (pTi); TrbE (RP4) |
| TraW | Pilus extension | |
| TraU | DNA transfer | |
| TraN | Mating pair stabilization; Exclusion system | |
| TraF | Pilus extension | |
| TraQ | Pilin maturation | |
| TraH | Pilus extension | |
| TraG | Pilus assembly; Mating pair stabilization; | VirB6/VirB8 (pTi) |
| | Exclusion | |
| TraS | Entry Exclusion (Eex) | |
| TraT | Surface exclusion (Sfx) | |
| TraD | T4CP | VirD4 (pTi); TraG (RP4); TrwB |
| | | (R388) |
| TraI | Relaxosome | VirD2 (pTi); TrwC (R388) |
| TraX | Pilin maturation | TrbP (RP4) |

In some embodiments, the first and/or second delivery module comprises or is derived from a bacterial conjugation system. In some embodiments, the bacterial conjugation system is an RP4 (InCP), pKM101 (IneN), R388 (IneW), F, or Tumor-inducing (Ti) system.

In some embodiments, the bacterial conjugation system is an RP4 (IncP) plasmid system, and wherein: the at least one cis-acting component comprises an RP4 origin of transfer (oriT) sequence. The oriT sequence can comprise or consist of a sequence of SEQ ID NO: 384 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 384; and the one or more trans-acting components comprise or are derived from gene products of the RP4 plasmid.

In some embodiments, the bacterial conjugation system is a Ti plasmid system, and wherein: the at least one cis-acting component comprises a Ti origin of transfer (oriT) sequence; and the one or more trans-acting components comprise or are derived from gene products of the Ti plasmid. In some embodiments, the bacterial conjugation system is a pKM101 (IncN) plasmid system, and wherein: the at least one cis-acting component comprises a pKM101 (IncN) origin of transfer (oriT) sequence; and the one or more trans-acting components comprise or are derived from gene products of the pKM101 (IncN) plasmid. In some embodiments, the bacterial conjugation system is an R388 (IncW) plasmid system, and wherein: the at least one cis-acting component comprises an R388 (IncW) origin of transfer (oriT) sequence; and the one or more trans-acting components comprise or are derived from gene products of the R388 (IncW) plasmid. In some embodiments, the bacterial conjugation system is an F plasmid system, and wherein: the at least one cis-acting component comprises an F plasmid origin of transfer (oriT) sequence; and the one or more trans-acting components comprise or are derived from gene products of the F plasmid.

### Integrator Module

Provided herein are integrator modules. The integrator module can be derived from a transposase system or a site-directed DNA recombinase system.

Integrator modules of the disclosure (e.g., integrator module, and first and/or second orthogonal integrator modules) can comprise or be derived from CRISPR-associated transposons or CASTs. CASTs are mobile genetic elements (MGEs) that have evolved to make use of minimal CRISPR systems for RNA-guided transposition of their DNA. Unlike traditional CRISPR systems that contain interference mechanisms to degrade targeted DNA, CASTs lack proteins and/or protein domains responsible for DNA cleavage. Specialized transposon machinery, similar to that of Tn7 transposon, complexes with the CRISPR RNA (crRNA) and associated Cas proteins for transposition. CAST systems have been characterized in a wide range of bacteria and make use of variable CRISPR configurations including Type I-F, Type I-B, Type I-C, Type I-D, Type I-E, Type IV, and Type V-K.

Many CRISPR-associated transposons are similar to the Tn7 transposon that functions with a cut and paste mechanism. It contains a heteromeric transposase consisting of TnsA and TnsB proteins, and a regulator protein TnsC. Structural analysis has shown binding of the TnsB protein and sequence specific motifs on the ends of the transposon which allows for excision and mobility. Targeting for integration is done by the TnsD or TnsE proteins which preferentially target safe sites within the host chromosome or mobile elements (plasmids or bacteriophages), respectively. TnsE is not found in CASTs but a TnsD homolog, TniQ, is present and functions to bridge the gap between the transposase and CRISPR-Cas. Multiple CRISPR types have been found to associate with transposons with two of the most studied being Type I-F, which makes use of a multi-subunit effector, and Type V-K, which makes use of a single Cas12k effector. In both cases, Tn7 transposons have evolved to make use of these effectors to create R loops for site-specific integration. While TnsA is present in Type I-F systems, it is notably absent in Type V-K systems which showed higher off-target integrations during initial characterization.

A Type I-F3 CAST (Tn6677) was initially identified in Vibrio Cholerae and has been extensively studied. This system contains proteins TnsA, TnsB, and TnsC that complex with Cas6, Cas7, and a Cas5-Cas8 fusion through interactions with TniQ. Initial integration steps include TniQ complexed with Cas proteins, which binds at the target site, and TnsA and TnsB excision of the transposon, which is followed by TnsC binding to TniQ and transposase binding to TnsC. There can be off-targeting prior to this final step, but TnsB and TnsC binding leads to a final proofreading step to maintain a high on-target percentage. Tn6677 integration has been validated at near 100% on-target efficiency at site specific locations in multiple points in the host genome. Other systems have also been characterized and validated in this class with varying ranges of efficiency, and include orthogonal systems for multiplexed insertions up to 10 kb.

A Type V-K system was originally characterized from a cyanobacteria, Scytonema hofmanni, and contains a single Cas effector, Cas12k, that functions with a tracrRNA. This system functions similarly to Tn7 but does not have a TnsA protein, which can result in off-targeting and chimera formation during over-expression. The Cas12k and tracrRNA complex bind to the target site and TnsC is polymerized directly adjacent prior to TniQ attachment and TnsB recognition and integration. While these systems use traditional tracrRNA characteristic of Type II CRISPR systems, they can also target with short crRNA located adjacent to the transposon end. Type V-K spacers preferentially target locations near tRNA genes, but other sites have been observed in these short crRNA guides.

The integrator module can be derived from a Type I-B, Type I-D, Type I-F, or Type V-K CRISPR-associated transposase system of a bacteria. The bacteria can comprise Vibrio cholera (Vch), Pseudoalteromonas (Pse), or Scytonema hofmanni (Sho). The integrator module can be derived from the Type I-F CRISPR-associated transposase system.

The Type I-F CRISPR-associated transposase system can be Tn6677, Tn7000, Tn7001, Tn7002, Tn7003, Tn7004, Tn7005, Tn7006, Tn7007, Tn7008, Tn7009, Tn6900, Tn7010, Tn7011, Tn7012, Tn7013, Tn7014, Tn7015, Tn7016, Tn7017. The Type I-F CRISPR-associated transposase system can be Tn6677.

In some embodiments: the one or more components of the integrator module comprise: i) an RNA-guided DNA binding complex, wherein the RNA-guided DNA binding complex comprises one or more Cas proteins, a transposase, one or more crRNAs, or any combination thereof, and ii) a transposition complex comprising one or more transposases; and the first integration sequence of the first and/or second Cassette^{START} comprises an R-TE (RE) and the second integration sequence of the first and/or second Cassette^{STOP} comprises an L-TE (LE).

The RE can comprise the sequence of any one of SEQ ID NOs: 361 or a sequence having one, two or three mismatches relative to the sequence of any one of SEQ ID NOs: 361. The LE can comprise the sequence of any one of SEQ ID NOs: 362 or a sequence having one, two or three mismatches relative to the sequence of any one of SEQ ID NOs: 362.

The one or more Cas proteins can comprise a Cas6 protein, a Cas7 protein, and a Cas8 protein. The Cas6 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 363. The Cas7 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 364. The Cas8 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 365. The transposase of the RNA-guided DNA binding complex can comprise or consist of a TniQ protein. The TniQ protein can comprise an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 366. The RNA-guided DNA binding complex can comprise one or more helper accessory proteins comprising ClpX and/or ClpP.

The one or more transposases of the transposition complex can comprise a TnsA protein, a TnsB protein, and a TnsC protein. The one or more transposases of the transposition complex can comprise a TnsAB fusion protein and a TnsC protein. The TnsA protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 367. The TnsB protein can comprise or consist of an amino acid sequences that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 368. The TnsC protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 369.

The integrator module can be derived from a Type V-K CRISPR-associated transposase system. In some embodiments: the one or more components of the integrator module comprise: i) an RNA-guided DNA binding complex, wherein the RNA-guided DNA binding complex comprises a Cas protein and one or more crRNAs, or any combination thereof, and ii) a transposition complex comprising one or more transposases; and wherein the first integration sequence of the first and/or second Cassette^{START} comprises an R-TE (RE) and the second integration sequence of the first and/or second Cassette^{STOP} comprises an L-TE (RE).

The Cas protein can comprise Cas12k. The one or more transposases of the transposition complex can comprise TniQ, TnsB, TnsC, or any combination thereof. In some embodiments, at least one of the one or more crRNAs comprise a spacer that is complementary to a search target sequence on a first strand of the first double stranded target sequence. In some embodiments, at least one of the one or more crRNAs comprise a spacer that is complementary to a search target sequence on a first strand of the second double-stranded target sequence.

crRNAs

The terms “gRNA,” “guide RNA”, “CRISPR guide sequence” or “crRNA” may be used interchangeably throughout and refer to a nucleic acid comprising a sequence that determines the binding specificity of the RNA-guided DNA-binding complex. A crRNA hybridizes to (complementary to, partially or completely) a double-stranded target sequence (e.g., in the genome) in a cell. The crRNA can comprise a spacer that hybridizes to a search target sequence on a first strand of the double stranded target sequence (a target site). The spacer may be between 15-35 nucleotides, 18-33 nucleotides, or 19-35 nucleotides in length. In some embodiments, the crRNA sequence that hybridizes to the target site is 15, 16, 17, 18, 19, 20, 21, 22, 23, 24, 25 26, 27, 28, 29, 30, 31, 32, or 33 nucleotides in length. In some embodiments, the crRNA sequence that hybridizes to the target site is between 10-30, or between 15-25, or 25-35 nucleotides in length.

To facilitate crRNA design, many computational tools have been developed (See Prykhozhij et al (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2); 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of crRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid (e.g., a spacer), in some embodiments, the gRNA may also comprise a scaffold sequence. In some embodiments, such a gRNA may be referred to as a single guide RNA (sgRNA) or a crRNA. Exemplary scaffold sequences will be evident to one of skill in the art and can be found, for example, in Jinek, et al. Science (2012) 337(6096):816-821, and Ran, et al. Nature Protocols (2013) 8:2281-2308. crRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence. The crRNA can comprise a [repeat scaffold]-[spacer]-[repeat scaffold] structure. The first strand of the double stranded target sequence can be the sense strand.

In some embodiments, the spacer sequence of the crRNA is at least 50%, 55%, 60%, 65%, 70%, 75%, 80%, 85%, 90%, 95%, 96%, 97%, 98%, 99%, or at least 100% complementary to a search target sequence on a first strand of the double stranded target sequence. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the search target sequence on a first strand of the double stranded target sequence (e.g., the last 5, 6, 7, 8, 9, or 10 nucleotides of the 3′ end of the target sequence). The crRNA can comprise a spacer that is complementary to a search target sequence on a first strand of the double stranded target sequence. The first strand of the double stranded target sequence can be the sense strand.

The target sequence may be flanked by a protospacer adjacent motif (PAM). A PAM site is a nucleotide sequence in proximity to a target sequence. For example, a PAM may be a DNA sequence immediately following the DNA sequence targeted by the RNA-guided DNA binding complex.

The target sequence may or may not be flanked by a protospacer adjacent motif (PAM) sequence. In some embodiments, a nucleic acid-guided nuclease can only bind a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In some embodiments, the target sequence is immediately flanked on the 3′ end by a PAM sequence. A PAM can be 1, 2, 3, 4, 5, 6, 7, 8, 9, 10 or more nucleotides in length. In some embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems and Type II CRISPR/Cas systems). In some embodiments, e.g., Type I systems, the PAM is on the alternate side of the protospacer (the 5′ end). Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as ITT, TTG, TTC, TTTT, NGG, NGA, NAG, NGGNG and NNAGAAW (W=A or T), NNNNGATT, NAAR (R=A or G), NNGRR (R=A or G), NNAGAA and NAAAAC, where “N” is any nucleotide. In some embodiments, e.g., for Type I-F CAST systems and the systems, nucleic acid compositions and methods described herein, the PAM sequence can be 5′-CN-3′, where “N” is any nucleotide.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

A donor genomic segment can be capable of being integrated at an integration site following binding of the RNA-guided DNA binding complex to the search target sequence. The integration site can be about 48 to 52 base pairs (e.g., 48, 49, 50, 51, or 52 base pairs) downstream of the double stranded target sequence.

In some embodiments, the one or more crRNAs of the integrator module comprise a scaffold comprising or consisting of the sequence of SEQ ID NO: 385 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to the sequence of SEQ ID NO: 385. In some embodiments, the nucleic acid sequence encoding the scaffold of the one or more crRNAs of the integrator module comprises or consists of the sequence of SEQ ID NO: 388 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to the sequence of SEQ ID NO: 388.

In some embodiments, at least one of the one or more crRNAs comprise a spacer that is complementary to a search target sequence on a first strand of the first double stranded target sequence. In some embodiments, at least one of the one or more crRNAs comprise a spacer that is complementary to a search target sequence on a first strand of the second double-stranded target sequence.

### Integrases and Recombinases

The integrator module can be derived from a transposase system or a site-directed DNA recombinase system. The site-directed DNA recombinase system can comprise a site-directed integrase system.

The one or more components of the integrator module can comprise an integrase. The integrase can be Bxb1 and the first and/or second integration sequences can comprise an attB site. The genome of the recipient cell can comprise at least one attP site. The one or more components of the integrator module can comprise an integrase. The integrase can be phiC31 and the first and/or second integration sequences can comprise an attB site. The genome of the recipient cell can comprise at least one attP site. The one or more components of the integrator module can comprise a recombinase. The recombinase can be Cre or FLP and the first and/or second integration sequences can comprise a loxP site or an FRT site.

The genome of the recipient cell can comprise at least one loxP site or FRT site.

### Orthogonal CAST Sequential Integration System (OASIS) Systems

Provided herein are compositions and methods for generation of donor cells. In some embodiments, Cassette^{START} and Cassette^{STOP} can be integrated into the genome of a cell using methods known in the art, e.g., lambda red recombineering system. There is also provided herein, OASIS (Orthogonal ACE Sequential Integration System) for generation of donor cells. OASIS leverages the diversity of orthogonal Type I-F CASTs to rapidly construct non-enterobacterial ACE donors.

Disclosed herein include nucleic acid compositions for producing a chimeric or trimeric genome. In some embodiments, the nucleic acid composition comprises: i) a first and/or second start cassette (Cassette^{START}) each comprising, from 5′ to 3′: at least one cis-acting component of a first and/or second delivery module and a first integration sequence; ii) a first and/or second stop cassette (Cassette^{STOP}) each comprising, from 5′ to 3′: a second integration sequence and at least one cis-acting component of the first and/or second delivery module; iii) one or more first helper polynucleotides each comprising a sequence encoding a trans-acting component of the first and/or second delivery module, wherein the first and/or second delivery module comprise one or more trans-acting components; and optionally iv) one or more second helper polynucleotides each comprising a sequence encoding a component of an integrator module, wherein the integrator module comprises one or more components.

In some embodiments: the first and/or second Cassette^{START} is comprised within a first orthogonal integration vector further comprising one or more third helper polynucleotides each comprising a sequence encoding a component of a first orthogonal integrator module, wherein the first orthogonal integrator module comprises one or more components; and the first and/or second Cassette^{STOP} is comprised within a second orthogonal integration vector further comprising one or more fourth helper polynucleotides each comprising a sequence encoding a component of a second orthogonal integrator module, wherein the second orthogonal integrator module comprises one or more components. In some embodiments, the first and/or second Cassette^{START} is flanked by a 5′ RE and a 3′ LE and the first and/or second Cassette^{STOP} is flanked by a 5′ RE and a 3′ LE, and the integrator module, the first orthogonal integrator module, and the second orthogonal integrator module are orthogonal to each other.

In some embodiments, the first orthogonal integrator module and/or the second orthogonal integrator module are each derived from a Type I-B, Type I-D, Type I-F, or Type V-K CRISPR-associated transposase system of a bacteria. The bacteria can comprise Vibrio cholera (Vch), Pseudoalteromonas (Pse), or Scytonema hofmanni (Sho). In some embodiments, the first orthogonal integrator module and/or the second orthogonal integrator module are each derived from the Type I-F CRISPR-associated transposase system. The Type I-F CRISPR-associated transposase system can be Tn6677, Tn7000, Tn7001, Tn7002, Tn7003, Tn7004, Tn7005, Tn7006, Tn7007, Tn7008, Tn7009, Tn6900, Tn7010, Tn7011, Tn7012, Tn7013, Tn7014, Tn7015, Tn7016, or Tn7017.

In some embodiments: the one or more components of the first orthogonal integrator module and/or the one or more components of the second orthogonal integrator module comprise: i) an RNA-guided DNA binding complex, wherein the RNA-guided DNA binding complex comprises a Cas protein, a transposase, one or more crRNAs, or any combination thereof, and ii) a transposition complex comprising one or more transposases.

The RE can comprise the sequence of any one of SEQ ID NOs: 343-344 or a sequence having one, two or three mismatches relative to the sequence of any one of SEQ ID NOs: 343-344. The LE can comprise the sequence of any one of SEQ ID NOs: 345-346 or a sequence having one, two or three mismatches relative to the sequence of any one of SEQ ID NOs: 345-346.

The one or more Cas proteins can comprise a Cas6 protein, a Cas7 protein, and a Cas8 protein. The Cas6 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 347 or SEQ ID NO: 348. The Cas7 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 349 or SEQ ID NO: 350. The Cas8 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 351 or SEQ ID NO: 352. The transposase of the RNA-guided DNA binding complex can comprise a TniQ protein. The TniQ protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 353 or SEQ ID NO: 354. The RNA-guided DNA binding complex can comprise one or more helper accessory proteins comprising ClpX and/or ClpP.

The one or more transposases of the transposition complex can comprise a TnsA protein, a TnsB protein, and a TnsC protein. The one or more transposases of the transposition complex can comprise a TnsAB fusion protein and a TnsC protein. The TnsA protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 355 or SEQ ID NO: 356. The TnsB protein can comprise or consist of an amino acid sequences that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 357 or SEQ ID NO: 358. The TnsC protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 359 or SEQ ID NO: 360.

In some embodiments: the one or more crRNAs of the first orthogonal integrator module comprise a scaffold comprising the sequence of SEQ ID NO: 389 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to SEQ ID NO: 389. In some embodiments, the nucleic acid sequence encoding the scaffold of the one or more crRNAs of the first orthogonal integrator module comprises the sequence of SEQ ID NO: 386 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to SEQ ID NO: 386.

In some embodiments, the one or more crRNAs of the second orthogonal integrator module comprise a scaffold comprising the sequence of SEQ ID NO: 390 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to SEQ ID NO: 390. In some embodiments, the nucleic acid sequence encoding the scaffold of the one or more crRNAs of the second orthogonal integrator module comprises the sequence of SEQ ID NO: 387 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to SEQ ID NO: 387.

In some embodiments, one of the one or more crRNAs of the first orthogonal integrator module comprises a spacer that is complementary to a search target sequence on a first strand of an upstream double stranded target sequence of a cell and/or one of the one or more the crRNAs of the second orthogonal integrator module comprises a spacer that is complementary to a search target sequence on a first strand of a downstream double stranded target sequence of the cell, wherein

upon expression of the one or more components of the first orthogonal integrator module and/or the one or more components of the second orthogonal integrator module in the cell, the first and/or second Cassette^{START} is capable of being inserted into the upstream double-stranded target sequence in the genome of the cell and the first and/or second Cassette^{STOP} is capable of being inserted into the downstream double stranded target sequence in the genome of the cell, thereby generating a donor cell.

### Screening and Selection Markers

The polynucleotides described herein can comprise one or more screen and/or selection markers. In some embodiments, the first and/or second Cassette^{START}, the first and/or second Cassette^{STOP} or any combination thereof are labeled with screening and/or selection markers As used herein, the term “selectable marker” shall be given its ordinary meaning and shall also refer a gene introduced into cells, e.g., bacteria or cells in culture, which confers one or more traits suitable for artificial selection. As used herein, the term “screenable marker” shall be given its ordinary meaning and shall also refer to a gene introduced into cells, e.g., bacteria or cells in culture, which confers one or more traits suitable for phenotyping screening, e.g., a fluorescent protein.

The first and/or second Cassette^{START}, the first and/or second Cassette^{STOP} or any combination thereof can comprise: one or more marker polynucleotides each comprising a sequence encoding a positive or negative screening and/or selection marker. In some embodiments, the one or more marker polynucleotides are situated 5′ of the at least one cis-acting component of the first and/or second delivery module in the first and/or second Cassette^{START} and/or the one or more marker polynucleotides are situated 5′ of the second integration sequence in the first and/or second Cassette^{STOP}.

In some embodiments: the positive screening and/or selection marker is selected from the group comprising: a fluorescent protein, an antibiotic resistance cassette, an enzyme, or any combination thereof. The enzyme can be 0-galactosidase. In some embodiments, the negative screening and/or selection marker is selected from the group comprising: a fluorescent protein, an enzyme, or a combination thereof. The enzyme can be encoded by the sacB gene and/or the pheS^{mut} gene.

In some embodiments, the antibiotic resistance cassette confers resistance to an antibiotic comprising phleomycin D1 (ZEOCIN™), kanamycin, spectinomycin, streptomycin, ampicillin, carbenicillin, bleomycin, erythromycin, polymyxin B, tetracycline, chloramphenicol, or any combination thereof. The fluorescent protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof.

The first and/or second Cassette^{START}, the first and/or second Cassette^{STOP} can be configured such that the positive screening and/or selection marker can be removed. At least one of the one or more marker polynucleotides can be flanked by recombinase sites. In some embodiments, the one or more marker polynucleotides of the first Cassette^{START} and/or the first Cassette^{STOP} can be flanked by recombinase sites. In some embodiments, the at least one of the one or more marker polynucleotides of the first Cassette^{START} and/or the first Cassette^{STOP} flanked by recombinase sites are capable of being removed from the genome of the recipient cell comprising the chimeric genome upon expression of at least one recombinase or integrase in the recipient cell comprising the chimeric genome. The at least one recombinase or integrase can comprise phiC31, Bxb1, Cre, and/or FLP.

### Polynucleotides

Disclosed herein include systems for producing a chimeric or trimeric genome. In some embodiments, the system comprises: a) a donor cell comprising: (i) a first start cassette (Cassette^{START}) comprising, from 5′ to 3′, at least one cis-acting component of a first delivery module and a first integration sequence, and (ii) a first stop cassette (Cassette^{STOP}) comprising, from 5′ to 3′, a second integration sequence and at least one cis-acting component of the first delivery module, wherein the first Cassette^{START} is integrated at a first site in the genome of the donor cell and the first Cassette^{STOP} is integrated at a second site in the genome of the donor cell, wherein intervening sequence between the first Cassette^{START} and the first Cassette^{STOP} defines a first genomic transfer window; and (iii) one or more trans-acting components of the first delivery module or one or more first helper polynucleotides each comprising a sequence encoding a trans-acting component of the first delivery module, wherein the first delivery module comprises the one or more trans-acting components; and b) a recipient cell comprising one or more components of an integrator module or one or more second helper polynucleotides each comprising a sequence encoding a component of an integrator module, wherein the integrator module comprises the one or more components.

Disclosed herein include nucleic acid compositions for producing a chimeric or trimeric genome. In some embodiments, the nucleic acid composition comprises: i) a first and/or second start cassette (Cassette^{START}) each comprising, from 5′ to 3′: at least one cis-acting component of a first and/or second delivery module and a first integration sequence; ii) a first and/or second stop cassette (Cassette^{STOP}) each comprising, from 5′ to 3′: a second integration sequence and at least one cis-acting component of the first and/or second delivery module; iii) one or more first helper polynucleotides each comprising a sequence encoding a trans-acting component of the first and/or second delivery module, wherein the first and/or second delivery module comprise one or more trans-acting components; and optionally iv) one or more second helper polynucleotides each comprising a sequence encoding a component of an integrator module, wherein the integrator module comprises one or more components.

In some embodiments: the first and/or second Cassette^{START} is comprised within a first orthogonal integration vector further comprising one or more third helper polynucleotides each comprising a sequence encoding a component of a first orthogonal integrator module, wherein the first orthogonal integrator module comprises one or more components; and the first and/or second Cassette^{STOP} is comprised within a second orthogonal integration vector further comprising one or more fourth helper polynucleotides each comprising a sequence encoding a component of a second orthogonal integrator module, wherein the second orthogonal integrator module comprises one or more components. In some embodiments, the first and/or second Cassette^{START} is flanked by a 5′ RE and a 3′ LE and the first and/or second Cassette^{STOP} is flanked by a 5′ RE and a 3′ LE, and the integrator module, the first orthogonal integrator module, and the second orthogonal integrator module are orthogonal to each other.

One or more of the one or more first helper polynucleotides can comprise a first promoter operably linked to the sequence encoding a trans-acting component of the first and/or second delivery module. One or more of the one or more second helper polynucleotides can comprise a second promoter operably linked to the sequence encoding a component of an integrator module. One or more of the one or more third helper polynucleotides can comprise a third promoter operably linked to the sequence encoding a component of a first orthogonal integrator module. One or more of the one or more fourth helper polynucleotides can comprise a fourth promoter operably linked to the sequence encoding a component of a second orthogonal integrator module. The first, second, third, and/or fourth promoters can be the same or different.

The first, second, third, and/or fourth promoters can comprise a constitutive promoter, an inducible promoter, or a combination thereof. The first, second, third, and/or fourth promoters can comprise the TlpA operator/promoter, lambda phage pL, lambda phage pR, lambda phage pRM, or any combination thereof. In some embodiments, the first, second, third, and/or fourth promoters comprise a promoter selected from the group comprising: a bacteriophage promoter, (e.g., Pls1con, T3, T7, SP6, or PL); a bacterial promoter, (e.g., Pbad, PmgrB, Ptrc2, Plac/ara, Ptac, or Pm); and/or a bacterial-bacteriophage hybrid promoter, (e.g., PLlacO or PLtetO). In some embodiments, the first, second, third, and/or fourth promoters comprise a positively regulated E. coli promoter selected from the group comprising: a σ⁷⁰ promoter, (e.g., inducible pBad/araC promoter, Lux cassette right promoter, modified lambda Prm promoter, plac Or2-62 (positive), pBad/AraC with extra REN sites, pBad, P(Las) TetO, P(Las) CIO, P(Rhl), Pu, FecA, pRE, cadC, hns, pLas, or pLux); a as promoter, (e.g., Pdps); a σ³² promoter, (e.g., heat shock); and/or a σ⁵⁴ promoter, (e.g., glnAp2).

In some embodiments, the first, second, third, and/or fourth promoters comprise a negatively regulated E. coli promoter selected from the group comprising: a σ⁷⁰ promoter, (e.g., Promoter (PRM+), modified lambda Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modifed Pr, modifed Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLaclq, rrnB P1, cadC, hns, PfhuA, pBad/araC, nhaA, OmpF, or RcnR); a as promoter, (e.g., Lutz-Bujard LacO with alternative sigma factor σ³⁸); a σ³² promoter, (e.g., Lutz-Bujard LacO with alternative sigma factor σ³²); and/or a σ⁵⁴ promoter, (e.g., glnAp2).

The first, second, third, and/or fourth promoters can comprise a P7 promoter. The first, second, third, and/or fourth promoters can comprise a heat-shock promoter, (e.g., pTSR, pR-pL, GrpE, HtpG, Lon, RpoH, Clp, and/or DnaK). The first, second, third, and/or fourth promoters can comprise a constitutive Escherichia coli as promoter, (e.g., osmY promoter (BBa_J45993)); a constitutive Escherichia coli σ³² promoter, (e.g., htpG heat shock promoter (BBa_J45504)); a constitutive Escherichia coli σ⁷⁰ promoter, (e.g., lacq promoter (BBa_J54200 or BBa_J56015), E. coli CreABCD phosphate sensing operon promoter (BBa_J64951), GlnRS promoter (BBa_K088007), lacZ promoter (BBa_K119000 or BBa_K119001), M13K07 gene I promoter (BBa_M13101), M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), or M13110 (BBa_M13110)); a constitutive Bacillus subtilis σ^{A} promoter, (e.g., promoter veg (BBa_K143013), promoter 43 (BBa_K143013), P_{liaG} (BBa_K823000), P_{lepA} (BBa_K823002), or P_{veg} (BBa_K823003)); a constitutive Bacillus subtilis σ^{B} promoter, (e.g., promoter ctc (BBa_K143010) or promoter gsiB (BBa_K143011)); a Salmonella promoter, (e.g., Pspv2 from Salmonella (BBa_K112706) or Pspv from Salmonella (BBa_K112707)); a bacteriophage T7 promoter (e.g., BBa_I712074, BBa 1719005, BBa_J34814, BBa_J64997, BBa_K113010, BBa_K113011, BBa_K113012, BBa_R0085, BBa_R0180, BBa_R0181, BBa_R0182, BBa_R0183, BBa_Z0251, BBa_Z0252, or BBa_Z0253); and/or a bacteriophage SP6 promoter (e.g., SP6 promoter (BBa_J64998)). The first, second, third, and/or fourth promoters can comprise an Arabinose inducible promoter (P^{Ara}).

In some embodiments: the one or more first helper polynucleotides are situated on the same nucleic acid or different nucleic acids; and/or the one or more second helper polynucleotides are situated on the same nucleic acid or different nucleic acids. In some embodiments: two or more of the one or more first helper polynucleotides situated on the same nucleic acid, two or more of the one or more second helper polynucleotides situated on the same nucleic acid, two or more of the one or more third helper polynucleotides comprised within the first orthogonal integration vector, and/or two or more of the one or more fourth helper polynucleotides comprised within the second orthogonal integration vector, are comprised within an operon or operably linked via a tandem expression element. In some embodiments, the tandem expression element is selected from the group comprising a bacterial ribosome binding site (RBS), an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof.

Any of the one or more first helper polynucleotides, the one or more second helper polynucleotides, the one or more third helper polynucleotides, the one or more fourth helper polynucleotides, and/or the bacterial operon can comprise a 5′UTR and/or a 3′ UTR. The one or more first helper polynucleotides, the one or more second helper polynucleotides, the one or more third helper polynucleotides, and/or the one or more fourth helper polynucleotides can comprise a transcript stabilization element. The one or more first helper polynucleotides or the one or more second helper polynucleotides can be comprised within one or more vectors. The one or more vectors, the first orthogonal vector, and/or the second orthogonal vector can comprise an RNA viral vector, a DNA viral vector, a plasmid vector, an artificial chromosome, or any combination thereof.

The first and/or second Cassette^{START} can comprise or consist of the sequence of SEQ ID NO: 370 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 370. The first and/or second Cassette^{STOP} can comprise or consist of the sequence of SEQ ID NO: 371 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 371. The first and/or second Cassette^{START} flanked by a 5′ RE and a 3′ LE and can comprise or consist of the sequence of any one of SEQ ID NOs: 372-373 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 372-373.

The first and/or second Cassette^{STOP} flanked by a 5′ RE and a 3′ LE can comprise or consist of the sequence of any one of SEQ ID NOs: 374-375 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 374-375.

In some embodiments: the two or more first helper polynucleotides comprised within an operon or operably linked via a tandem expression element comprise or consist of the sequence of SEQ ID NO: 376 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NO: 376; the two more second helper polynucleotides comprised within an operon or operably linked via a tandem expression element comprise or consist of the sequence of SEQ ID NO: 377 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NO: 377; the two or more third helper polynucleotides comprised within an operon or operably linked via a tandem expression element comprise or consist of the sequence of SEQ ID NO: 378 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NO: 378; and/or the two or more fourth helper polynucleotides comprised within an operon or operably linked via a tandem expression element comprise or consist of the sequence of SEQ ID NO: 379 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NO: 379.

In some embodiments, the two or more first helper polynucleotides are comprised within a plasmid, and the plasmid comprises the sequence of SEQ ID NO: 380 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to the sequence of SEQ ID NO: 380. In some embodiments, the two more second helper polynucleotides are comprised within a plasmid, and the plasmid comprises the sequence of SEQ ID NO: 381 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to the sequence of SEQ ID NO: 381. In some embodiments, the first orthogonal integration vector comprises the sequence of SEQ ID NO: 382 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to the sequence of SEQ ID NO: 382. In some embodiments, the second orthogonal integration vector comprises the sequence of SEQ ID NO: 383 or a sequence having 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identity to the sequence of SEQ ID NO: 383.

The nucleic acid compositions provided herein can comprise one or more components/features of a disclosed sequence (e.g., promoter, oriT, RE/LE, selectable/screenable marker, component of a delivery module, component of an integrator module).

Provided herein are nucleic acids that are at least 80%, 85%, 90%, 95%, 98%, 99%, or 100% identical to SEQ ID NOS: 1-390, portions thereof, and/or complements thereof. Also provided herein are nucleic acids that comprise at least about 5 consecutive nucleotides (e.g., about 5 nt, 10 nt, 15 nt, 20 nt, 25 nt, 30 nt, 35 nt, 40 nt, 45 nt, 50 nt, 60 nt, 70 nt, 80 nt, 90 nt, 100 nt, 110 nt, 120 nt, 128 nt, 130 nt, 140 nt, 150 nt, 160 nt, 170 nt, 180 nt, 190 nt, 200 nt, 210 nt, 220 nt, 230 nt, 240 nt, 250 nt, 260 nt, 270 nt, 280 nt, 290 nt, 300 nt, 310 nt, 320 nt, 330 nt, 340 nt, 350 nt, 360 nt, 370 nt, 380 nt, 390 nt, 400 nt, 410 nt, 420 nt, 430 nt, 440 nt, 450 nt, 460 nt, 470 nt, 480 nt, 490 nt, 500 nt, 510 nt, 520 nt, 530 nt, 540 nt, 550 nt, 560 nt, 570 nt, 580 nt, 590 nt, 600 nt, 610 nt, 620 nt, 630 nt, 640 nt, 650 nt, 660 nt, 670 nt, 680 nt, 690 nt, 700 nt, 710 nt, 720 nt, 730 nt, 740 nt, 750 nt, 760 nt, 770 nt, 780 nt, 790 nt, 800 nt, 810 nt, 820 nt, 830 nt, 840 nt, 850 nt, 860 nt, 870 nt, 880 nt, 890 nt, 900 nt, 910 nt, 920 nt, 930 nt, 940 nt, 950 nt, 960 nt, 970 nt, 980 nt, 990 nt, 1000 nt, 1500 nt, 2000 nt, 5000 nt, 10000 nt, 50000 nt, or a number or a range between any two of these values) of a sequence described by SEQ ID NOS: 1-390, or a complement thereof.

### METHODS

Disclosed herein are methods for producing a chimeric genome. In some embodiments, the method comprises: contacting a recipient cell disclosed herein with a donor cell disclosed herein, and: expressing the one or more trans-acting components of the first delivery module in the donor cell, thereby the sequence defined by the genomic transfer window is transferred from the donor cell to the recipient cell and thereby the first donor genomic segment comprising the first donor sequence flanked by the first integration sequence and the second integration sequence of the first Cassette^{START} and the first Cassette^{STOP} is generated in the recipient cell; and expressing the one or more components of the integrator module in the recipient cell, thereby the first donor genomic segment is inserted into the first double-stranded target sequence in the genome of the recipient cell, thereby generating the chimeric genome in the recipient cell.

The method can comprise repeating the contacting step one or more times, wherein each contacting step comprises contacting an nth-donor cell with the recipient cell, thereby generating a recipient cell comprising an n-meric genome, wherein each nth-donor cell comprises: (i) an nth start cassette (Cassette^{START}) comprising, from 5′ to 3′, at least one cis-acting component of an nth delivery module and an nth integration sequence, and (ii) an nth stop cassette (Cassette^{STOP}) comprising, from 5′ to 3′, an nth integration sequence and at least one cis-acting component of the nth delivery module, wherein the nth Cassette^{START} is integrated at a first site in the genome of the nth donor cell and the nth Cassette^{STOP} is integrated at a second site in the genome of the nth donor cell, wherein intervening sequence between the nth Cassette^{START} and the nth Cassette^{STOP} defines an nth genomic transfer window; and (iii) one or more trans-acting components of the nth delivery module or one or more first helper polynucleotides each comprising a sequence encoding a trans-acting component of the nth delivery module, wherein the nth delivery module comprises the one or more trans-acting components, and optionally wherein the sequence of each nth genomic transfer window is different from each other.

Disclosed herein are methods for producing a trimeric genome. In some embodiments, the method comprises: (a) contacting a recipient cell of the disclosure with the donor cell of the disclosure, and: expressing the one or more trans-acting components of the first delivery module in the donor cell, thereby the sequence defined by the genomic transfer window is transferred from the donor cell to the recipient cell and thereby the first donor genomic segment comprising the first donor sequence flanked by the first integration sequence and the second integration sequence of the first Cassette^{START} and the first Cassette^{STOP} is generated in the recipient cell; and expressing the one or more components of the integrator module in the recipient cell, thereby the first donor genomic segment is inserted into the first double-stranded target sequence in the genome of the recipient cell, thereby generating the chimeric genome in the recipient cell, and (b) contacting the recipient cell comprising the chimeric genome with a second donor cell of the disclosure, and: expressing the one or more trans-acting components of the second delivery module in the second donor cell, thereby the sequence defined by the second genomic transfer window is transferred from the second donor cell to the recipient cell comprising the chimeric genome and thereby the second donor genomic segment comprising the second donor sequence flanked by the first integration sequence and the second integration sequence of the second Cassette^{START} and the second Cassette^{STOP} is generated in the recipient cell comprising the chimeric genome; and expressing the one or more components of the integrator module in the recipient cell comprising the chimeric genome, thereby the second donor genomic segment is inserted into the second double-stranded target site in the genome of the recipient cell comprising the chimeric genome, thereby generating the trimeric genome in the recipient cell comprising the chimeric genome, optionally wherein the second double-stranded target site is comprised within the first donor genomic segment.

Prior to the contacting of step (b), the method can comprise expressing at least one recombinase or integrase in the recipient cell comprising the chimeric genome, thereby the at least one of the one or more marker polynucleotides of the first Cassette^{START} and/or the first Cassette^{STOP} flanked by recombinase sites are removed from the genome of the recipient cell comprising the chimeric genome.

The ratio of donor cell (e.g., donor cell or second donor cell) to recipient cells in the contacting steps can vary. The ratio of the donor cell to the recipient cell can be approximately 4:1 (v/v) and/or the ratio of the second donor cell to the recipient cell comprising the chimeric genome can be approximately 4:1 (v/v). In some embodiments, the ratio can be 1:1, 1.1:1, 1.2:1, 1.3:1, 1.4:1, 1.5:1, 1.6:1, 1.7:1, 1.8:1, 1.9:1, 2:1, 2.5:1, 3:1, 4:1 (v/v), or a number or a range between any two of the values.

The first and/or second donor genomic segment can be inserted into the first and/or second double-stranded target sequence in the genome of the recipient cell and/or the genome of the recipient cell comprising the chimeric genome at an integration efficiency of at least 10⁴ CFU. In some embodiments, the method has an on-target insertion rate of at least 90% (e.g., 90%, 91%, 92%, 93%, 94%, 95%, 96%, 97%, 98%, 99%, 100%, or a number or a range between any two of these values).

Disclosed herein are genome-expanded organisms. In some embodiments, the genome-expanded organism comprises a chimeric genome, trimeric genome, or n-meric genome generated by a method provided herein. The chimeric, trimeric genome, or n-meric genome can be stable following at least 100 generations. As provided above, the expanded genomes (e.g., chimeric, trimeric genome, or n-meric genome) can be at least about 4 MB (e.g., megabases) in length. The expanded genome can be at least about 4 MB, 4.5 MB, 5 MB, 6 MB, 6.5 MB, 7 MB, 7.5 MB, 8 MB, 8.5 MB, 9 MB, 9.5 MB, or 10 MB in length. The expanded genome can be, can be at least, or can be at least about 4.0 MB, 4.1 MB, 4.2 MB, 4.3 MB, 4.4 MB, 4.5 MB, 4.6 MB, 4.7 MB, 4.8 MB, 4.9 MB, 5.0 MB, 5.1 MB, 5.2 MB, 5.3 MB, 5.4 MB, 5.5 MB, 5.6 MB, 5.7 MB, 5.8 MB, 5.9 MB, 6.0 MB, 6.1 MB, 6.2 MB, 6.3 MB, 6.4 MB, 6.5 MB, 6.6 MB, 6.7 MB, 6.8 MB, 6.9 MB, 7.0 MB, 7.1 MB, 7.2 MB, 7.3 MB, 7.4 MB, 7.5 MB, 7.6 MB, 7.7 MB, 7.8 MB, 7.9 MB, 8.0 MB, 8.1 MB, 8.2 MB, 8.3 MB, 8.4 MB, 8.5 MB, 8.6 MB, 8.7 MB, 8.8 MB, 8.9 MB, 9.0 MB, 9.1 MB, 9.2 MB, 9.3 MB, 9.4 MB, 9.5 MB, 9.6 MB, 9.7 MB, 9.8 MB, 9.9 MB, 10.0 MB, in length, or a number or range between any two of these values. In some embodiments, the expanded genomes can be can be, can be at least, or can be at least about 3.0 MB, 3.1 MB, 3.2 MB, 3.3 MB, 3.4 MB, 3.5 MB, 3.6 MB, 3.7 MB, 3.8 MB, 3.9 MB, 4.0 MB, 4.1 MB, 4.2 MB, 4.3 MB, 4.4 MB, 4.5 MB, 4.6 MB, 4.7 MB, 4.8 MB, 4.9 MB, 5.0 MB, 5.1 MB, 5.2 MB, 5.3 MB, 5.4 MB, 5.5 MB, 5.6 MB, 5.7 MB, 5.8 MB, 5.9 MB, 6.0 MB, 6.1 MB, 6.2 MB, 6.3 MB, 6.4 MB, 6.5 MB, 6.6 MB, 6.7 MB, 6.8 MB, 6.9 MB, 7.0 MB, 7.1 MB, 7.2 MB, 7.3 MB, 7.4 MB, 7.5 MB, 7.6 MB, 7.7 MB, 7.8 MB, 7.9 MB, 8.0 MB, 8.1 MB, 8.2 MB, 8.3 MB, 8.4 MB, 8.5 MB, 8.6 MB, 8.7 MB, 8.8 MB, 8.9 MB, 9.0 MB, 9.1 MB, 9.2 MB, 9.3 MB, 9.4 MB, 9.5 MB, 9.6 MB, 9.7 MB, 9.8 MB, 9.9 MB, 10.0 MB, or longer, in length, or a number or range between any two of these values.

### Kits

Disclosed herein include kits comprising a system or nucleic acid composition described herein, and a set of instructions for use. For example, the kit can comprise a nucleic acid composition comprising one or more polynucleotides of the disclosure (e.g., the first, second, third, and/or fourth helper polynucleotides).

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

Some aspects of the embodiments discussed above are disclosed in further detail in the following examples, which are not in any way intended to limit the scope of the present disclosure.

### Example 1

### Creation of Genomic Chimeras Via Megabase Scale Genome Expansion

To address the need for new technologies specifically enabling programmable megabase scale genomic recombination-independent integration, e.g., in a single step, there is described in this Example Additive Conjugative-CAST Engineering (ACE). ACE combines conjugation from a non-self-propagating broad host range RP4 plasmid with targeted transposition from Type I-F Tn6677 CRISPR-associated transposition (CAST) from Vibrio cholerae HE-45 to precisely transfer and insert defined donor genomic fragments into the recipient's genome in a programmable manner. CAST has been previously demonstrated to perform CRISPR RNA (crRNA) dependent DNA transposition of up to 10-kb into target genomic loci and function in a variety of bacterial species. Inspired by its programmability and versatility, it is described herein that CASTs, as a component of ACE, can perform efficient, precise, and robust DNA integrations ranging from 100-kb to 2-mb per step into the E. coli genome, and genome expansions of at least ˜3-mb possible in two consecutive ACE steps. ACE is demonstrated herein as a tool for interrogating the genome architecture and the transcriptomic landscape of these chimeric E. coli cells with massive complementation of their DNA genome. Going beyond constructing merodiploid E. coli strains, the diversity and range of bacterial genomic material transferred by ACE was expanded by developing OASIS (Orthogonal ACE Sequential Integration System), leveraging the diversity of orthogonal Type I-F CASTs to rapidly construct non-enterobacterial ACE donors. With ACE and OASIS, there is showcased transfer of large >500-kb genome fragments from Pseudomonas into E. coli and begin multi-omic evaluations of select genome chimeras of increasing phylogenetic distance. Design, validation, and characterization of ACE

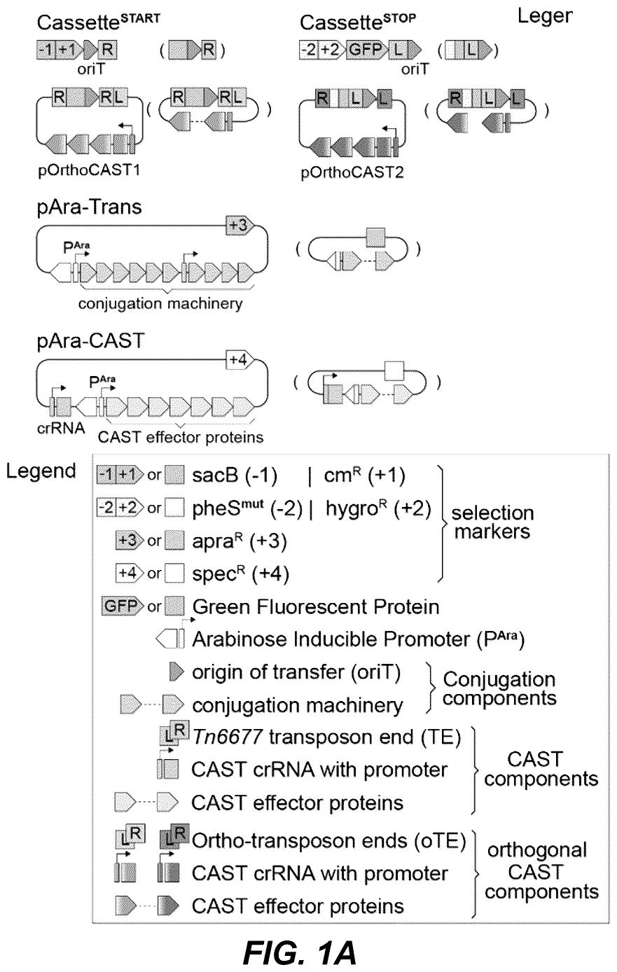

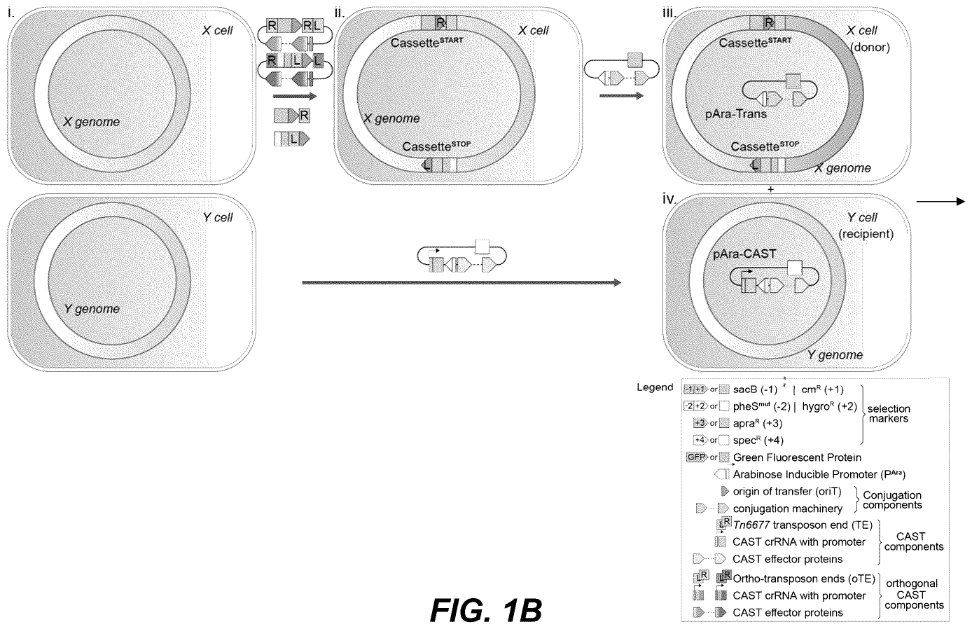

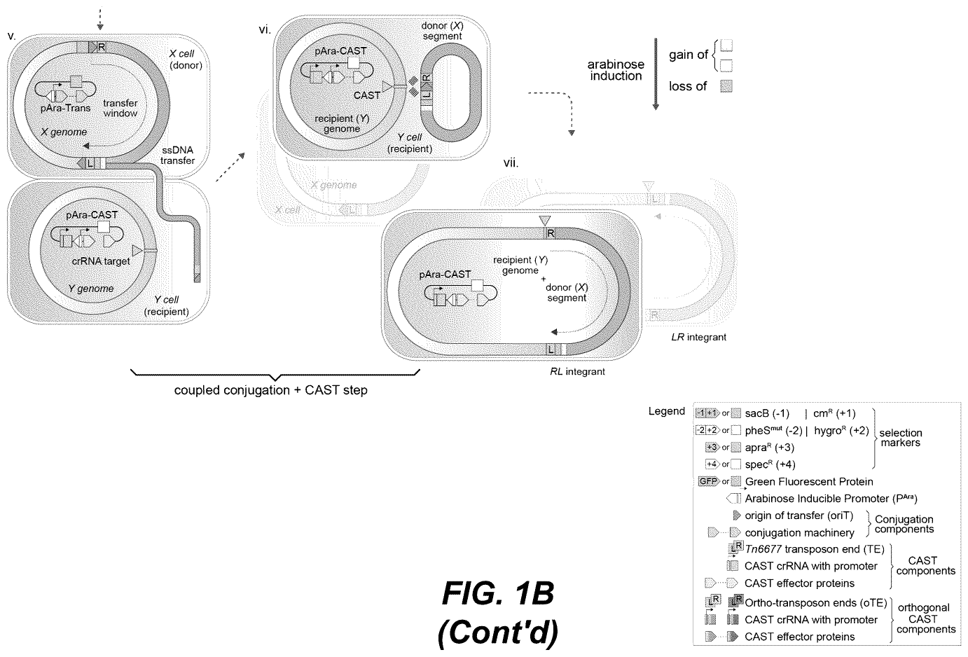

In the ACE system, the engineered bacterial conjugation and CAST components (FIG. 1A-FIG. 1B) are incorporated within four modular parts: Cassette^{START} Cassette^{STOP} pAra-Trans, and pAra-CAST. Cassette^{START} and Cassette^{STOP} each encode one origin of transfer (oriT) sequences from the RP4 conjugative plasmid and either the Right or Left transposon end (R-TE and L-TE respectively) sequences from the Tn6677 CAST system, together with corresponding negative and positive selection markers and green fluorescent protein (FIG. 1A). R-TE is placed downstream to the oriT in Cassette^{START} to mark the start of conjugation, while L-TE is placed upstream to the second oriT in Cassette^{STOP} to mark the end of conjugation. Together, Cassette^{START} and Cassette^{STOP} mark the precise start and the end of the transferred genomic fragment from the donor and retain the R-TE and L-TE sequences after the conjugation into the recipient cell to guide the genomic integration. Plasmid pAra-Trans holds the components of a minimized conjugation system to facilitate DNA conjugation from the donor cell (FIG. 1A). Plasmid pAra-CAST encodes the rest of the CAST components, including the crRNA dictating the location of genomic integration in the recipient cell (FIG. 1A).

The donor cell is defined starting with the consecutive integrations of Cassette^{START} and Cassette^{STOP} to define the boundaries of conjugation (FIG. 1B, panel ii) and completed with the introduction of the pAra-Trans (FIG. 1, panel iii). The recipient cell is defined with the introduction of pAra-CAST with a specific crRNA (FIG. 1B, panel iv). Upon induction of pAra-Trans, donor DNA is conjugated from Cassette^{START} to Cassette^{STOP} into the recipient cell (FIG. 1B, panel v). This conjugated DNA transiently circularizes in the recipient cell (FIG. 1B, panel vi) before it is subsequently integrated in a CAST and crRNA dependent manner, facilitated by the R-TE and L-TE sequences just within the boundaries of the transferred DNA (FIG. 1i, panel vii). Selection of the gain and loss of defined antibiotic markers enables selection for the desired transfer starting at Cassette^{START} and terminating at Cassette^{STOP} Alternative transfer starting at Cassette^{STOP} and terminating at Cassette^{START} is theoretically possible but is selected against and does not yield a viable intermediate for subsequent integration with the correctly positioned R-TE and L-TE. CASTs have been described to carry out bidirectional transposition, leading to two possible integration orientations, right-to-left (R-L) and left-to-right (L-R) orientations (FIG. 1, panel vii).

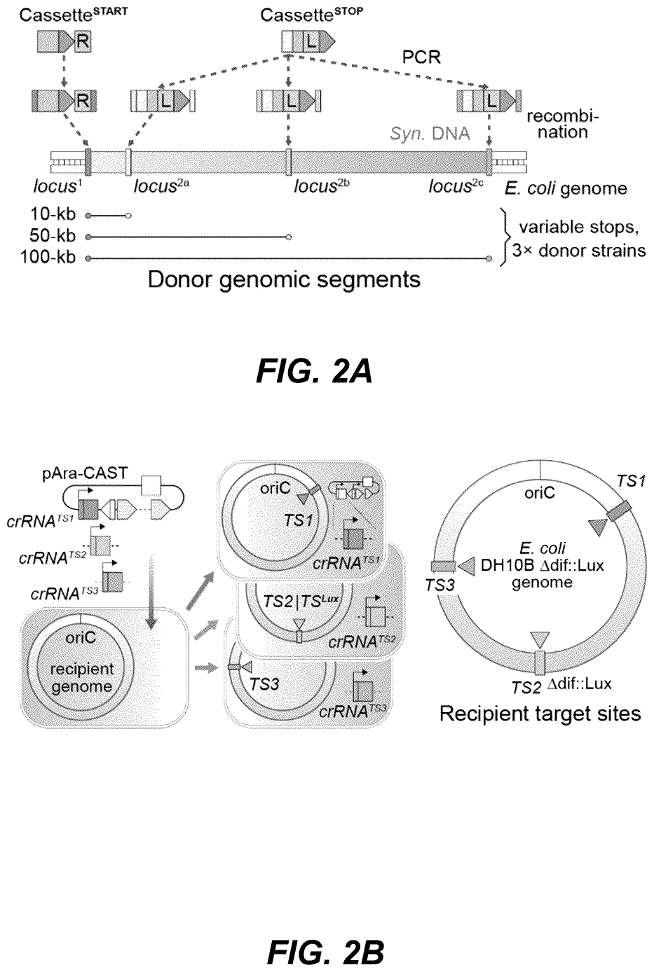

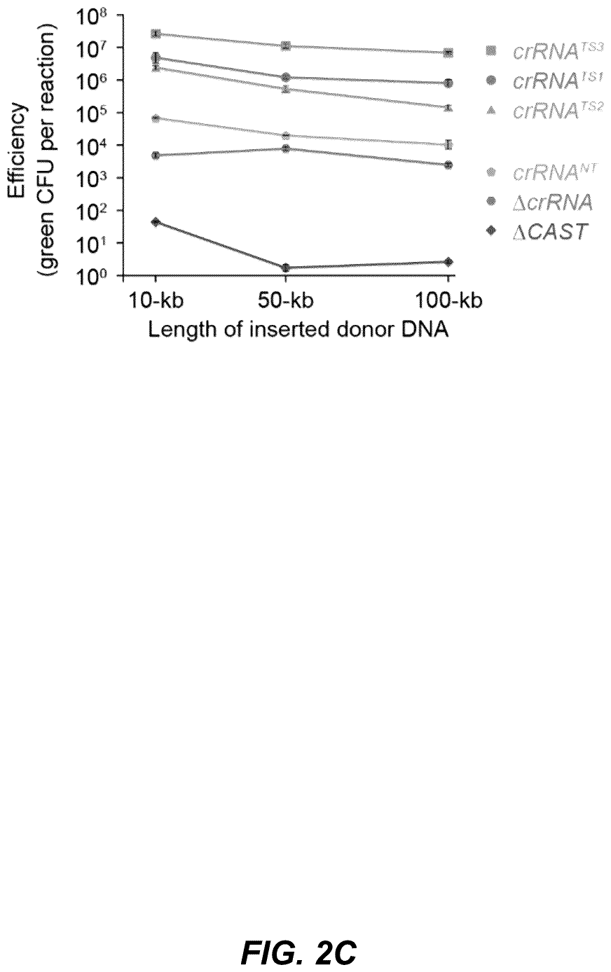

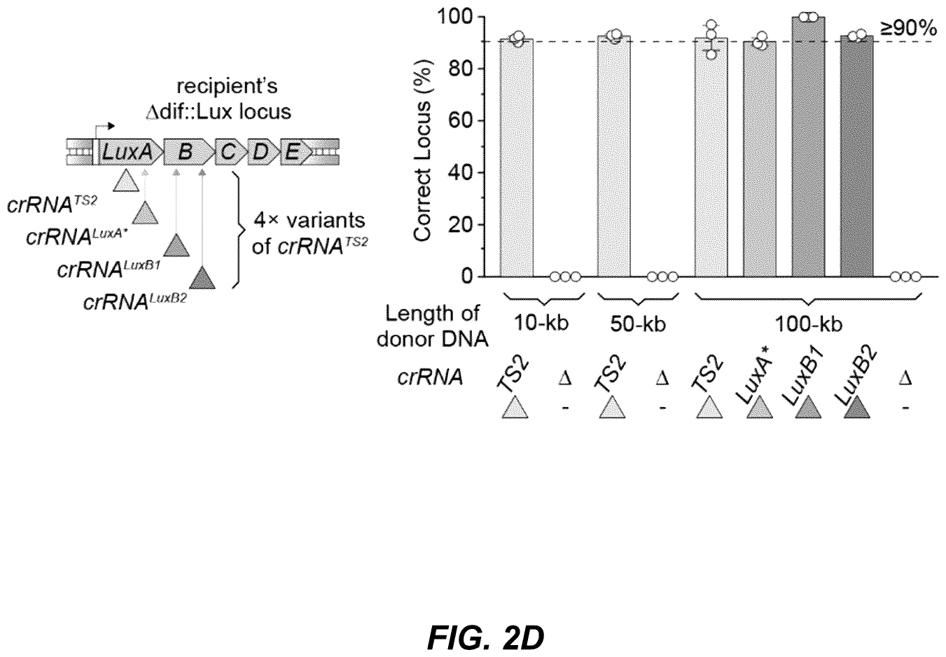

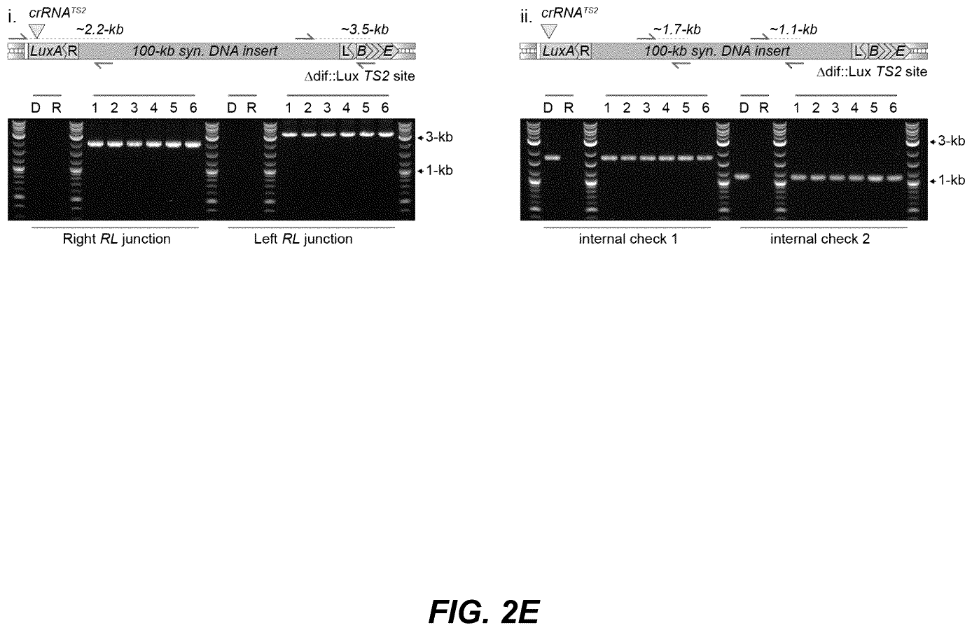

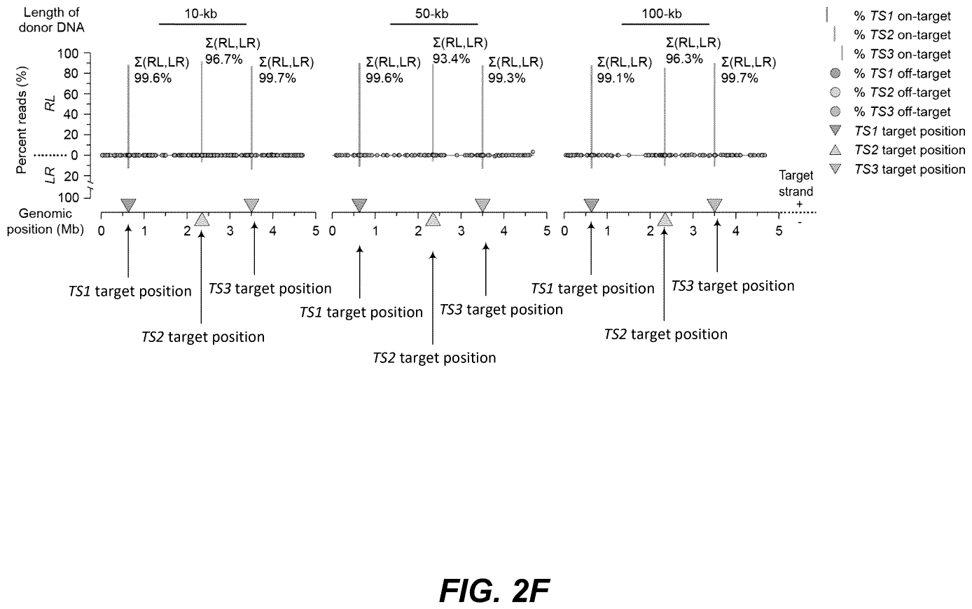

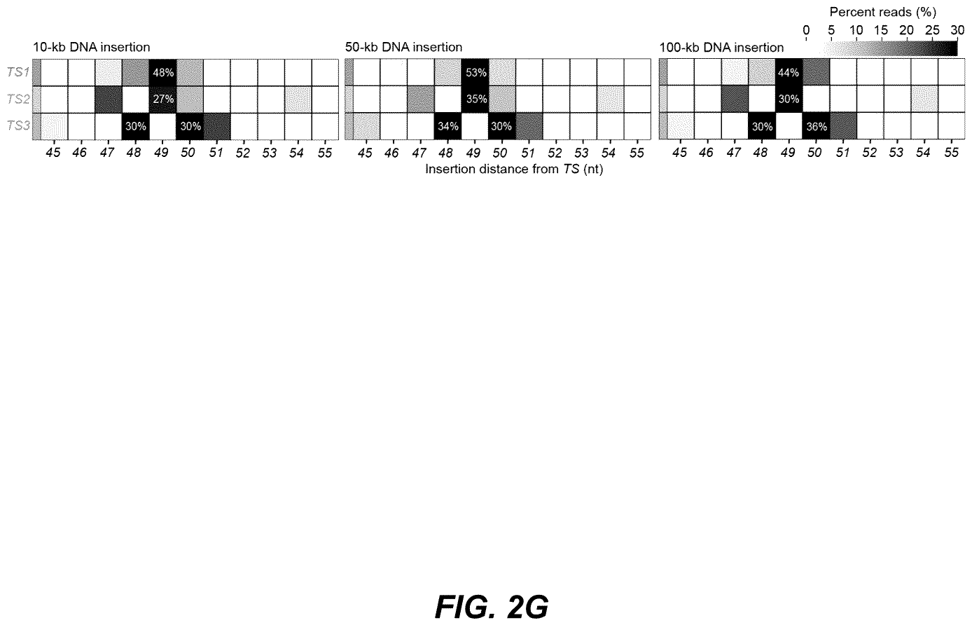

We first validated ACE to transfer short, synthetic donor sequences up to 100 kb. To minimize any potential interference by homologous recombination, we first constructed the E. coli DH10B-Syn100ΔlacZ strain as the donor strain which harbors a 100-kb synthetic DNA sequence Syn100 bearing no homology to E. coli genomic sequence (FIG. 2A). ACE was accomplished with three distinct lengths of Syn100 (10 kb, 50 kb and 100 kb) transferred from the donor to one of three target sites (TS1, TS2|TS^{lux} and TS3) into the recipient E. coli DH10B (Δdif::lux) strain (FIG. 2A-FIG. 2B). TS1 and TS3 were arbitrarily chosen on opposing sides relative to the position of the origin of replication (oriC), while TS2|TS^{lux} (referred from now on as TS2) was situated to target a luxABCDE luminesence operon replacing the original dif site (Δdif::lux) in the terminus region of the chromosome. Integrations at TS2 lead to loss of chemiluminescence in this recipient strain as it knocks out the luxA gene in the lux operon.

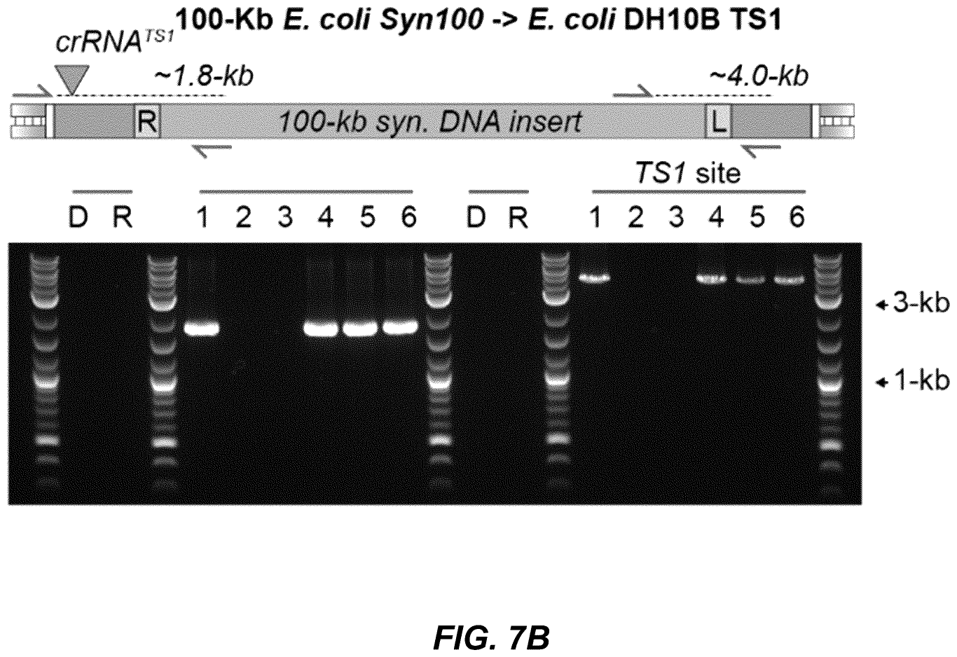

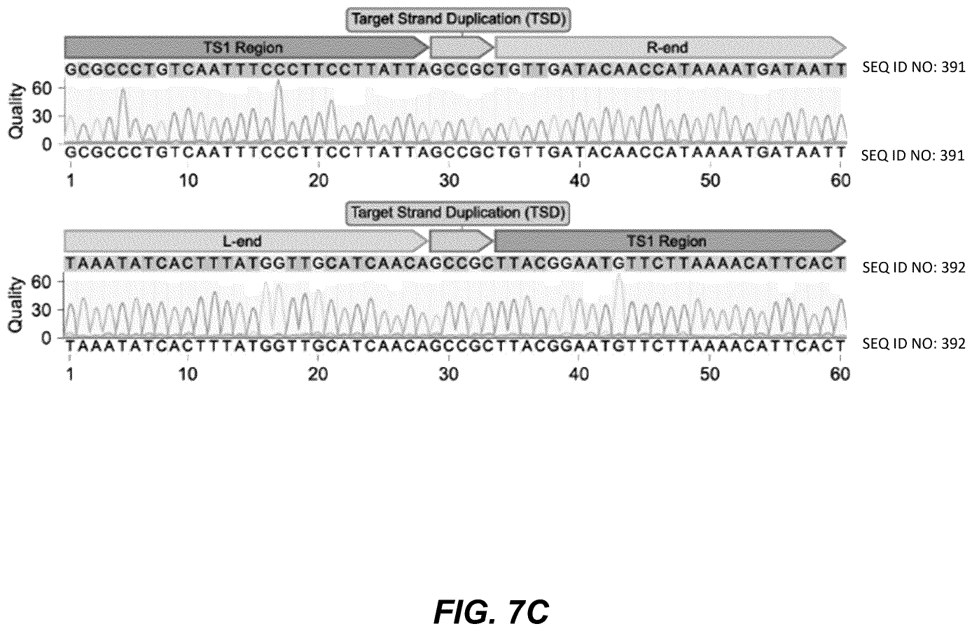

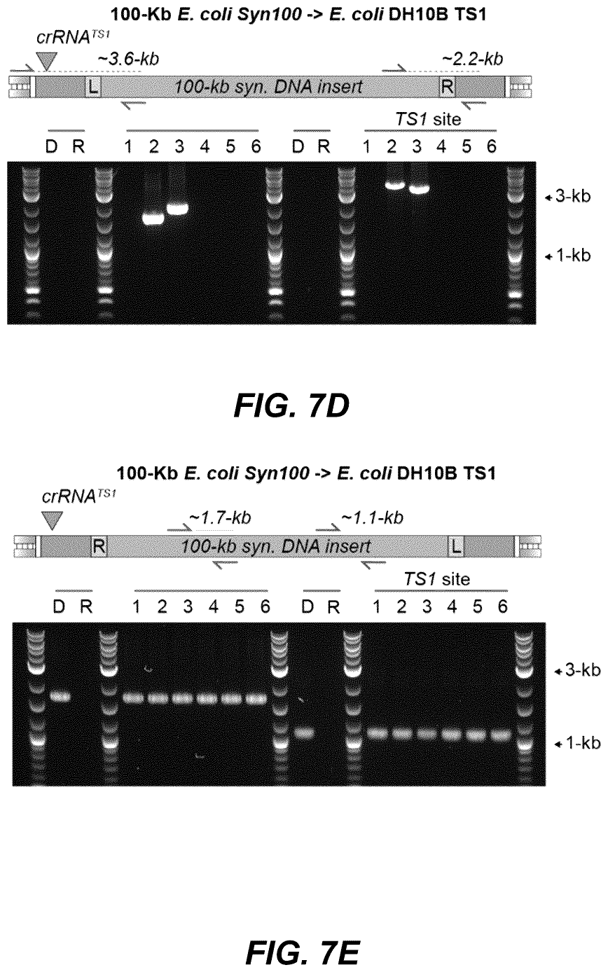

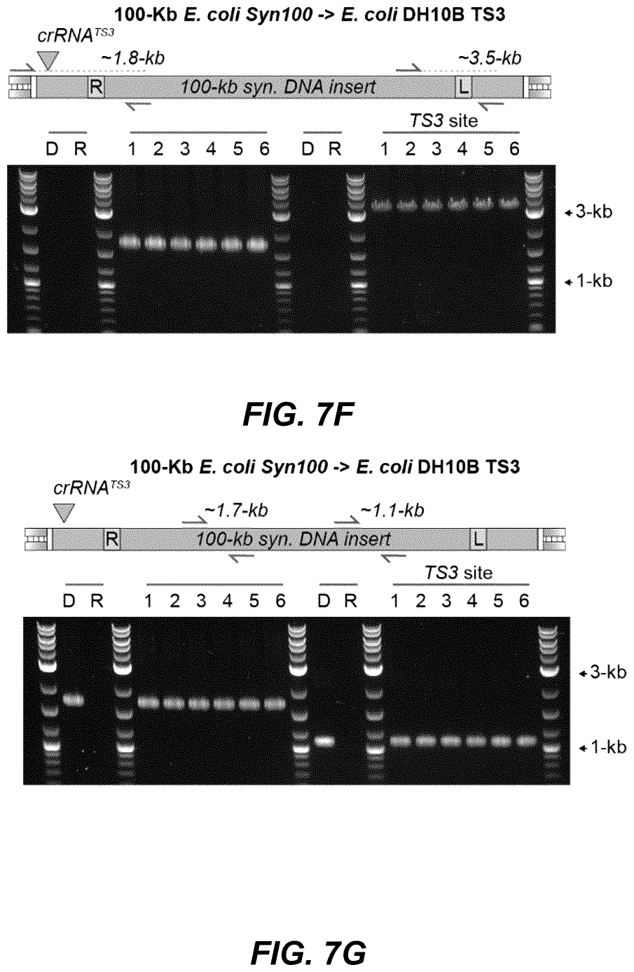

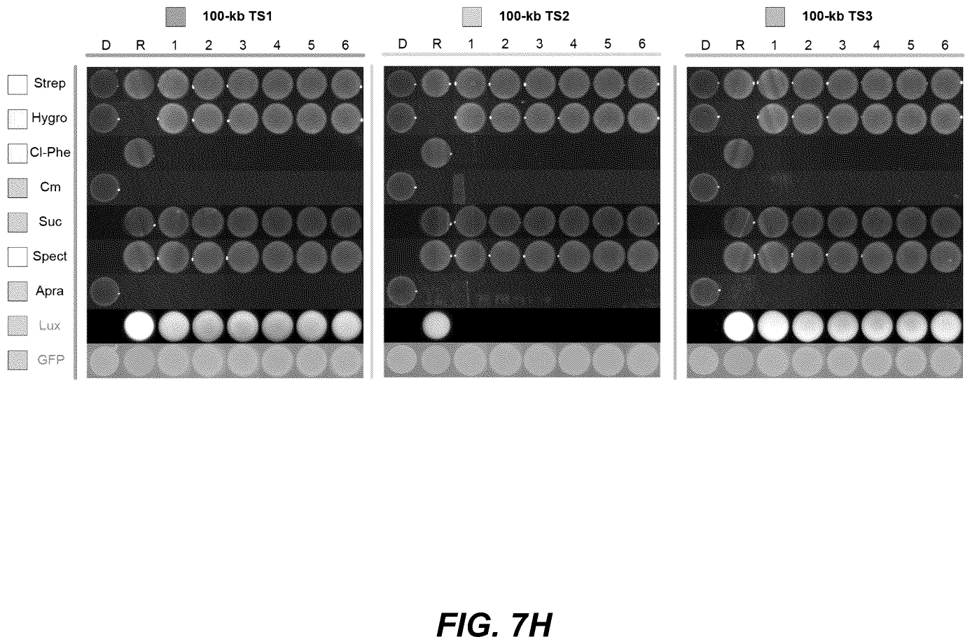

ACE transfers of the Syn100 are stable, specific, and efficient. With the presence of corresponding crRNAs (crRNA^{TS1}, crRNA^{TS2}, crRNA^{TS3}) integration efficiencies at all target sites were high, ranging from 10⁶ to 10⁸ GFP+c.f.u. per experiment for all sizes transferred (FIG. 2C). With non-targeting crRNA (crRNANT) or absence of the crRNA (ΔcrRNA), the c.f.u. was dramatically reduced by several magnitudes to 103 to 105, and further reduced to almost zero in the absence of CAST components (ΔCAST) (FIG. 2C). To demonstrate the precision of ACE, an in vivo screen was performed leveraging concomitant loss of recipient chemiluminescence with gain of donor cargo fluorescence when integrating into the Δdif::lux at TS2 to assay for on-target transposition (FIG. 2D, FIG. 7A). For all transferred lengths, a >90% ACE on-target rate was observed, consistent with previous assessments of Tn6677 CAST specificity at shorter transfer size less than 10 kb. This assay was repeated with three additional lux-targeting crRNAs (crRNA^{luxA*}, crRNA^{luxB1} and crRNA^{luxB2}) with the 100-kb donor to control for variations caused by individual crRNA specificity (FIG. 2D). All measured on-target rates were consistently high. We further verified the success of ACE events and the stable presence of the imported Syn100 DNA at the correct target site by genotyping PCR of the junctions and multiple internally distributed segments across the length of Syn100. (FIG. 2E and FIG. 7B-FIG. 7D). The junction PCRs also uncovered an R-L integration bias for these donors for all target locations in agreement with previous characterizations of Tn6677 CAST system at shorter transfer size (FIG. 2E and FIG. 7B-FIG. 7D, FIG. 7F).

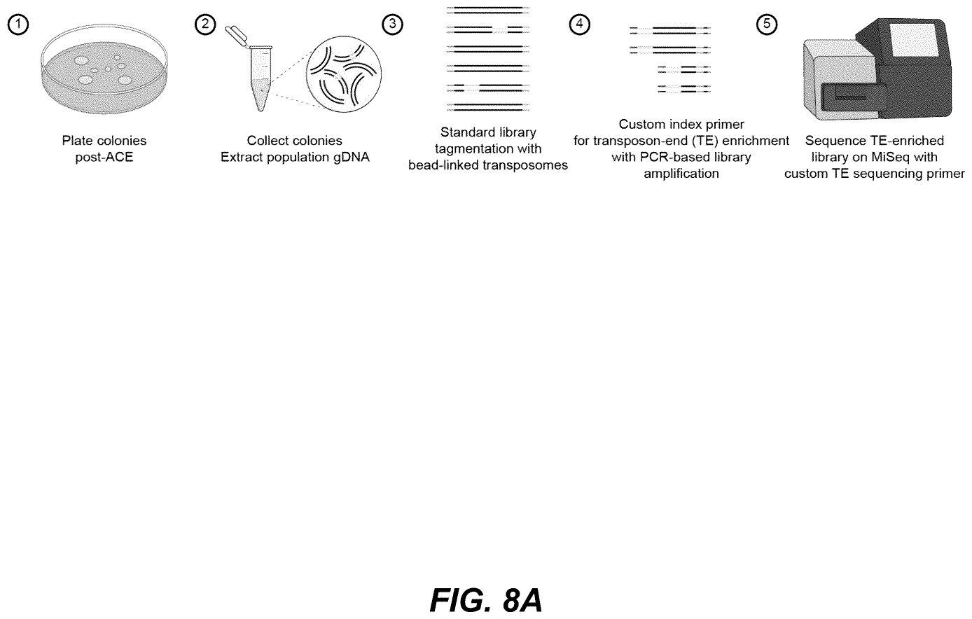

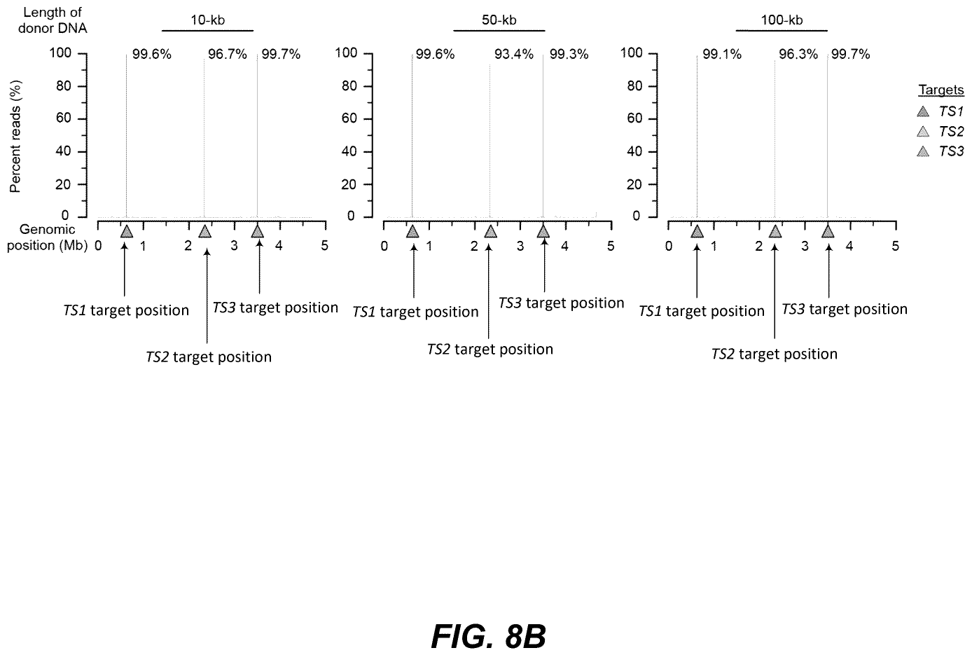

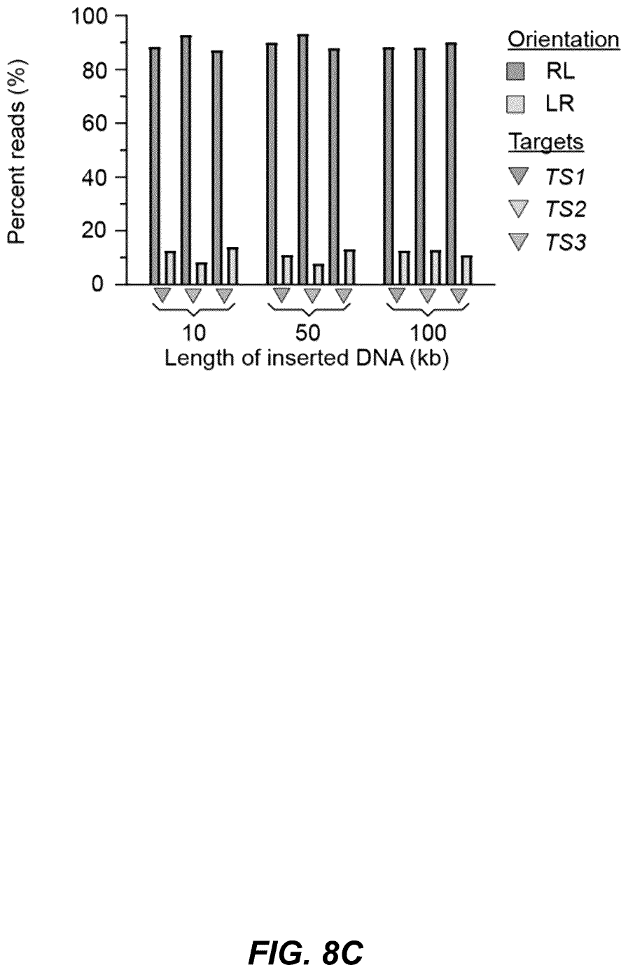

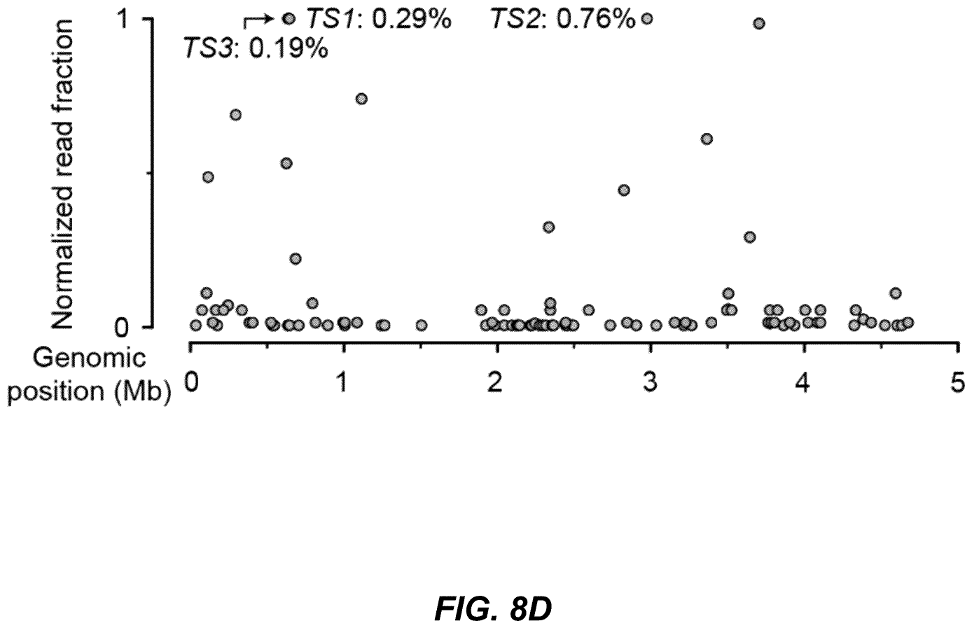

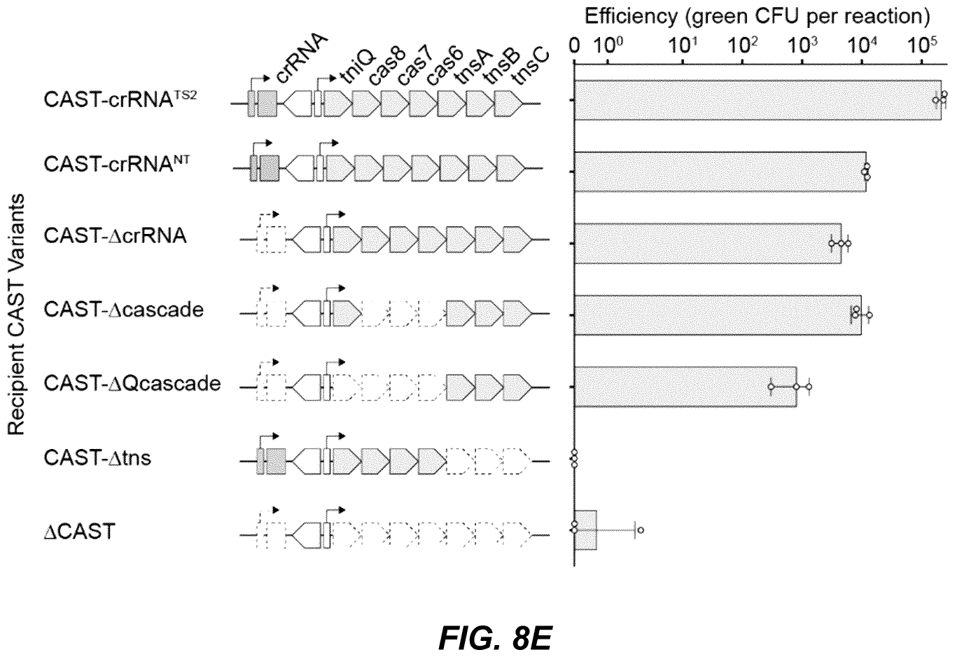

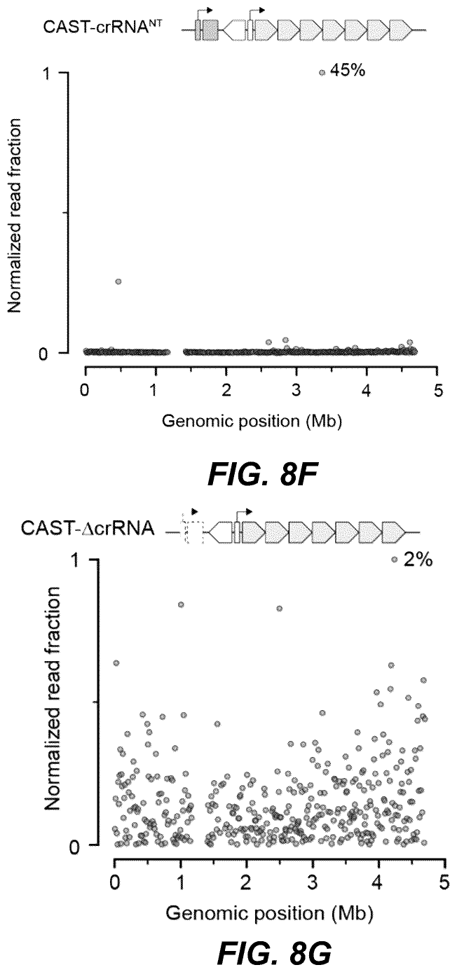

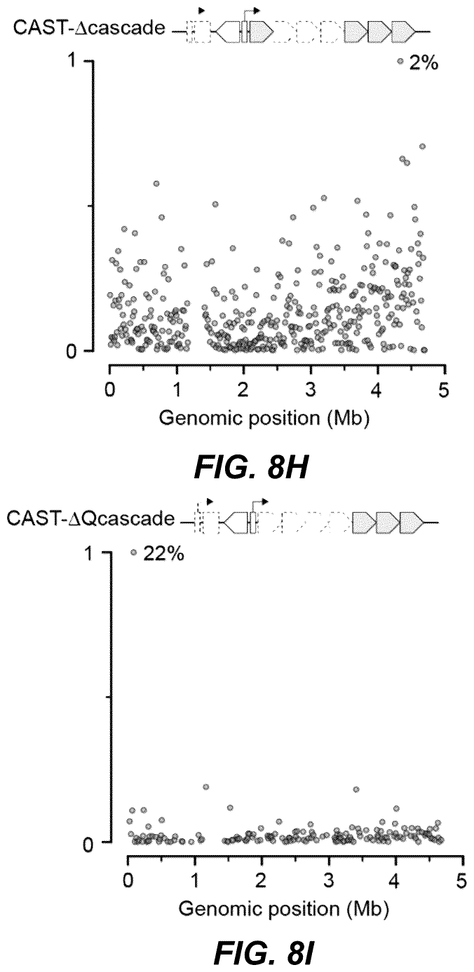

To both quantify the observed R-L/L-R bias and improve the resolution on the on-target ACE results, a tagmentation-based transposon-sequencing (Tn-Seq) protocol was developed for genome-wide interrogation of all on and off-target ACE events (FIG. 8A). Tn-Seq of Syn100 10-kb, 50-kb and 100-kb transfers into TS1, TS2 and TS3 reveal on-target rate ranging from 93.4% to 99.7%, consistent with our in vivo lux screen on TS2 (FIG. 2F, FIG. 2D and FIG. 8B and FIG. 8D). The data also corroborates the significant R-L integration bias for all lengths transferred and locations targeted (FIG. 2F and FIG. 8C). Each target had an integration distribution around the expected 49 bp insertion distance that remained constant over all tested donor sizes (FIG. 2G).

ACE efficiency and specificity necessitate the presence of both crRNA and CAST components. The ability for unbiased detection of transposition throughout the genome afforded by Tn-Seq motivated us to attempt Syn100 transfers with CAST variants containing deletions of key modules. Around a 10-fold decrease in efficiency was observed for Syn100 transfers using CAST-crRNA^{NT}, CAST-ΔcrRNA and CAST-Δcascade and nearly a 100-fold decrease in efficiency for transfers using CAST-ΔQcascade when compared to CAST-crRNA^{TS2} (FIG. 8E). Transfers employing CAST-Δtns or ΔCAST abrogated nearly all activity, demonstrating the importance of the tnsABC module for integration (FIG. 8E). Tn-Seq of these transfer experiments showed different patterns of genome-wide non-specific integrations (FIG. 8F-FIG. 8I).

### Demonstrating ACE at Mega-Base Scale and the Importance of Genome Architecture

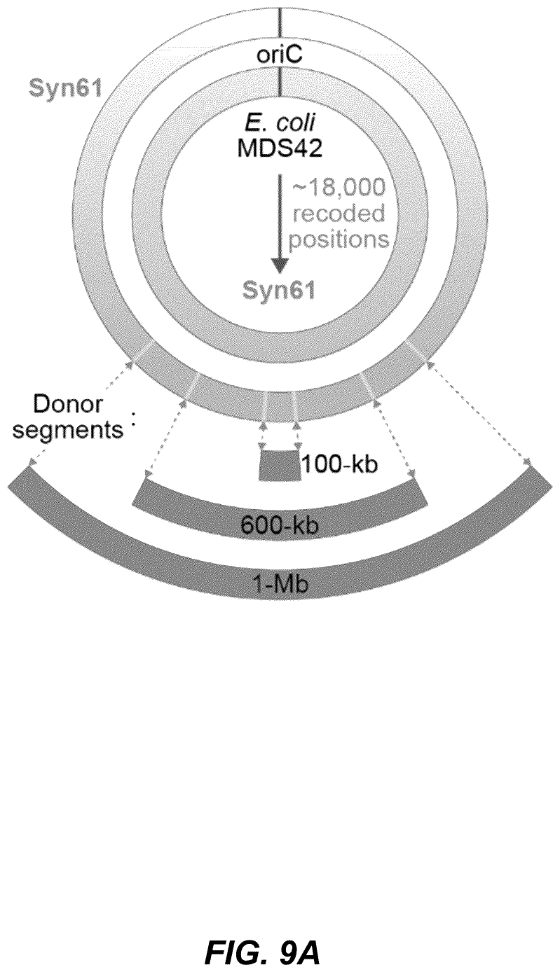

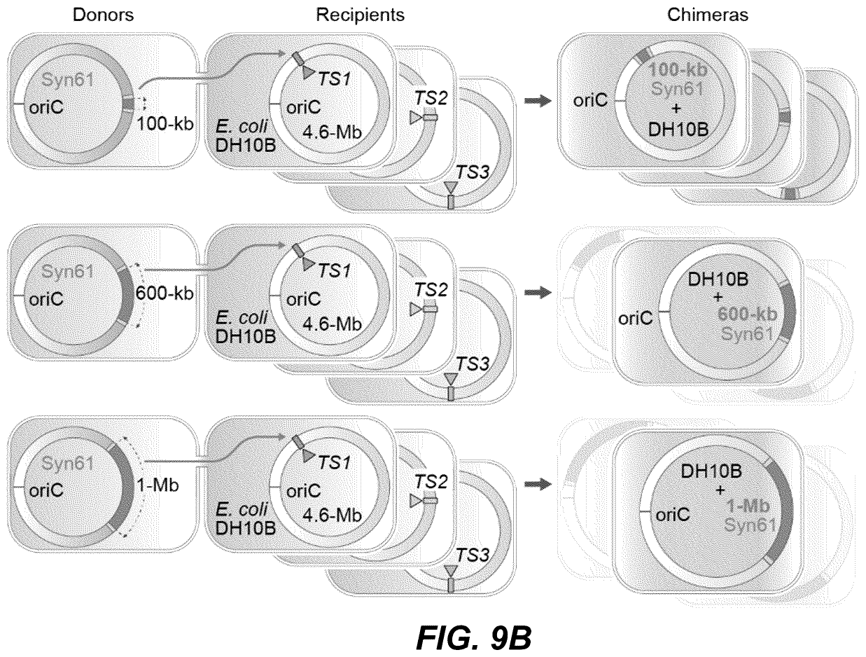

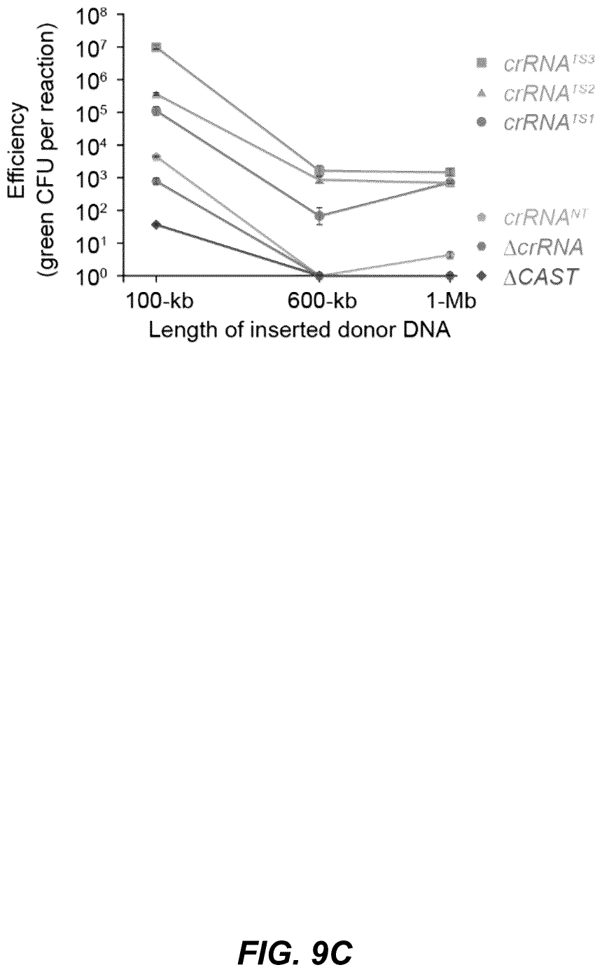

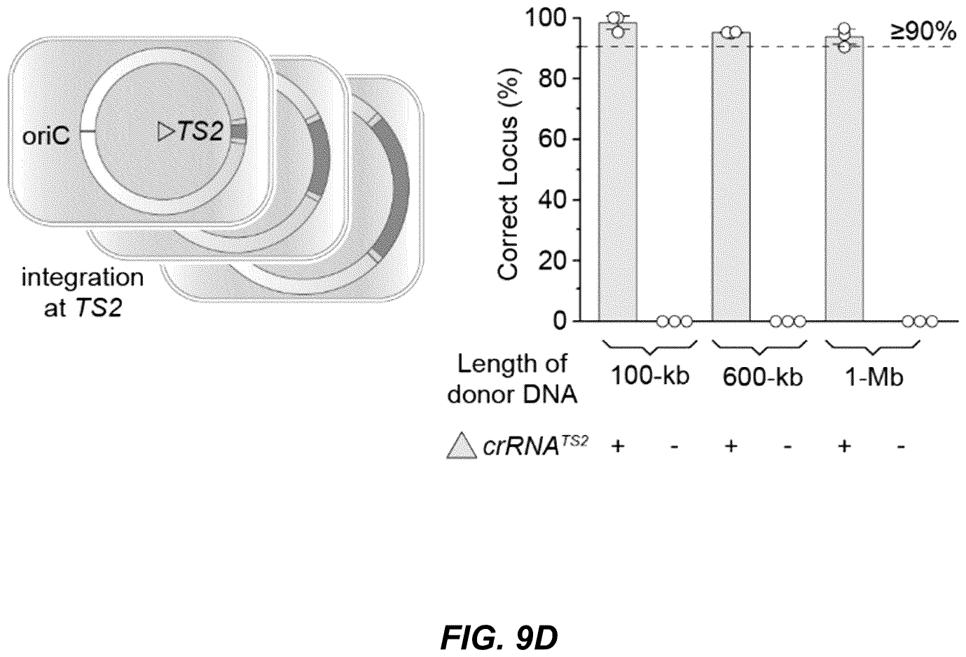

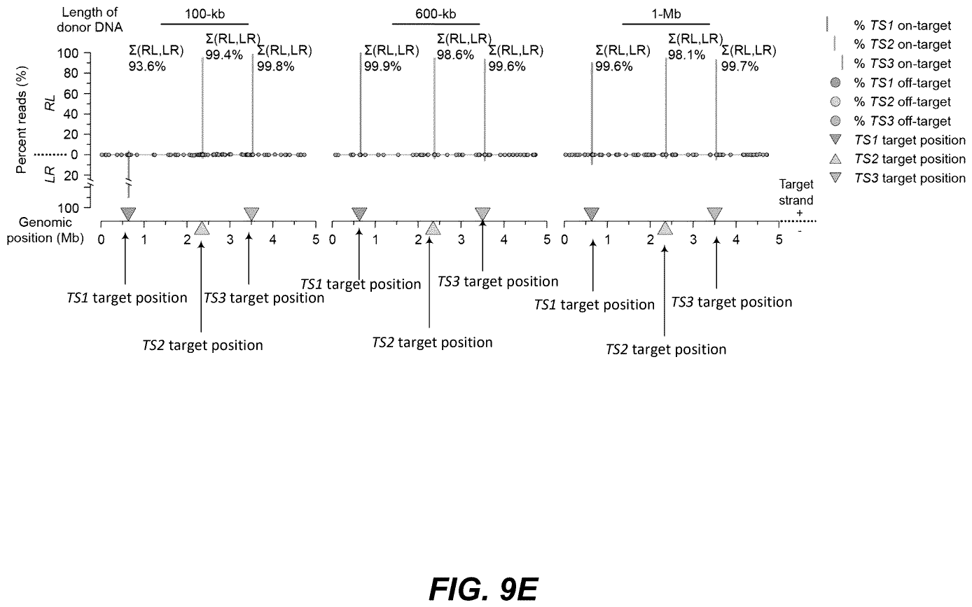

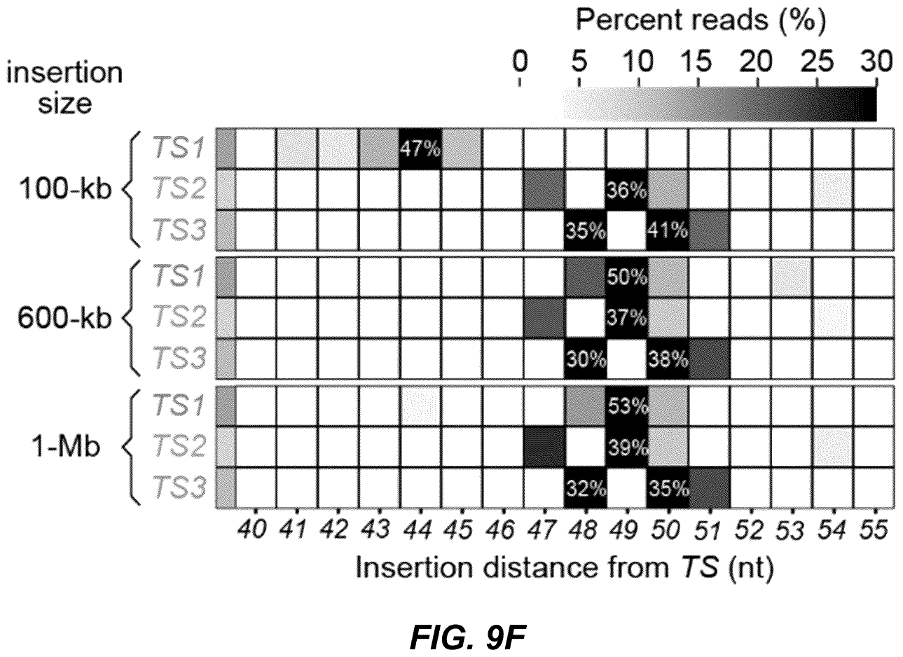

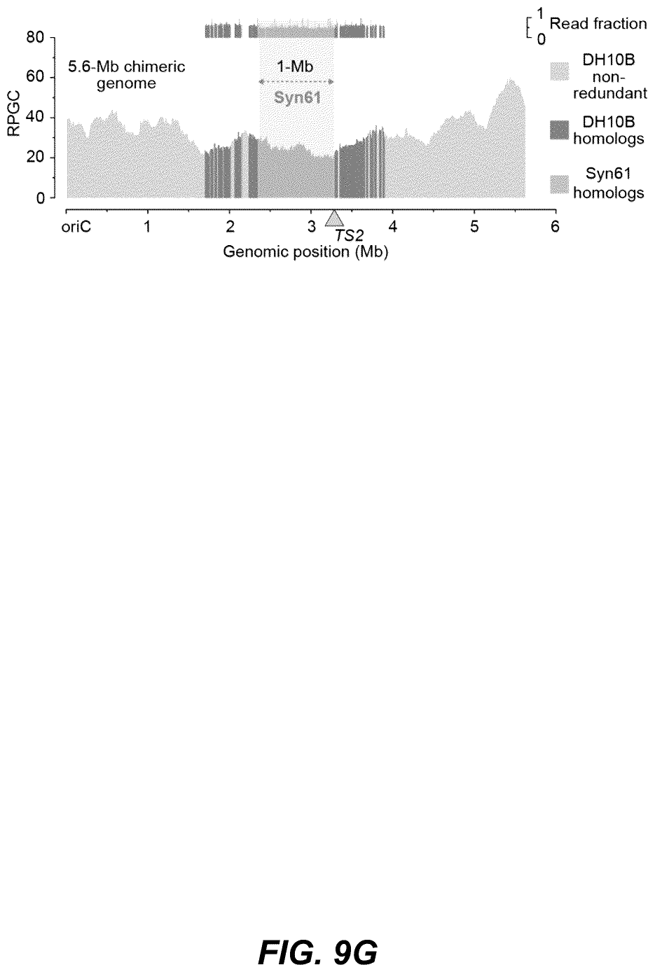

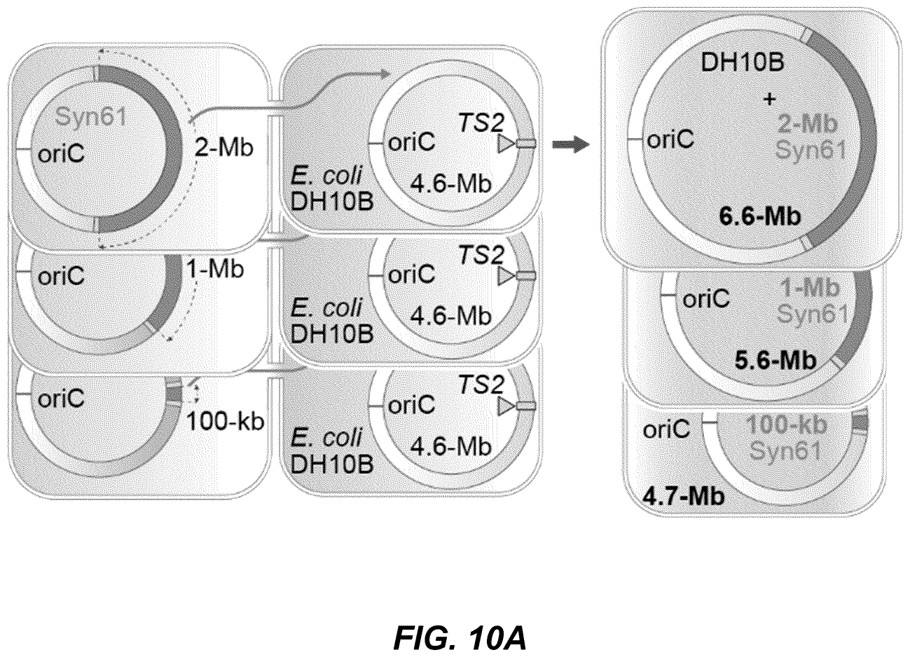

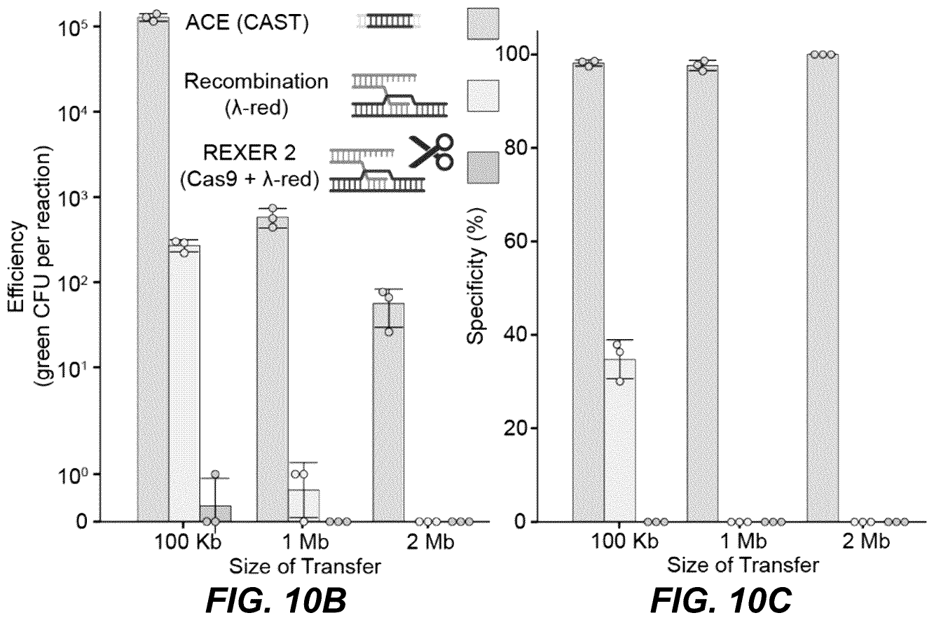

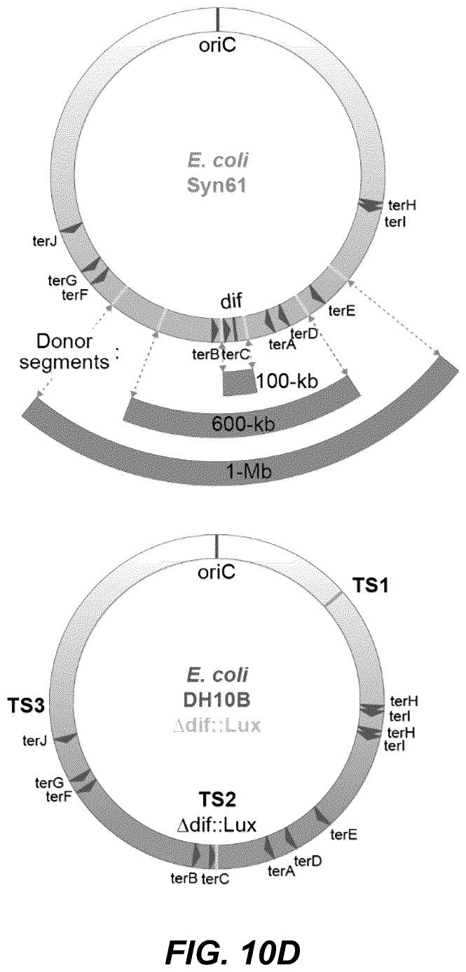

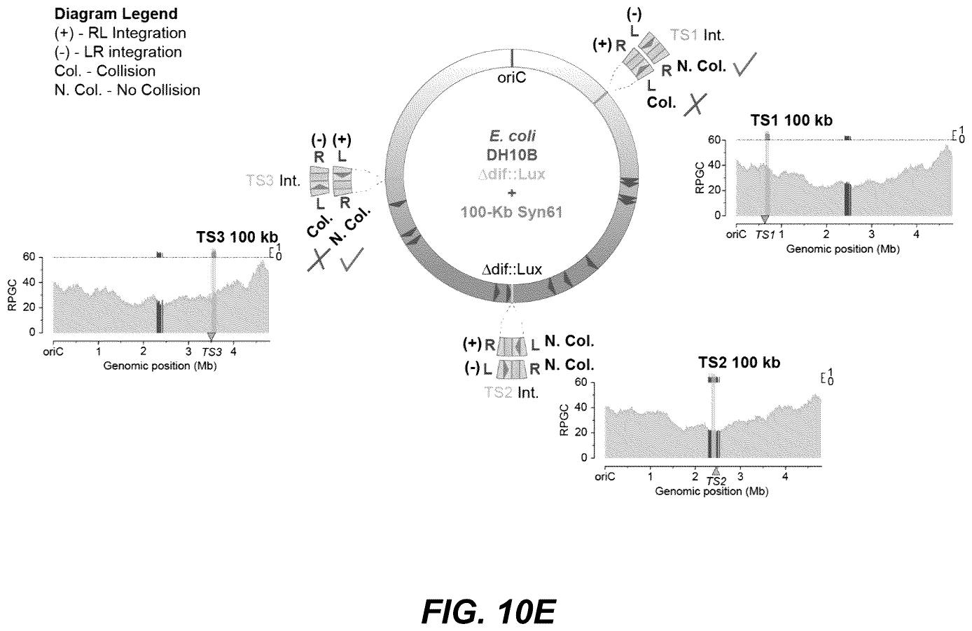

The success of ACE motivated us to further scale up donor size to megabase-scale. The same E. coli DH10B (Δdif::lux) strain was used as the recipient with crRNA^{TS1}, crRNA^{TS2}, or crRNA^{TS3} defining the integration site at TS1, TS2, or TS3. The synthetic recoded E. coli Syn6l was chosen as the donor strain for its ˜18,000 SNPs in the form of recoding and refactoring of genes throughout its genome, enabling us to reliably differentiate between the incoming donor segment and the recipient genome upon whole genome sequencing. Syn61 donors were constructed for transfer of 100-kb, 600-kb and 1-Mb genomic DNA with Cassette^{START} and Cassette^{STOP} spaced roughly equidistant from the terminus region (FIG. 9A). Three different donor sequences together with three different integration sites into the recipient genomes can yield nine possible chimeric genomes (FIG. 9B). 10⁵-10⁷ GFP+c.f.u. was observed for transfers of the 100-kb donor segment into various parts of the genome. The efficiencies dropped to 10²-10³ GFP+c.f.u. for transfers of the 600-kb and 1-Mb donor segments (FIG. 9C). As with the Syn100 donor transfers, crRNA^{NT} or ΔcrRNA controls showed a 10² to 10³-fold decrease in GFP+c.f.u., suggesting a low off-target rate. At all lengths, ΔCAST controls produced close to zero GFP+c.f.u. (FIG. 9C). Notably, comparable methods of achieving large genome additions by recombination fail to attain the efficiency and specificity of ACE, especially for larger megabase-sized insertions (FIG. 10A-FIG. 10C). Observed genome-wide on-target rates were consistently high for ACE at megabase scale as measured by both in vivo chemiluminescence screen at TS2 (FIG. 9D) and Tn-Seq at all three target positions (FIG. 9E).

R-L integration bias was again observed for all lengths and locations transferred, except in the unique exception of 100-kb donor segment into recipient genome position TS1, which had a predominantly flipped L-R integration bias (FIG. 9E). Examination of the 100-kb donor segment reveals the presence of a single ter site, terC. Ter sequences situate throughout the terminus region of the E. coli genome and have been implicated in the termination of genome replication by forming unidirectional replication fork traps when bound by the protein tus (FIG. 10D). Consistent with the required orientation of the ter sites, we realized that L-R integration of the 100-kb donor in TS1 orients the terC in that segment in alignment with the required DNA replication directionality, suggesting that R-L integration would lead to replication fork arrest at the non-permissive end of the inverted terC (FIG. 10E). This deviation from R-L integration bias for this condition was also accompanied by a different integration distribution centered around 44 bp (FIG. 9F). Otherwise, all other conditions had expected distributions in agreement with previous Syn100 measurements (FIG. 9F, in comparison to FIG. 2G).

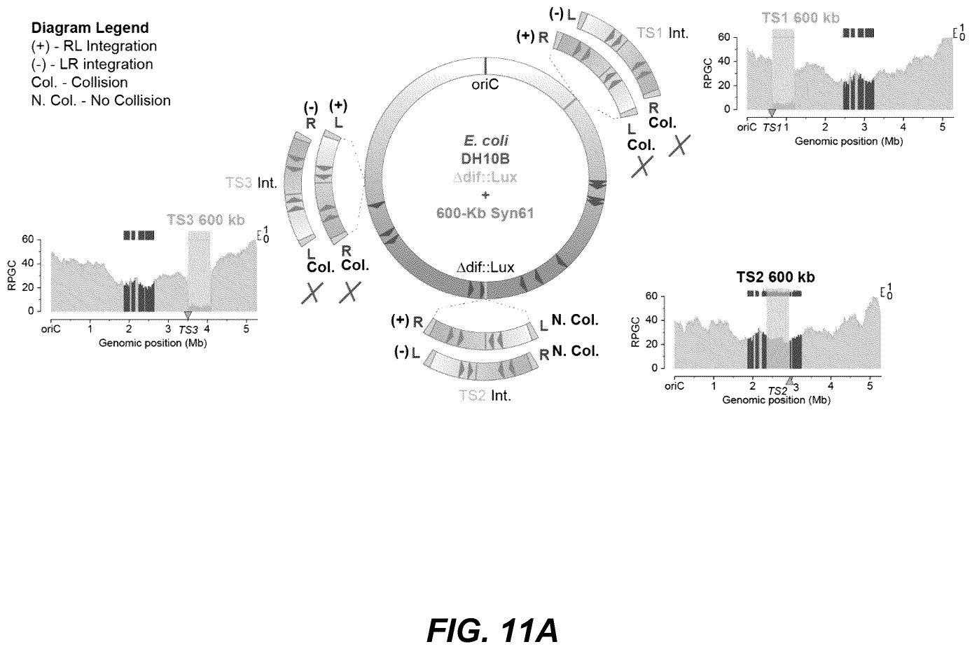

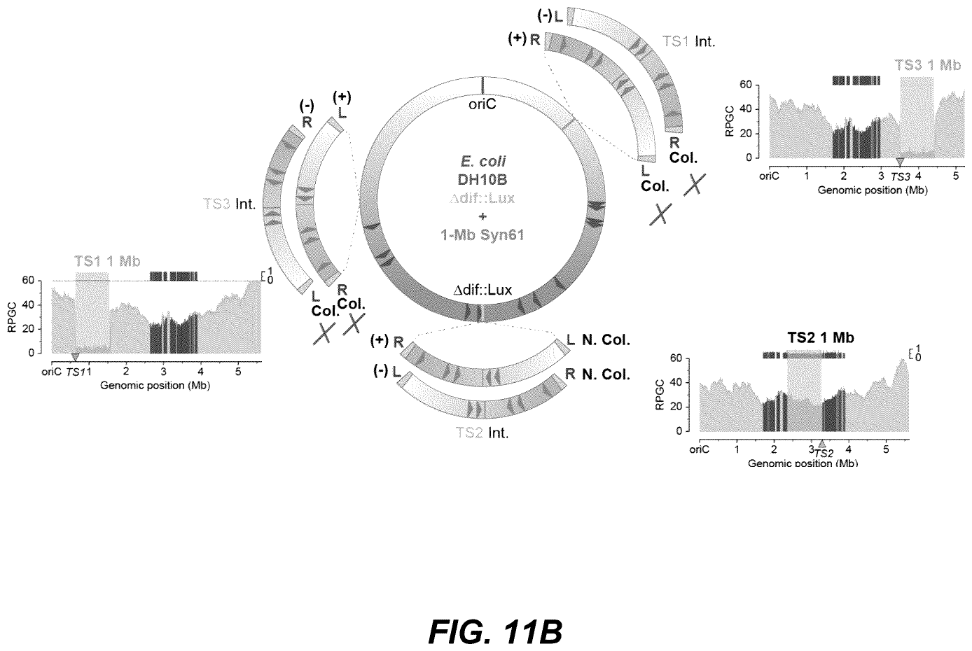

Successful ACE events are necessary but may not be sufficient for long-term stability of the fused chimeric genome. The stability of post-ACE clones was verified with next generation sequencing. Whole genome sequencing showcased the presence and integrity of the entire transferred segment in the correct location for 100-kb, 600-kb and 1-Mb Syn61 donor segments when integrated into the recipient's TS2 position (FIG. 9G and FIG. 10E, FIG. 11A-FIG. 11B). The 100-kb donor segment was also stable in the recipient's TS1 and TS3 genomic positions with the requirement of a properly oriented terC site. In contrast, the 600-kb and 1-Mb donor segments, centering the terminus region of the donor genome and both containing multiple ter sites facing opposite directions, were not maintained in the recipient genome upon integration into TS1 or TS3 positions (FIG. 10E and FIG. 11A-FIG. 11B). As in the case of R-L 100-kb TS1 integration, this observation is consistent with the hypothesis that integration of 600-kb and 1-Mb donor segments in either orientation presents the post-ACE chimeric chromosome with numerous opposing ter sites orientated towards their non-permissive directions, trapping the advance of the replisome during normal chromosome replication (FIG. 11A-FIG. 11B). We thus noted the important distinction between on-target ACE integration and stable post-ACE maintenance of the donor DNA. While the Tn-Seq data shows that the transfers are generally on-target in all conditions, we infer, without being bound by any particular theory, that a majority, if not all, of those on-target integrations of donated segments with non-permissive architectures involve the subsequent loss of the whole or part of the internal donor segments possibly due to genomic instability from premature replication fork trapping.

The SNPs distributed in the coding sequences of the incoming Syn61 donor were utilized to differentiate its recoded genes from their wild-type DH10B homologs. Through this variant calling analysis, the identity of successfully integrated and stably maintained donor segments of 100-kb, 600-kb and 1-Mb Syn61 genomic segments at the recipient DH10B's TS2 target was confirmed, revealing general parity between the frequency of the wild-type DH10B genes and their recoded Syn61 homologs, each at their expected locations within the chimeric genome (FIG. 9G and FIG. 10E, FIG. 11A-FIG. 11B).

### ACE at Multi-Megabase Scale and Modularity Across the Entire Donor Genome

We next systematically explored the possibility of mobilizing DNA from all regions of the donor genome. To enable this, multiple Syn61 donors were engineered that each would mobilize a very large discrete segment of their synthetic genomes in an ACE event.

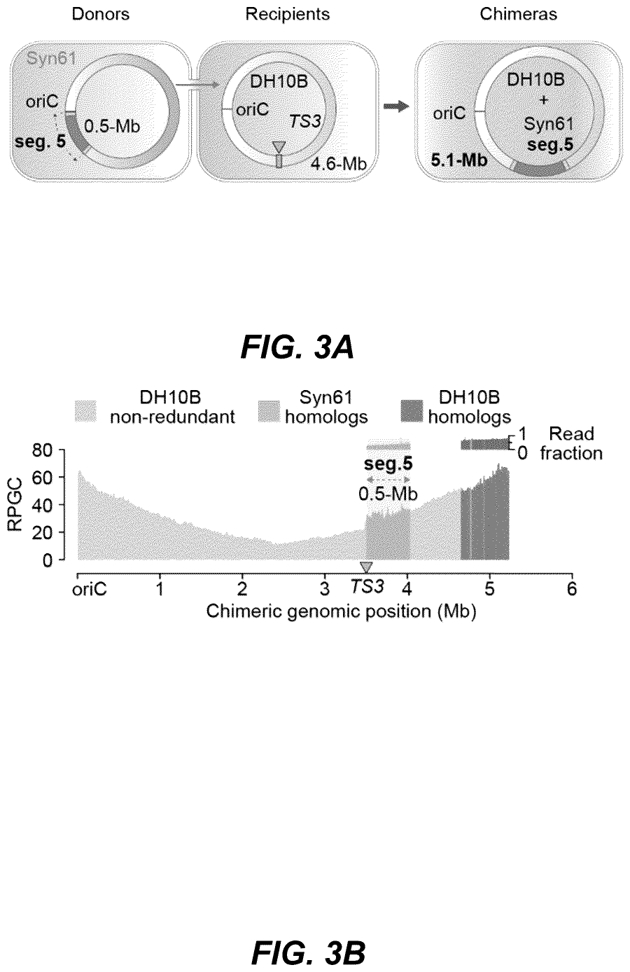

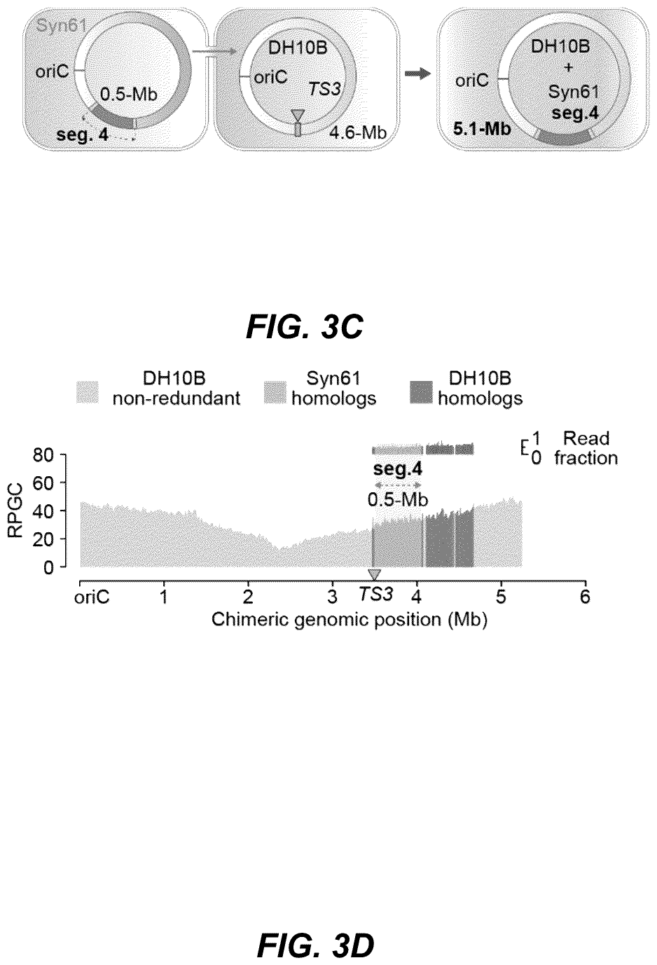

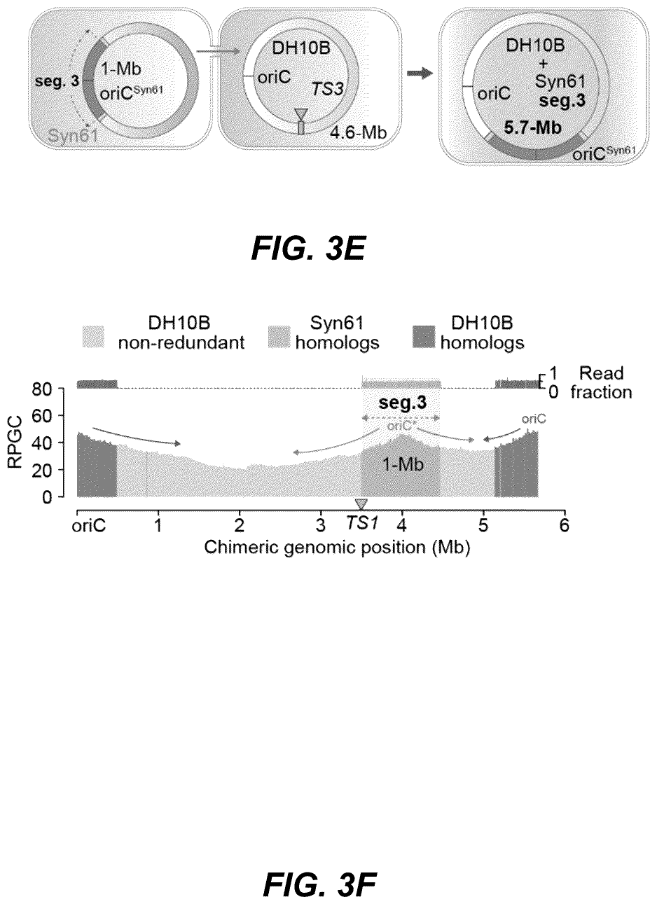

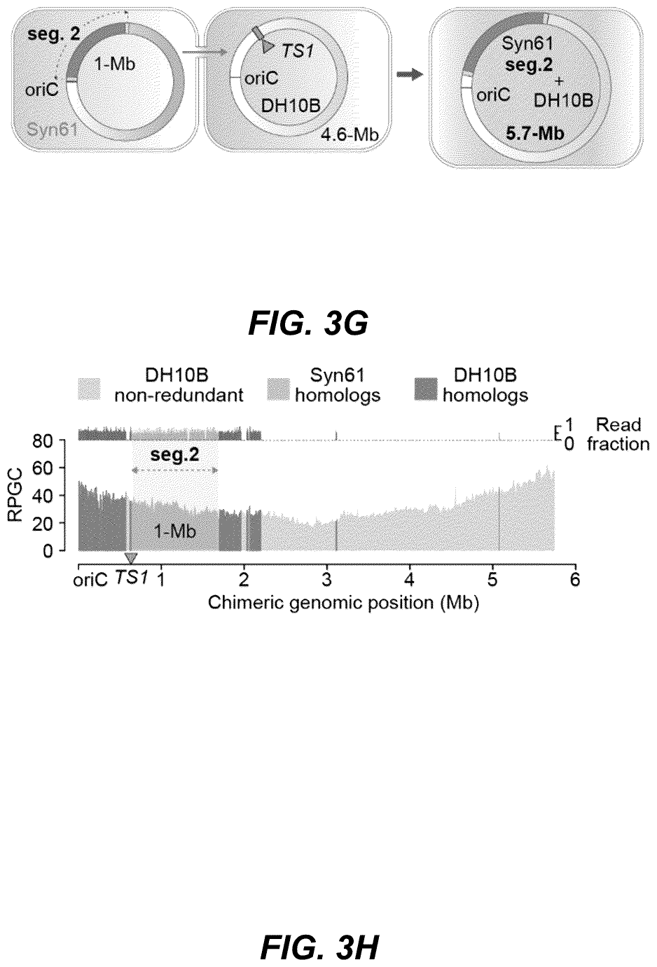

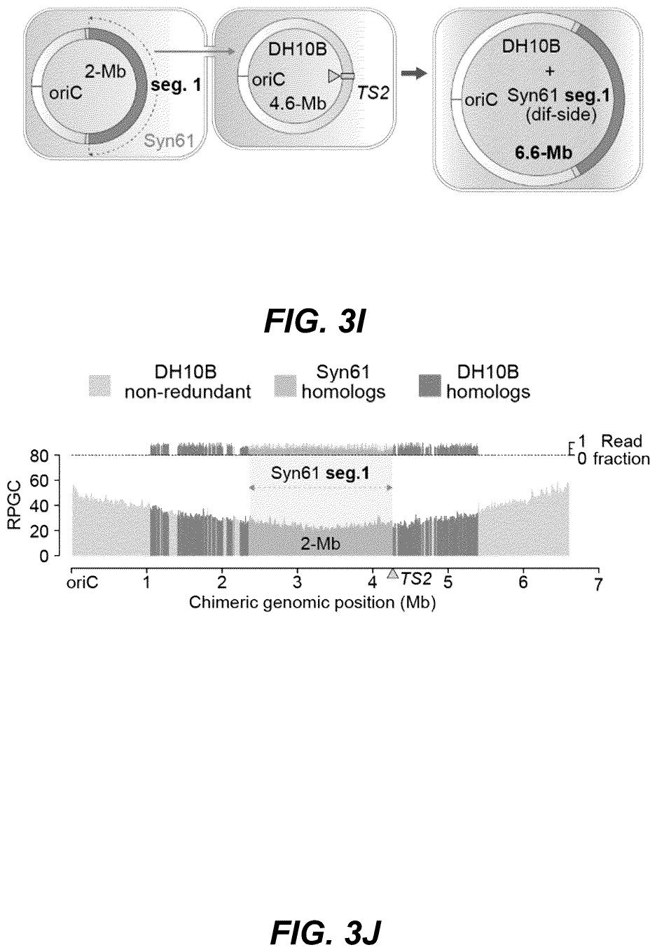

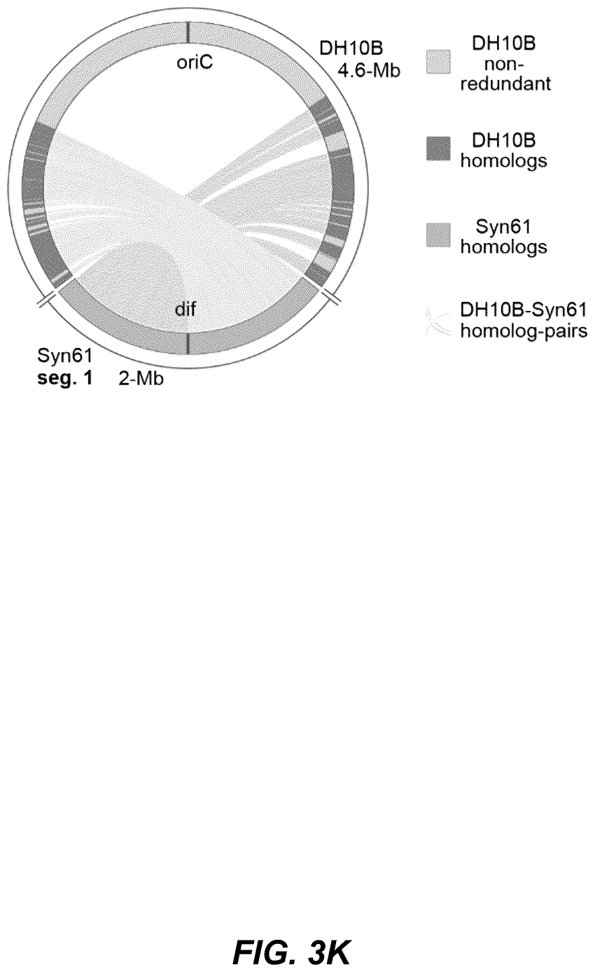

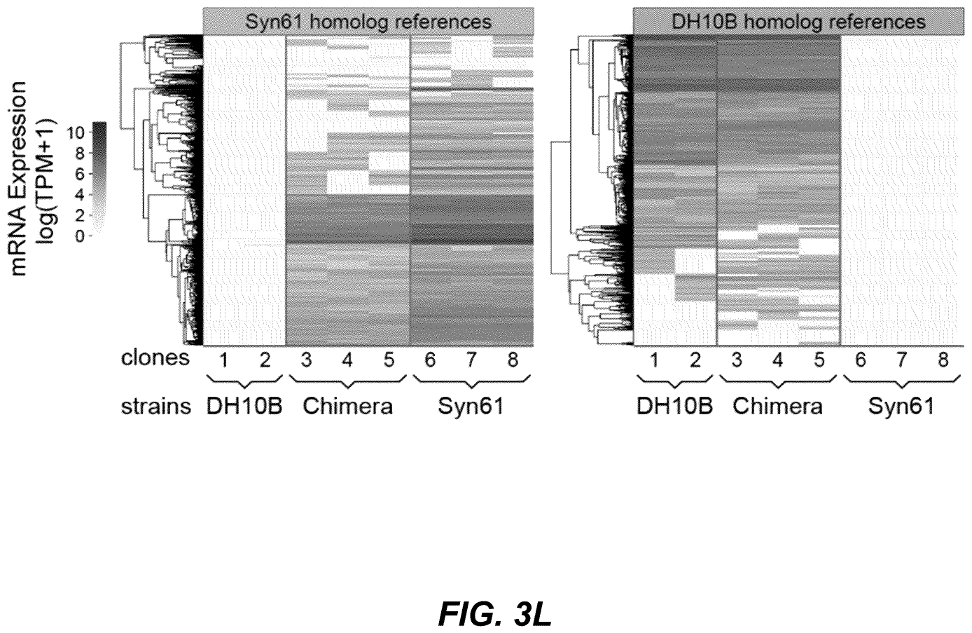

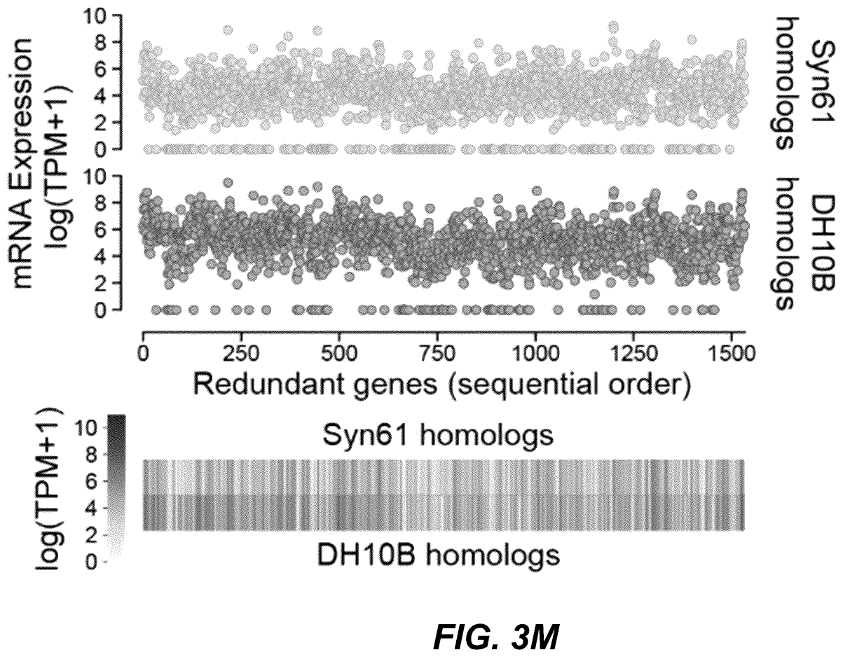

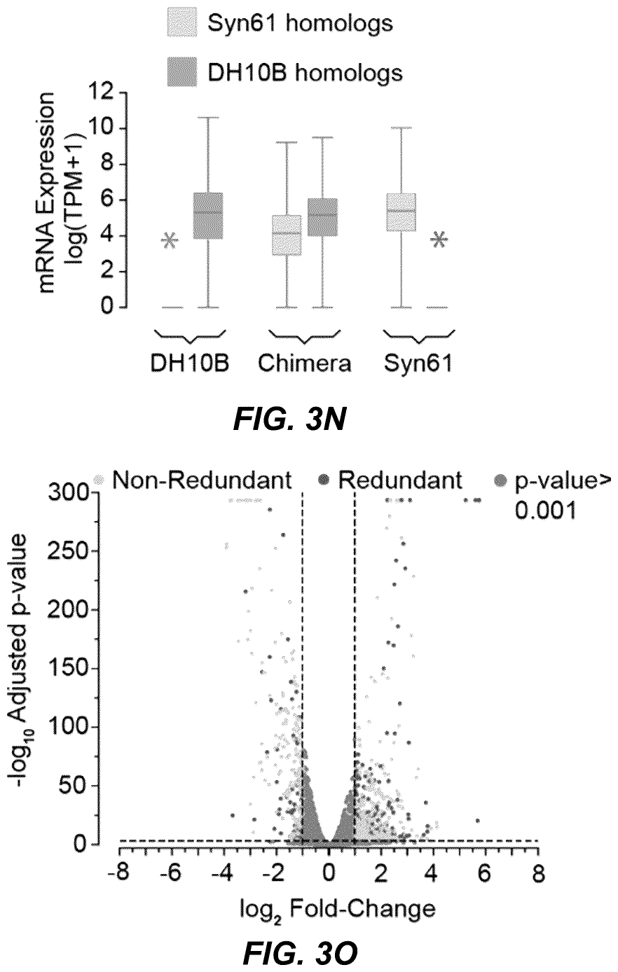

To push the limits of ACE, the first donor segment (seg. 1) spans 2-Mb, or half of its genome, centering around the dif terminus region (FIG. 3I). Donor seg. 2 covers 1-Mb segment downstream from the oriC, and seg. 4 and 5 covers two contiguous 500-kb segments upstream of oriC (FIG. 3A, FIG. 3C, FIG. 3G). An additional 1-Mb donor segment seg. 3 is defined spanning across oriC (FIG. 3E). Segments seg. 2, 3, 4 and 5 do not contain any primary ter sequences. Together, these donor segments span the entirety of the 4-Mb donor genome.

The integration of the 2-Mb seg. 1 donor segment into the recipient's TS2 genomic position would constitute an almost 50% expansion of the E. coli genome. Amazingly, this chimeric genome was characterized to be successful and stable via whole genome sequencing, (FIG. 3J). Efforts to integrate the same 2-Mb seg. 1 into the TS1 and TS3 sites failed to yield stable clones (data not shown). Without being bound by any particular theory, this may be due to the same forbidden genomic architecture with opposing directions of ter sites previously described.

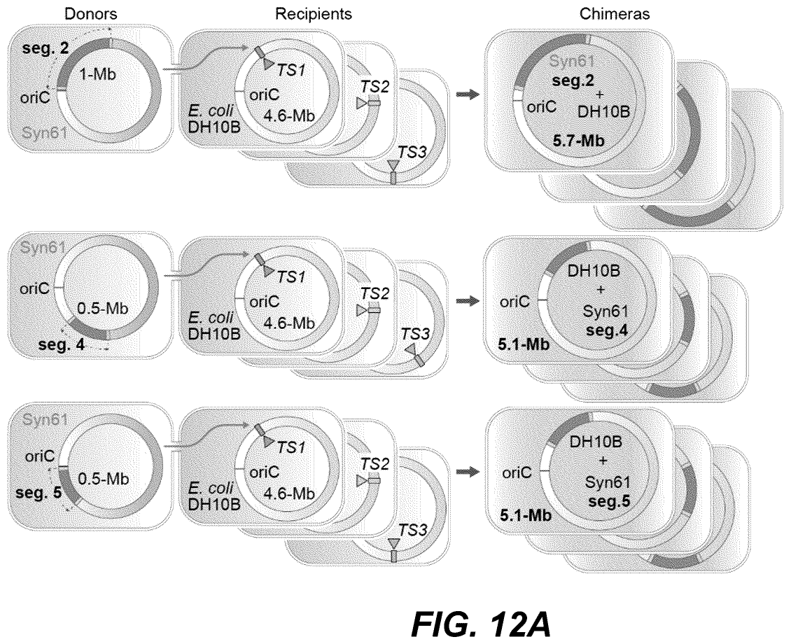

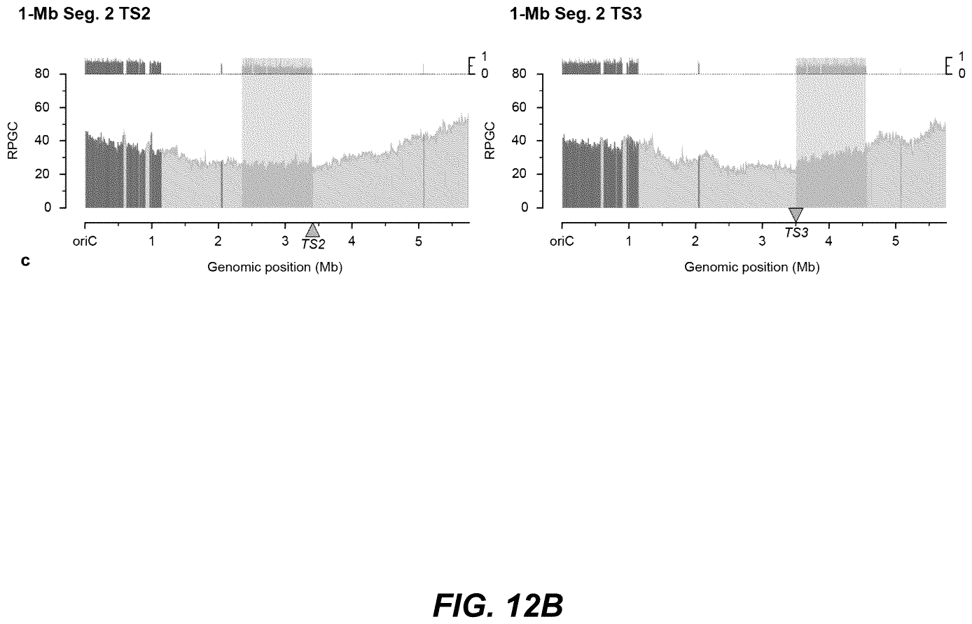

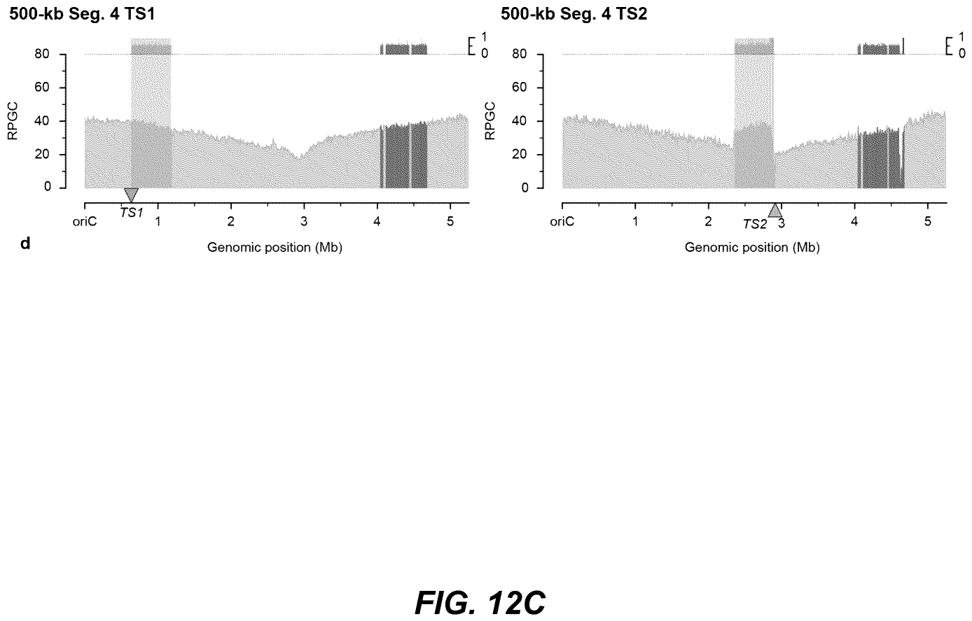

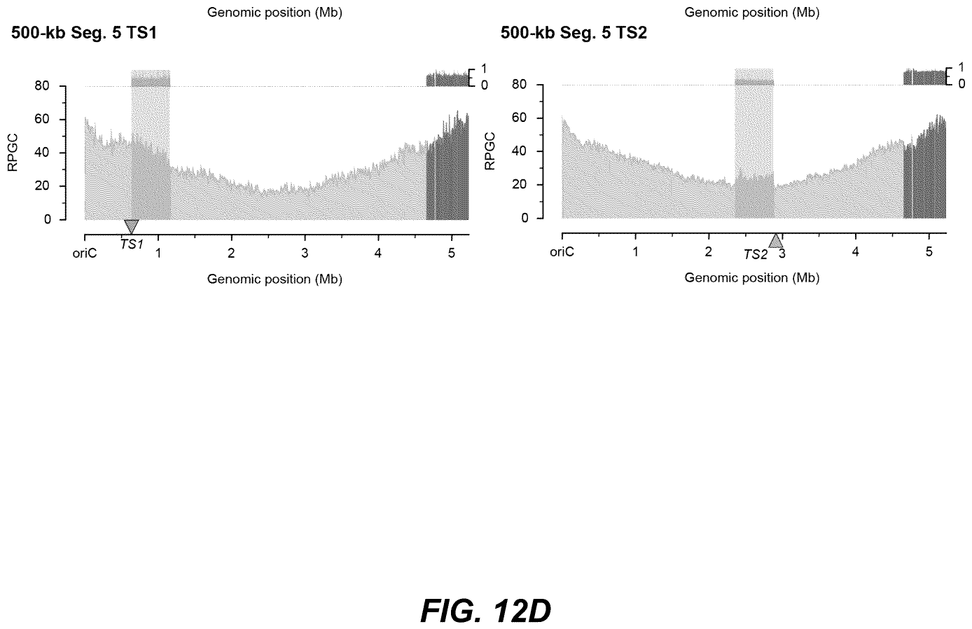

We tested the transfer of remaining distinct donor segments into recipient DH10B Δdif::lux genome at TS1, TS2 or TS3 positions (FIG. 3A-FIG. 3H and FIG. 12A). The 1-Mb seg. 2 can be stably integrated into all three sites in the recipient's genome (FIG. 3H, FIG. 12A-FIG. 12B). Moreover, the two separate 500-kb segments seg.4 and seg. 5 yielded stable post-ACE clones into all three sites in the recipient's genome (FIG. 3B, FIG. 3D and FIG. 12A, FIG. 12C-FIG. 12D). While it has been suggested that maintaining symmetry between the replichores is important for bacterial genome stability, our observations suggest, without being bound by any particular theory, that the bacterial genome is robust to at least tolerate large megabase-scale insertions at various positions on the chromosome, provided the absence of misaligned ter sites relative to the direction of the replichore.

Additionally, described herein is the successful transfer of the 1-Mb seg. 5 centered around the oriC of Syn61 into TS3 of a DH10B recipient. This procedure generated genome chimeras that have an additional, ectopic replication origin. Whole genome sequencing indicates that this inserted secondary oriC is active, due the presence of a maxima of read coverage at this oriC (FIG. 3F).

Collectively taken, it shown herein that the entirety of a bacterial genome can be transferred in discrete sections into other bacteria to build genome chimeras.

### Transcriptomics of Chimeric Genomes Fused by ACE

The creation of a 4.6-Mb E. coli DH10B+2-Mb Syn61 genome chimera presents a unique opportunity to investigate its transcriptomic profile. This first-in-class artificial bacterial strain has nearly half of its gene content complemented by recoded homologs (FIG. 3K). This will allow us to understand whether these recoded homologs express and how their presence influences native genomic context.

RNA-Seq measurements were performed on said chimeric genome alongside donor and recipient controls. An initial inspection of the data suggests that imported recoded complements and their native homologs are both transcriptionally active when comparing individual replicates through hierarchical clustering and grouped datasets through positional expression (FIG. 3L-FIG. 3M). A comparison of the expression distribution for both the DH10B genes and Syn61 genes uncovers no significant difference between the levels of expression for Syn6l and DH10B gene homolog categories (FIG. 3N). Differential expression of the chimeric genome compared to the DH10B background reveals an overexpression bias of at least 1-fold change for a subset of genes that were duplicated and made redundant via ACE (FIG. 3O). Nonetheless, the strength of expression changes observed in our data are not solely determined by the copy number of genes, and deeper exploration is required to understand the regulatory dynamics of chimeric genomes constructed via ACE. We can consequently conclude that the massive complementation of the DH10B genome with recoded genes from Syn6l leads to expression of the introduced genes and perturbation of the native genomic background.

### Cross-Genus Megabase Genomic Additions

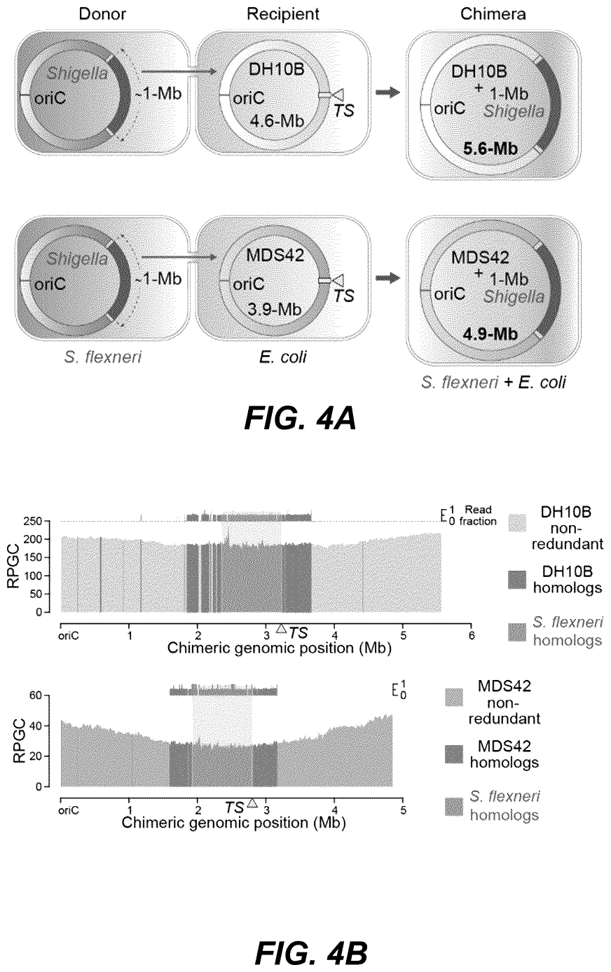

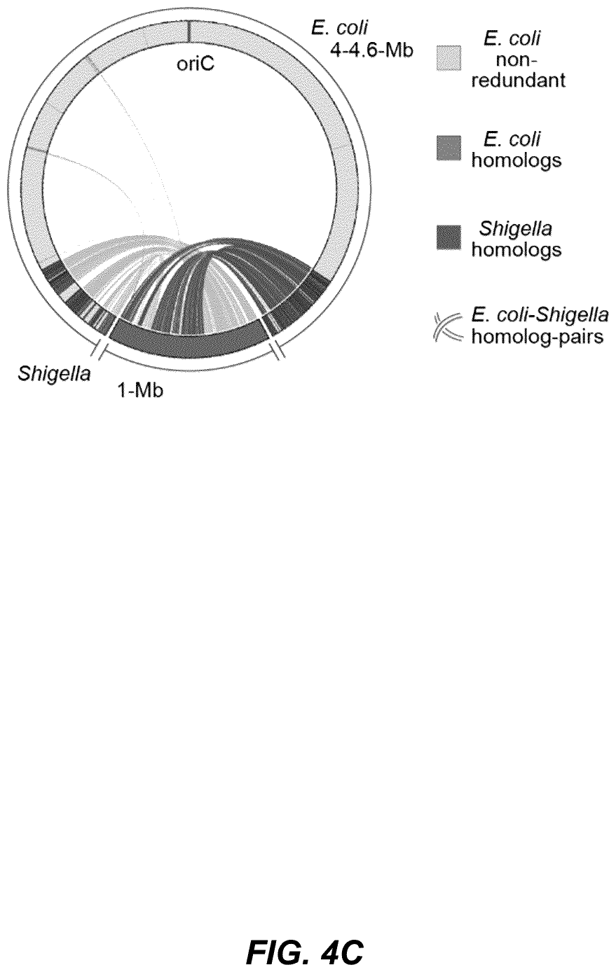

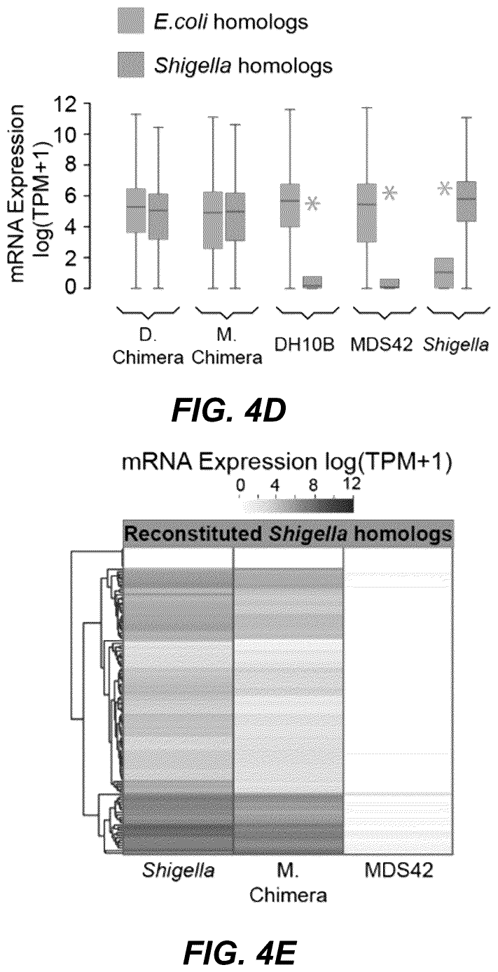

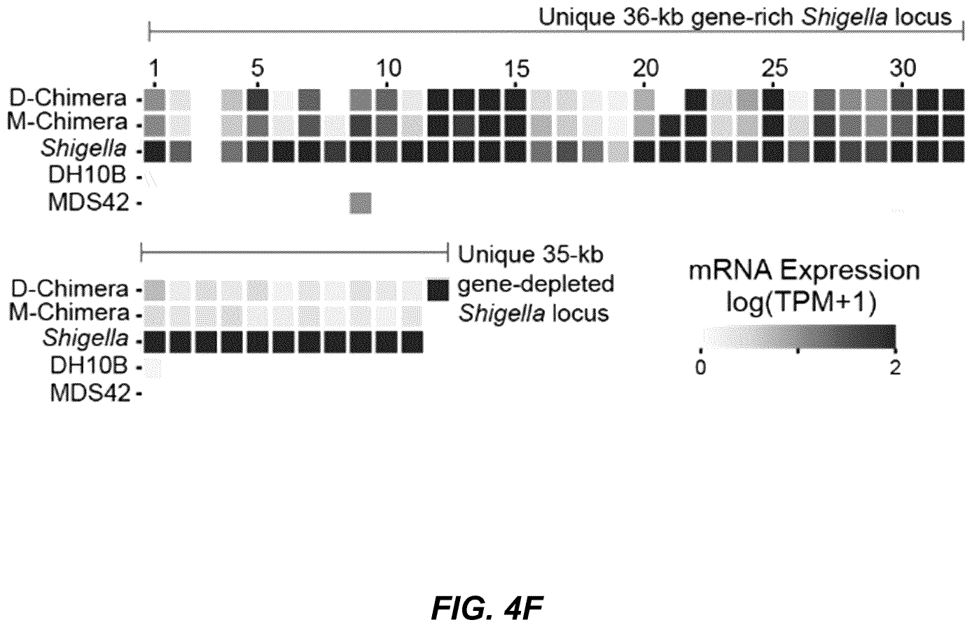

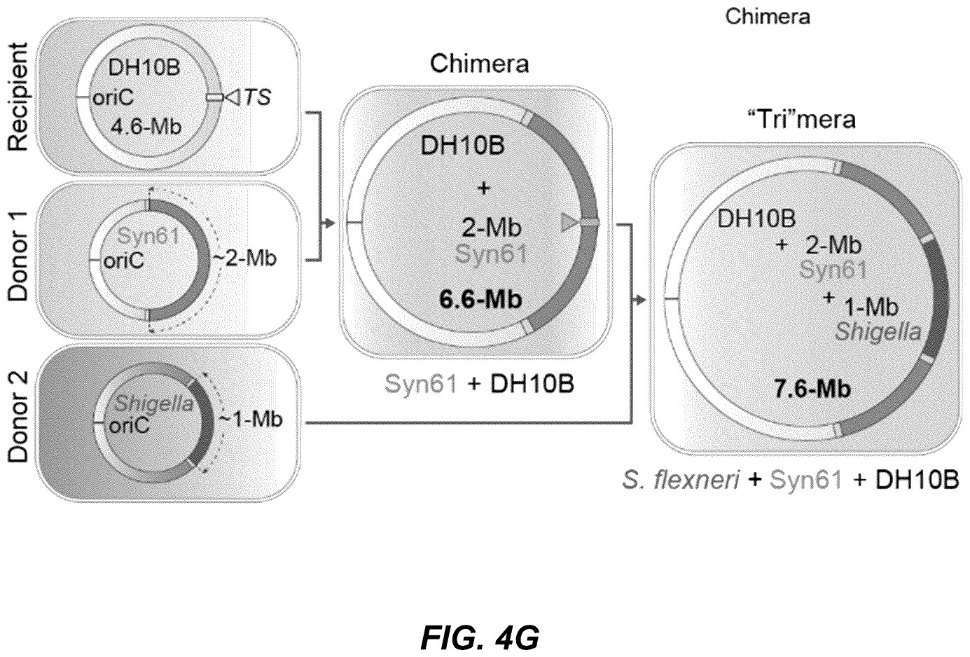

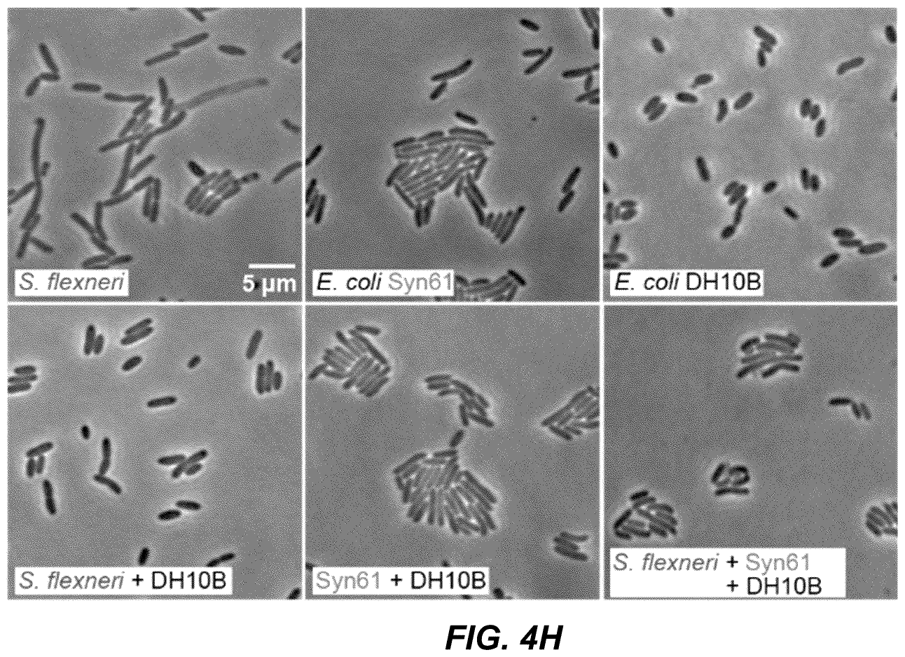

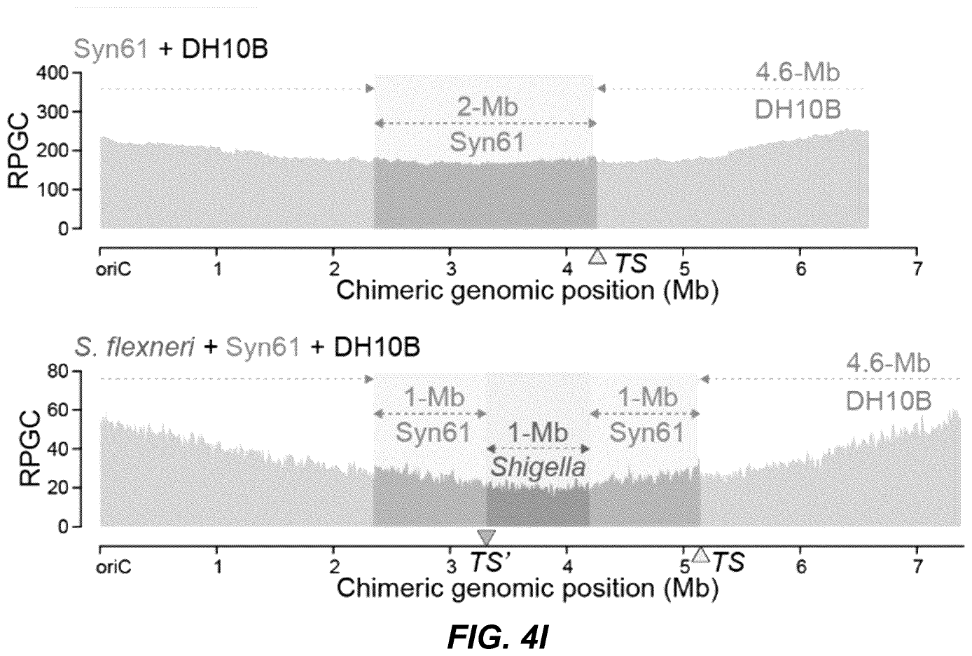

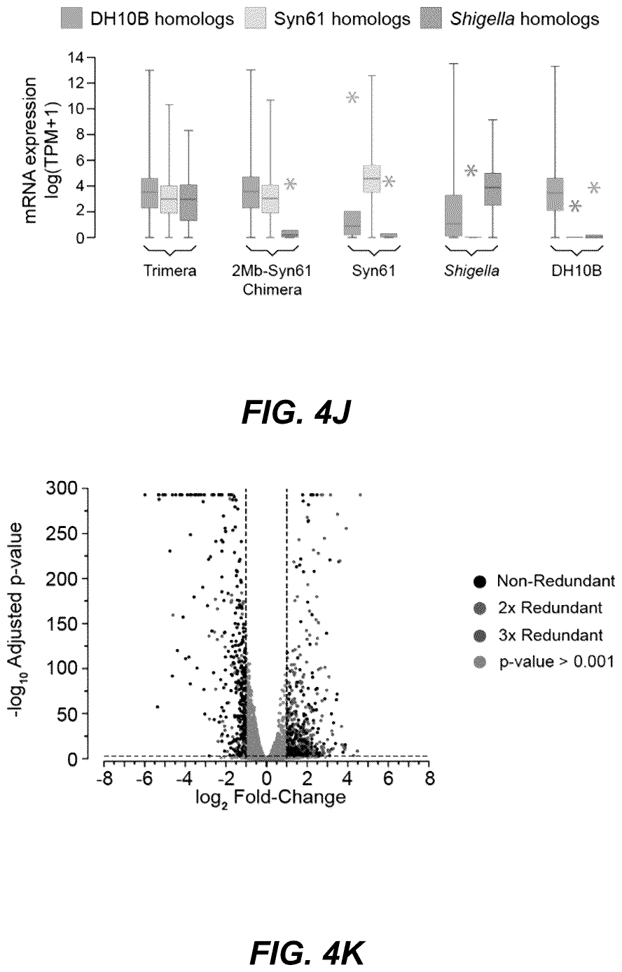

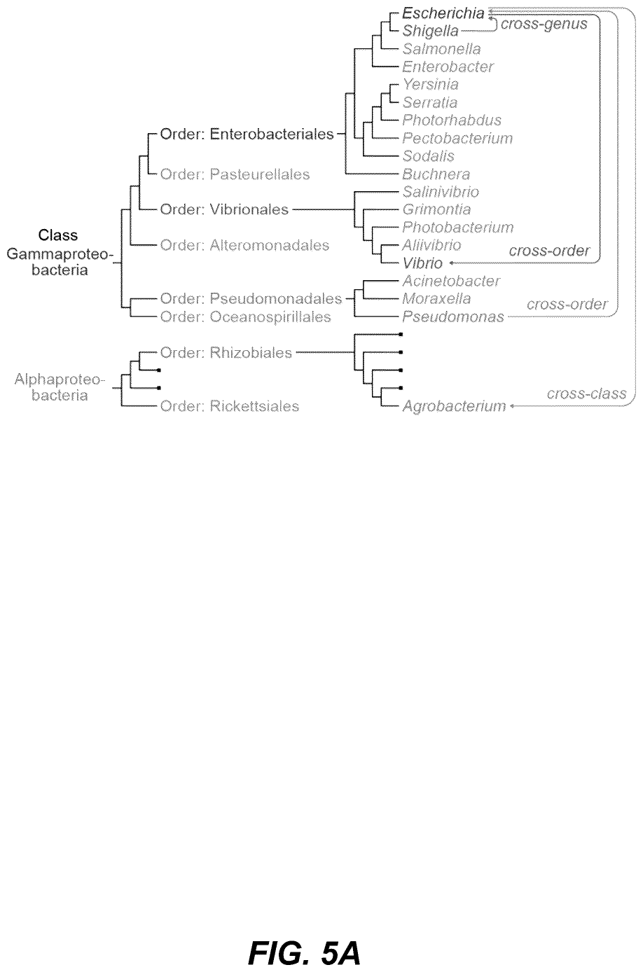

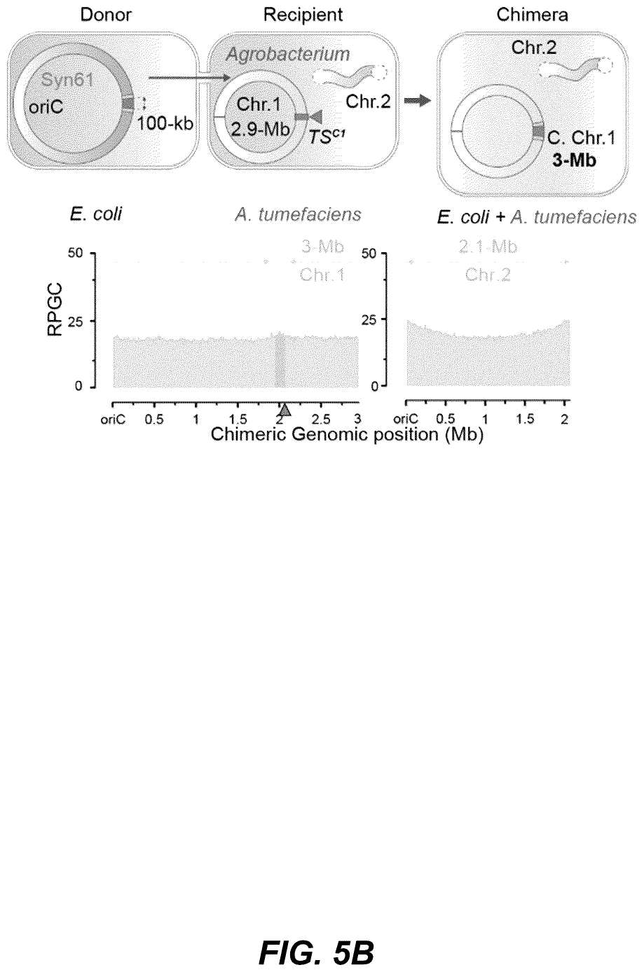

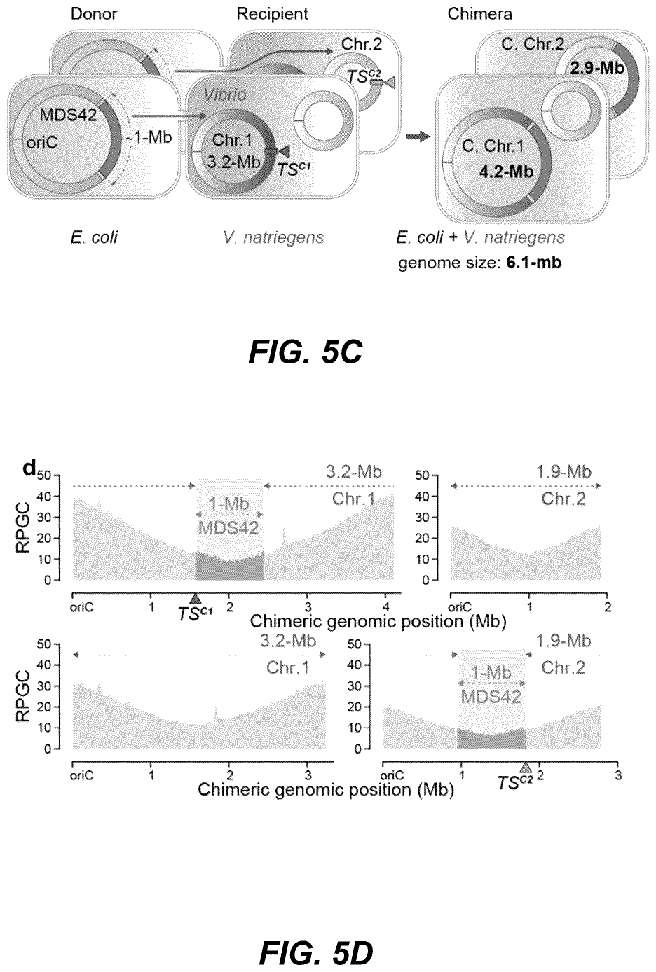

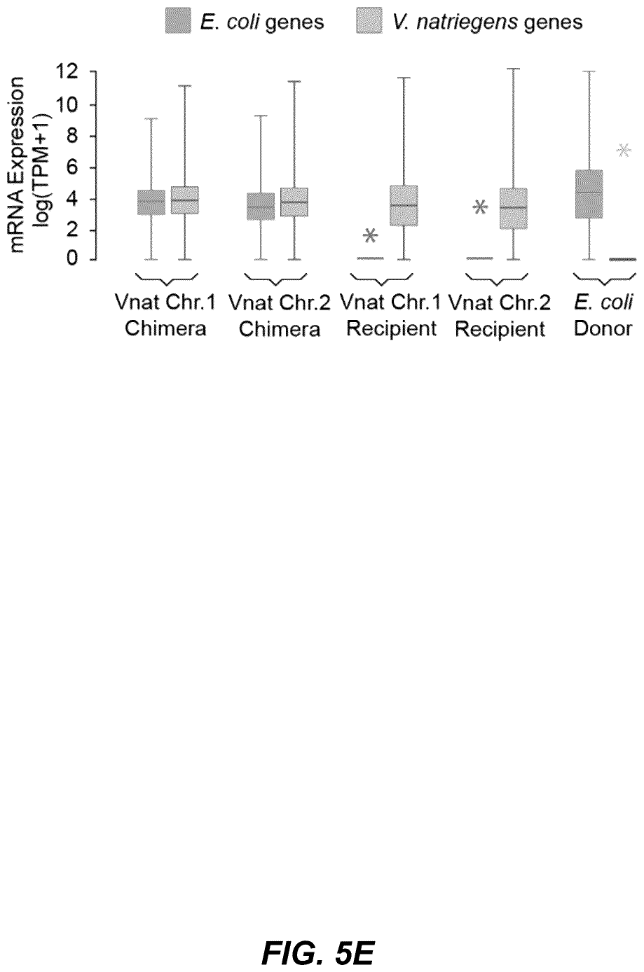

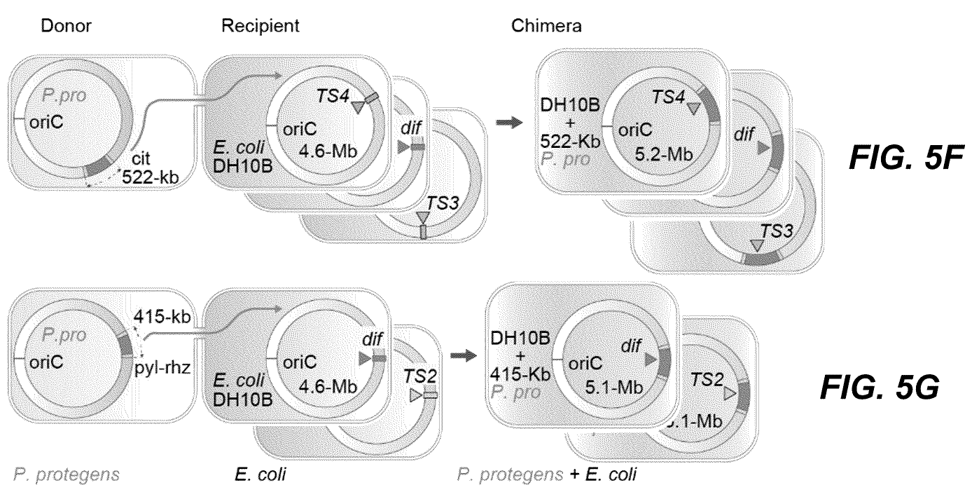

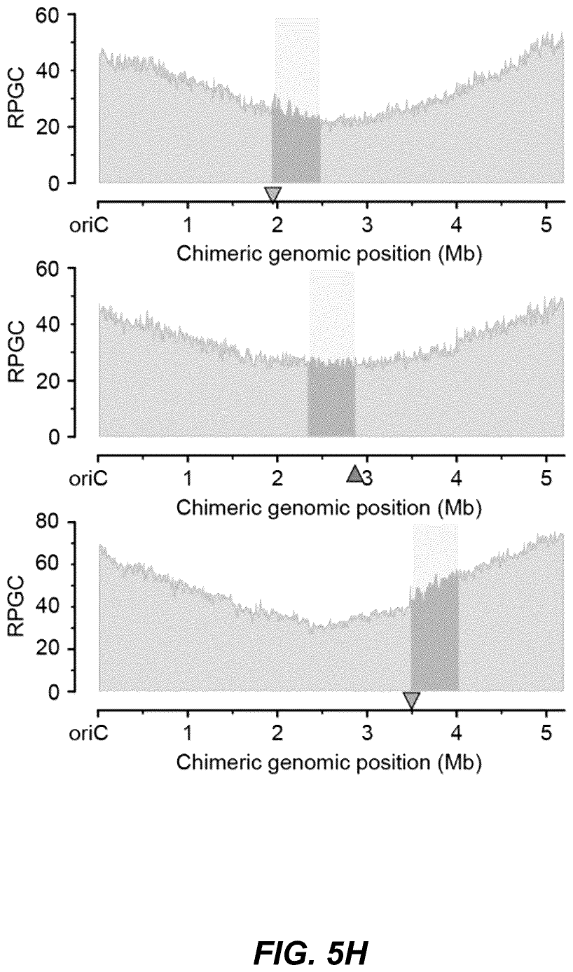

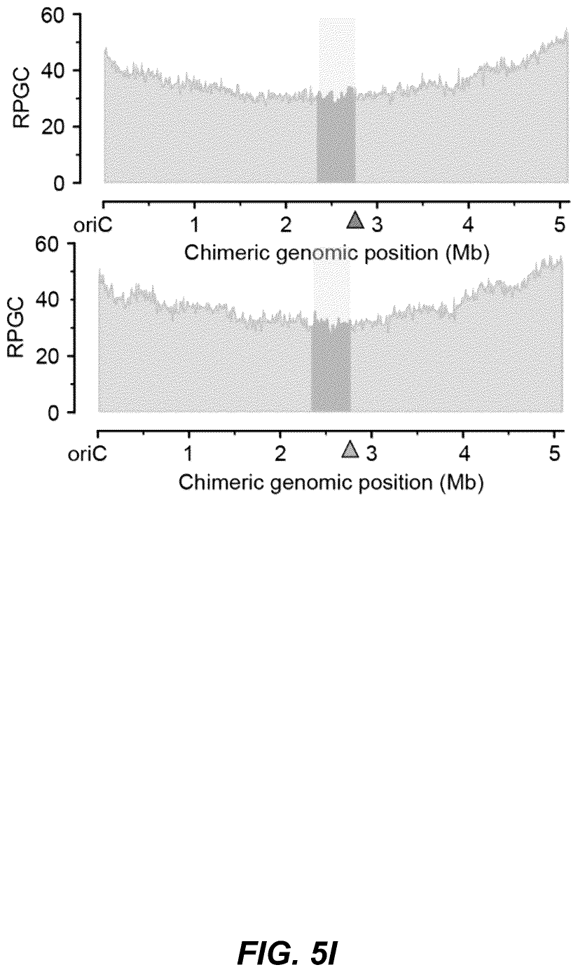

It was next investigated whether ACE was generalizable to donor and recipient bacteria beyond E. coli strains for the creation of cross-genus genome chimeras. Towards this aim, we engineered an attenuated Shigella flexneri strain (S. flexneri CFS100) into a donor to donate about 1 Mb of its genome. S. flexneri was chosen as Shigella is classified as the genus closest related to the genus of E. coli (FIG. 5A). In a single ACE step, the engineered S. flexneri donor strain successfully transferred 900-kb of its genome centered around the dif into the dif site of an E. coli DH10B recipient (FIG. 4A). The success and stability of this cross-genus chimeric genome was validated by whole genome sequencing (FIG. 4B). A corresponding transfer of the S. flexneri donor using a different E. coli strain MDS42 dif was also performed and confirmed to have been successful (FIG. 4A-FIG. 4B).

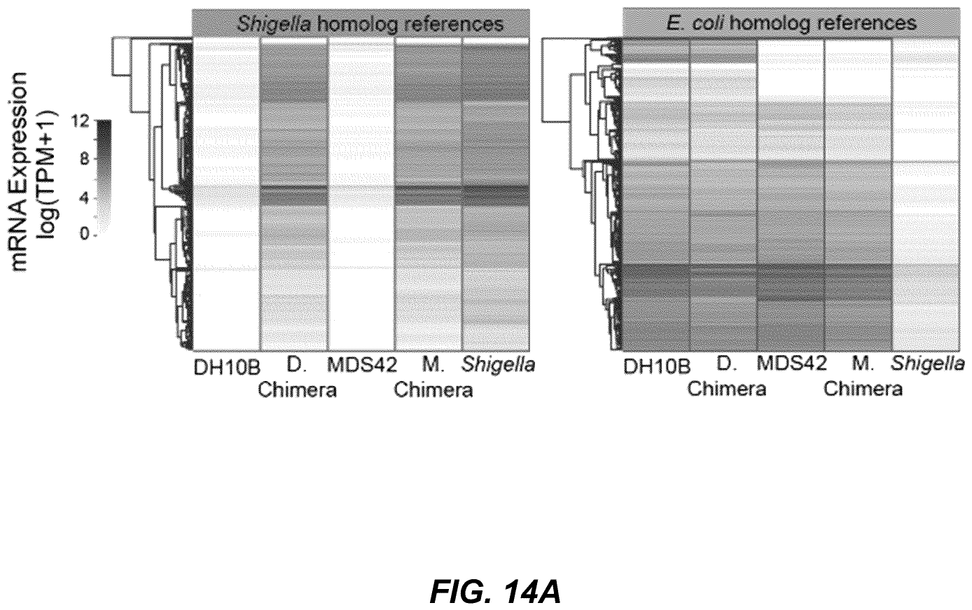

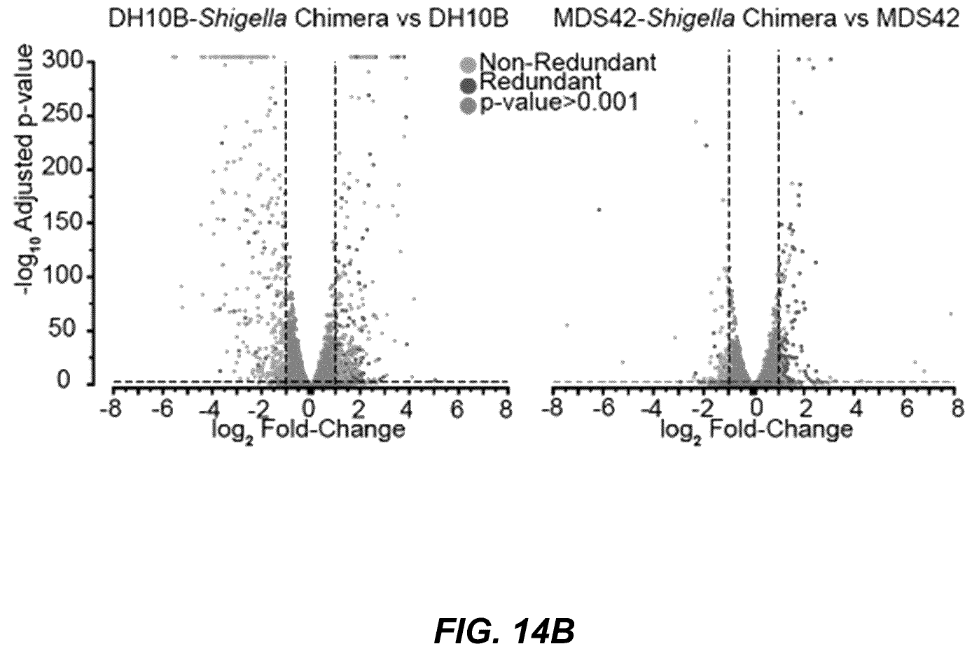

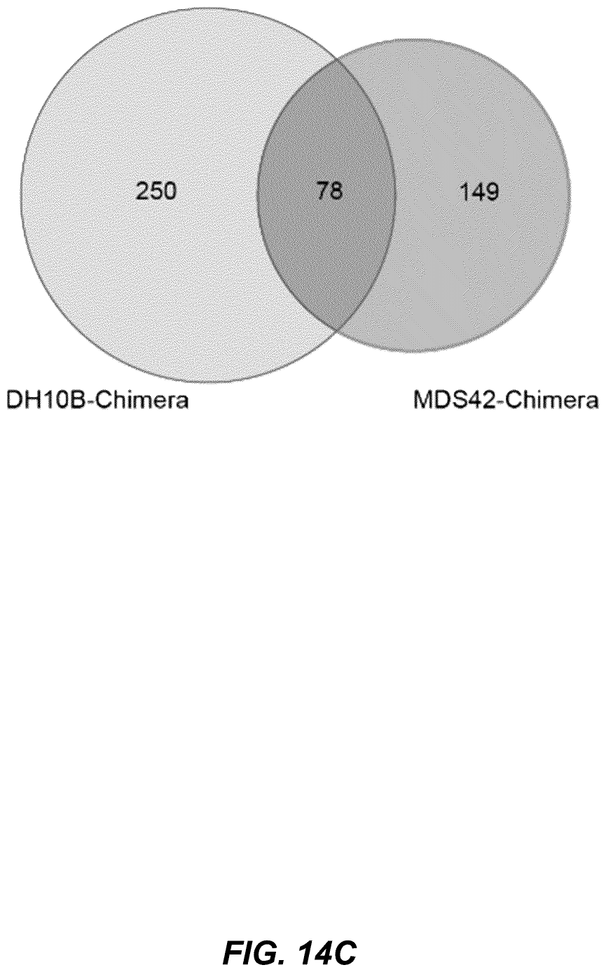

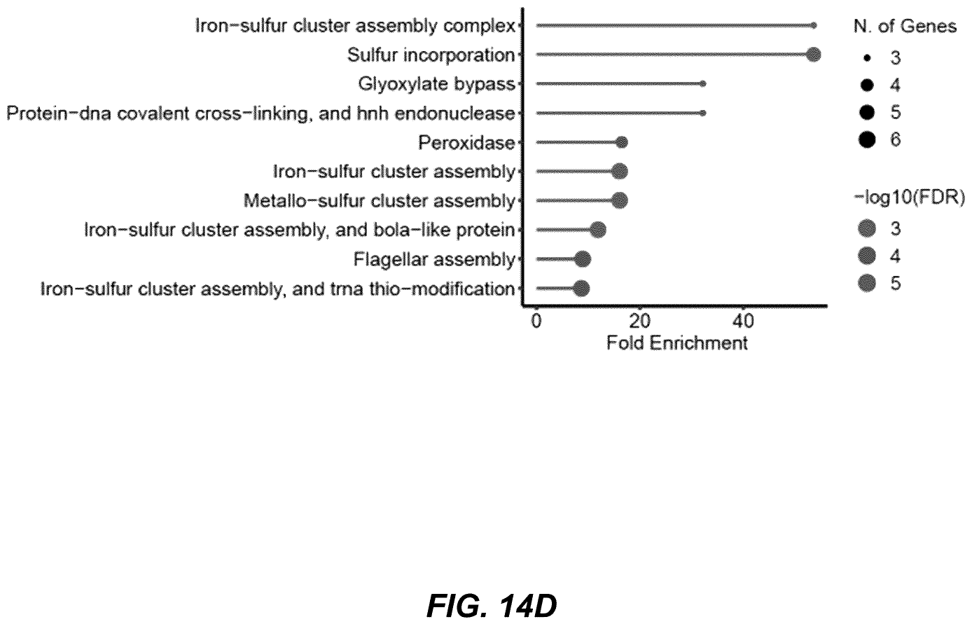

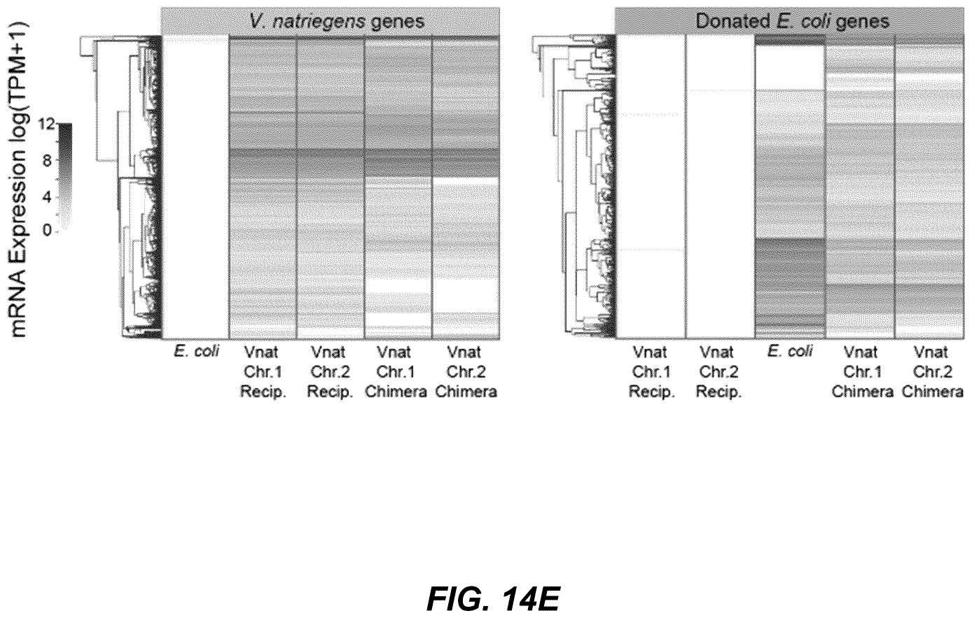

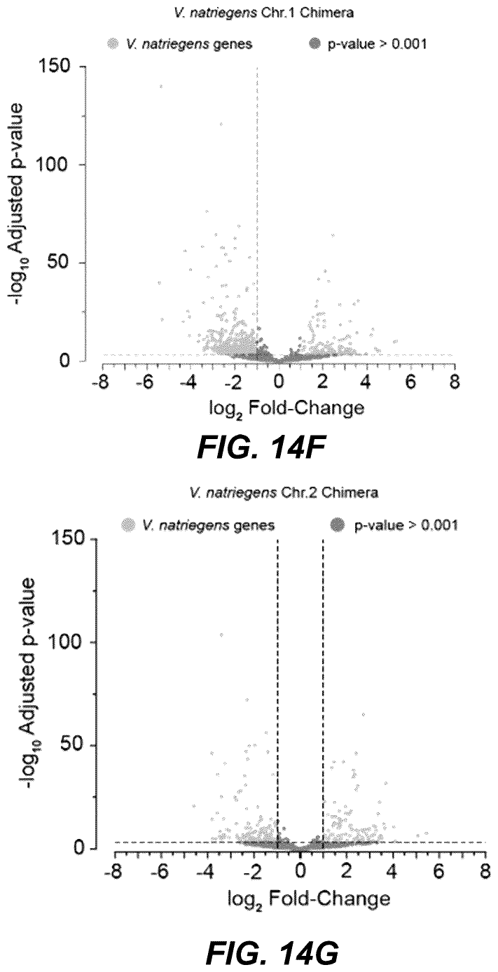

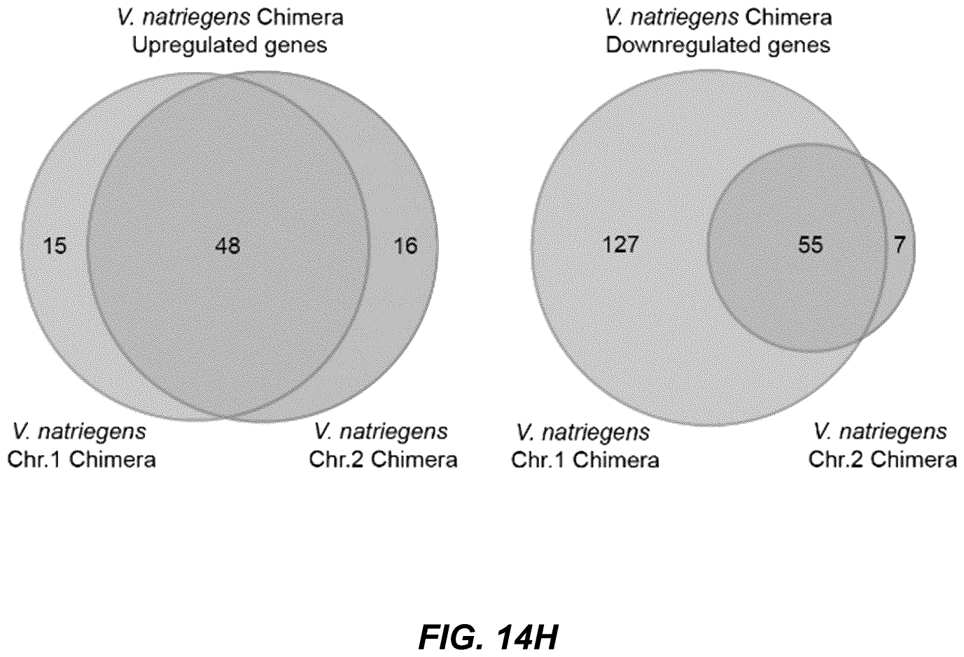

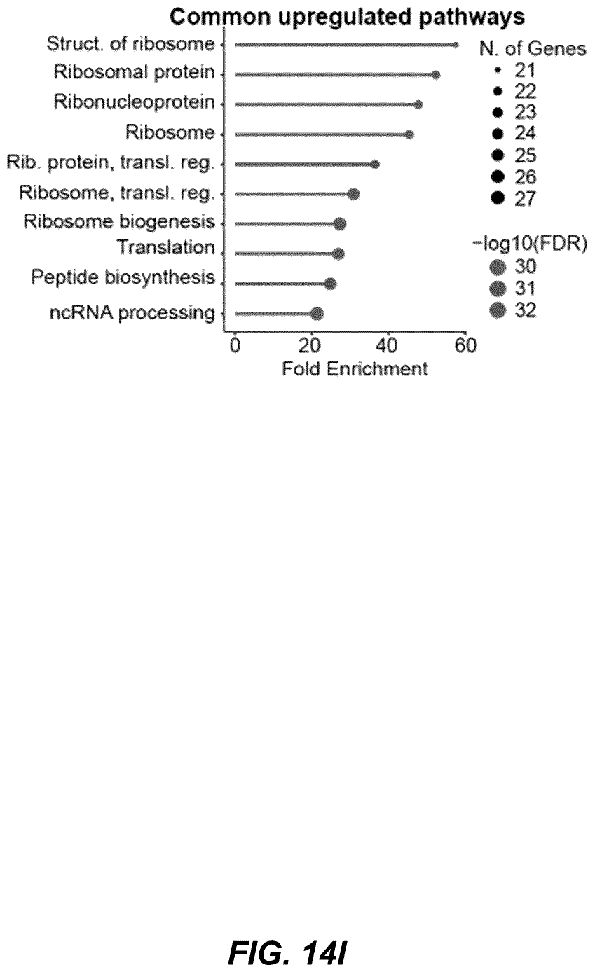

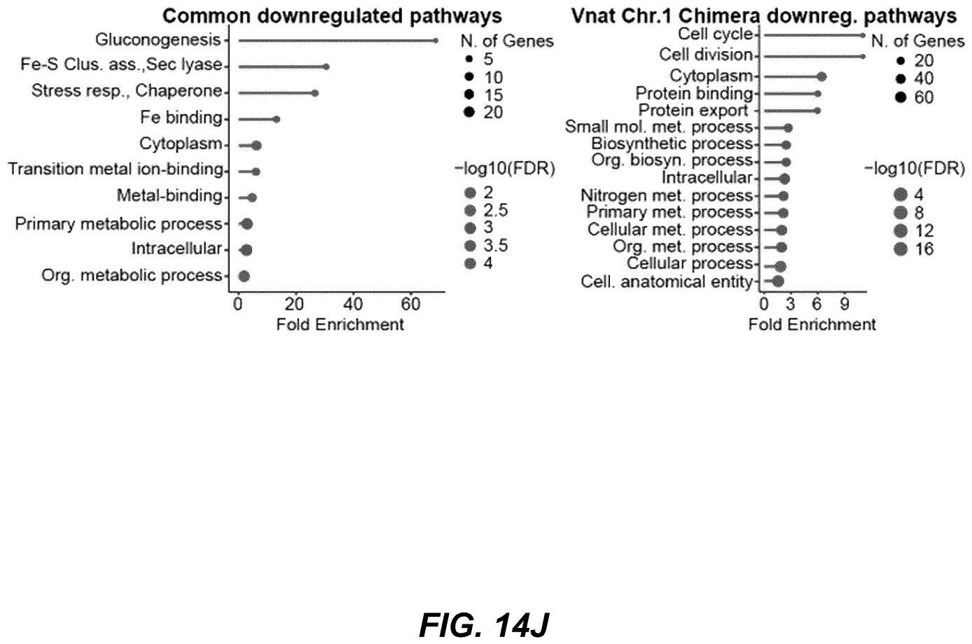

RNA expression profiles of both the DH10B-Shigella and the MDS42-Shigella ACE chimeras were interrogated in this work. Similar to our observations for the DH10B-Syn61 ACE chimera, DH10B- and MDS42-Shigella ACE chimeras showcase robust transcriptional expression for both imported gene homologs and their native complements (FIG. 4C-FIG. 4D, FIG. 14A). Novel Shigella genes that were gained through ACE expressed readily in both DH10B and MDS42 recipient backgrounds (FIG. 4F). Expression of genes originally deleted in the MDS42 strain background were reconstituted upon chimerization via ACE (FIG. 4E). Differential expression analysis was additionally carried our for the DH10B- and MDS42-Shigella ACE chimeras compared against DH10B background. This revealed a strong bias of at least 1-fold change for genes that were duplicated and made redundant via ACE (FIG. 14B). Of note, gene ontology analysis of the intersection of overexpressed genes in the DH10B- and MDS42-Shigella ACE chimera showcased strong upregulation of genes involved in iron-sulfur complex assembly, glyoxylate bypass, peroxidase, and flagellar assembly (FIG. 14C-FIG. 14D).

### Iteration of Megabase Transfers: Creation of a Genomic Trimera

We further demonstrated the iteration of ACE to enable even larger scale sequential additions. The ability to iteratively transfer multiple megabases from different donor bacteria would significantly expand the number and complexity of artificial genome chimeras attainable through this method. It was therefore asked whether multiple large insertions with ACE are possible on the same recipient.

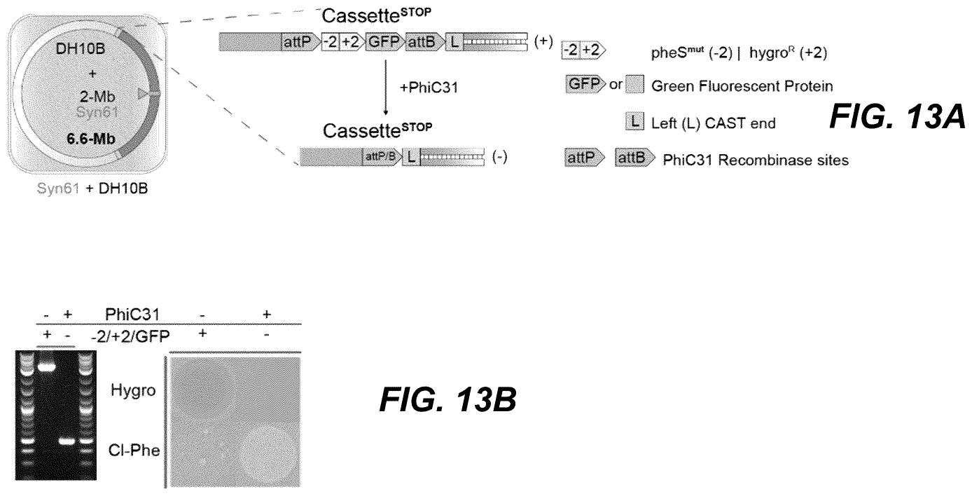

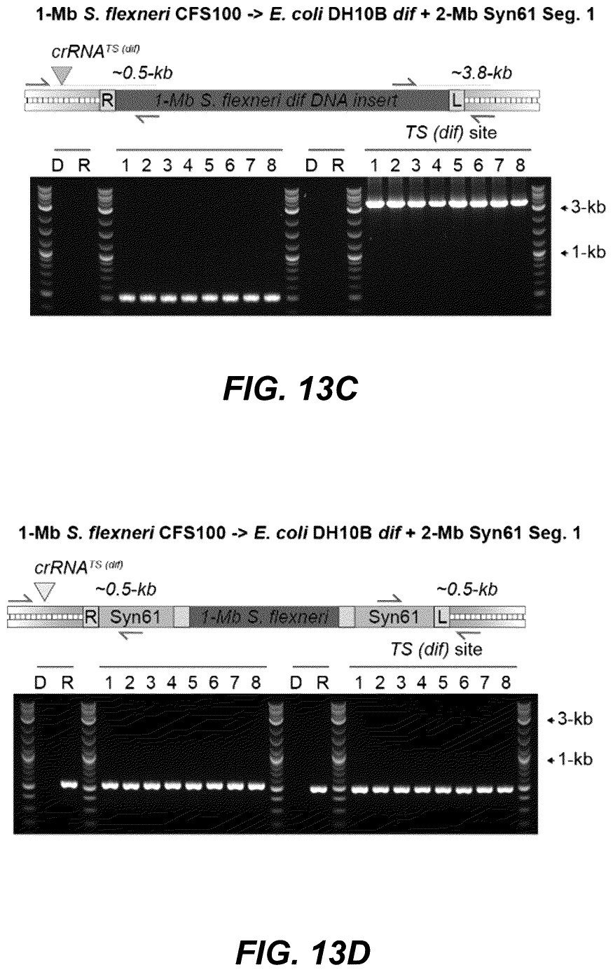

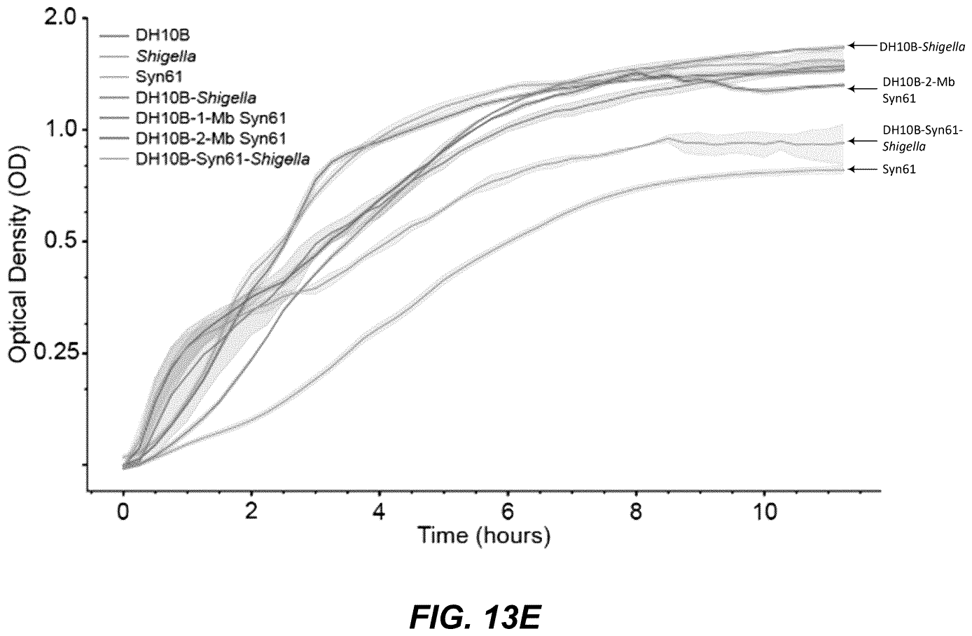

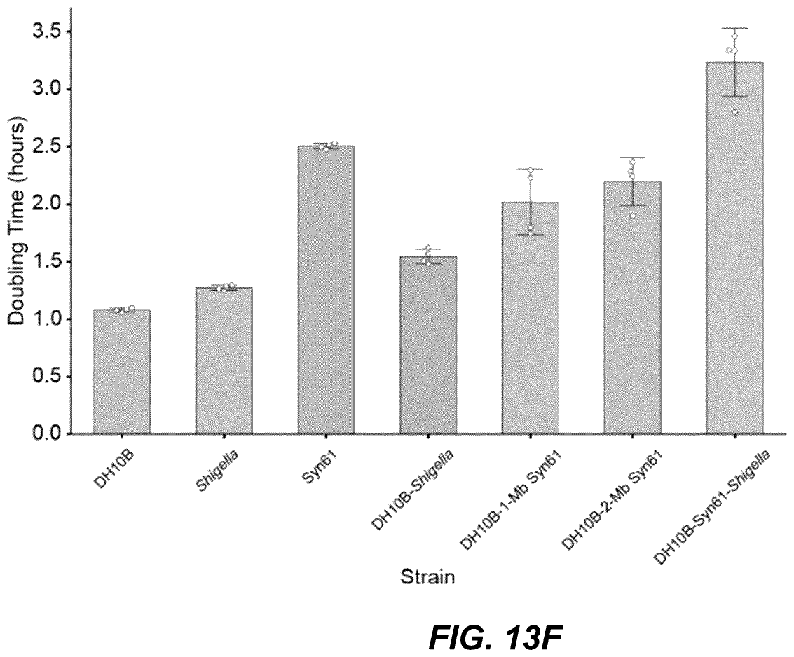

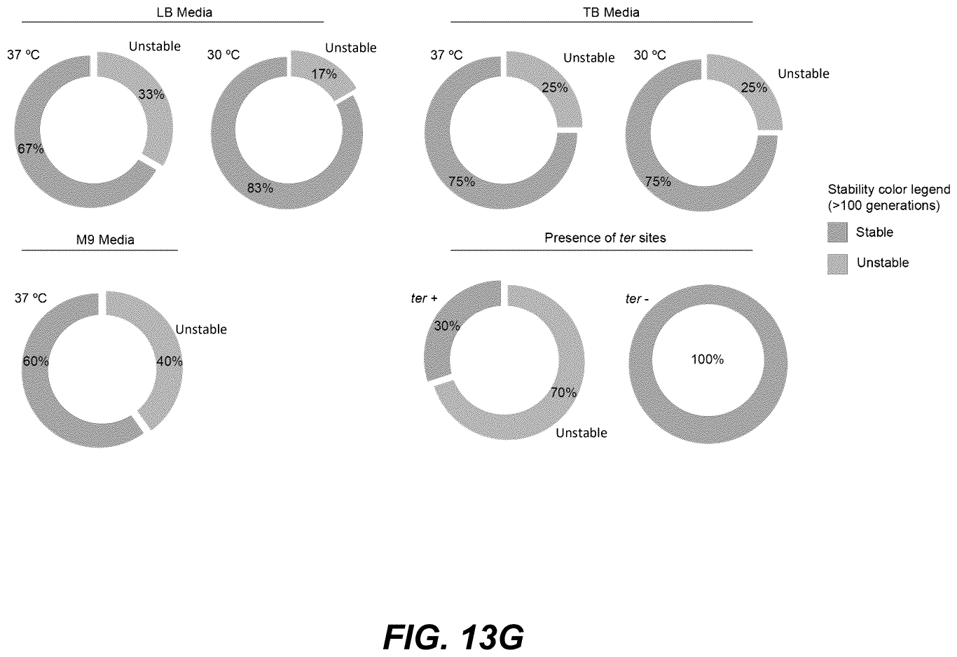

Towards this end, we decided to test the iterative integration of the 900-kb S. flexneri donor segment into the previously validated E. coli DH10B+2-Mb Syn61 seg. 1 chimeric strain (FIG. 4G). Anticipating this, we had previously flanked the +2/−2 GFP selection/screening cassette adjacent to the L-TE in the Cassette^{STOP} with recombinase sites of the PhiC31 integrase. Expression of the integrase successfully excised the +2/−2 markers in addition to the GFP from the genome of E. coli DH10B+2-Mb Syn61 seg. 1, priming this chimeric strain for subsequent megabase-scale ACE transfer (FIG. 13A-FIG. 13B). It was reasoned that we would not encounter the phenomenon of Tn7-like transposon target immunity if we integrated the S. flexneri donor into the middle of the 2-Mb Syn61 segment of the chimera using the dif crRNA. The R-TE and L-TE of the newly integrated donor would be approximately 1 Mb from both the R-TE and L-TE of the previous integration, thus avoiding a reduction of transposition efficiency. The sequential integration of a 1-Mb S. flexneri donor segment into the dif site of the Syn61-DH10B chimeric genome yielded stable trimeric genomes (FIG. 4I and FIG. 13C-FIG. 13D).

This stable trimeric genome was then interrogated in the context of RNA transcriptomics, and all three homolog gene variants were shown to be expressed robustly (FIG. 4J). Interestingly, the RNA count distribution highlights an order of preference for the strength of expression of the homolog variants, where DH10B native variants are expressed more than Syn61 homolog variants, and where Syn61 homolog variants are expressed more than Shigella variants. Differential expression was carried out comparing the trimeric strain to the DH10B background; our findings suggest that—alike previous ACE chimeric strains—there is a non-deterministic enrichment in gene overexpression above 1-fold for both doubly redundant and triply redundant genes (FIG. 4K).

Confocal microscopy of these chimeric and trimeric strains revealed rod-shaped morphologies akin to their wild-type bacterial progenitors (FIG. 4H). Growth assays of these strains also revealed that larger and successive transfers slow the growth rate of these strains to a moderate extent (FIG. 13E-FIG. 13F). Long-term stability of chimeric genomes was also demonstrated through prolonged passaging in different standard media and growth conditions exceeding 100 generations (FIG. 13G). Taken together, it is provided herein the successful creation of a cross-strain cross-genus genome trimera with a remarkably expanded genome of ˜7.5 Mb, a ˜3 Mb addition of genetic information in two successive ACE steps.

### Cross-Class and Cross-Order Genome Chimeras

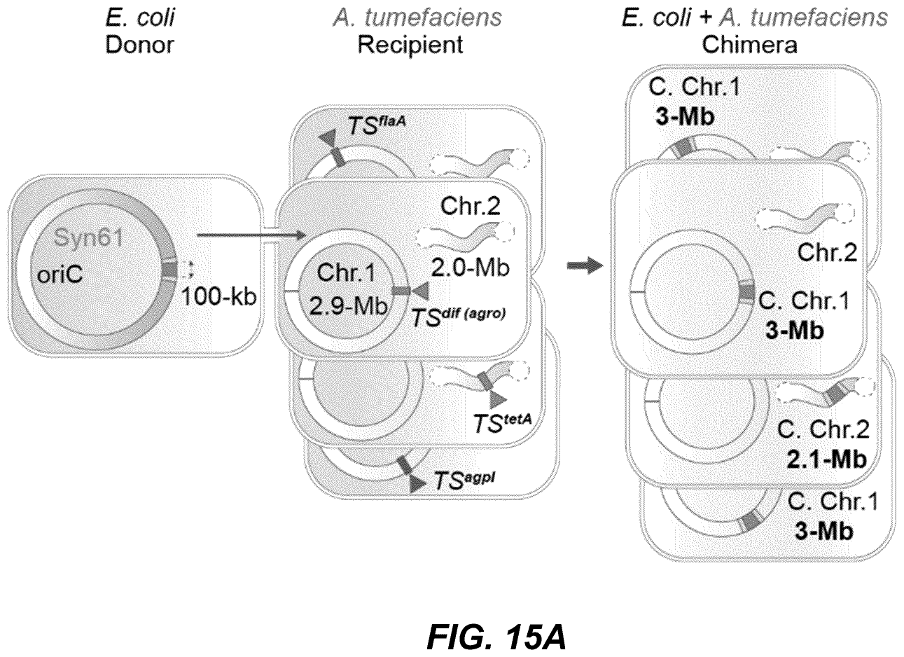

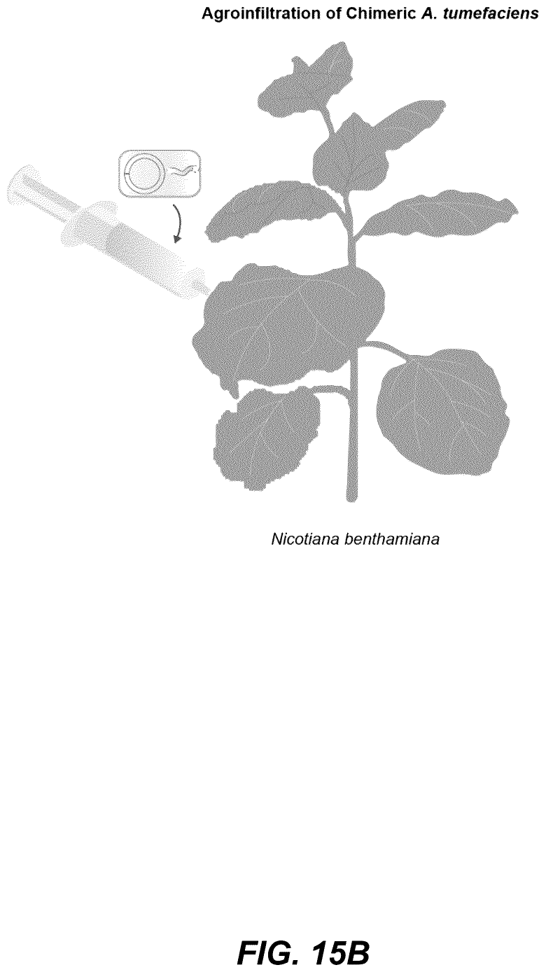

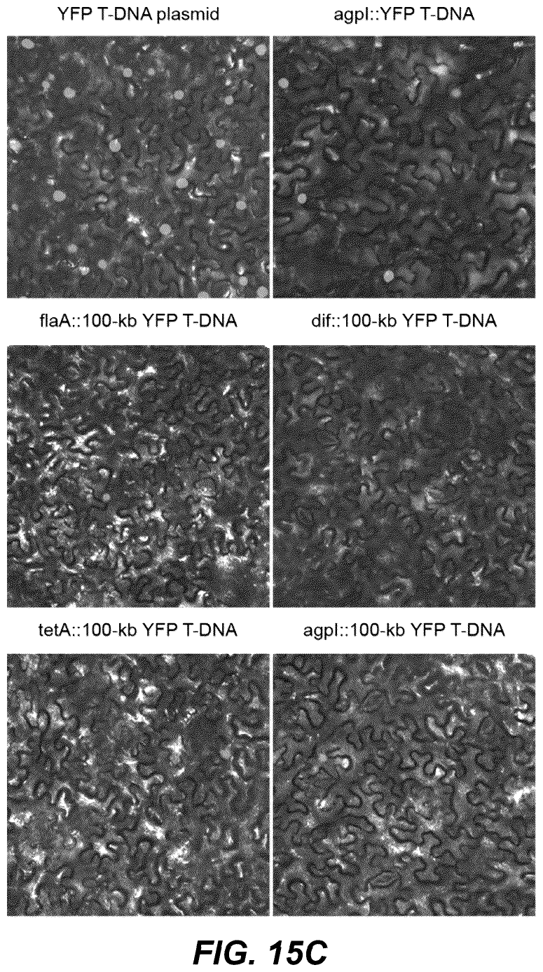

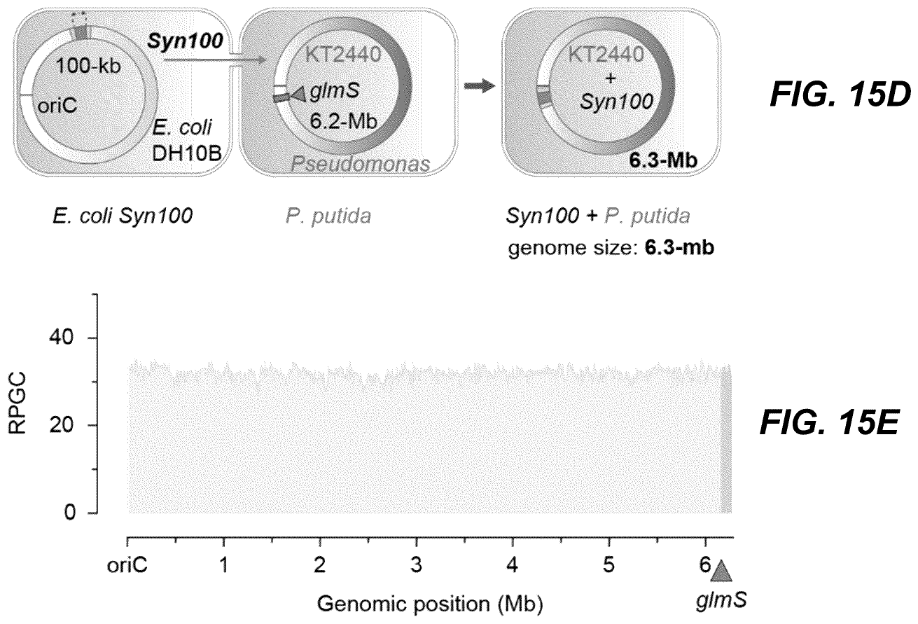

ACE was also tested across more distantly related bacteria. ACE transfers into industrially and biotechnology relevant bacteria such as Agrobacterium tumefaciens, Vibrio natriegens and Pseudomonas putida were tested. Compared to S. flexneri, these strains are more phylogenetically distant to E. coli and are classified in distinct orders (FIG. 5A). First, sub-megabase scale transfers from E. coli Syn61 to A. tumefaciens was demonstrated (FIG. 5B and FIG. 15A). Remarkably, these chimeric Agrobacterium were shown to be viable for agroinfiltration of Nicotiana benthamiana leaves, capable of transferring the YFP embedded in the Syn61 cargo from its genome in a T-DNA dependent manner (FIG. 15B-FIG. 15C). Next, with Vibrio natriegens we were able to achieve transfers of a 1-Mb MDS42 donor segment centered around the dif into both the dif site of chromosome 1 and of chromosome 2 in V. natriegens (FIG. 5C-FIG. 5D). However, although being viable and stable from room temperature to 37° C., these chimeric strains suffered decreased fitness at 4° C. and were not culturable after long term cryogenic storage, consistent with previous reports of the susceptibility V natriegens to cold temperatures. Nevertheless, we were able to conduct transcriptomic expression analysis, demonstrating expression of the E. coli genes gained through this transfer in the chimera and uncovering upregulation and downregulation of various Vibrio genes in the chimera relative to the background strain (FIG. 5E and FIG. 14E-FIG. 14J). Pilot transfers of 100-kb Syn100 were also successful from E. coli donor into P. putida recipient when targeting downstream of the glmS gene (FIG. 15D-FIG. 15E). In summary, demonstrated herein is proof of concept ACE transfers of large donors across diverse bacteria spanning different species, genus, orders and even class, providing first-in-class evidence towards the functional expression of megabase-scale cross-species genomic hybrids.

### Attenuation of Pathogenic Properties and Emergence of Novel Metabolites in Pseudomonas Chimeras

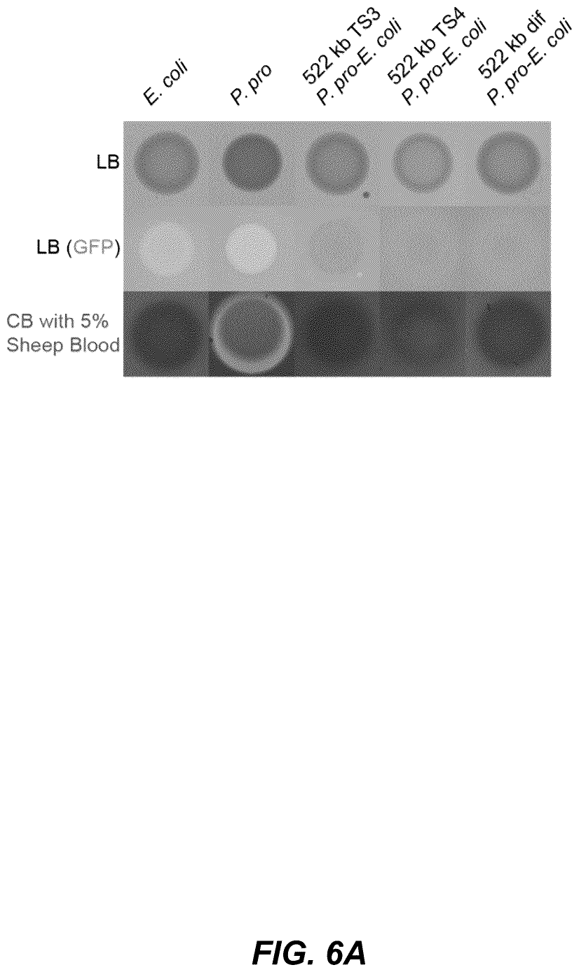

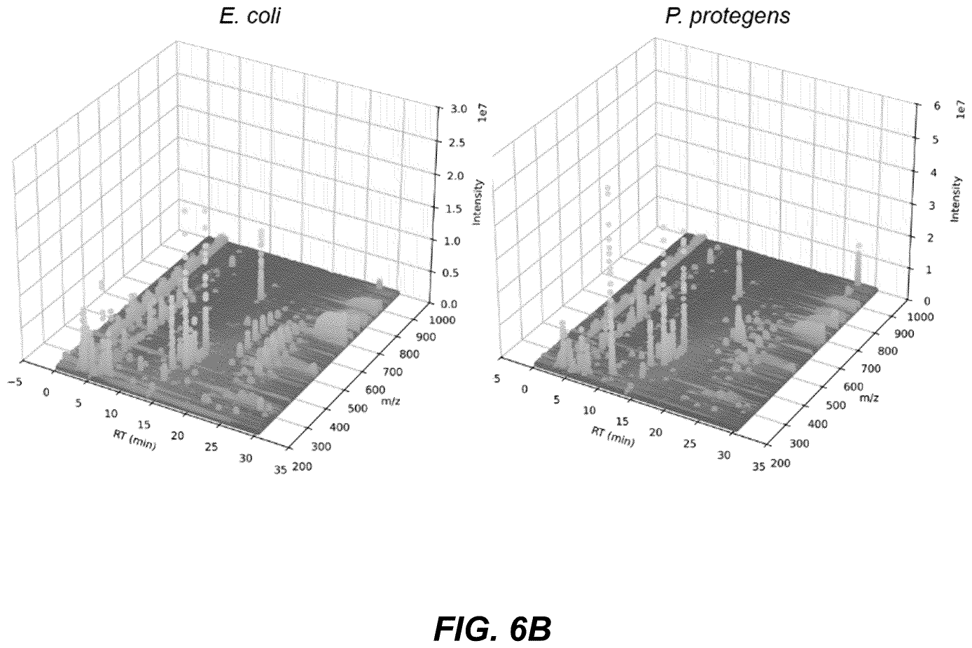

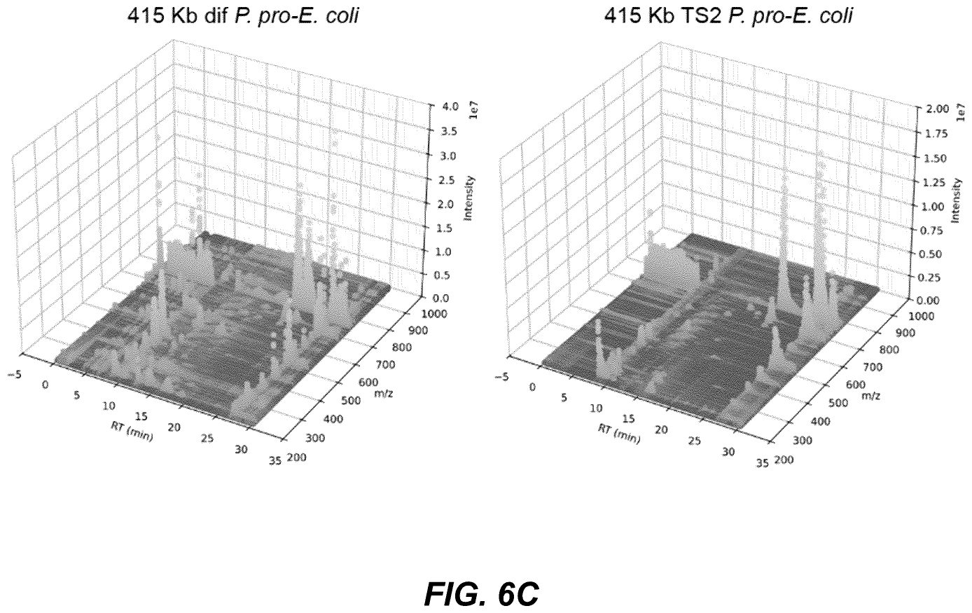

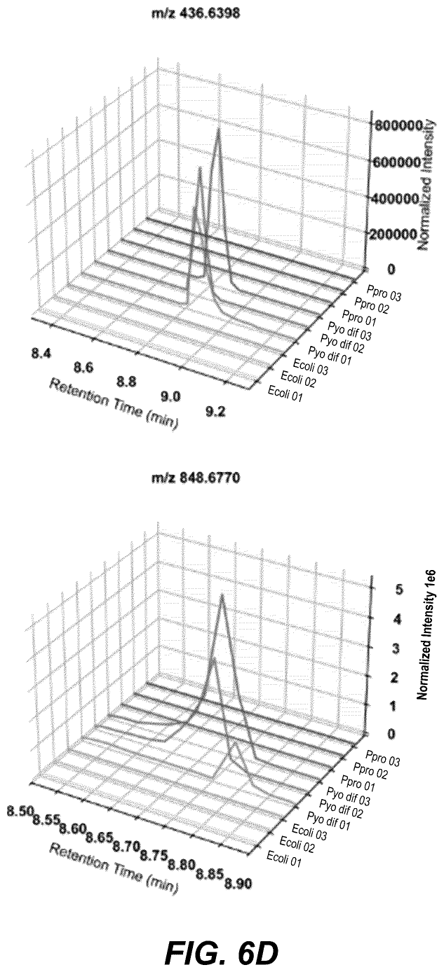

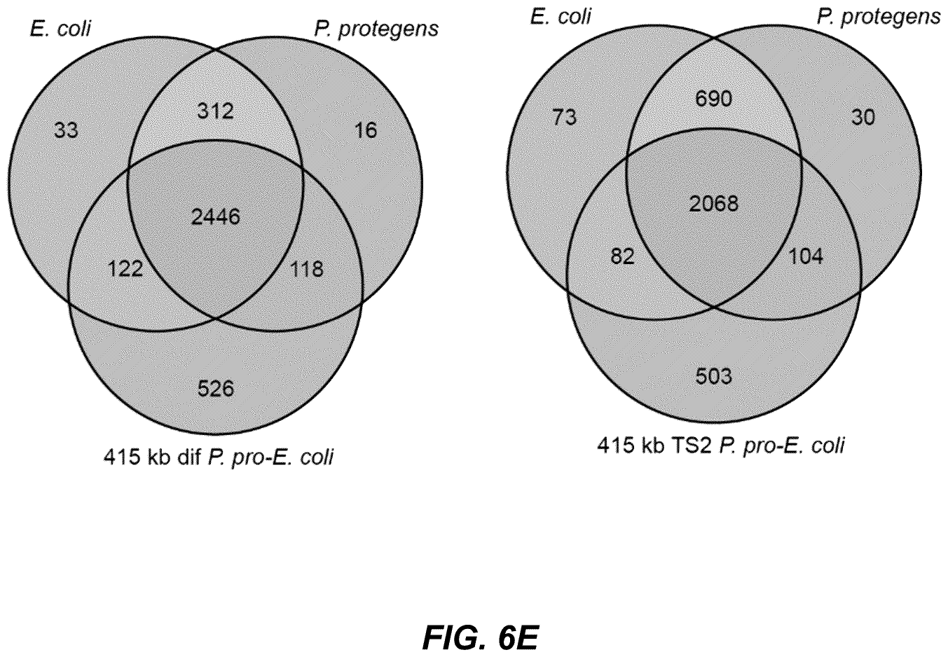

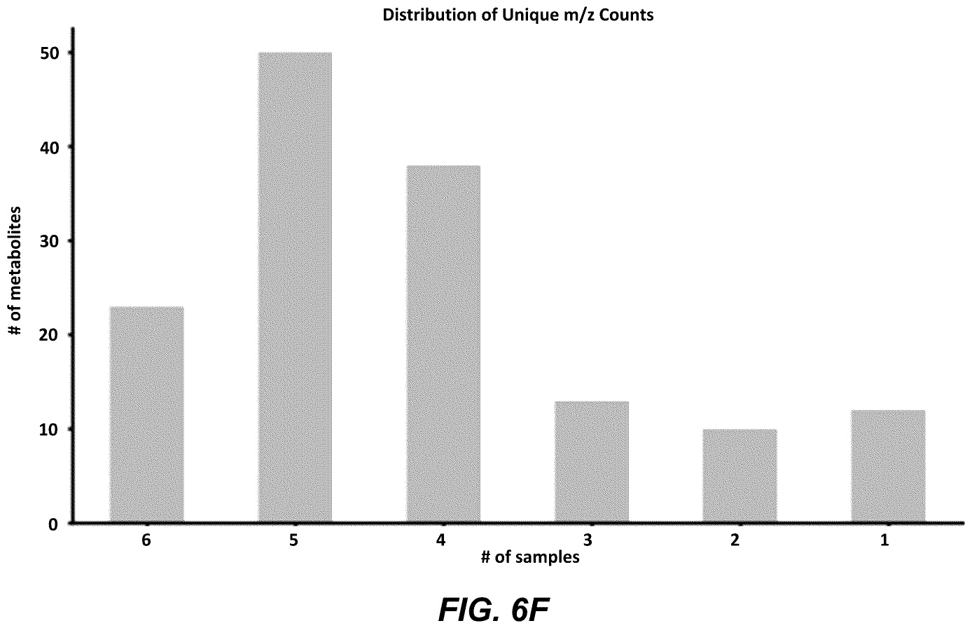

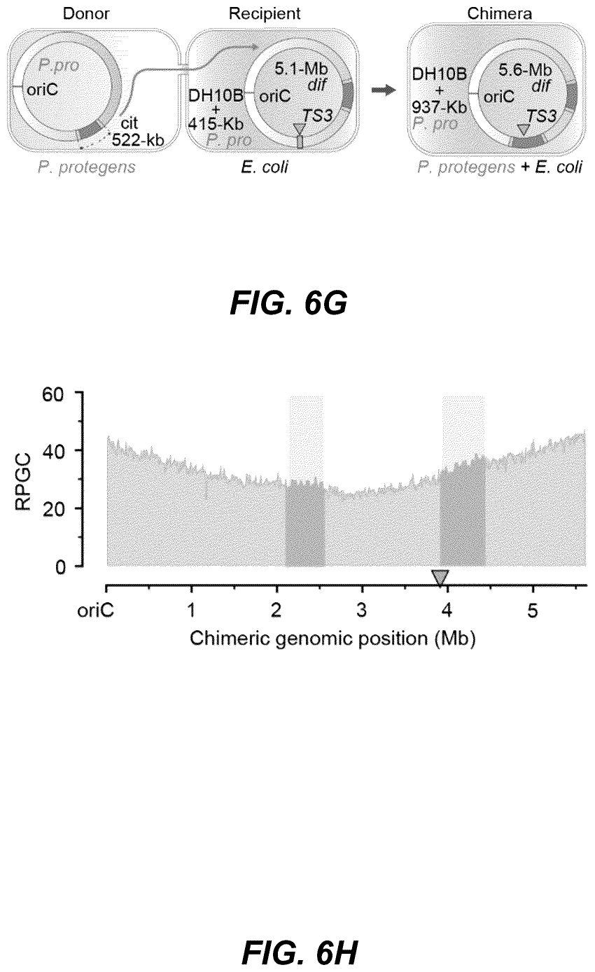

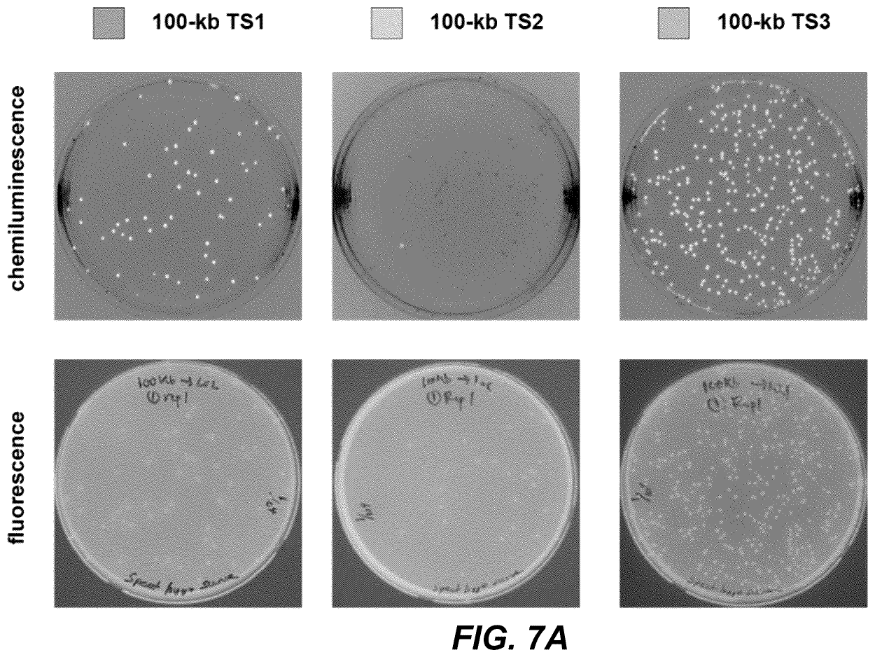

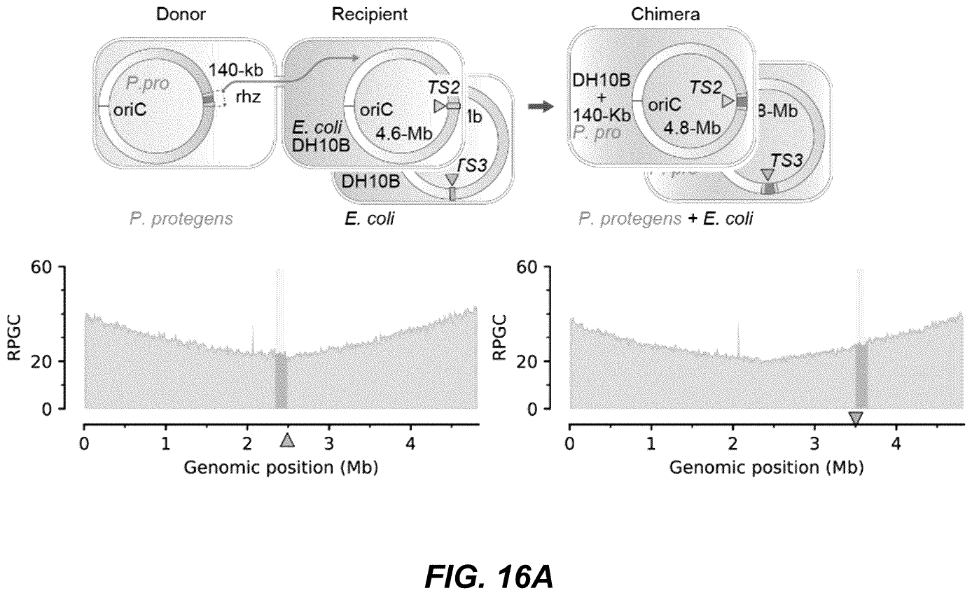

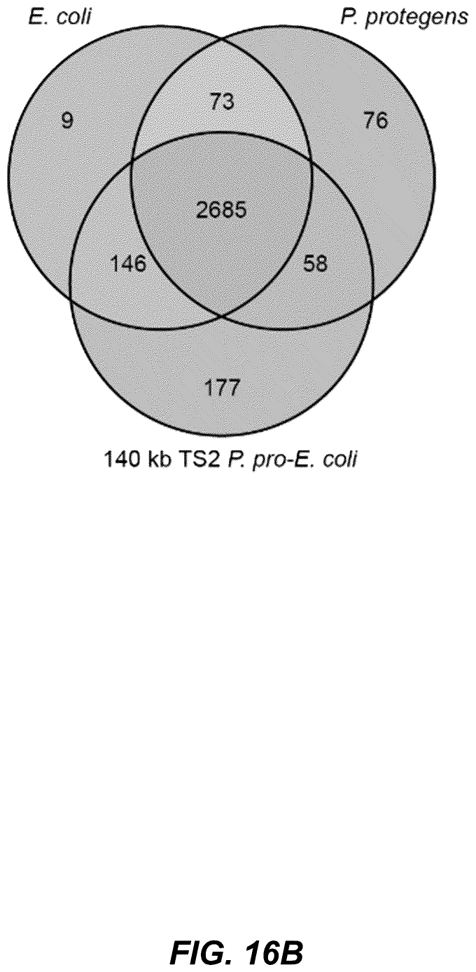

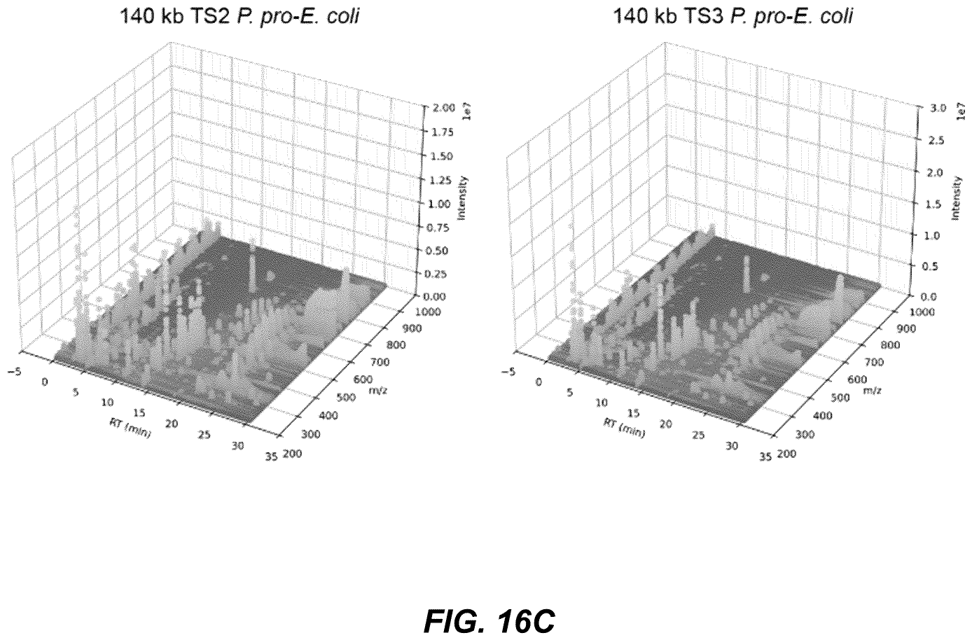

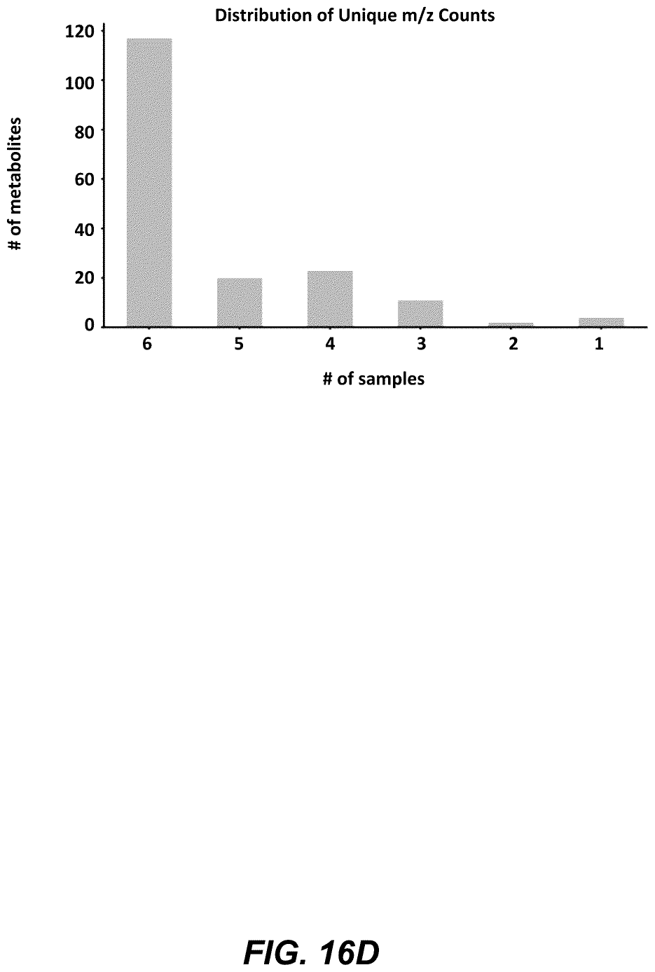

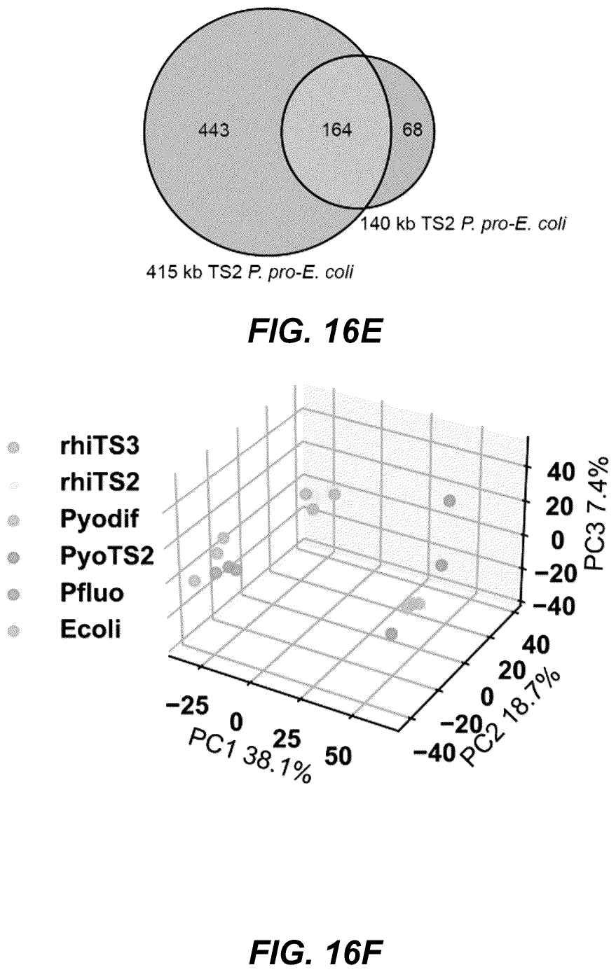

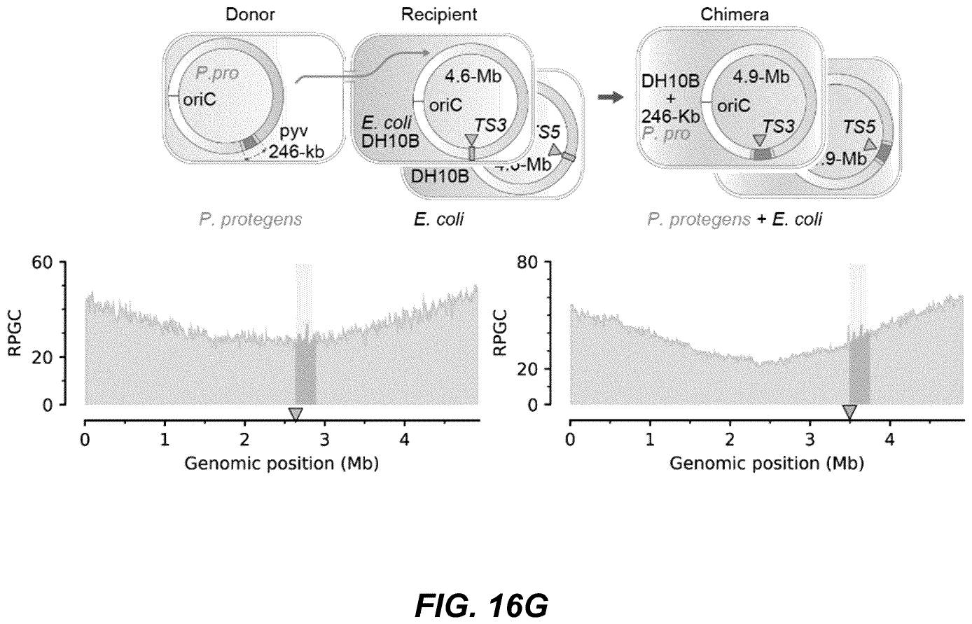

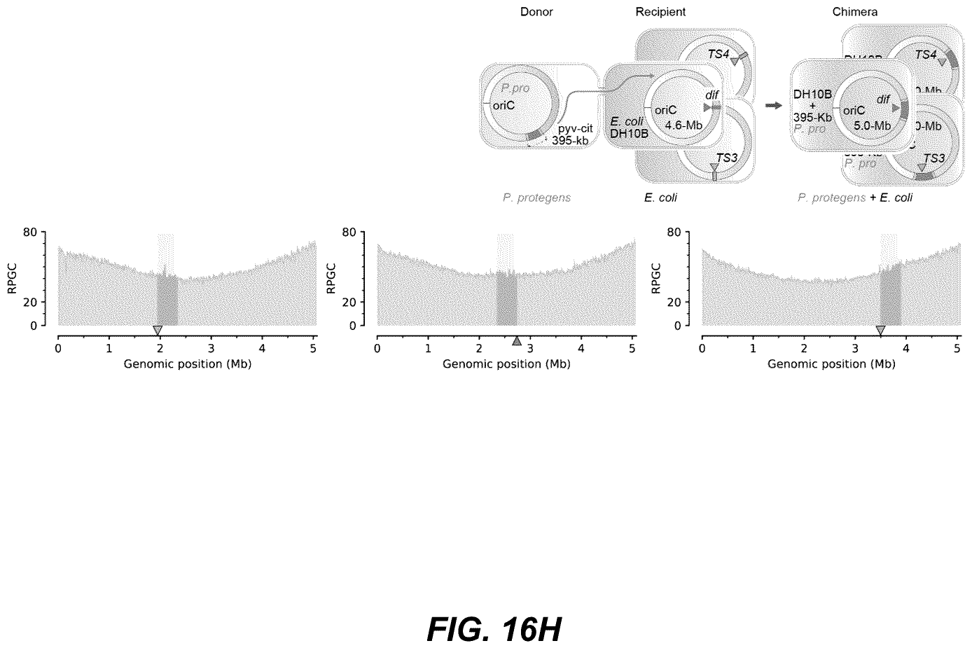

The use of orthogonal CASTs to insert Cassette^{START} and Cassette^{STOP} in bacteria that are much less amenable to recombination of heterologous DNA serves as the foundation of the OASIS method to construct donors, which unlocks the use of P. protegens pf-5 as a donor for ACE. A panel of P. protegens donors were then rapidly constructed, ranging from 140-, 246-, 395-, 415- and 522-kb, that were successfully transferred into E. coli DH10B at a wide range of loci (FIG. 5F-FIG. 5I and FIG. 16A, FIG. 16G, and FIG. 16H). Several chimeras from this panel were selected for downstream functional and omics experiments. Firstly, given the presence of hemolytic genes shlA and shlB in the 522-kb segment, we set out to assay the hemolysis activity of the chimeras relative to the donor and recipient. It was determined that the chimeras exhibit no hemolytic activity, in contrast with P. protegens, providing the use of ACE as a method for attenuating pathogenic phenotypes through chimerization (FIG. 6A). Next, unbiased metabolomic profiling of the 140-kb and 415-kb chimeras was performed (FIG. 6B-FIG. 6F and FIG. 16B-FIG. 16F). Notably, for the 415-kb chimeras, we uncovered a preponderance of unique m/z peaks that are not present in either the donor P. protegens or the recipient E. coli, suggesting the emergence of novel metabolic signatures through the process of chimerization (FIG. 6B-FIG. 6E). Indeed, PCA of the metabolomic profiles of the chimeras relative to the donor and recipient uncovers discrete clustering of the chimeras away from the donor and recipient profiles (FIG. 16F). Finally, we attained through iterative ACE of 522-kb and 415-kb chimeric segments of P. protegens into E. coli DH10B a near 1-Mb consolidation of P. protegens DNA stably inserted into the E. coli genome (FIG. 6G-FIG. 6H).

### Discussion

ACE enables multi-megabase genome integrations to create synthetic bacterial chimeras in a single step. Moreover, iterations of ACE can create trimeric strains larger than any prior reports. These created GEOs were stable and transcriptionally active. ACE functions across strains, species, genus, and orders, illustrating the feasibility of rapid, robust and scalable construction of artificial life.

The specificity and efficiency of ACE depends on the unique design coupling the oriT and TE sequences to specify the transfer window and facilitate the crRNA guided integration. Initiation of conjugation at the first starting oriT in Cassette^{START} and termination of conjugation at the second ending oriT in Cassette^{STOP} enables tight regulation of what is transferred and integrated to the recipient, and subsequently the oriT-adjacent TEs are used for targeted insertion.

Without being bound by any particular theory, this double-oriT configuration leads to two possible stretches of transferred DNA, hence the specific positions of the R-TE/L-TE and introduction of markers to counter-select against the undesired product starting from Cassette^{TO} and ending at Cassette^{START}. While this is effective for substrates of lengths equal to or less than half the size of the donor genome, in some embodiments, this double-oriT approach may reduce the efficiency of transfer for substrates larger than half the size of the donor genome. Engineering the conjugation machinery and the oriT sequences may pave the way to higher efficiency and greater robustness especially for greater than 1%-genome ACE operations.

The ACE design is also conducive to biocontainment as it decouples conjugation of the donor genomic fragment and integration of the transferred fragment into two separate plasmids in two cellular compartments. The donor DNA does not encode the means of its propagation after the conjugation into another cell without pAra-Trans. Only the designated recipients that carry the pAra-CAST will be able to incorporate the donated DNA, otherwise the transferred DNA cannot replicate and maintain itself. Hence, ACE has been inherently structured to limit the inadvertent propagation of transferred DNA outside of its intended target.

ACE demonstrates the capacity of CAST on the multi-megabase scale. By incorporating type I-F CAST as part of the disclosed ACE system, its targeted genomic integration capacity was expanded from 10-kb all the way to 2-Mb, ˜200-fold greater than previously reported. Without being bound by any particular theory, the findings reveal that the multi-stage recruitment of the cascade-tniQ-tnsC-transposome complex in the type I-F CAST, shown to mediate licensing of accurate target site selection and transposition, remains functional even in the context of genome-scale donors.

ACE offers a powerful alternative to homologous recombination-based methods. Genome integration by ACE via CAST inherently bypasses the need for double strand breaks (DSBs) and homologous recombination (HR) for incorporating changes into a chromosome. By building cross-strain genomes via ACE, we largely minimized genomic instability and undesired random recombination events, the occurrence of which is typically elevated when using HR and DSB dependent techniques to transfer megabase-sized DNA containing long redundant stretches homologous to the recipient genome.

While specific, CAST mediated transposition is also programmable. The reported flexibility in PAM requirements of Type I-F CASTs enables use of crRNAs with non-canonical PAMs. Indeed, in the studies described herein, we deviated in certain occasions from the 5′-CC-3′ PAM requirement, such as using a 5′-CA-3′ PAM when targeting the dif site, without noticeable reductions in ACE efficiency. Portability of Type I-F CASTs, as demonstrated in human cells, may also enable translation of ACE into higher eukaryotes, despite the challenge of megabase DNA cargo delivery. We are nonetheless curious of the possibilities of translating this technology into more complex biological systems for engineering synthetic eukaryotic genomes.

ACE can also be configured to use other types of CASTs. Although a Type I-F multi-subunit effector CAST for ACE was chosen here, single-effector CASTs, such as those of the Type V-K system, may, in some embodiments, replace the Type I-F system as the integration module in this protocol. Experiments using Cas12k-TniQ-TnsBC in a similar conjugative workflow have been conducted using Cas12k coupled transposases to successfully transfer Syn100 but chose to continue with the Type I-F system on account of its greater integration fidelity and absence of co-integrate formation (data not shown). However, numerous advances in engineering target selection and eliminating co-integrate products of Type V-K CASTs may elevate conjugation coupled Type V-K transposition as a suitable alternative to Type I-F CAST in ACE.

With its numerous benefits, ACE offers improved alternatives to all steps of the current genome synthesis paradigm. Artificial genomes with designated properties may not need to be designed from scratch and then painstakingly synthesized and assembled, but rather melded together from diverse natural genomes in a single ACE or an iteration of several ACE steps. Moreover, in contrast to the low efficiencies of in vitro delivery of 100-kb or larger synthetic DNA, megabases of DNA can be transferred between bacteria via conjugation in a single ACE step. Finally, ACE is the first reported use of CASTs for building synthetic genomes, enabling stable addition and maintenance of megabases of highly homologous genomic segments.

ACE has immediate applications for exploring microbial genome architecture through programmed large genomic transfers. Large genomic transfers with ACE reveals patterns in genome architecture. Integration experiments of segments centered around the dif and adjacent to the oriC into targets around the recipient E. coli genome enabled us to uncover the role of length and identity of the donor fragment in determining permissive transfer sites. We observed that donor segments centered around the dif and containing ter sites can be integrated but were unstable in target regions where the ter sites have their non-permissive ends orientated towards the oriC. Without being bound by any particular theory, we hypothesize that these ectopic ter sites prematurely arrest replication forks before they can reach the natural replication fork trap region of the genome opposite of the oriC, leading to incomplete genome replication, chromosomal instability, and subsequent rearrangement. Moreover, donors chosen adjacent to the oriC lacking ter sequences were largely stably integrated throughout the genome, although at variable rates, but nonetheless suggest a tolerance of the E. coli chromosome for large replichore asymmetries. Elucidating more precise rules beyond ter sites and their underlying mechanisms governing allowed and disallowed donors and insertion site pairings beyond preliminary observations will require the ability to perform systematic, high-throughput integration screens between many variant donor and recipient libraries in future studies. The ability of ACE to test the plasticity and requirement in the positional organization of information on the architectural genome provides a unique opportunity to comprehend, probe and reverse engineer the genomic underpinnings of life.

By encoding complex engineered functions into GEOs, ACE can spawn applications where these large truly genomic scale insertions can confer novel phenotypes and physiology for therapeutic and industrial applications. For example, ACE allows for transfers of large biosynthetic gene clusters into a variety of heterologous hosts for cluster expression, characterization, and refactoring for metabolic engineering. Beyond transferring natural DNA, ACE can also transfer synthetic DNA, interfacing with a plethora of in vitro and in vivo DNA assembly techniques. The ability to integrate large de novo designed and assembled DNA, combined with downstream transcriptomic, proteomic and physiological characterization can accelerate exploration of synthetic genome-scale modules for encoding desired functionalities. This may lead to the use of large language model guided design of GEOs for bespoke functions and applications.

ACE, and iterations of ACE, can integrate multi-megabase scale DNA across arbitrarily chosen donors and recipients to create artificial chimeric genomes. It is demonstrated herein that genomes of GEOs can be stable and can express the fused genomic pieces from multiple distinct parental species. With this novel ability, we echo the question first raised in Greek mythology: would it be possible to piece together a “chimeric” lifeform combining different biological traits across species, genus, or even order segregations? Robust and systematic generation of such “chimera” organisms would provide valuable insights on how a rationally designed synthetic chimeric genome directs the physiology and behavior of the underlying hybrid lifeform. Importantly, these techniques can lay the foundation of a new paradigm of artificial lifeforms to enable novel functions and future applications beyond the available designs of nature.

### Methods

### General Methods

DNA amplification was performed using PrimeSTAR™ GXL DNA Polymerase (TaKaRa Bio USA cat #R050) or Q5® High-Fidelity 2× Master Mix (New England BioLabs (NEB) cat #M0492) unless otherwise stated. DNA oligonucleotides were obtained from Integrated DNA Technologies (IDT) and Millipore Sigma. Gene fragments (gBlocks) were obtained from IDT or Twist Biosciences. Nucleic acid purification and cleanup was done with QIAquick® PCR Purification Kit (QIAGEN™ cat #28106). DNA extraction from agarose gels was conducted with GeneJET Gel Extraction Kit (Thermo Scientific™ cat #K0691). All plasmids were assembled using Gibson assembly using NEBuilder® 2× HiFi Assembly mix (NEB cat #E2621) or Golden Gate assembly using NEBridge® 3× Ligase Master mix (NEB cat #M1100) and BsaI-HF© v2 (NEB cat #R3733). Standard plasmid DNA cloning was performed using electrocompetent E. coli DH10B, which was made electrocompetent as previously described. Plasmid DNA extraction from E. coli DH10B was done with QIAprep© Spin Miniprep Kit (QIAGEN™ cat #27106). All antibiotic and counter-selection concentrations when used are as follows: 100 μg/mL streptomycin, 200 μg/mL hygromycin, 2.5 mM 4-chlorophenylalanine, 25 g/mL chloramphenicol, 7.5% w/v sucrose, 50 μg/mL spectinomycin, 75 μg/mL apramycin, 50 g/mL kanamycin, 100 μg/mL carbenicillin, 10 μg/mL tetracycline and 20 μg/mL gentamicin.

### Bacteria Culturing Conditions

All E. coli and S. flexneri bacterial strains were grown at 37° C. in selective Lysogeny Broth (LB) with shaking unless otherwise stated. Individual colonies were grown overnight on selective LB-agar plates at 37° C. prior to liquid growth. All P. putida, P. protegens and A. tumefaciens bacterial strains were grown at 30° C. in LB with shaking or on solid LB-agar plates. For overnight culturing, V. natriegens bacterial strains were grown at 30° C. in LBv2 for liquid growth or on LB-agar plates for solid growth. For daytime culturing for electrocompetency, V. natriegens was grown at 37° C. in LBv2. V natriegens electrocompetency was performed according to methods known in the art. Antibiotic concentrations were modified when selecting against P. protegens with hygromycin (500 μg/mL) and tetracycline (30 μg/mL) and when selecting against S. flexneri with spectinomycin (100 μg/mL).

Detailed documentation of all bacteria strains and their derivatives used in this study are listed in Table 2A-Table 2D. Briefly, E. coli DH10B rpsL K43R, E. coli DH10B rpsL K43R Δdif::lux, E. coli MDS42 rpsL K43R ΔrecA, Vibrio natriegens ATCC 14048 DSM759, Pseudomonas putida KT2440 and Agrobacterium tumefaciens GV3101 were used as base strains for the construction of ACE recipient strains. Donor strain construction was carried out in E. coli DH10B rpsL K43R Syn100, E. coli MDS42 rpsL K43R ΔrecA, E. coli MDS42 rpsL K43R ΔrecA Δtus, E. coli Syn61 rpsL K43R ΔpheS*-hygroR, Shigella flexneri CF S100 rpsL K43R ΔrecA Δtus ΔoriT ΔhigB Δhok and Pseudomonas protegens pf-5 backgrounds.

### Plasmid Construction

All plasmids and oligonucleotides used in their construction are listed in Table 3-Table 4D. Most plasmid constructs were constructed via Gibson assembly. In short, pAra-CAST was derived from pSL1777 (Addgene #160731) and variant constructs used for control experiments were subcloned from this starting plasmid. pAra-CAST variants under the RSF backbone were constructed for experiments carried out in V. natriegens and P. putida. pAra-CAST variants under the VS1 backbone were constructed for experiments carried out in A. tumefaciens. CAST variants with hygroR resistance gene were used for experiments involving P. protegens donors. The appropriate crRNAs were cloned into pAra-CAST acceptor plasmids using Golden Gate assembly using annealed DNA oligonucleotides.

The principal conjugation component used in this work (pAra-Trans) was derived from pBAD-traRP4 min and the commercial pBelo ori2 BAC. Plasmid templates for Cassette^{START} (pSC101-spectR-cassette1-sC-oriT-RE; pCassette^{START} for short) and Cassette^{STOP} (pSC101-spectR-cassette2-pHGFP-LE-oriT; pCassette^{STOP} for short) were cloned under a pSC101 backbone to facilitate propagation for downstream amplification and λ-red mediated recombination. pCassette^{START-RECM}, pCassette^{STOP-ECM} was subcloned from pCassette^{START} and pCassette^{STOP}, respectively. pSTART-RB^{T-DNA}-STOP-LB^{T-DNA} was subcloned from pCassette^{START}, pCassette^{STOP} and pGoldenBraid-p35S-TMV-2×YFP NLS-tHSP-KanR. For constructs pertaining to OASIS, pLac-OrthoCAST1 (Tn7007) was derived from pSL2361 and pCassette^{START}, while pLac-OrthoCAST2 (Tn7011) was derived from pSL2353 and pCassette^{STOP}. Likewise, pXyl-OrthoCAST1 (Tn7016) was derived from pSL2364 and pCassette^{START}, while pXyl-OrthoCAST2 (Tn7011) was also derived from pSL2353 and pCassette^{STOP}

Donor Strain Construction with Homologous Recombination

Classical λ-red mediated homologous recombination was also used to integrate Cassette^{START} and Cassette^{STOP} into the genome to generate donor strains. Briefly, pCassette^{START} was amplified with PCR using primer pairs bearing homology to the start and end of the transferred region and transformed into pre-induced electrocompetent cells of the relevant bacterial strain (see Table 4A-Table 4D for primer pairs used and Table 5 for Cassette homology sequences). Cells were plated on selective media and genotyped by PCR for cassette integration with the relevant primers in Table 4A-Table 4D. Colonies with the correct integration were sequenced. Cassette^{STOP} was integrated into colonies with sequence verified Cassette^{START} using the same procedure. Strains with both Cassettes integrated into their genome were made electrocompetent for transformation of pAra-Trans by electroporation as previously described to make finalized donor strains. Likewise, to generate recipient strains, base bacterial strains were made electrocompetent and transformed with pAra-CAST by electroporation to finalize recipient strains.

### Conjugative-Transposition Experiments (ACE)

All ACE events were performed at 30° C. based on past reports that CAST-directed transposition is more efficient at 30° C. than at 37° C.

For ACE transfer experiments between E. coli DH10B Syn100 or S. flexneri donor strains and E. coli DH10B, V. natriegens or P. putida recipient strains, donor and recipient cultures were inoculated into 10 mL of LB with appropriate antibiotics and 2% (w/v) glucose for overnight growth (12-16 hours). Overnight cultures were then pelleted by centrifugation and diluted into 100 mL of LB with the appropriate antibiotics at a starting OD₆₀₀ of 0.05. Cultures were grown at 37° C. to OD₆₀₀ of 0.10-0.20 before inducing with 0.5% (w/v) arabinose until cultures reach an OD₆₀₀ of 0.5. Cultures were then pelleted and resuspended in 400 μL of LB. Resuspended donors and recipients were then mixed in a 4:1 donor to recipient ratio (320 μL+80 μL) for a total of 400 μL and spotted in 13 μL aliquots onto a LB-agar plate with no antibiotics. Spots were dried under a flame before incubating at 30° C. for 4-8 hours to allow for conjugation coupled transposition activity. Spots were then washed from the plate using 5 mL of LB and added to a culture 95 mL of LB with the appropriate antibiotics and 0.5% (w/v) arabinose. Cultures were recovered at 37° C. for 3-6 hours before pelleting by centrifugation and then plating by serial dilution on selective LB-agar plates with appropriate antibiotics. Plates were incubated at 37° C. overnight for 12-16 hours, except for transfers into P. putida recipient, which were incubated at 30° C.

For ACE experiments involving E. coli Syn61 donors and for iterative ACE for trimera generation, an abridged ACE protocol that forgoes the growth to log phase was used. Donor and recipient cultures were inoculated into 10 mL of LB with appropriate antibiotics for overnight growth (12-16 hours). Overnight cultures were then pelleted by centrifugation and replenished with new 10 mL of LB with the appropriate antibiotics and 0.5% (w/v) arabinose and incubated at 37° C. for 2 hours. After induction, the remainder of this abbreviated ACE procedure is the same as described above. A comparison of the two described ACE methods is described below.

There are differences between the ACE protocol used in FIG. 2A-FIG. 2G and FIG. 4A-FIG. 4K and that which is used in FIG. 9A-FIG. 9G and FIG. 3A-FIG. 3O (also see above). While both versions of the protocol feature a solid agar conjugation step followed by a recovery and outgrowth step before selective plating, in FIG. 2A-FIG. 2G and FIG. 4A-FIG. 4K experiments were additionally performed with cultures grown to OD600 0.5 from a dilution of the overnight culture. However, this initial culturing contributed a significant amount of additional time to complete the entire protocol, limiting experimental throughput and necessitating the work of two researchers to sustainably complete one experiment. Moreover, the donor and recipient strains used in the experiments typically had different doubling times, so it was challenging to coordinate the growth of both cultures to the OD600 0.5 growth endpoint. By replacing this initial growth step with induction of the overnight culture in fresh media, the protocol time can be reduced by an average of three hours while also decreasing handling volumes, enabling higher experimental throughput and less burden on individual researchers to reasonably carry out megabase-scale ACE in a single day. This reasoning is what led to adoption of the modified protocol for FIG. 3A-FIG. 3O experiments.

On top of these practical experimental considerations, an increase in experimental efficiency (measured by GFP+c.f.u.) was also observed when carrying out the modified protocol compared to the new original protocol with 1-Mb Syn61 dif donor transfers with ACE. Without being bound by any particular theory, this may be due to larger populations of donor and recipient bacteria in the overnight culture compared to daytime culture. Little to no instability was observed when performing this protocol for generating DH10B+1-Mb Syn61 and DH10B+2-Mb Syn61 Syn-seg 1, leading to adoption of this protocol for experiments in both FIG. 9A-FIG. 9G and FIG. 3A-FIG. 3O (FIG. 4A-FIG. 4K experiments were already done with the original FIG. 2A-FIG. 2G protocol). Shortening or outright eliminating the post conjugation recovery and outgrowth step and directly plating the conjugation spots on selective media further reduced of protocol time and was done for some of the experiments of FIG. 7A-FIG. 16H.

For the ACE, conjugative recombination (CR) and conjugative Cas9-coupled recombination (CCR) comparison experiments, the abridged ACE protocol above was used. For 100 Kb ACE, CR and CCR transfers, donor and recipient conjugation spots were incubated for 4 hours at 30° C. with 3 hours of post-conjugation liquid culturing prior to plating on selective solid media. For 1 Mb and 2 Mb transfers, incubation of spots at 30° C. were for 6 hours and 8 hours, respectively, with 3 hours and 6 hours of post-conjugation outgrowth, respectively, prior to plating.

For ACE experiments with E. coli Syn100 donors and recipient E. coli DH10B pAra-CAST variants (FIG. 8E-FIG. 8I), E. coli MDS42 and Syn61 donors with A. tumefaciens GV3101 recipients, as well as P. protegens RK24 donors with E. coli DH10B recipients, the abridged ACE protocol was further shortened to eliminate the post-conjugation liquid culturing step. Donor and recipient cultures were inoculated into 10 mL of LB with appropriate antibiotics for overnight growth (12-16 hours). Overnight cultures induced as in the abbreviated ACE procedure above. After induction, cultures were pelleted and resuspended in 400 μL of LB. Resuspended donors and recipients were then mixed in a 4:1 donor to recipient ratio (100 μL+25 μL) for a total of 125 μL and four spots of 20 μL aliquots were made onto a LB-agar plate with no antibiotics. Spots were dried and incubated for 4 hours at 30° C. Spots were resuspended with 200 μL of LB for direct plating with serial dilutions on selective LB-agar plates. Plates were incubated as with conventional ACE. ACE transfers into Agrobacterium recipient were performed with conjugation spots incubated overnight and incubation of the final selective plates at 30° C.

For ACE experiments with P. protegens donors and E. coli DH10B recipients conducted with triparental mating, donor, recipient and conjugation helper strains were each inoculated into 10 mL of LB with appropriate antibiotics for overnight growth. The abridged ACE protocol without post-conjugation liquid culturing was used for the rest of the steps, with exception of conjugation spotting. Resuspended donor, recipient and helper strains were mixed in a ratio of 2:2:1 (200 μL+200 μL+50 μL) and 20 μL aliquots were spotted on LB-agar plates for 30° C. overnight incubation for triparental conjugation.

For generating stable clonal populations of GEOs for downstream analysis, colonies were picked from selective post-ACE plates and streaked out on fresh selective LB-agar plates for overnight incubation at 37° C. Colonies were picked from the streak-out plate and grown overnight in selective LB for long-term storage in 15% (v/v) glycerol at −80° C. A list of targets used in ACE experiments is given in Table 6A-Table 6B, a list of ACE experiments and selection schemes used is given in Table 7A-Table 7B and a list of ACE chimeric genomes (GEOs) made is given in Table 8. For GFP+c.f.u. ACE efficiency plots and in vivo ACE specificity screens, colonies of the correct phenotypes were counted on post-ACE plates.

### Phenotyping and Genotyping PCR Analysis

Single colonies of post-ACE were picked from selective LB-agar streak-out plates and resuspended in 40 μL of milli-Q H₂O. For colony phenotyping assays, 3-4 μL of the resuspension is spotted onto LB-agar plates with the target antibiotic. For colony genotyping PCR, reactions were performed with 2λ Red Taq Master Mix (Apex Bioresearch products cat #42-138) with 1 μL of the resuspension used as template per 20 μL of reaction volume, which contains 0.2 μM of each primer. Cycling protocol starts with 2 minutes of initial cell lysis and denaturation at 98° C. followed by 36 cycles of 95° C. denaturation, 60-65° C. primer annealing and 72° C. extension (at 1 kb/minute) with a final extension of 5 minutes at 72° C. For junction detection, primer pairs contained one donor-specific primer and one recipient-specific primer and were varied so that all integration orientation could be assayed for both the LE and RE transposon junctions. For internal region detection of the transferred sequence, primer pairs containing two donor-specific primers were used. PCR amplicons were resolved by 1% agarose gel electrophoresis using SYBR Safe staining for visualization.

All oligonucleotides used in analysis and validation of post-ACE bacteria are listed in Table 4A-Table 4D.

### Marker Excision for Iterative ACE

For +2/−2 and GFP marker excision for E. coli DH10B Δdif+2-Mb Syn61 Syn-seg. 1, the chimeric cells were made electrocompetent and transformed with pCS20 and plated on gentamicin selective LB-agar plates. Leaky expression of the PhiC31 recombinase enabled low levels of marker excision in the population, which was purified by streaking single colonies the next day on 4-chlorophenylalanine counter-selective plates. GFP-colonies were spotted on selective solid LB-agar containing hygromycin or counter-selection to verify the loss of markers. Excision of the markers were also assessed with genotyping colony PCR of the LE. Confirmed colonies were made electrocompetent and transformed with pAra-CAST-dif for downstream trimeric genome generation with iterative ACE.

Donor Strain Construction with OASIS

Competent E. coli DH10B pAra-Trans were electroporated with pLac-oCAST or pXyl-oCAST for inserting Cassette^{START} or Cassette^{STOP} in a specific genomic locus of the base strain to be engineered into an ACE donor. The E. coli oCAST integrator and base strain are grown in 10 mL of LB supplemented with appropriate antibiotics for overnight growth (12-16 hours). 2% (w/v) glucose is added to integrator strain cultures for conjugation suppression. Overnight integrator cultures were then pelleted by centrifugation and replenished with new 10 mL of LB with the appropriate antibiotics and 0.5% (w/v) arabinose, as well as 100 μM IPTG for pLac integrators or 2.5 mM m-toluate for pXyl integrator and incubated at 37° C. for 2 hours. The base strain overnight culture is also pelleted by centrifugation and replenished with new 10 mL of LB with appropriate antibiotics prior to incubation at the appropriate culturing temperature for 2 hours. Cultures were then pelleted and resuspended in 400 μL of LB. Resuspended donors and recipients were then mixed in a 1:1 donor to recipient ratio (200 μL+200 μL) for a total of 400 μL and spotted in 20 μL aliquots onto a LB-agar plate with the appropriate inducers (0.5% (w/v) arabinose and 100 μM IPTG or 2.5 mM m-toluate) and no antibiotics. Spots were dried under a flame before incubating at 30° C. for 4-8 hours and up to overnight to allow for conjugation coupled transposition activity. Spots were resuspended with 200-750 μL of 0.5× LB for direct plating on selective LB-agar plates that would only allow base strains with the desired genome integration to survive. Plates were incubated at the appropriate culturing temperature of the base strain overnight. Colonies were picked from plates and streaked out on fresh selective LB-agar plates for genotyping PCR before the subsequent round of OASIS conjugative transposition of the final Cassette. Strains with both Cassettes inserted and validated were either transformed with pAra-Trans by electroporation or with RK24 by conjugation.

### Agroinfiltration Experiments

N. benthamiana plants were grown from seed in a Vivosun growth chamber (Amazon) at a constant temperature of 25° C., humidity between 40-50% and a 16/8 day/night cycle. Plants were grown in soil (3 parts Propagation mix (Sphagnum Peat Moss), 1 part Perlite, 1 part Vermiculite, 2 μg/L 14-14-14 Osmocote and 0.6 μg/L Micromax). Plants were grown for 7-8 weeks prior to infiltration.

To prepare engineered and control Agrobacterium strains for N. benthamiana infiltration, glycerol stock resuspensions were struck out on LB/agar plates with kanamycin (50 μg/μL, pCT001-YPET/genomic insertions), gentamicin (pTi, 15 μg/μL) and tetracycline (15 μg/μL, pSOUP) and incubated at 30° C. for 2-3 days. Following incubation, a single isolated colony was picked, per strain, and grown overnight in 3 mL LB with kanamycin, gentamicin and tetracycline. The following day, cultures were centrifuged at 4000 g for 10 min and resuspended in 3 mL freshly made induction media (10 mM MES pH 5.5, 10 mM MgCl2 and 200 μM acetosyringone (diluted in Ethanol)). Cultures were then incubated in a shaking incubator at 200 rpm for 4 hours at room temperature. Prior to infiltration, cultures were diluted to OD 600 of 0.4.

A 1 mL needleless syringe was used to infiltrate N. benthamiana leaves with diluted and induced Agrobacterium cultures. Roughly 200 μL of culture, per leaf, was delivered to 3 individual leaves on a one agrobacteria strain per plant basis. For each infiltration, culture was added to the syringe and the tip of the syringe was directed to the abaxial surface of the leaf. Counter pressure from the adaxial surface was applied using a finger to encourage infiltration. Following infiltration, the day/night cycle was switched to 12/12.

Leaf tissue samples were harvested 72-96 hours following infiltration using a standard hole-punch. Fluorescent images of tissue samples were acquired using a confocal laser scanning microscope (LSM-800, Zeiss) with a 20× objective.

### Solid Agar Hemolysis Assay

Bacterial cultures were grown overnight at 30° C. in LB with appropriate antibiotics. The next day, cultures were pelleted and resuspended with their supernatant to an OD600 of 10, after which 10 μL of the resuspension was spotted on solid agar plates consisting of Columbia media supplement with 5% v/v defibrinated sheep blood (Lampire Biological Laboratories) and incubated at 30° C. for 24 hours. Characterization of promoter types housed within imported regions of chimeric genomes.

Chimeric genomes made between V. natriegens as a recipient and E. coli as a donor were chosen to screen and identify promoter classes found within imported genomic segments of chimeric genomes. Briefly, three hypothetical promoter classes (here, I. Strong promoters in both V natriegens and E. coli, II. Strong promoters in V natriegens and weak promoters in E. coli, and III. Weak promoters in V. natriegens and strong promoters in E. coli) were screened by sorting TPM values obtained from RNA-Seq of GEO: 1219067-1110693.1 (V. natriegens-E. coli Chr. 1 chimera), GEO: 1219067-1110693.2 (V. natriegens-E. coli Chr. 2 chimera), V. natriegens and E. coli based on values reported for each. A TPM filter cutoff of at least 50 was selected for strong, detectable promoter expression, and of less than 5 for weak, hard to detect promoter expression. The top 10 genes were selected from resulting gene lists, and their respective expression profiles were visualized through a heatmap. For Promoter Class I, a subset of promoters identified in the unfiltered list were selected and validated to be functional and equivalent to strong synthetic promoters in both V natriegens and E. coli. Briefly, class I promoters were cloned upstream of a riboJ insulator sequence and a green fluorescent reporter gene, and relative fluorescence units (r.f.u.) were quantified following normalization at OD600 1 using a Tecan Spark plate reader.

### Growth Curve Measurements

Bacterial cultures were inoculated the night before in LB and appropriate antibiotics and grown overnight for 12-16 hours. Cultures were pelleted by centrifugation (4000 rpm for 10 minutes) and spent media was replaced with the same volume of fresh LB without antibiotics. Cultures were then diluted to 0.04 into 1 mL of fresh LB without antibiotics and 200 μL of the dilution was aliquoted onto a 96-well plate. Growth experiments were performed with three technical replicates per strain. Growth experiment was performed on a Tecan Spark® Multimode Microplate Reader for 20 hours at 37° C. with double orbital shaking at 108 rpm. OD₆₀₀ readings were taken every 15 minutes. Data was truncated at 12 hours to account for evaporation of the wells causing disturbance of the OD₆₀₀ readings late into the experiment.

### Validation of Genome Integrity of Chimeric Genomes Throughout Growth Generations.

Representative chimeric genomes were grown for over 100 generations at either 30° C. or 37° C. in rich media of different complexities (here, LB and TB), or M9 minimal media conditions using 0.4% w/v glucose as a carbon source. Briefly, strains were initially inoculated from glycerol stock and continuously grown in 10 mL of media for 24 hours in the aforementioned conditions. The following day, the cultures were back diluted in 10 mL of media and re-grown overnight under the same conditions. This procedure was repeated over the course of 5 days, assuming a conservative estimate of 20 generations per day based on the lowest generation time reported for chimeric genomes tested in this study.

### Confocal Microscope Imaging

Morphology and phenotype of chimeric strains were observed with a confocal laser scanning microscope (Zeiss LSM 800). Bacteria cells are seeded on the LB agarose gel pads and then mounted on the glass-bottom confocal dish. Phase contrast and fluorescence images were acquired using an oil immersion objective plan-apochromat 100×/1.4.

### Whole Genome Sequencing (WGS)

Bacterial cultures were grown in LB supplemented with the appropriate antibiotics until confluent. Cultures were pelleted by centrifugation and genomic DNA (gDNA) was extracted from the pellets using DNeasy Blood & Tissue Kit (QIAGEN™ cat #69504) per manufacturer instructions. Concentrations of gDNA were quantified with the Invitrogen Qubit™ 4 Fluorometer using the 1×dsDNA High Sensitivity (HS) assay kit (Thermo Fisher Scientific cat #Q33231). For library preparation, Nextera™ DNA Flex Library Prep Kit (Illumina) was used per manufacturer instructions to tag, barcode, amplify and add index primers (i5 and i7) to the library. 200 ng of starting gDNA was used per barcoded library. Prepared libraries were quantified with the Qubit™ 4 Fluorometer before pooling 15 ng per barcoded library. Pooled library denaturation and flow cell loading was performed per manufacturer instructions. Sequencing was performed on an Illumina MiSeq using MiSeq reagent kit v3 (600-cycles) with 300 bp paired-end reads and automated demultiplexing and adapter trimming.

All WGS of strains was performed as above with the exception of E. coli DH10B Δdif::S. flexneri-1Mbdif Sequencing libraries for this strain were prepared and sequenced on an Illumina NextSeq2000 with 50 bp paired-end reads by the Millard and Muriel Jacobs Genetics and Genomics Laboratory at the California Institute of Technology.

### WGS Analysis

Sequencing reads from whole-genome samples were aligned to expected reference genomes using the Bowtie2 short-read aligner. Alignment files were further processed with Samtools and deepTools was used to estimate RPGC throughout the expected reference genome in 10-kb bins. References for gene homolog variants for the recipient and donor strains were identified via a reciprocal alignment approach using Blast. Identified gene homolog variants were used as references to estimate, independent of short-read sequence alignment, the read fraction allocated to each respective reference position. The read fraction estimation per reference was calculated as the sum of counts across 14-bp bins encompassing distinct nucleotide polymorphisms detected via multiple-sequence alignment using MUSCLE.

### Transposon Sequencing (Tn-Seq)

Colonies on post-ACE selection plates were scraped into 4 mL of LB and grown in appropriate antibiotics for 4 hours before 1 mL of culture was pelleted by centrifugation. gDNA was extracted from the pelleted cell populations using DNeasy Blood & Tissue Kit (QIAGEN™ cat #69504) per manufacturer instructions. Concentrations of gDNA were quantified with the Invitrogen Qubit™ 4 Fluorometer. For Tn-Seq library preparation, 200 ng of starting gDNA was tagmented and washed with Nextera™ DNA Flex Library Prep Kit as per manufacturer instructions. For library amplification and barcoding, custom index 2 (i5) oligonucleotides annealing to the RE of Tn6677 were used instead of the standard i5 indices (see Table 4A). These custom i5 oligos were used alongside standard index 1 (i7) oligos provided Illumina with 15× cycles of library amplification PCR at 65° C. annealing (all else according to the manufacturer). Barcoded libraries were cleaned up as per manufacturer instructions and Qubit™ 4 Fluorometer before 30 ng per library was pooled together. Pooled library denaturation and flow cell loading was performed per manufacturer instructions. Prior to sequencing, a custom sequencing primer for the RE of Tn6677 was spiked into the reagent kit in well 12 (Read 1 HP10) at a final concentration of 0.5 μM to enable targeted sequencing of enriched transposon sequences within the libraries. Sequencing was performed on an Illumina MiSeq using MiSeq reagent kit v3 (600-cycles) with 300 bp paired-end reads and automated demultiplexing and adapter trimming.

### Tn-Seq Analysis

Custom Python pipelines were developed to process Tn-Seq data generated from ACE experiments, according to previously described methods. Briefly, reads containing the 15-bp 5′-terminal sequence of the enriched Tn arm end were then selected and the upstream 17-bp region was used as a genomic footprint corresponding to the integration site in the genome. Fingerprint sequences were then aligned to expected reference genomes using Bowtie2. Alignment positions documented per read were later grouped into unique 10-kb bins, and unless stated otherwise insertion frequencies were plotted as a percentage of total mapping reads. The on-target percentage was calculated as the percentage of reads corresponding to integration events within the 10-kb window flanking the target integration site. The integration orientation bias is defined as the ratio of number of reads corresponding to T-RL insertions to those corresponding to T-LR insertions.

### RNA Sequencing (RNA-Seq)

For RNA-Seq of E. coli DH10B Δdif+2-Mb Syn61 Syn-seg. 1 and its progenitor donor and recipient strains, bacterial cultures were grown in LB supplemented with the appropriate antibiotics overnight. The next day, the cultures were back diluted to OD₆₀₀ of 0.05 in fresh 10 mL of LB without any antibiotics and grown to an OD₆₀₀ of 0.6. 1 mL of the culture was pelleted by centrifugation for RNA extraction using RNeasy Protect Bacteria Mini Kit (QIAGEN™ cat #74524). Extracted RNA was quantified using Invitrogen Qubit™ 4 Fluorometer using the RNA High Sensitivity (HS) assay kit (Thermo Fisher Scientific cat #Q32852). Sequencing libraries from extracted RNA samples were prepped and sequenced on an Illumina NextSeq2000 with 50 bp paired-end reads by the Millard and Muriel Jacobs Genetics and Genomics Laboratory at the California Institute of Technology. RNA-Seq experiments were performed with three technical replicates per strain.

For RNA-Seq and differential gene expression of E. coli DH10B Δdif::Syn. Seg 1-S. flexneri-1Mbdif E. coli DH10B Δdif::S. flexneri-1Mbdif E. coli MDS42 Δdif::S. flexneri-1Mbdif and associated donor and recipient strains, and additionally for differential gene expression of E. coli DH10B Δdif+2-Mb Syn61 Syn-seg. 1 and associated donor and recipient strains, bacterial cultures were grown in LB supplemented with the appropriate antibiotics overnight. The next day, the cultures were back diluted to OD₆₀₀ of 0.05 in fresh 10 mL of LB without any antibiotics and grown to an OD₆₀₀ of 0.6. 500 μL of the culture was pelleted by centrifugation and frozen for submission to GENEWIZ (USA; genewiz.com), for RNA extraction with rRNA depletion, library preparation and standard RNA sequencing on an Illumina NovaSeq6000 with 150 bp paired-end reads. RNA-Seq experiments were performed with three technical replicates per strain.

### RNA-Seq Analysis

Custom Python pipelines were developed to quantify RNA sequencing reads uniquely mapping to gene homolog references. Sequencing reads from whole-transcriptome RNA samples were first aligned to expected transcriptome references per expected genome reference using the Bowtie2 short-read aligner. Because reads can map to multiple gene homolog variants, counts were allocated to specific gene homolog references by using discriminatory 14-bp bins per reference (identified via MUSCLE). The raw counts per gene homolog reference was then transformed to log(TPM+1) and plotted using various visualization and analysis pipelines written in Python. For differential expression analysis, raw counts of triplicate samples were further processed via DESeq2, internally normalizing samples and calculating fold-change and adjusted p-values per transcript reference. DESeq2 results were illustrated as a volcano plot, highlighting enrichment of redundant genes within expected references. Gene ontology pathway enrichment analysis was performed using ShinyGO on the intersection of over-expressed genes in chimeric ACE samples, as defined by an adjusted p-value<0.001 and a fold-change ≥1.

### Sample Preparation for Proteomic Analysis

E. coli cell pellets were resuspended in 5% sodium dodecyl sulfate (Sigma-aldrich) in 50 mM HEPES, and were homogenized using BeatBox (Preomics) for 10 min under ‘High’ settings. Protein concentration was measured using Pierce BCA protein assay kit (Pierce), and 100 g of protein was used for further sample preparation. The samples were reduced using 5 mM tris(2-carboxyethyl)phosphine (Sigma-Aldrich) under 55° C. for 10 min, and then alkylated with chloroacetamide (Sigma-Aldrich) under room temperature for 15 min. The samples were further acidified to a final concentration of 2.5% phosphoric acid, and 25 μl of sample was combined with 165 μl of 90% methanol with 10% of 1 M triethylammonium bicarbonate (TEAB, Thermo Scientific) according to protocols known in the art. The samples were then loaded onto S-trap (Protifi) devices, and the S-trap devices were washed using the same buffer for loading (90% methanol with 10% of 1 M TEAB) for 3 times. For each loading or washing steps, the S-trap devices were centrifuge at 4000 g for 30 seconds to remove the elute. After washing steps, 20 μl of 100 mM TEAB containing 10 μg of TPCK-trypsin (Thermo Scientific) was added into each sample, and the digestion was allowed to proceed overnight. The digested peptides were eluted using 40 μl of 50 mM TEAB in water, 0.2% formic acid in water, and 50% acetonitrile in water sequentially with 4000 g centrifugation for 1 min for each elution. The eluate for 3 steps were pooled together and dried using a refrigerated CentiVap concentrator (Labconco). The dried samples were stored in −20° C. before resuspended in mobile phase A (2% acetonitrile, 0.2% formic acid, and 97.8% water) for LC-MS/MS analysis.

### LC-MS/MS for Proteomic Analysis

For proteomic samples, LC-MS/MS experiments were performed by loading 500 ng sample onto an EASY-nLC 1200 (ThermoFisher Scientific, San Jose, CA) connected to an Q Exactive HF Quadrupole—Orbitrap Hydrid mass spectrometer (Thermo Fisher Scientific, San Jose, CA). Peptides were separated on an Aurora Ultimate XT UHPLC column (25 cm×75 m, 1.6 m C18, AUR3-25075C18-XT, Ion Opticks) with a flow rate of 0.35 μL/min and for a total duration of 131 min. The gradient was composed of 3% Solvent B for 1 min, 3-19% B for 72 min, 19-29% B for 28 min, 29-41% B for 20 min, 41-95% B for 3 min, and 95-98% B for 7 min. Solvent A consists of 97.8% H₂O, 2% ACN, and 0.2% formic acid, and solvent B of 19.8% H₂O, 80% ACN, and 0.2% formic acid. MS1 scans were acquired with a range of 375-1500 m/z in the Orbitrap at 60 k resolution. The maximum injection time was 15 ms, and the AGC target was 3×10⁶. MS2 scans were acquired at 30 k resolution with a first scan mass as 100 Da. The maximum injection time was 45 ms, and the AGC target was 3×10⁶. The isolation window was 1.2 m/z, collision energy was 28 NCE, and loop count is 12. Other global settings were set to the following: ion source type, NSI; spray voltage, 2000 V; ion transfer tube temperature, 300° C. Method modification and data collection were performed using Xcalibur software (Thermo Scientific).

### Proteomics Data Analysis

Proteomic analysis was performed using Proteome Discoverer 2.5 (PD 2.5, Thermo Scientific) software, and SequestHT with Percolator validation. The raw data was searched against Escherichia coli proteome (retrieved from Uniprot Knowledgebase on 2020 Nov. 2) with the list of inserted proteins for each insertion, respectively. Percolator FDRs were set at 0.01 (strict) and 0.05 (relaxed). Peptide FDRs was set at 0.01 (strict) and 0.05 (relaxed), with medium confidence and a minimum peptide length of 6. Carbamidomethyl (C) was set as a static modification; oxidation (M) was set as a dynamic modification; acetyl (protein N-term), Met-loss (Protein N-term M) and Met-loss+acetyl (Protein N-term M) were set as dynamic N-Terminal modifications.

Further analysis was performed using in-house python script. Briefly, only the data with raw intensity higher than 100000 was subjected to further analysis, and all the quantitation from match-between-run analysis was eliminated to prevent false discovery. The data was further normalized to each sample's median value for the PCA analysis and heatmap. PCA analysis was performed using scikit-learn v1.3.0 python package.

Metabolite Extraction from Cell Pellet

E. coli cell pellets were resuspended in 1 mL acetonitrile (MS grade, Fisher Scientific), and homogenized using BeatBox (Preomics). The homogenized samples were centrifuged under 16000 g for 10 min, and the supernatant was dried using a refrigerated CentiVap concentrator (Labconco). The dried samples were stored in −80° C. before being resuspended in 50 μl of mobile phase A (2% acetonitrile, 0.2% formic acid, and 97.8% water) for LC-MS/MS analysis.

Metabolite Extraction from Culture Medium

5 ml of culture medium was extracted with equivalent amount of ethyl acetate (Fisher Scientific) at 37° C. for 1 h. The ethyl acetate extraction was dried and resuspended in 50 μl of mobile phase A (2% acetonitrile, 0.2% formic acid in water).

### LC-MS/MS for Metabolomic Analysis

For metabolic samples, LC-MS/MS experiments were performed by loading 5 μl of metabolite extract onto an Vanquish HPLC (ThermoFisher Scientific, San Jose, CA) connected to an Orbitrap Fusion Tribrid mass spectrometer (Thermo Fisher Scientific, San Jose, CA). Peptides were separated on a Kinetex C18 column (100×4.6 mm, 2.6 m C18, 100 Å, H17-133371, Phenomenex) with a flow rate of 0.3 mL/min and for a total duration of 30 min. The gradient was composed of 5-30% B for 2 min, 30-95% B for 24 min, 95% B for 2 min, 95-5% B for 1 min, and 5% B for 3 min. Solvent A consists of 99.8% H₂O, and 0.2% formic acid, and solvent B of 99.8% ACN, and 0.2% formic acid. MS1 scans were acquired with a range of 375-1500 m/z in the Orbitrap at 50 k resolution. The maximum injection time was 100 ms, and the AGC target was 1×10⁶. MS2 scans were acquired at 7.5 k resolution. The maximum injection time and the AGC target was set as auto. The isolation window was 1.2 m/z, collision energy was 30 NCE, and loop time was 2 seconds. Other global settings were set to the following: ion source type, H-ESI; spray voltage, 4200 V (positive) and 3500 V (negative); ion transfer tube temperature, 350° C.; and vaporizer temperature, 200° C. Method modification and data collection were performed using Xcalibur software (Thermo Scientific).

### Metabolomics Data Analysis

Peak alignment and quantitation were performed with Compound Discoverer 3.3 (Thermo Scientific) using an untargeted metabolomics workflow. All the identified features were listed and subjected to similar in-house python script as the proteomic analysis. PCA analysis was performed using scikit-learn v1.3.0 python package. Annotation of orfamide B, orfamide C, pyoluteorin, and rhizoxins were performed manually by assigning the experimental m/z to the theoretical m/z with a tolerance of 10 ppm.

### Data Reporting

No statistical methods were used to predetermine sample size. The experiments were not randomized, and investigators were not blinded to allocation during experiments and outcome assessment.

### Statistical Analysis

Unless stated otherwise, statistical analysis on triplicates for experiments describing the efficiency and precision of ACE were performed by calculating the mean and standard deviation (SD) of each experimental sample group. For transcriptomic analysis, raw counts were converted to log(TPM+1), where each transcript count was normalized by the expected transcript reference length. Differential expression analysis was carried out using DESeq2 using raw counts of samples as input. The following cutoffs were applied to demark significant changes in RNA expression in the context of massively expanded genomes: a base expression value (raw count) >5, an adjusted p-value f 0.001, and a fold-change of 1. Gene ontology pathway enrichment analysis was performed using standard parameters in ShinyGO 0.77, only showing up to 10 pathways.

Table 2A-Table 9 below display exemplary sequences and data of the present disclosure.

TABLE 2A
Parental Strains (Base Strains)

| ID | Strain* | Description# |
|---|---|---|
| Base 1 | E. coli DH10B rpsL K43R | Standard E. coli cloning strain with |
| | | streptomycin resistance |
| Base 2† | E. coli DH10B Δdif::lux | Derived from (1) E. coli cloning strain for ACE |
| | | recipients |
| Base 3† | E. coli DH10B Syn100ΔlacZ | Derived from (1) E. coli cloning strain using |
| | | REXER mediated insertion of Syn 100 for ACE |
| | | donors |
| Base 4 | E. coli MDS42 rpsL K43R | Minimized E. coli strain with streptomycin |
| | | resistance |
| Base 5† | E. coli MDS42 ΔrecA | Derived from (4) minimized E. coli strain with |
| | | deletion of recA for ACE recipients and donors |
| Base 6† | E. coli MDS42 ΔrecA Δtus | Derived from (5) minimized E. coli strain with |
| | | deletion of recA and tus for ACE donors |
| Base 7 | E. coli Syn61 rpsL K43R pheS*-hygroR | Derived from (4) minimized E. coli with a |
| | | completely recoded genome |
| Base 8† | E. coli Syn61 rpsL K43R ΔpheS*-hygroR | Derived from (7) minimized, recoded E. coli |
| | | with deletion of pheS *-hygroR for ACE donors |
| Base 9 | Shigella flexneri CFS100 | Derived from Shigella flexneri str. 247T |
| | | serotype 2a (ATCC 700930) with the virulence |
| | | plasmid cured (ApVir) |
| Base 10† | Shigella flexneri CFS100 rpsL K43R ΔrecA | Derived from (9) avirulent S. flexneri strain |
| | Δtus ΔoriT ΔhigB Δhok | with streptomycin resistance, deletion of recA |
| | | and tus, in addition deletions of genome oriT |
| | | sequence and deletion of higB toxin of the |
| | | higAlhigB antitoxin-toxin system and deletion |
| | | of hok toxin of the hok/sok antitoxin-toxin |
| | | system stabilizing an episome found in S. |
| | | flexneri CFS100, deletion of these toxins |
| | | enabled curing of said episome, which shares |
| | | the same plasmid incompatability group as the |
| | | pBelo bacterial artificial chromosome (BAC) |
| | | backbone. This strain was used for construction |
| | | of ACE donors |
| Base 11 | Vmax Express | Derived from Vibrio natriegens ATCC 14048 |
| | | DSM759, fast growing bacterial strain for ACE |
| | | recipients |
| Base 12 | Pseudomonas putida KT2440 (ATCC | Sourced from American Type Culture |
| | 47054) | Collection (ATCC), for ACE recipients |
| Base 13 | Pseudomonas protegens pf-5 | Also known as Pseudomonas protegens pf-5 |
| | | ATCC BAA-477, for ACE donors |
| Base 14 | Agrobacterium tumefaciens GV3101 | Derived from the A. tumefaciens C58 |
| | | chromosomal background with Ti plasmid |
| | | pMP90 (pTiC58DT-DNA) for ACE recipients |
| | | and plant transformation |

*Note that all strains of E. coli DH10B, E. coli MDS42 and E. coli Syn61 as well as S. flexneri are built on rpsL K43R background. All S. flexneri strains are built on attenuated multiple deletion strain. rpsL K43R is omitted except in the source strain to avoid redundacy.
#Any genomic modification performed on each strain was done with homologous recombination unless otherwise stated. Recombination for gene deletions or donor strain engineering was done with the pKW20 helper plasmid in the case of E. coli DH10B or E. coli MDS42 or with pCDFkan-pAraRed-recA for E. coli Syn61.
†This study

TABLE 2B
Parental Strains (Donor and Helper Strains) †

| ID | Strain |
|---|---|
| Donor 1 | E. coli DH10B-Syn100ΔlacZ-1R-10Kb-2L |
| Donor 2 | E. coli DH10B-Syn100ΔlacZ-1R-50Kb-2L |
| Donor 3 | E. coli DH10B-Syn100ΔlacZ-1R-100Kb-2L |
| Donor 4 | E. coli MDS42-lacZ-1R-200Kb-2L |
| Donor 5 | E. coli Syn61-1R-100Kbdif-2L |
| Donor 6 | E. coli Syn61-1R-600Kbdif-2L |
| Donor 7 | E. coli Syn61-1R-1Mbdif-2L |
| Donor 8 | E. coli Syn61-1R-2Mbdif-2L Syn Seg. 1 |
| Donor 9 | E. coli Syn61-1R-1Mb-2L Syn Seg. 2 |
| Donor 10 | E. coli Syn61-1R-500Kb-2L Syn Seg. 3 |
| Donor 11 | E. coli Syn61-1R-500Kb-2L Syn Seg. 4 |
| Donor 12 | E. coli Syn61-1R-1MboriC-2L Syn Seg. 5 |
| Donor 13 | S. flexneri 2a-1R-1Mbdif-2L |
| Donor 14 | E. coli MDS42-1R-1Mbdif-2L |
| Donor 15 | E. coli MDS42-1RRB-100Kbdif-2LBL (T-DNA) |
| Donor 16 | S. flexneri 2a-1R-1500Kbdif-2L |
| Donor 17 | P. protegens pf-5-1R-orfamide-2L (80Kb) |
| Donor 18 | P. protegens pf-5-1R-rhizoxin-2L (140Kb) |
| Donor 19 | P. protegens pf-5-1R-pyrrolnitrin-2L (60Kb) |
| Donor 20 | P. protegens pf-5-1R-pyoluteorin-2L (66Kb) |
| Donor 21 | P. protegens pf-5-1R-pyoverdine-2L (246Kb) |
| Donor 22 | P. protegens pf-5-1R-citronellol-pyoverdine-2L (373Kb) |
| Donor 23 | P. protegens pf-5-1R-pyoverdine-citronellol-2L (395Kb) |
| Donor 24 | P. protegens pf-5-1R-pyoluteorin-rhizoxin-2L (415Kb) |
| Donor 25 | P. protegens pf-5-1R-citronellol-2L (522Kb) |
| Donor 26 | E. coli Syn61-1luxA-100Kbdif-2luxA |
| Donor 27 | E. coli Syn61-1luxA-1Mbdif-2luxA |
| Donor 28 | E. coli Syn61-1luxA-2Mbdif-2luxA |
| Helper 1* | E. coli Pirl pEVS104 |
| Helper 2* | E. coli ECNR2.ΔtolC. mutS:zeo pRK24 |

*From addgene
† Note that all strains of E. coli DH10B, E. coli MDS42 and E. coli Syn61 as well as S. flexneri are built on rpsL K43R background. All S. flexneri strains are built on attenuated multiple deletion strain. rpsL K43R is omitted except in the source strain to avoid redundacy.

TABLE 2C
Parental Strains (Recipient Strains) †

| ID | Strain |
|---|---|
| Recipient 1 | E. coli DH10B Δdif::lux CAST-TS1 |
| Recipient 2 | E. coli DH10B Δdif::lux CAST-TS2 |
| Recipient 3 | E. coli DH10B Δdif::lux CAST-TS3 |
| Recipient 4 | E. coli DH10B Δdif::lux CAST-luxA* |
| Recipient 5 | E. coli DH10B Δdif::lux CAST-luxB1 |
| Recipient 6 | E. coli DH10B Δdif::lux CAST-luxB2 |
| Recipient 7 | E. coli DH10B Δdif::lux CAST-NT |
| Recipient 8 | E. coli DH10B Δdif::lux CAST-ΔcrRNA |
| Recipient 9 | E. coli DH10B Δdif::lux CAST-Δcascade |
| Recipient 10 | E. coli DH10B Δdif::lux CAST-ΔQcascade |
| Recipient 11 | E. coli DH10B Δdif::lux CAST-Δtns |
| Recipient 12 | E. coli DH10B Δdif::lux ΔCAST |
| Recipient 13 | E. coli DH10B CAST-TS1 |
| Recipient 14 | E. coli DH10B CAST-TS3 |
| Recipient 15 | E. coli DH10B CAST-dif |
| Recipient 16 | E. coli MDS42 CAST-dif |
| Recipient 17 | P. putida KT2440 kanRSFCAST-glmS |
| Recipient 18 | V. natriegens DSM759 carbRSFCAST-wbfF |
| Recipient 19 | V. natriegens DSM759 carbRSFCAST-difch1 |
| Recipient 20 | V. natriegens DSM759 carbRSFCAST-difch2 |
| Recipient 21 | E. coli DH10B Δdif::Syn. Seg 1 CAST-dif |
| Recipient 22 | A. tumefaciens spectVS1CAST-flaA |
| Recipient 23 | A. tumefaciens spectVS1CAST-agpI |
| Recipient 24 | A. tumefaciens spectVS1CAST-tetA |
| Recipient 25 | A. tumefaciens spectVS1CAST-dif(agro) |
| Recipient 26 | E. coli DH10B Δdif::lux hygroCDFCAST-TS2 |
| Recipient 27 | E. coli DH10B Δdif::lux hygroCDFCAST-TS3 |
| Recipient 28 | E. coli DH10B hygroCDFCAST-dif |
| Recipient 29 | E. coli DH10B hygroCDFCAST-TS2 |
| Recipient 30 | E. coli DH10B hygroCDFCAST-TS3 |
| Recipient 31 | E. coli DH10B hygroCDFCAST-TS4 |
| Recipient 32 | E. coli DH10B hygroCDFCAST-TS5 |
| Recipient 33 | E. coli DH10B Δdif::lux pKW20 |
| Recipient 34 | E. coli DH10B Δdif::lux pKW20 sgRNA-luxA |

† Note that all strains of E. coli DH10B, E. coli MDS42 and E. coli Syn61 as well as S. flexneri are built on rpsL K43R background. All S. flexneri strains are built on attenuated multiple deletion strain. rpsL K43R is omitted except in the source strain to avoid redundacy.

TABLE 2D
Parental Strains (OASIS Strains) †

| ID | Strain |
|---|---|
| OASIS 1 | E. coli DH10B pBAD-traRP4min pLac-OrthoCAST1-1500KbShigella-START |
| OASIS 2 | E. coli DH10B pBAD-traRP4min pLac-OrthoCAST2-1500KbShigella-STOP |
| OASIS 3 | E. coli DH10B pBAD-traRP4min pXyl-OrthoCAST1-orfamide-START |
| OASIS 4 | E. coli DH10B pBAD-traRP4min pXyl-OrthoCAST2-orfamide-STOP |
| OASIS 5 | E. coli DH10B pBAD-traRP4min pXyl-OrthoCAST1-rhizoxin-START |
| OASIS 6 | E. coli DH10B pBAD-traRP4min pXyl-OrthoCAST2-rhizoxin-STOP |
| OASIS 7 | E. coli DH10B pBAD-traRP4min pXyl-OrthoCAST1-pyrrolnitrin-START |
| OASIS 8 | E. coli DH10B pBAD-traRP4min pXyl-OrthoCAST2-pyrrolnitrin-STOP |
| OASIS 9 | E. coli DH10B pBAD-traRP4min pXyl-OrthoCAST1-pyoluteorin-START |
| OASIS 10 | E. coli DH10B pBAD-traRP4min pXyl-OrthoCAST2-pyoluteorin-STOP |
| OASIS 11 | E. coli DH10B pBAD-traRP4min pXyl-OrthoCAST1-pyoverdine-START |
| OASIS 12 | E. coli DH10B pBAD-traRP4min pXyl-OrthoCAST2-pyoverdine-STOP |
| OASIS 13 | E. coli DH10B pBAD-traRP4min pXyl-OrthoCAST1-citronellol-START |
| OASIS 14 | E. coli DH10B pBAD-traRP4min pXyl-OrthoCAST2-citronellol-STOP |

*Note that all strains of E. coli DH10B, E. coli MDS42 and E. coli Syn61 as well as S. flexneri are built on rpsL K43R background. All S. flexneri strains are built on attenuated multiple deletion strain. rpsL K43R is omitted except in the source strain to avoid redundacy.

TABLE 3
Plasmids

| | | SEQ ID |
|---|---|---|
| Plasmid | Description | NO: |
| CDFtet-AraBADRedCas9- | CDF tetR backbone carrying araC and pBAD inducible | N/A |
| tracrRNA (pKW20) | Cas9 and lambda alpha and beta genes | |
| CDFkan-AraBADRed-recA | CDF kanR backbone carrying araC and pBAD inducible | 1 |
| | recA and lambda alpha and beta genes | |
| pAraBAD-traRP4min | pMB1 carbR backbone carrying araC and pBAD inducible | N/A |
| | minimized tra operons for the RP4 family conjugation | |
| | system | |
| Belo-apraR-AraBAD-traRP4min | pBelo apraR backbone carrying araC and pBAD inducible | 2 |
| (pAra-Trans) | minimized tra operons for the RP4 family conjugation | |
| | system | |
| pSL1777 (pEffector) | CDF spectR backbone carrying constitutively expressed | N/A |
| | Type I-F Tn6677 CAST system with empty spacer DNA | |
| | for crRNA golden gate assembly | |
| CDF-spectR-AraBAD-CAST-GG | CDF spectR backbone carrying araC and pBAD inducible | 3 |
| (pAra-CAST-GG) | Type I-F Tn6677 CAST system with golden gate acceptor | |
| | site | |
| CDF-spectR-AraBAD-CAST-TS1 | As with pAra-CAST-GG but with constitutive TS1 | 4 |
| (pAra-CAST-TS1) | targeting crRNA | |
| CDF-spectR-AraBAD-CAST-TS2 | As with pAra-CAST-GG but with constitutive TS2 | 5 |
| (pAra-CAST-TS2) | targeting crRNA | |
| CDF-spectR-AraBAD-CAST-TS3 | As with pAra-CAST-GG but with constitutive TS3 | 6 |
| (pAra-CAST-TS3) | targeting crRNA | |
| CDF-spectR-AraBAD-CAST- | As with pAra-CAST-GG but with constitutive luxA* | 7 |
| luxA* (pAra-CAST-luxA*) | targeting crRNA | |
| CDF-spectR-AraBAD-CAST- | As with pAra-CAST-GG but with constitutive luxB1 | 8 |
| luxB1 (pAra-CAST-luxB1) | targeting crRNA | |
| CDF-spectR-AraBAD-CAST- | As with pAra-CAST-GG but with constitutive luxB2 | 9 |
| luxB2 (pAra-CAST-luxB2) | targeting crRNA | |
| CDF-spectR-AraBAD-CAST-dif | As with pAra-CAST-GG but with constitutive dif targeting | 10 |
| (pAra-CAST-dif) | crRNA | |
| pSC101-spectR-cassette 1-sC-oriT-pSC101 | spectR backbone carrying sacB cmR oriT and | 11 |
| RE (pCassette(START)) | Tn6677 right trasposon end (R-TE) in consecutive order | |
| pSC101-spectR-cassette2-pHGFP-pSC101 | spectR backbone carrying pheS* hygroR GFP and | 12 |
| LE-oriT (pCassette(STOP)) | Tn6677 left transposon end (L-TE) and oriT in consecutive | |
| | order | |
| CDF-spectR-AraBAD-CAST-NT | As with pAra-CAST-GG but with constitutive NT | 13 |
| (pAra-CAST-NT) | targeting crRNA | |
| CDF-spectR-AraBAD-CAST- | As with pAra-CAST-GG but with deleted crRNA | 14 |
| AcrRNA (pAra-CAST-AcrRNA) | | |
| CDF-spectR-AraBAD-CAST- | As with pAra-CAST-GG but with deleted crRNA cas6 | 15 |
| Acascade (pAra-CAST-Acascade) | cas7 cas8 | |
| CDF-spectR-AraBAD-CAST- | As with pAra-CAST-GG but with deleted crRNA tniQ | 16 |
| AQcascade (pAra-CAST- | cas6 cas7 cas8 | |
| AQcascade) | | |
| CDF-spectR-AraBAD-CAST-Atns | As with pAra-CAST-GG but with constitutive TS2 | 17 |
| (pAra-CAST-Atns) | targeting crRNA and deleted tnsA tnsB and tnsC | |
| CDF-spectR-AraBAD-ACAST | empty CDF spectR araC and pBAD promoter backbone | 18 |
| (pAra-ACAST) | | |
| RSF-carbR-AraBAD-CAST-GG | RSF carbR backbone carrying araC and pBAD inducible | 19 |
| (pAra-RSFcarbR-CAST-GG) | Type I-F Tn6677 CAST system with golden gate acceptor | |
| | site | |
| RSF-carbR-AraBAD-CAST-wbfF | As with pAra-RSFcarbR-CAST-GG but with constitutive | 20 |
| (pAra-RSFcarbR-CAST-wbfF) | wbfF targeting crRNA | |
| RSF-carbR-AraBAD-CAST- | As with pAra-RSFcarbR-CAST-GG but with constitutive | 21 |
| difch1 (pAra-RSFcarbR-CAST- | difch 1 targeting crRNA | |
| difch 1) | | |
| RSF-carbR-AraBAD-CAST- | As with pAra-RSFcarbR-CAST-GG but with constitutive | 22 |
| difch2 (pAra-RSFcarbR-CAST- | difch2 targeting crRNA | |
| difch2) | | |
| RSF-kanR-AraBAD-CAST-GG | RSF kanR backbone carrying araC and pBAD inducible | 23 |
| (pAra-RSFkanR-CAST-GG) | Type I-F Tn6677 CAST system with golden gate acceptor | |
| | site | |
| RSF-kanR-AraBAD-CAST-glmS | As with pAra-RSFkanR-CAST-GG but with constitutive | 24 |
| (pAra-RSFkanR-CAST-glmS) | glmS targeting crRNA | |
| CDF-gentR-AraBAD-Cre-lacI- | CDF gentR backbone carrying araC and pBAD inducible | 25 |
| Bxb1-PhiC31 (pCS20) | Cre recombinase and carrying lacI and IPTG inducible | |
| | Bxb1 and PhiC31 integrases | |
| pSC101-sC-oriT-RE-RB-N7- | | 26 |
| YPETmini-YPET-CaMV35S-LB- | | |
| kanR-LE-oriT (pSTART- | | |
| RBTDNA-STOP-LBTDNA) | | |
| pSC101-spectR-cassette 1-sC-oriT- | | 27 |
| luxA-homology-up | | |
| (pCassette(START-RECM)) | | |
| pSC101-spectR-cassette2-pHGFP- | | 28 |
| luxA-homology-down-oriT | | |
| (pCassette(STOP-RECM)) | | |
| pMB1-ampR-LuxA-sgRNA | | 29 |
| VS1-spectR-AraBAD-CAST-GG | VS1 spectR backbone carrying araC and pBAD inducible | 30 |
| (pAra-VS1spectR-CAST-GG) | Type I-F Tn6677 CAST system with golden gate acceptor | |
| | site | |
| VS1-spectR-AraBAD-CAST-flaA | As with pAra-VS1spectR-CAST-GG but with constitutive | 31 |
| (pAra-VS1spectR-CAST-flaA) | flaA targeting crRNA | |
| VS1-spectR-AraBAD-CAST-agpI | As with pAra-VS1spectR-CAST-GG but with constitutive | 32 |
| (pAra-VS1spectR-CAST-agpI) | agpI targeting crRNA | |
| VS1-spectR-AraBAD-CAST- | As with pAra-VS1spectR-CAST-GG but with constitutive | 33 |
| agrodif (pAra-VS1spectR-CAST- | dif targeting crRNA for agrobacterium | |
| agrodif) | | |
| VS1-spectR-AraBAD-CAST- | As with pAra-VS1spectR-CAST-GG but with constitutive | 34 |
| lineartetA (pAra-VS1spectR- | tetA targeting crRNA for agrobacterium linear | |
| CAST-lineartetA) | chromosome | |
| CDF-spectR-Lac-Tn7007-GG- | CDF spectR backbone carrying lacI and PLac inducible | 35 |
| START (pLac-OrthoCAST1-GG- | Type I-F Tn7007 CAST system with golden gate acceptor | |
| START) | site and Cassette(START) between Tn7007 RE and LE | |
| CDF-spectR-Lac-Tn7011-GG- | CDF spectR backbone carrying lacI and PLac inducible | 36 |
| STOP (pLac-OrthoCAST2-GG- | Type I-F Tn7011 CAST system with golden gate acceptor | |
| STOP) | site and Cassette(STOP) between Tn7011 RE and LE | |
| CDF-spectR-Lac-Tn7007- | As with pLac-OrthoCAST1-GG-START but with 1500kb | 37 |
| 1500kbShigella-START (pLac- | Shigella START targeting crRNA | |
| OrthoCAST1- | | |
| 1500kbShigellaSTART) | | |
| CDF-spectR-Lac-Tn7011- | As with pLac-OrthoCAST2-GG-STOP but with 1500kb | 38 |
| 1500kbShigella-STOP (pLac- | Shigella STOP targeting crRNA | |
| OrthoCAST2- | | |
| 1500kbShigellaSTOP) | | |
| CDF-spectR-Xyl-Tn7016-GG-ST- | CDF spectR backbone carrying xylS and Pm inducible | 39 |
| START (pXyl-OrthoCAST1-GG- | Type I-F Tn7016 CAST system with golden gate acceptor | |
| START) | site and Cassette(START) between Tn7016 RE and LE | |
| CDF-spectR-Xyl-Tn7011-GG- | CDF spectR backbone carrying xylS and Pm inducible | 40 |
| pKGFP-STOP (pXyl- | Type I-F Tn7011 CAST system with golden gate acceptor | |
| OrthoCAST2-GG-STOP) | site and Cassette(STOP) between Tn7011 RE and LE | |
| CDF-spectR-Xyl-Tn7016-orf-sT- | As with pXyl-OrthoCASTI-GG-START but with orfamide | 41 |
| START (pXyl-OrthoCAST1- | START targeting crRNA | |
| orfamideSTART) | | |
| CDF-spectR-Xyl-Tn7011-orf- | As with pXyl-OrthoCAST2-GG-STOP but with orfamide | 42 |
| pKGFP-STOP (pXyl- | STOP targeting crRNA | |
| OrthoCAST2-orfamideSTOP) | | |
| CDF-spectR-Xyl-Tn7016-rhz-sT- | As with pXyl-OrthoCAST1-GG-START but with rhizoxin | 43 |
| START (pXyl-OrthoCAST1- | START targeting crRNA | |
| rhizoxinSTART) | | |
| CDF-spectR-Xyl-Tn7011-rhz- | As with pXyl-OrthoCAST2-GG-STOP but with rhizoxin | 44 |
| pKGFP-STOP (pXyl- | STOP targeting crRNA | |
| OrthoCAST2-rhizoxinSTOP) | | |
| CDF-spectR-Xyl-Tn7016-pyn-sT- | As with pXyl-OrthoCAST1-GG-START but with | 45 |
| START (pXyl-OrthoCASTI- | pyrrolnitrin START targeting crRNA | |
| pyrrolnitrinSTART) | | |
| CDF-spectR-Xyl-Tn7011-pyn- | As with pXyl-OrthoCAST2-GG-STOP but with | 46 |
| pKGFP-STOP (pXyl- | pyrrolnitrin STOP targeting crRNA | |
| OrthoCAST2-pyrrolnitrinSTOP) | | |
| CDF-spectR-Xyl-Tn7016-pyl-sT- | As with pXyl-OrthoCASTI-GG-START but with | 47 |
| START (pXyl-OrthoCAST1- | pyoluteorin START targeting crRNA | |
| pyoluteorinSTART) | | |
| CDF-spectR-Xyl-Tn7011-pyl- | As with pXyl-OrthoCAST2-GG-STOP but with | 48 |
| pKGFP-STOP (pXyl- | pyoluteorin STOP targeting crRNA | |
| OrthoCAST2-pyoluteorinSTOP) | | |
| CDF-spectR-Xyl-Tn7016-pyv-sT- | As with pXyl-OrthoCAST1-GG-START but with | 49 |
| START (pXyl-OrthoCAST1- | pyoverdine START targeting crRNA | |
| pyoverdineSTART) | | |
| CDF-spectR-Xyl-Tn7011-pyv- | As with pXyl-OrthoCAST2-GG-STOP but with | 50 |
| pKGFP-STOP (pXyl- | pyoverdine STOP targeting crRNA | |
| OrthoCAST2-pyoverdineSTOP) | | |
| CDF-spectR-Xyl-Tn7016-cit-sT- | As with pXyl-OrthoCAST1-GG-START but with | 51 |
| START (pXyl-OrthoCAST1- | citronellol START targeting crRNA | |
| citronellolSTART) | | |
| CDF-spectR-Xyl-Tn7011-cit- | As with pXyl-OrthoCAST2-GG-STOP but with citronellol | 52 |
| pKGFP-STOP (pXyl- | STOP targeting crRNA | |
| OrthoCAST2-citronellolSTOP) | | |
| RK24 | | N/A |
| pEVS104 | | N/A |
| CDF-hygroR-AraBAD-CAST-GG | CDF hygroR backbone carrying araC and pBAD inducible | 53 |
| (pAra-hygroR-CAST-GG) | Type I-F Tn6677 CAST system with golden gate acceptor | |
| | site | |
| CDF-hygroR-AraBAD-CAST- | As with pAra-hygroR-CAST-GG but with constitutive TS1 | 54 |
| TS1 (pAra-hygroR-CAST-TS1) | targeting crRNA | |
| CDF-hygroR-AraBAD-CAST- | As with pAra-hygroR-CAST-GG but with constitutive TS2 | 55 |
| TS2 (pAra-hygroR-CAST-TS2) | targeting crRNA | |
| CDF-hygroR-AraBAD-CAST- | As with pAra-hygroR-CAST-GG but with constitutive TS3 | 56 |
| TS3 (pAra-hygroR-CAST-TS3) | targeting crRNA | |
| CDF-hygroR-AraBAD-CAST- | As with pAra-hygroR-CAST-GG but with constitutive TS4 | 57 |
| TS4 (pAra-hygroR-CAST-TS4) | targeting crRNA | |
| CDF-hygroR-AraBAD-CAST- | As with pAra-hygroR-CAST-GG but with constitutive TS5 | 58 |
| TS5 (pAra-hygroR-CAST-TS5) | targeting crRNA | |
| CDF-hygroR-AraBAD-CAST-dif | As with pAra-hygroR-CAST-GG but with constitutive dif | 59 |
| (pAra-hygroR-CAST-dif) | targeting crRNA | |

TABLE 4A
Oligonucleotides and Gene Blocks (Plasmid Construction and TnSeg)

| Name | Description | SEQ ID NO: |
|---|---|---|
| P5_i5-H503_CSP_VchR_v2 | i5-H503 index for RE TnSeq library amplification | 60 |
| P5_i5-H516_CSP_VchR_v2 | i5-H516 index for RE TnSeq library amplification | 61 |
| P5_i5-H502_CSP_VchR_v2 | i5-H502 index for RE TnSeq library amplification | 62 |
| P5_i5-H504_CSP_VchR_v2 | i5-H504 index for RE TnSeq library amplification | 63 |
| CSP_VchR_read1_v2 | custom sequencing of RE during TnSeq | 64 |

TABLE 4B
Oligonucleotides and Gene Blocks (Strain Construction Oligonucleotides)

| | Cassette (START) | | | |
|---|---|---|---|---|
| | Forward | Reverse | Cassette (STOP) | |
| Donor | Primer | Primer | Forward Primer | Reverse Primer |
| E. coli DH10B-Syn100ΔlacZ-1R-10Kb-2L | 65 | 76 | 87 | 101 |
| E. coli DH10B-Syn100ΔlacZ-1R-50Kb-2L | 65 | 76 | 88 | 102 |
| E. coli DH10B-Syn100ΔlacZ-1R-100Kb-2L | 65 | 76 | 89 | 103 |
| E. coli MDS42-lacZ-1R-200Kb-2L | 66 | 77 | 90 | 104 |
| E. coli Syn61-1R-100Kbdif-2L | 67 | 78 | 91 | 105 |
| E. coli Syn61-1R-600Kbdif-2L | 68 | 79 | 92 | 106 |
| E. coli Syn61-1R-1Mbdif-2L | 69 | 80 | 93 | 107 |
| E. coli Syn61-1R-2Mbdif-2L Syn Seg. 1 | 70 | 81 | 94 | 108 |
| E. coli Syn61-1R-1Mb-2L Syn Seg. 2 | 71 | 82 | 95 | 109 |
| E. coli Syn61-1R-1MboriC-2L Syn Seg. 3 | 72 | 83 | 96 | 110 |
| E. coli Syn61-1R-500Kb-2L Syn Seg. 4 | 73 | 84 | 97 | 111 |
| E. coli Syn61-1R-500Kb-2L Syn Seg. 5 | 74 | 85 | 98 | 112 |
| S. flexneri 2a-1R-1Mbdif-2L | 75 | 86 | 99 | 113 |
| E. coli MDS42-1R-1Mbdif-2L | 69 | 80 | 93 | 107 |
| E. coli MDS42-1RRB-100Kbdif-2LBL | 67 | 78 | 100 | 105 |
| E. coli Syn61-1luxA-100Kbdif-2luxA | 67 | 78 | 91 | 105 |
| E. coli Syn61-1luxA-1Mbdif-2luxA | 69 | 80 | 93 | 107 |
| E. coli Syn61-1luxA-2Mbdif-2luxA | 70 | 81 | 94 | 108 |

TABLE 4C
Oligonucleotides and Gene Blocks (Strain Construction Genotyping
Oligonucleotides)

| | Cassette START | | | |
|---|---|---|---|---|
| | Forward | Reverse | Cassette (STOP) | |
| Donor | Primer | Primer | Forward Primer | Reverse Primer |
| E. coli DH10B-Syn100ΔlacZ-1R-10Kb-2L | 114 | 133 | 151 | 169 |
| E. coli DH10B-Syn100ΔlacZ-1R-50Kb-2L | 114 | 133 | 152 | 170 |
| E. coli DH10B-Syn100ΔlacZ-1R-100Kb-2L | 114 | 133 | 153 | 171 |
| E. coli MDS42-lacZ-1R-200Kb-2L | 115 | 134 | 154 | 172 |
| E. coli Syn61-1R-100Kbdif-2L | 116 | 135 | 155 | 173 |
| E. coli Syn61-1R-600Kbdif-2L | 117 | 136 | 156 | 174 |
| E. coli Syn61-1R-1Mbdif-2L | 118 | 137 | 157 | 175 |
| E. coli Syn61-1R-2Mbdif-2L Syn Seg. 1 | 119 | 138 | 158 | 176 |
| E. coli Syn61-1R-1Mb-2L Syn Seg. 2 | 120 | 139 | 159 | 177 |
| E. coli Syn61-1R-1MboriC-2L Syn Seg. 3 | 121 | 140 | 160 | 178 |
| E. coli Syn61-1R-500Kb-2L Syn Seg. 4 | 122 | 141 | 158 | 176 |
| E. coli Syn61-1R-500Kb-2L Syn Seg. 5 | 123 | 142 | 122 | 141 |
| S. flexneri 2a-1R-1Mbdif-2L | 124 | 143 | 161 | 179 |
| E. coli MDS42-1R-1Mbdif-2L | 125 | 137 | 157 | 175 |
| E. coli MDS42-1RRB-100Kbdif-2LBL | 116 | 135 | 155 | 173 |
| S. flexneri 2a-1R-1500Kbdif-2L | 126 | 144 | 162 | 180 |
| P. protegens pf-5-1R-orfamide-2L | 127 | 145 | 163 | 181 |
| P. protegens pf-5-1R-rhizoxin-2L | 128 | 146 | 164 | 182 |
| P. protegens pf-5-1R-pyrrolnitrin-2L | 129 | 147 | 165 | 183 |
| P. protegens pf-5-1R-pyoluteorin-2L | 130 | 148 | 166 | 184 |
| P. protegens pf-5-1R-pyoverdine-2L | 131 | 149 | 167 | 185 |
| P. protegens pf-5-1R-citronellol-pyoverdine-2L | 132 | 150 | 167 | 185 |
| P. protegens pf-5-1R-pyoverdine-citronellol-2L | 131 | 149 | 168 | 186 |
| P. protegens pf-5-1R-pyoluteorin-rhizoxin-2L | 130 | 148 | 164 | 182 |
| P. protegens pf-5-1R-citronellol-2L | 132 | 150 | 168 | 186 |
| E. coli Syn61-1luxA-100Kbdif-2luxA | 116 | 135 | 155 | 173 |
| E. coli Syn61-1luxA-1Mbdif-2luxA | 118 | 137 | 157 | 175 |
| E. coli Syn61-1luxA-2Mbdif-2luxA | 119 | 138 | 158 | 176 |

TABLE 4D
Oligonucleotides and Gene Blocks (ACE Genotyping Oligonucleotides)

| | Forward | Reverse | |
|---|---|---|---|
| Genotype | Primer | Primer | Expected Size |
| R + TS2-Syn100 100Kb Junction | 114 | 187 | 2209 |
| L + TS2-Syn100 100Kb Junction | 188 | 171 | 3545 |
| Syn 100 100Kb Internal 1 | 189 | 190 | 1723 |
| Syn 100 100Kb Internal 2 | 191 | 192 | 1090 |
| R + TS1-Syn100 100Kb Junction | 193 | 114 | 1825 |
| L + TS1-Syn100 100Kb Junction | 171 | 194 | 4037 |
| R − TS1-Syn100 100Kb Junction | 114 | 194 | 2242 |
| L − TS1-Syn 100 100Kb Junction | 193 | 171 | 3620 |
| R + TS3-Syn100 100Kb Junction | 195 | 114 | 1818 |
| L + TS3-Syn 100 100Kb Junction | 171 | 196 | 3509 |
| R + dif-S. flexneri 1Mbdif Junction | 197 | 143 | 519 |
| L + dif-S. flexneri 1Mbdif Junction | 161 | 198 | 3837 |
| R + dif-Syn61 2Mbdif Syn Seg. 1 Junction | 138 | 197 | 546 |
| L + dif-Syn61 2Mbdif Syn Seg. 1 Junction patched | 198 | 158 | 465 |
| L + dif-Syn61 2Mbdif Syn Seg. 1 Junction unpatched | 198 | 158 | 3580 |

TABLE 5
Donor Boundaries

| | Cassette (START) | | Cassette (STOP) | |
|---|---|---|---|---|
| | Upstream | Downstream | Upstream | Downstream |
| Donor | Homology | Homology | Homology | Homology |
| E. coli DH10B-Syn100ΔlacZ-1R-10Kb-2L | 199 | 210 | 223 | 234 |
| E. coli DH10B-Syn100ΔlacZ-1R-50Kb-2L | 199 | 210 | 224 | 235 |
| E. coli DH10B-Syn100ΔlacZ-1R-100Kb-2L | \|199 | \|210 | 225 | 236 |
| E. coli MDS42-lacZ-1R-200Kb-2L | 200 | 211 | 226 | 237 |
| E. coli Syn61-1R-100Kbdif-2L | 201 | 212 | 227 | 238 |
| E. coli Syn61-1R-600Kbdif-2L | 202 | 213 | 228 | 239 |
| E. coli Syn61-1R-1Mbdif-2L | 203 | 214 | 229 | 240 |
| E. coli Syn61-1R-2Mbdif-2L Syn Seg. 1 | 204 | 215 | 230 | 241 |
| E. coli Syn61-1R-1Mb-2L Syn Seg. 2 | 205 | 216 | 231 | 242 |
| E. coli Syn61-1R-1MboriC-2L Syn Seg. 3 | 206 | 217 | 232 | 243 |
| E. coli Syn61-1R-500Kb-2L Syn Seg. 4 | 207 | 218 | 230 | 241 |
| E. coli Syn61-1R-500Kb-2L Syn Seg. 5 | 208 | 219 | 207 | 218 |
| S. flexneri 2a-1R-1Mbdif-2L | 209 | 220 | 233 | 244 |
| E. coli MDS42-1R-1Mbdif-2L | 203 | 214 | 229 | 240 |
| E. coli MDS42-1RRB-100Kbdif-2LBL | 201 | 221 | 227 | 245 |
| E. coli Syn61-1luxA-100Kbdif-2luxA | 201 | 221 | 227 | 245 |
| E. coli Syn61-1luxA-1Mbdif-2luxA | 203 | 222 | 229 | 246 |
| E. coli Syn61-1luxA-2Mbdif-2luxA | 204 | 215 | 230 | 241 |

TABLE 6A
ACE crRNA related sequences

| | | | | | Forward | Reverse |
|---|---|---|---|---|---|---|
| | | | | | Primer | Primer |
| | | | | | (SEQ | (SEQ |
| Target | | | | | ID | ID |
| Site | crRNA | PAM | Strand | Species/Strain | NO:) | NO:) |
| TS1 | accagacccgcgagcattaattcttgcctoca | cc | + | E. coli DH10B, E. coli | 279 | 311 |
| | SEQ ID NO: 247) | | | DH10B Δdif::lux | | |
| TS2 | ttagcggaatgctggtacgggctgataaagaa | cc | − | E. coli DH10B | 280 | 312 |
| (luxA1) | (SEQ ID NO: 248) | | | Δdif::lux | | |
| TS3 | agctgcaacaatgttgaaaatgccagccaact | cc | + | E. coli DHIOB, E. coli | 281 | 313 |
| | (SEQ ID NO: 249) | | | DH10B Δdif::lux | | |
| NT | GTTGTCTGACACTTGTCACAA | n/a | n/a | n/a | 282 | 314 |
| | ACCGCTAGGAG (SEQ ID NO: | | | | | |
| | 250) | | | | | |
| luxA* | catattcttgagccacttcattataaagctca | cc | + | E. coli DH10B | 283 | 315 |
| | (SEQ ID NO: 251) | | | Δdif::lux | | |
| luxB1 | gcatagcggaggaagcttgcttattggatcag | cc | − | E. coli DH10B | 284 | 316 |
| | (SEQ ID NO: 252) | | | Δdif::lux | | |
| luxB2 | cacttaaagatgagaggaataccttttttggc | cc | + | E. coli DH10B | 285 | 317 |
| | (SEQ ID NO: 253) | | | Δdif::lux | | |
| dif | ttgttaatgagcatgacaatcatgaccgccaa | ca | − | E. coli DH10B, E. coli | 286 | 318 |
| | (SEQ ID NO: 254) | | | MDS42 | | |
| glmS | gtgctaaaggggaccgacgttgaccagcctcg | cc | − | P. putida | 287 | 319 |
| | (SEQ ID NO: 255) | | | | | |
| wbfF | aaaaccatgcaggcagttaaagacatcacgca | cc | + | V. natriegens | 288 | 320 |
| | (SEQ ID NO: 256) | | | | | |
| chr.1 | tttaaagaagcaaaagccaaatacggtgtaat | cc | + | V. natriegens | 289 | 321 |
| dif | (SEQ ID NO: 257) | | | | | |
| chr.2 | acaaatttaaaatctgagagggataggagact | cc | − | V. natriegens | 290 | 322 |
| dif | (SEQ ID NO: 258) | | | | | |
| flaA | atgctttacacggaaggtacaccgggcacgat | cc | − | A. tumefaciens | 291 | 323 |
| | (SEQ ID NO: 259) | | | | | |
| agpI | gttgcgagcggcattatcgcgatgcgggtttc | cc | − | A. tumefaciens | 292 | 324 |
| | (SEQ ID NO: 260) | | | | | |
| dif | aaagtgaagccattgaaatcattggcatgtcg | cc | + | A. tumefaciens | 293 | 325 |
| (agro) | (SEQ ID NO: 261) | | | | | |
| tetA | gtcatgcaatttgtgttctcgccgatccttgg | cc | + | A. tumefaciens | 294 | 326 |
| (linear) | (SEQ ID NO: 262) | | | | | |
| TS4 | tggtggcggtggtgggagctattctcgttctg | cc | + | E. coli DH10B, E. coli | 295 | 327 |
| | (SEQ ID NO: 263) | | | DH10B Δdif::lux | | |
| TS5 | gaaaacacctgatatgaaaggcaatgccacca | cc | + | E. coli DHIOB, E. coli | 296 | 328 |
| | (SEQ ID NO: 264) | | | DH10B Δdif::lux | | |

TABLE 6B
OASIS crRNA related sequences

| | | | | Forward | Reverse |
|---|---|---|---|---|---|
| | | | | Primer | Primer |
| | | | | (SEQ ID | (SEQ ID |
| Target Site | crRNA | Strand | Species/Strain | NO:) | NO:) |
| 1500KbShigella- | ggtattcgatggggaa | + | S. flexneri | 297 | 329 |
| START | gaacttcacgcgagct | | CFS100 rpsL | | |
| | (SEQ ID NO: 265) | | K43R ΔrecA | | |
| | | | Δtus ΔoriT | | |
| | | | ΔhigB Ahok | | |
| 1500KbShigella- | aggagatggactactt | + | S. flexneri | 298 | 330 |
| STOP | catggaagtatttgtt | | CFS100 rpsL | | |
| | (SEQ ID NO: 266) | | K43R ΔrecA | | |
| | | | Δtus ΔoriT | | |
| | | | ΔhigB Δhok | | |
| orfamide-START | tggcggcctgtcagagc | + | P. protegens | 299 | 331 |
| | tacgtcaaagaaatc | | pf-5 | | |
| | (SEQ ID NO: 267) | | | | |
| orfamide-STOP | ttccatcgcgcgttcaa | + | P. protegens | 300 | 332 |
| | gaaatggaccggcga | | pf-5 | | |
| | (SEQ ID NO: 268) | | | | |
| rhizoxin-START | tggagtacgagacgttc | + | P. protegens | 301 | 333 |
| | atgctgtcgcgttga | | pf-5 | | |
| | (SEQ ID NO: 269) | | | | |
| rhizoxin-STOP | gttgcgacgcttagcag | + | P. protegens | 302 | 334 |
| | ctctcaagcaagagg | | pf-5 | | |
| | (SEQ ID NO: 270) | | | | |
| pyrrolnitrin- | gatcaagagcaagttcg | + | P. protegens | 303 | 335 |
| START | gctatcacctggtgc | | pf-5 | | |
| | (SEQ ID NO: 271) | | | | |
| pyrrolnitrin- | tgctgggaggtttctac | + | P. protegens | 304 | 336 |
| STOP | ctgttgtggttgctg | | pf-5 | | |
| | (SEQ ID NO: 272) | | | | |
| pyoluteorin- | ttcaggccggcaagct | + | P. protegens | 305 | 337 |
| START | ccacaaggtcatcact | | pf-5 | | |
| | (SEQ ID NO: 273) | | | | |
| pyoluteorin- | gatcaaccgctgatca | + | P. protegens | 306 | 338 |
| STOP | gcggttgcctgtgttg | | pf-5 | | |
| | (SEQ ID NO: 274) | | | | |
| pyoverdine- | aacaagaactacccca | + | P. protegens | 307 | 339 |
| START | acgaggaacggatcaa | | pf-5 | | |
| | (SEQ ID NO: 275) | | | | |
| pyoverdine- | tgggcctgaaaagaaa | + | P. protegens | 308 | 340 |
| STOP | gctcggcatgtacaag | | pf-5 | | |
| | SEQ ID NO: 276) | | | | |
| citronellol- | ttcggaccatgcgccg | + | P. protegens | 309 | 341 |
| START | atctggctcgaactgg | | pf-5 | | |
| | (SEQ ID NO: 277) | | | | |
| citronellol- | atcacctgcctggtgg | + | P. protegens | 310 | 342 |
| STOP | cctttgcggtgttcca | | pf-5 | | |
| | (SEQ ID NO: 278) | | | | |

TABLE 7A
ACE Transfers

| | | | | Genome Size |
|---|---|---|---|---|
| | | | Genome | (rounded) |
| Donor | Recipient | Chimera | Size (bp) | (Mb) |
| E. coli DH10B- | E. coli DH10B Δdif::lux | E. coli DH10B Δdif::lux | 4707021 | 4.71 |
| Syn100ΔlacZ-1R- | CAST-TS1 | TS1-Syn100-10Kb | | |
| 10Kb-2L | | | | |
| E. coli DH10B- | E. coli DH10B Δdif::lux | E. coli DH10B Δdif::lux | 4707021 | 4.71 |
| Syn100ΔlacZ-1R- | CAST-TS2 | TS2-Syn100-10Kb | | |
| 10Kb-2L | | | | |
| E. coli DH10B- | E. coli DH10B Δdif::lux | E. coli DH10B Δdif::lux | 4707021 | 4.71 |
| Syn100ΔlacZ-1R- | CAST-TS3 | TS3-Syn100-10Kb | | |
| 10Kb-2L | | | | |
| E. coli DH10B- | E. coli DH10B Δdif::lux | E. coli DH10B Δdif::lux | 4747023 | 4.75 |
| Syn100ΔlacZ-1R- | CAST-TS1 | TS1-Syn100-50Kb | | |
| 50Kb-2L | | | | |
| E. coli DH10B- | E. coli DH10B Δdif::lux | E. coli DH10B Δdif::lux | 4747023 | 4.75 |
| Syn100ΔlacZ-IR- | CAST-TS2 | TS2-Syn100-50Kb | | |
| 50Kb-2L | | | | |
| E. coli DH10B- | E. coli DH10B Δdif::lux | E. coli DH10B Δdif::lux | 4747023 | 4.75 |
| Syn100ΔlacZ-1R- | CAST-TS3 | TS3-Syn100-50Kb | | |
| 50Kb-2L | | | | |
| E. coli DH10B- | E. coli DH10B Δdif::lux | E. coli DH10B Δdif::lux | 4801035 | 4.8 |
| Syn100ΔlacZ-1R- | CAST-TS1 | TS1-Syn100-100Kb | | |
| 100Kb-2L | | | | |
| E. coli DH10B- | E. coli DH10B Δdif::lux | E. coli DH10B Δdif::lux | 4801035 | 4.8 |
| Syn100ΔlacZ-1R- | CAST-TS2 | TS2-Syn100-100Kb | | |
| 100Kb-2L | | | | |
| E. coli DH10B- | E. coli DH10B Δdif::lux | E. coli DH10B Δdif::lux | 4801035 | 4.8 |
| Syn100ΔlacZ-1R- | CAST-TS3 | TS3-Syn100-100Kb | | |
| 100Kb-2L | | | | |
| E. coli DH10B- | E. coli DH10B Δdif::lux | E. coli DH10B Δdif::lux | 4801035 | 14.8 |
| Syn100ΔlacZ-1R- | CAST-luxA* | luxA*-Syn100-100Kb | | |
| 100Kb-2L | | | | |
| E. coli DH10B- | E. coli DH10B Δdif::lux | E. coli DH10B Δdif::lux | 4801035 | 4.8 |
| Syn100ΔlacZ-1R- | CAST-luxB1 | luxBl-Syn100-100Kb | | |
| 100Kb-2L | | | | |
| E. coli DH10B- | E. coli DH10B Δdif::lux | E. coli DH10B Δdif::lux | 4801035 | 4.8 |
| Syn100ΔlacZ-1R- | CAST-luxB2 | luxB2-Syn100-100Kb | | |
| 100Kb-2L | | | | |
| E. coli MDS42-lacZ- | E. coli DH10B CAST-TSI | E. coli DH10B TS1- | 4900215 | 14.9 |
| 1R-200Kb-2L | | MDS42-lacZ-200Kb | | |
| E. coli Syn61-1R- | E. coli DH10B Δdif::lux | E. coli DH10B Δdif::lux | 4803858 | 4.8 |
| 100Kbdif-2L | CAST-TS1 | TS1-Syn61-100Kbdif | | |
| E. coli Syn61-1R- | E. coli DH10B Δdif::lux | E. coli DH10B Δdif::lux | 4803858 | 14.8 |
| 100Kbdif-2L | CAST-TS2 | TS2-Syn61-100Kbdif | | |
| E. coli Syn61-1R- | E. coli DH10B Δdif::lux | E. coli DH10B Δdif::lux | 4803858 | 4.8 |
| 100Kbdif-2L | CAST-TS3 | TS3-Syn61-100Kbdif | | |
| E. coli Syn61-1R- | E. coli DH10B Δdif::lux | E. coli DH10B Δdif::lux | 5289866 | 5.29 |
| 600Kbdif-2L | CAST-TS1 | TS1-Syn61-600Kbdif | | |
| E. coli Syn61-1R- | E. coli DH10B Δdif::lux | E. coli DH10B Δdif::lux | 5289866 | 5.29 |
| 600Kbdif-2L | CAST-TS2 | TS2-Syn61-600Kbdif | | |
| E. coli Syn61-1R- | E. coli DHIOB Δdif::lux | E. coli DH10B Δdif::lux | 5289866 | 5.29 |
| 600Kbdif-2L | CAST-TS3 | TS3-Syn61-600Kbdif | | |
| E. coli Syn61-1R- | E. coli DH10B Δdif::lux | E. coli DH10B Δdif::lux | 5630492 | 5.63 |
| 1Mbdif-2L | CAST-TS1 | TS1-Syn61-1Mbdif | | |
| E. coli Syn61-1R- | E. coli DH10B Δdif::lux | E. coli DH10B Δdif::lux | 5630492 | 5.63 |
| 1 Mbdif-2L | CAST-TS2 | TS2-Syn61-1Mbdif | | |
| E. coli Syn61-1R- | E. coli DH10B Δdif::lux | E. coli DH10B Δdif::lux | 5630492 | 5.63 |
| 1Mbdif-2L | CAST-TS3 | TS3-Syn61-1Mbdif | | |
| E. coli Syn61-1R- | E. coli DH10B Δdif::lux | E. coli DH10B Δdif::lux | 6609266 | 6.61 |
| 2Mbdif-2L Syn Seg. 1 | CAST-TS1 | TS1-Syn61-2Mbdif Syn | | |
| | | Seg. 1 | | |
| E. coli Syn61-1R- | E. coli DH10B Δdif::lux | E. coli DH10B Δdif::lux | 6609266 | 6.61 |
| 2Mbdif-2L Syn Seg. 1 | CAST-TS2 | TS2-Syn61-2Mbdif Syn | | |
| | | Seg. 1 | | |
| E. coli Syn61-1R- | E. coli DH10B Δdif::lux | E. coli DH10B Δdif::lux | 6609266 | 6.61 |
| 2Mbdif-2L Syn Seg. 1 | CAST-TS3 | TS3-Syn61-2Mbdif Syn | | |
| | | Seg. 1 | | |
| E. coli Syn61-1R- | E. coli DH10B Δdif::lux | E. coli DH10B Δdif::lux | 5756079 | 5.76 |
| 1Mb-2L Syn Seg. 2 | CAST-TS1 | TS1-Syn61-1Mb Syn | | |
| | | Seg. 2 | | |
| E. coli Syn61-1R- | E. coli DH10B Δdif::lux | E. coli DH10B Δdif::lux | 5756079 | 5.76 |
| 1Mb-2L Syn Seg. 2 | CAST-TS2 | TS2-Syn61-1Mb Syn | | |
| | | Seg. 2 | | |
| E. coli Syn61-1R- | E. coli DH10B Δdif::lux | E. coli DH10B Δdif::lux | 5756079 | 5.76 |
| 1Mb-2L Syn Seg. 2 | CAST-TS3 | TS3-Syn61-1Mb Syn | | |
| | | Seg. 2 | | |
| E. coli Syn61-1R- | E. coli DH10B CAST-TS3 | E. coli DH10B Δdif::lux | 5675263 | 5.68 |
| 1MboriC-2L Syn | | TS3-Syn61-1MboriC Syn | | |
| Seg. 3 | | Seg. 5 | | |
| E. coli Syn61-1R- | E. coli DH10B Δdif::lux | E. coli DH10B Δdif::lux | 5255474 | 5.26 |
| 500Kb-2L Syn Seg. 4 | CAST-TS1 | TS1-Syn61-500Kb Syn | | |
| | | Seg. 4 | | |
| E. coli Syn61-1R- | E. coli DH10B Δdif::lux | E. coli DH10B Δdif::lux | 5255474 | 5.26 |
| 500Kb-2L Syn Seg. 4 | CAST-TS2 | TS2-Syn61-500Kb Syn | | |
| | | Seg. 4 | | |
| E. coli Syn61-1R- | E. coli DH10B Δdif::lux | E. coli DH10B Δdif::lux | 5255474 | 5.26 |
| 500Kb-2L Syn Seg. 4 | CAST-TS3 | TS3-Syn61-500Kb Syn | | |
| | | Seg. 4 | | |
| E. coli Syn61-1R- | E. coli DH10B Δdif::lux | E. coli DH10B Δdif::lux | 5232612 | 5.23 |
| 500Kb-2L Syn Seg. 5 | CAST-TS1 | TS1-Syn61-500Kb Syn | | |
| | | Seg. 3 | | |
| E. coli Syn61-1R- | E. coli DH10B Δdif::lux | E. coli DH10B Δdif::lux | 5232612 | 5.23 |
| 500Kb-2L Syn Seg. 5 | CAST-TS2 | TS2-Syn61-500Kb Syn | | |
| | | Seg. 3 | | |
| E. coli Syn61-1R- | E. coli DH10B Δdif::lux | E. coli DH10B Δdif::lux | 5232612 | 5.23 |
| 500Kb-2L Syn Seg. 5 | CAST-TS3 | TS3-Syn61-500Kb Syn | | |
| | | Seg. 3 | | |
| S. flexneri 2a-1R- | E. coli DH10B CAST-dif | E. coli DH10B Δdif::S. | 5570081 | 5.57 |
| 1 Mbdif-2L | | flexneri-1Mbdif | | |
| S. flexneri 2a-1R- | E. coli MDS42 CAST-dif | E. coli MDS42 Δdif::S. | 4860139 | 4.86 |
| 1Mbdif-2L | | flexneri-1Mbdif | | |
| E. coli DH10B- | P. putida KT2440 CAST- | P. putida KT2440 | 6292437 | 6.29 |
| Syn100ΔlacZ-1R- | glmS | ΔglmS::Syn100-100Kb | | |
| 100Kb-2L | | | | |
| E. coli MDS42-lacZ- | V. natriegens DSM759 | V. natriegens DSM759 | 3462101 | 3.46 |
| 1R-200Kb-2L | CAST-wbfF | ΔglmS::MDS42-lacZ- | | |
| | | 200Kb | | |
| E. coli MDS42-1R- | V. natriegens DSM759 | V. natriegens DSM759 | 4117792 | 4.12 |
| 1 Mbdif-2L | CAST-difch1 | Δdifch1::MDS42-1Mbdif | | |
| E. coli MDS42-1R- | V. natriegens DSM759 | V. natriegens DSM759 | 2796899 | 2.8 |
| 1Mbdif-2L | CAST-difch2 | Δdifch2::MDS42-1Mbdif | | |
| E. coli Syn61-1R- | E. coli DH10B CAST-dif | E. coli DH10B Δdif::Syn. | 6601793 | 6.6 |
| 2Mbdif-2L Syn Seg. 1 | | Seg 1 | | |
| S. flexneri 2a-1R- | E. coli DH10B Δdif::Syn. | E. coli DH10B Δdif::Syn. | 7485737 | 7.49 |
| 1 Mbdif-2L | Seg 1 CAST-dif | Seg 1-S. flexneri-1Mbdif | | |
| E. coli Syn61-1R- | A. tumefaciens | A. tumefaciens | 2951828 | 2.95 |
| 100Kbdif-2L | spectVS1CAST-agpI | ΔagpI::Syn61-100Kbdif | | |
| E. coli MDS42- | A. tumefaciens | A. tumefaciens | 2952351 | 2.95 |
| 1RRB-100Kbdif- | spectVS1CAST-agpI | ΔagpI::MDS42-100Kbdif- | | |
| 2LBL (T-DNA) | | TDNA | | |
| E. coli MDS42- | A. tumefaciens | A. tumefaciens | 2952351 | 2.95 |
| 1RRB-100Kbdif- | spectVS1CAST-flaA | ΔflaA::MDS42-100Kbdif- | | |
| 2LBL (T-DNA) | | TDNA | | |
| E. coli MDS42- | A. tumefaciens | A. tumefaciens | 2186348 | 2.19 |
| 1RRB-100Kbdif- | spectVS1CAST-tetA | ΔtetA::MDS42-100Kbdif- | | |
| 2LBL (T-DNA) | | TDNA | | |
| E. coli MDS42- | A. tumefaciens | A. tumefaciens | 2952351 | 2.95 |
| 1RRB-100Kbdif- | spectVS1CAST-dif(agro) | Δdif::MDS42-100Kbdif- | | |
| 2LBL (T-DNA) | | TDNA | | |
| E. coli Syn61-1luxA- | E. coli DH10B Δdif::lux | E. coli DH10B Δdif::lux | 4803853 | 4.8 |
| 100Kbdif-2luxA | pKW20 | TS2-Syn61-100Kbdif | | |
| E. coli Syn61-1luxA- | E. coli DH10B Δdif::lux | E. coli DH10B Δdif::lux | 5630487 | 5.63 |
| 1Mbdif-2luxA | pKW20 | TS2-Syn61-1Mbdif | | |
| E. coli Syn61-1luxA- | E. coli DHIOB Δdif::lux | E. coli DH10B Δdif::lux | 6609261 | 6.61 |
| 2Mbdif-2luxA | pKW20 | TS2-Syn61-2Mbdif Syn | | |
| | | Seg. 1 | | |
| E. coli Syn61-1luxA- | E. coli DH10B Δdif::lux | E. coli DH10B Δdif::lux | 4803853 | 4.8 |
| 100Kbdif-2luxA | pKW20 sgRNA-luxA | TS2-Syn61-100Kbdif | | |
| E. coli Syn61-1luxA- | E. coli DH10B Δdif::lux | E. coli DH10B Δdif::lux | 5630487 | 5.63 |
| 1Mbdif-2luxA | pKW20 sgRNA-luxA | TS2-Syn61-1Mbdif | | |
| E. coli Syn61-1luxA- | E. coli DH10B Δdif::lux | E. coli DH10B Δdif::lux | 6609261 | 6.61 |
| 2Mbdif-2luxA | pKW20 sgRNA-luxA | TS2-Syn61-2Mbdif Syn | | |
| | | Seg. 1 | | |
| S. flexneri 2a-1R- | E. coli DH10B CAST-dif | E. coli DH10B Δdif::S. | 6149740 | 6.15 |
| 1500Kbdif-2L | | flexneri-1500Kbdif | | |
| P. protegens pf-5-1R- | E. coli DH10B Δdif::lux | E. coli DH10B Δdif::lux | 4774231 | 4.77 |
| orfamide-2L (80Kb) | hygroCDFCAST-TS2 | TS2-P. pro orf 80Kb | | |
| P. protegens pf-5-1R- | E. coli DH10B Δdif::lux | E. coli DH10B Δdif::lux | 4774231 | 4.77 |
| orfamide-2L (80Kb) | hygroCDFCAST-TS3 | TS3-P. pro orf 80Kb | | |
| P. protegens pf-5-1R- | E. coli DH10B Δdif::lux | E. coli DH10B Δdif::lux | 4833520 | 4.83 |
| rhizoxin-2L (140Kb) | hygroCDFCAST-TS2 | TS2-P. pro rhz 140Kb | | |
| P. protegens pf-5-1R- | E. coli DH10B Δdif::lux | E. coli DH10B Δdif::lux | 4833520 | 4.83 |
| rhizoxin-2L (140Kb) | hygroCDFCAST-TS3 | TS3-P. pro rhz 140Kb | | |
| P. protegens pf-5-1R- | E. coli DH10B Δdif::lux | E. coli DH10B Δdif::lux | 5108934 | 5.11 |
| pyoluteorin-rhizoxin- | hygroCDFCAST-TS2 | TS2-P. pro pyl-rhz 415Kb | | |
| 2L (415Kb) | | | | |
| P. protegens pf-5-1R- | E. coli DH10B | E. coli DH10B Δdif::P. pro | 5101461 | 5.1 |
| pyoluteorin-rhizoxin- | hygroCDFCAST-dif | pyl-rhz 415Kb | | |
| 2L (415Kb) | | | | |
| P. protegens pf-5-1R- | E. coli DH10B Δdif::lux | E. coli DH10B Δdif::lux | 4753426 | 4.75 |
| pyrrolnitrin-2L (60Kb) | hygroCDFCAST-TS3 | TS3-P. pro pyn 60Kb | | |
| P. protegens pf-5-1R- | E. coli DH10B Δdif::lux | E. coli DH10B Δdif::lux | 4759926 | 4.76 |
| pyoluteorin-2L (66Kb) | hygroCDFCAST-TS2 | TS2-P. pro pyl 66Kb | | |
| P. protegens pf-5-1R- | E. coli DH10B Δdif::lux | E. coli DH10B Δdif::lux | 4759926 | 4.76 |
| pyoluteorin-2L (66Kb) | hygroCDFCAST-TS3 | TS3-P. pro pyl 66Kb | | |
| P. protegens pf-5-1R- | E. coli DH10B | E. coli DH10B TS3-P. pro | 4931981 | 4.93 |
| pyoverdine-2L (246Kb) | hygroCDFCAST-TS3 | pyv 246Kb | | |
| P. protegens pf-5-1R- | E. coli DH10B | E. coli DH10B TS5-P. pro | 4931981 | 4.93 |
| pyoverdine-2L (246Kb) | hygroCDFCAST-TS5 | pyv 246Kb | | |
| P. protegens pf-5-1R- | E. coli DH10B | E. coli DH10B TS3-P. pro | 5059579 | 5.06 |
| citronellol- pyoverdine-2L | hygroCDFCAST-TS3 | cit-pyv 373Kb | | |
| (373Kb) | | | | |
| P. protegens pf-5-1R- | E. coli DH10B | E. coli DH10B TS3-P. pro | 5080897 | 5.08 |
| pyoverdine- citronellol-2L | hygroCDFCAST-TS3 | pyv-cit 395Kb | | |
| (395Kb) | | | | |
| P. protegens pf-5-1R- | E. coli DH10B | E. coli DH10B TS4-P. pro | 5080897 | 5.08 |
| pyoverdine- citronellol-2L | hygroCDFCAST-TS4 | pyv-cit 395Kb | | |
| (395Kb) | | | | |
| P. protegens pf-5-1R- | E. coli DH10B | E. coli DH10B Δdif::P. pro | 5080897 | 5.08 |
| pyoverdine- citronellol-2L | hygroCDFCAST-dif | pyv-cit 395Kb | | |
| (395Kb) | | | | |
| P. protegens pf-5-1R- | E. coli DH10B | E. coli DH10B TS3-P. pro | 5208495 | 5.21 |
| citronellol-2L (522Kb) | hygroCDFCAST-TS3 | cit 522Kb | | |
| P. protegens pf-5-1R- | E. coli DH10B | E. coli DH10B TS4-P. pro | 5208495 | 5.21 |
| citronellol-2L (522Kb) | hygroCDFCAST-TS4 | cit 522Kb | | |
| P. protegens pf-5-1R- | E. coli DH10B | E. coli DH10B Δdif::P. pro | 5208495 | 5.21 |
| citronellol-2L (522Kb) | hygroCDFCAST-dif | cit 522Kb | | |

TABLE 7B
ACE Transfers

| | | | | | | Solid Agar |
|---|---|---|---|---|---|---|
| | | | | | | Selection |
| Donor | Recipient | Chimera | Target | Target Sequence | PAM | Scheme |
| E. coli | E. coli DH10B | E. coli DH10B | TS1 | accagacccgcgagcatta | cc | hygromycin, |
| DH10B- | Δdif::lux | Δdif::lux TS1- | | attcttgcctcca (SEQ | | spectinomycin, |
| Syn100ΔlacZ | CAST-TS1 | Syn100-10Kb | | ID NO: 247) | | sucrose |
| -1R-10Kb- | | | | | | |
| 2L | | | | | | |
| E. coli | E. coli DH10B | E. coli DH10B | TS2 | ttagcggaatgctggtacgg | cc | hygromycin, |
| DH10B- | Δdif::lux | Δdif::lux TS2- | | gctgataaagaa (SEQ | | spectinomycin, |
| Syn100ΔlacZ | CAST-TS2 | Syn100-10Kb | | ID NO: 248) | | sucrose |
| -1R-10Kb- | | | | | | |
| 2L | | | | | | |
| E. coli | E. coli DH10B | E. coli DH10B | TS3 | agctgcaacaatgttgaaaa | cc | hygromycin, |
| DH10B- | Δdif::lux | Δdif::lux TS3- | | tgccagccaact (SEQ | | spectinomycin, |
| Syn100ΔlacZ | CAST-TS3 | Syn100-10Kb | | ID NO: 249) | | sucrose |
| -1R-10Kb- | | | | | | |
| 2L | | | | | | |
| E. coli | E. coli DH10B | E. coli DH10B | ITS1 | accagacccgcgagcatta | cc | hygromycin, |
| DH10B- | Δdif::lux | Δdif::lux TS1- | | attcttgcctcca (SEQ | | spectinomycin, |
| Syn100ΔlacZ | CAST-TS1 | Syn100-50Kb | | ID NO: 247) | | sucrose |
| -1R-50Kb- | | | | | | |
| 2L | | | | | | |
| E. coli | E. coli DH10B | E. coli DH10B | TS2 | ttagcggaatgctggtacgg | cc | hygromycin, |
| DH10B- | Δdif::lux | Δdif::lux TS2- | | gctgataaagaa (SEQ | | spectinomycin, |
| Syn100ΔlacZ | CAST-TS2 | Syn100-50Kb | | ID NO: 248) | | sucrose |
| -1R-50Kb- | | | | | | |
| 2L | | | | | | |
| E. coli | E. coli DH10B | E. coli DH10B | TS3 | agctgcaacaatgttgaaaa | cc | hygromycin, |
| DH10B- | Δdif::lux | Δdif::lux TS3- | | tgccagccaact (SEQ | | spectinomycin, |
| Syn100ΔlacZ | CAST-TS3 | Syn100-50Kb | | ID NO: 249) | | sucrose |
| -1R-50Kb- | | | | | | |
| 2L | | | | | | |
| E. coli | E. coli DH10B | E. coli DH10B | TS1 | accagacccgcgagcatta | cc | hygromycin, |
| DH10B- | Δdif::lux | Δdif::lux TS1- | | attcttgcctcca (SEQ | | spectinomycin, |
| Syn100ΔlacZ | CAST-TS1 | Syn100-100Kb | | ID NO: 247) | | sucrose |
| -1R-100Kb- | | | | | | |
| 2L | | | | | | |
| E. coli | E. coli DH10B | E. coli DH10B | TS2 | ttagcggaatgctggtacgg | cc | hygromycin, |
| DH10B- | Δdif::lux | Δdif::lux TS2- | | gctgataaagaa (SEQ | | spectinomycin, |
| Syn100ΔlacZ | CAST-TS2 | Syn100-100Kb | | ID NO: 248) | | sucrose |
| -1R-100Kb- | | | | | | |
| 2L | | | | | | |
| E. coli | E. coli DH10B | E. coli DH10B | TS3 | agctgcaacaatgttgaaaa | cc | hygromycin, |
| DH10B- | Δdif::lux | Δdif::lux TS3- | | tgccagccaact (SEQ | | spectinomycin, |
| Syn100ΔlacZ | CAST-TS3 | Syn100-100Kb | | ID NO: 249) | | sucrose |
| -1R-100Kb- | | | | | | |
| 2L | | | | | | |
| E. coli | E. coli DH10B | E. coli DH10B | luxA* | catattcttgagccacttca | cc | hygromycin, |
| DH10B- | Δdif::lux | Δdif::lux luxA*- | | ttataaagctca (SEQ ID | | spectinomycin, |
| Syn100ΔlacZ | CAST-luxA* | Syn100-100Kb | | NO: 251) | | sucrose |
| -1R-100Kb- | | | | | | |
| 2L | | | | | | |
| E. coli | E. coli DH10B | E. coli DH10B | luxB1 | gcatagcggaggaagcttg | cc | hygromycin, |
| DH10B- | Δdif::lux | Δdif::lux luxB1- | | cttattggatcag (SEQ | | spectinomycin, |
| Syn100ΔlacZ | CAST-luxB1 | Syn100-100Kb | | ID NO: 252) | | sucrose |
| -1R-100Kb- | | | | | | |
| 2L | | | | | | |
| E. coli DHIOB- | E. coli DH10B | E. coli DH10B | luxB2 | cacttaaagatgagaggaat | cc | hygromycin, |
| Syn100ΔlacZ | Δdif::lux | Δdif::lux luxB2- | | accttttttggc (SEQ ID | | spectinomycin, |
| -1R-100Kb-2L | CAST-luxB2 | Syn100-100Kb | | NO: 253) | | sucrose |
| E. coli | E. coli DH10B | E. coli DH10B | TS1 | accagacccgcgagcatta | cc | hygromycin, |
| MDS42- | CAST-TS1 | TS1-MDS42- | | attcttgcctcca (SEQ | | spectinomycin, |
| lacZ-1R- | | lacZ-200Kb | | ID NO: 247) | | sucrose |
| 200Kb-2L | | | | | | |
| E. coli | CAST-TS1 | E. coli DH10B | TS1 | accagacccgcgagcatta | cc | hygromycin, |
| Syn61-1R- | E. coli DH10B | Δdif::lux TS1- | | attcttgcctcca (SEQ | | spectinomycin, |
| 100Kbdif-2L | Δdif::lux | Syn61-100Kbdif | | ID NO: 247) | | sucrose |
| E. coli | CAST-TS2 | E. coli DH10B | TS2 | ttagcggaatgctggtacgg | cc | hygromycin, |
| Syn61-1R- | E. coli DH10B | Δdif::lux TS2- | | gctgataaagaa (SEQ | | spectinomycin, |
| 100Kbdif-2L | Δdif::lux | Syn61-100Kbdif | | ID NO: 248) | | sucrose |
| E. coli | E. coli DH10B | E. coli DH10B | TS3 | agctgcaacaatgttgaaaa | cc | hygromycin, |
| Syn61-1R- | Δdif::lux | Δdif::lux TS3- | | tgccagccaact (SEQ | | spectinomycin, |
| 100Kbdif-2L | CAST-TS3 | Syn61-100Kbdif | | ID NO: 249) | | sucrose |
| E. coli | CAST-TS1 | E. coli DH10B | TS1 | accagacccgcgagcatta | cc | hygromycin, |
| Syn61-1R- | E. coli DH10B | Δdif::lux TS1- | | attcttgcctcca (SEQ | | spectinomycin, |
| 600Kbdif-2L | Δdif::lux | Syn61-600Kbdif | | ID NO: 247) | | sucrose |
| E. coli | CAST-TS2 | E. coli DH10B | TS2 | ttagcggaatgctggtacgg | cc | hygromycin, |
| Syn61-1R- | E. coli DH10B | Δdif::lux TS2- | | gctgataaagaa (SEQ | | spectinomycin, |
| 600Kbdif-2L | Δdif::lux | Syn61-600Kbdif | | ID NO: 248) | | sucrose |
| E. coli | E. coli DH10B | E. coli DH10B | TS3 | agctgcaacaatgttgaaaa | cc | hygromycin, |
| Syn61-1R- | Δdif::lux | Δdif::lux TS3- | | tgccagccaact (SEQ | | spectinomycin, |
| 600Kbdif-2L | CAST-TS3 | Syn61-600Kbdif | | ID NO: 249) | | sucrose |
| E. coli | E. coli DH10B | E. coli DH10B | TS1 | accagacccgcgagcatta | cc | hygromycin, |
| Syn61-1R- | Δdif::lux | Δdif::lux TS1- | | attcttgcctcca (SEQ | | spectinomycin, |
| 1Mbdif-2L | CAST-TS1 | Syn61-1Mbdif | | ID NO: 247) | | sucrose |
| E. coli | E. coli DH10B | E. coli DH10B | TS2 | ttagcggaatgctggtacgg | cc | hygromycin, |
| Syn61-1R- | Δdif::lux | Δdif::lux TS2- | | gctgataaagaa (SEQ | | spectinomycin, |
| 1 Mbdif-2L | CAST-TS2 | Syn61-1Mbdif | | ID NO: 248) | | sucrose |
| E. coli | E. coli DH10B | E. coli DH10B | TS3 | agctgcaacaatgttgaaaa | cc | hygromycin, |
| Syn61-1R- | Δdif::lux | Δdif::lux TS3- | | tgccagccaact (SEQ | | spectinomycin, |
| 1 Mbdif-2L | CAST-TS3 | Syn61-1Mbdif | | ID NO: 249) | | sucrose |
| E. coli | E. coli DH10B | E. coli DH10B | TS1 | accagacccgcgagcatta | cc | hygromycin, |
| Syn61-1R- | Δdif::lux | Δdif::lux TS1- | | attcttgcctcca (SEQ | | spectinomycin, |
| 2Mbdif-2L | CAST-TS1 | Syn61-2Mbdif | | ID NO: 247) | | sucrose |
| Syn Seg. 1 | | Syn Seg. 1 | | | | |
| E. coli | E. coli DH10B | E. coli DH10B | TS2 | ttagcggaatgctggtacgg | cc | hygromycin, |
| Syn61-1R- | Δdif::lux | Δdif::lux TS2- | | gctgataaagaa (SEQ | | spectinomycin, |
| 2Mbdif-2L | CAST-TS2 | Syn61-2Mbdif | | ID NO: 248) | | sucrose |
| Syn Seg. 1 | | Syn Seg. 1 | | | | |
| E. coli | E. coli DH10B | E. coli DH10B | TS3 | agctgcaacaatgttgaaaa | cc | hygromycin, |
| Syn61-1R- | Δdif::lux | Δdif::lux TS3- | | tgccagccaact (SEQ | | spectinomycin, |
| 2Mbdif-2L | CAST-TS3 | Syn61-2Mbdif | | ID NO: 249) | | sucrose |
| Syn Seg. 1 | | Syn Seg. 1 | | | | |
| E. coli | E. coli DH10B | E. coli DH10B | TS1 | accagacccgcgagcatta | cc | hygromycin, |
| Syn61-1R- | Δdif::lux | Δdif::lux TS1- | | attcttgcctcca (SEQ | | spectinomycin, |
| 1 Mb-2L Syn | CAST-TS1 | Syn61-1Mb Syn | | ID NO: 247) | | sucrose |
| Seg. 2 | | Seg. 2 | | | | |
| E. coli | E. coli DH10B | E. coli DH10B | TS2 | ttagcggaatgctggtacgg | cc | hygromycin, |
| Syn61-1R- | Δdif::lux | Δdif::lux TS2- | | gctgataaagaa (SEQ | | spectinomycin, |
| 1Mb-2L Syn | CAST-TS2 | Syn61-1Mb Syn | | ID NO: 248) | | sucrose |
| Seg. 2 | | Seg. 2 | | | | |
| E. coli | E. coli DH10B | E. coli DH10B | TS3 | agctgcaacaatgttgaaaa | cc | hygromycin, |
| Syn61-1R- | Δdif: lux | Δdif::lux TS3- | | tgccagccaact (SEQ | | spectinomycin, |
| 1Mb-2L Syn | CAST-TS3 | Syn61-1Mb Syn | | ID NO: 249) | | sucrose |
| Seg. 2 | | Seg. 2 | | | | |
| E. coli | E. coli DH10B | E. coli DH10B | TS3 | agctgcaacaatgttgaaaa | cc | hygromycin, |
| Syn61-1R- | CAST-TS3 | Δdif::lux TS3- | | tgccagccaact (SEQ | | spectinomycin, |
| 1MboriC-2L | | Syn61-1MboriC | | ID NO: 249) | | sucrose |
| Syn Seg. 3 | | Syn Seg. 5 | | | | |
| E. coli | E. coli DH10B | E. coli DH10B | TS1 | accagacccgcgagcatta | cc | hygromycin, |
| Syn61-1R- | Δdif::lux | Δdif::lux TS1- | | attcttgcctcca (SEQ | | spectinomycin, |
| 500Kb-2L | CAST-TS1 | Syn61-500Kb | | ID NO: 247) | | sucrose |
| Syn Seg. 4 | | Syn Seg. 4 | | | | |
| E. coli | E. coli DH10B | E. coli DH10B | TS2 | ttagcggaatgctggtacgg | cc | hygromycin, |
| Syn61-1R- | Δdif::lux | Δdif::lux TS2- | | gctgataaagaa (SEQ | | spectinomycin, |
| | CAST-TS2 | | | ID NO: 248) | | sucrose |
| 500Kb-2L | | Syn61-500Kb | | | | |
| Syn Seg. 4 | | Syn Seg. 4 | | | | |
| E. coli | E. coli DH10B | E. coli DH10B | TS3 | agctgcaacaatgttgaaaa | cc | hygromycin, |
| Syn61-1R- | Δdif::lux | Δdif::lux TS3- | | tgccagccaact (SEQ | | spectinomycin, |
| 500Kb-2L | CAST-TS3 | Syn61-500Kb | | ID NO: 249) | | sucrose |
| Syn Seg. 4 | | Syn Seg. 4 | | | | |
| E. coli | E. coli DH10B | E. coli DH10B | TS1 | accagacccgcgagcatta | cc | hygromycin, |
| Syn61-1R- | Δdif::lux | Δdif::lux TS1- | | attcttgcctcca (SEQ | | spectinomycin, |
| 500Kb-2L | CAST-TS1 | Syn61-500Kb | | ID NO: 247) | | sucrose |
| Syn Seg. 5 | | Syn Seg. 3 | | | | |
| E. coli | E. coli DH10B | E. coli DH10B | TS2 | ttagcggaatgctggtacgg | cc | hygromycin, |
| Syn61-1R- | Δdif::lux | Δdif::lux TS2- | | gctgataaagaa (SEQ | | spectinomycin, |
| 500Kb-2L | CAST-TS2 | Syn61-500Kb | | ID NO: 248) | | sucrose |
| Syn Seg. 5 | | Syn Seg. 3 | | | | |
| E. coli | E. coli DH10B | E. coli DH10B | TS3 | agctgcaacaatgttgaaaa | cc | hygromycin, |
| Syn61-1R- | Δdif::lux | Δdif::lux TS3- | | tgccagccaact (SEQ | | spectinomycin, |
| 500Kb-2L | CAST-TS3 | Syn61-500Kb | | ID NO: 249) | | sucrose |
| Syn Seg. 5 | | Syn Seg. 3 | | | | |
| S. flexneri | E. coli DH10B | E. coli DH10B | dif | ttgttaatgagcatgacaatc | ca | hygromycin, |
| 2a-1R- | CAST-dif | Δdif::S. flexneri- | | atgaccgccaa (SEQ | | spectinomycin, |
| 1Mbdif-2L | | 1Mbdif | | ID NO: 254) | | sucrose |
| S. flexneri | E. coli MDS42 | E. coli MDS42 | dif | ttgttaatgagcatgacaatc | ca | hygromycin, |
| 2a-1R- | CAST-dif | Δdif::S. flexneri- | | atgaccgccaa (SEQ | | spectinomycin, |
| 1 Mbdif-2L | | 1 Mbdif | | ID NO: 254) | | sucrose |
| E. coli | P. putida | KT2440 | glmS | gtgctaaaggggaccgac | cc | hygromycin, |
| DH10B- | KT2440 CAST- | P. putida | | gttgaccagcctcg (SEQ | | kanamycin, |
| Syn100ΔlacZ | glmS | ΔglmS::Syn100- | | ID NO: 255) | | sucrose |
| 1R-100Kb- | | 100Kb | | | | |
| 2L | | | | | | |
| E. coli | V. natriegens | V. natriegens | wbfF | aaaaccatgcaggcagtta | cc | hygromycin, |
| MDS42- | DSM759 | DSM759 | | aagacatcacgca (SEQ | | carbenicillin, |
| lacZ-1R- | CAST-wbfF | ΔglmS::MDS42- | | ID NO: 256) | | sucrose |
| 200Kb-2L | | lacZ-200Kb | | | | |
| E. coli | V. natriegens | V. natriegens | chr. 1 | tttaaagaagcaaaagcca | cc | hygromycin, |
| MDS42-1R- | DSM759 | DSM759 | dif | aatacggtgtaat (SEQ | | carbenicillin, |
| 1 Mbdif-2L | CAST-difchl | Δdifch1::MDS4 | | ID NO: 257) | | sucrose |
| | | 2-1Mbdif | | | | |
| E. coli | V. natriegens | V. natriegens | chr. 2 | acaaatttaaaatctgagag | cc | hygromycin, |
| MDS42-1R- | DSM759 | DSM759 | dif | ggataggagact (SEQ | | carbenicillin, |
| 1Mbdif-2L | CAST-difch2 | Δdifch2::MDS4 | | ID NO: 258) | | sucrose |
| | | 2-1Mbdif | | | | |
| E. coli | E. coli DH10B | E. coli DH10B | dif | ttgttaatgagcatgacaatc | ca | hygromycin, |
| Syn61-1R- | CAST-dif | Δdif::Syn. Seg 1 | | atgaccgccaa (SEQ | | spectinomycin, |
| 2Mbdif-2L | | | | ID NO: 254) | | sucrose |
| Syn Seg. 1 | | | | | | |
| S. flexneri | E. coli DH10B | Δdif::Syn. Seg | dif | ttgttaatgagcatgacaatc | ca | hygromycin, |
| 2a-1R- | Δdif::Syn. Seg 1 | E. coli DH10B | | atgaccgccaa (SEQ | | spectinomycin, |
| 1Mbdif-2L | CAST-dif | 1-S. flexneri- | | ID NO: 254) | | sucrose |
| | | 1 Mbdif | | | | |
| E. coli | A. tumefaciens | A. tumefaciens | agpI | gttgcgagcggcattatcgc | cc | hygromycin, |
| Syn61-1R- | spectVSICAST | ΔagpI::Syn61- | | gatgcgggtttc (SEQ | | gentamcin, |
| 100Kbdif-2L | -agpI | 100Kbdif | | ID NO: 260) | | spectinomycin, |
| | | | | | | sucrose |
| E. coli | A. tumefaciens | A. tumefaciens | agpI | gttgcgagcggcattatcgc | cc | kanamycin, |
| MDS42- | spectVSICAST | ΔagpI::MDS42- | | gatgcgggtttc (SEQ | | gentamicin, |
| 1RRB- | -agpI | | | ID NO: 260) | | |
| 100Kbdif- | | 100Kbdif- | | | | spectinomycin, |
| 2LBL (T- | | TDNA | | | | sucrose |
| DNA) | | | | | | |
| E. coli | A. tumefaciens | A. tumefaciens | flaA | atgctttacacggaaggtac | cc | kanamycin, |
| MDS42- | spectVSICAST | ΔflaA::MDS42- | | accgggcacgat (SEQ | | gentamicin, |
| 1RRB- | -flaA | 100Kbdif-TDNA | | ID NO: 259) | | spectinomycin, |
| 100Kbdif- | | | | | | sucrose |
| 2LBL (T- | | | | | | |
| DNA) | | | | | | |
| E. coli | A. tumefaciens | A. tumefaciens | tetA | aaagtgaagccattgaaatc | cc | kanamycin, |
| MDS42- | spectVSICAST | ΔtetA::MDS42- | | attggcatgtcg (SEQ | | gentamicin, |
| 1RRB- | -tetA | 100Kbdif-TDNA | | ID NO: 261) | | spectinomycin, |
| 100Kbdif- | | | | | | sucrose |
| 2LBL (T- | | | | | | |
| DNA) | | | | | | |
| E. coli | A. tumefaciens | A. tumefaciens | dif | gtcatgcaatttgtgttctcg | cc | kanamycin, |
| MDS42- | spectVS1CAST | Δdif::MDS42- | (agro) | ccgatccttgg (SEQ ID | | gentamicin, |
| 1RRB- | -dif(agro) | 100Kbdif-TDNA | | NO: 262) | | spectinomycin, |
| 100Kbdif- | | | | | | sucrose |
| 2LBL (T- | | | | | | |
| DNA) | | | | | | |
| E. coli | E. coli DH10B | E. coli DH10B | TS2 | N/A | V/A | hygromycin, |
| Syn61- | Δdif::lux | Δdif::lux TS2- | (luxA) | | | tetracycline, |
| 1luxA- | pKW20 | Syn61-100Kbdif | | | | sucrose |
| 100Kbdif- | | | | | | |
| 2luxA | | | | | | |
| E. coli | E. coli DH10B | E. coli DH10B | TS2 | N/A | N/A | hygromycin, |
| Syn61- | Δdif::lux | Δdif::lux TS2- | (luxA) | | | tetracycline, |
| 1luxA- | pKW20 | Syn61-1Mbdif | | | | sucrose |
| 1 Mbdif- | | | | | | |
| 2luxA | | | | | | |
| E. coli | E. coli DH10B | E. coli DH10B | TS2 | N/A | N/A | hygromycin, |
| Syn61- | Δdif::lux | Δdif::lux TS2- | (luxA) | | | tetracycline, |
| 1luxA- | pKW20 | Syn61-2Mbdif | | | | sucrose |
| 2Mbdif- | | Syn Seg. 1 | | | | |
| 2luxA | | | | | | |
| E. coli | E. coli DH10B | E. coli DH10B | TS2 | N/A | N/A | hygromycin, |
| Syn61- | Δdif::lux | Δdif::lux TS2- | (luxA) | | | tetracycline, |
| 1luxA- | pKW20 | Syn61-100Kbdif | | | | carbenicillin, |
| 100Kbdif- | sgRNA-luxA | | | | | sucrose |
| 2luxA | | | | | | |
| E. coli | E. coli DH10B | E. coli DH10B | TS2 | N/A | N/A | hygromycin, |
| Syn61- | Δdif::lux | Δdif::lux TS2- | (luxA) | | | tetracycline, |
| 1luxA- | pKW20 | Syn61-1Mbdif | | | | carbenicillin, |
| 1 Mbdif- | sgRNA-luxA | | | | | sucrose |
| 2luxA | | | | | | |
| E. coli | E. coli DH10B | E. coli DH10B | TS2 | N/A | N/A | hygromycin, |
| Syn61- | Δdif::lux | Δdif::lux TS2- | (luxA) | | | tetracycline, |
| 1luxA- | pKW20 | Syn61-2Mbdif | | | | carbenicillin, |
| 2Mbdif- | sgRNA-luxA | Syn Seg. 1 | | | | sucrose |
| 2luxA | | | | | | |
| S. flexneri | E. coli DH10B | E. coli DH10B | dif | ttgttaatgagcatgacaatc | ca | kanamycin, |
| 2a-1R- | CAST-dif | Δdif:: | | tatgaccgccaa (SEQ | | hygromycin |
| 1500Kbdif- | | S. flexneri- | | ID NO: 254) | | |
| 2L | | 1500Kbdif | | | | |
| P. protegens | E. coli DH10B | E. coli DH10B | TS2 | ttagcggaatgctggtacgg | cc | kanamycin, |
| pf-5-1R- | hygroCDFCAS | Δdif::lux TS2-P. | | gctgataaagaa (SEQ | | hygromycin |
| orfamide-2L | Δdif::lux | pro orf 80Kb | | ID NO: 248) | | |
| (80 Kb) | T-TS2 | | | | | |
| P. protegens | E. coli DH10B | E. coli DH10B | TS3 | agctgcaacaatgttgaaaa | cc | kanamycin, |
| pf-5-1R- | hygroCDFCAS | Δdif::lux TS3-P. | | tgccagccaact (SEQ | | hygromycin |
| orfamide-2L | Δdif::lux | pro orf 80Kb | | ID NO: 249) | | |
| (80 Kb) | T-TS3 | | | | | |
| P. protegens | E. coli DH10B | E. coli DH10B | TS2 | ttagcggaatgctggtacgg | cc | kanamycin, |
| pf-5-1R- | Δdif::lux | Δdif::lux TS2-P. | | gctgataaagaa (SEQ | | hygromycin |
| rhizoxin-2L | hygroCDFCAS | pro rhz 140Kb | | ID NO: 248) | | |
| (140 Kb) | T-TS2 | | | | | |
| P. protegens | E. coli DH10B | E. coli DH10B | TS3 | agctgcaacaatgttgaaaa | cc | kanamycin, |
| pf-5-1R- | Δdif::lux | Δdif::lux TS3-P. | | tgccagccaact (SEQ | | hygromycin |
| rhizoxin-2L | hygroCDFCAS | pro rhz 140Kb | | ID NO: 249) | | |
| (140 Kb) | T-TS3 | | | | | |
| P. protegens | E. coli DH10B | E. coli DH10B | TS2 | ttagcggaatgctggtacgg | cc | kanamycin, |
| pf-5-1R- | Δdif::lux | Δdif::lux TS2-P. | | gctgataaagaa (SEQ | | hygromycin |
| pyoluteorin- | hygroCDFCAS | pro pyl-rhz | | ID NO: 248) | | |
| rhizoxin-2L | T-TS2 | 415Kb | | | | |
| (415 Kb) | | | | | | |
| P. protegens | E. coli DH10B | E. coli DH10B | dif | ttgttaatgagcatgacaatc | ca | kanamycin, |
| pf-5-1R- | hygroCDFCAS | Δdif::P. pro pyl- | | atgaccgccaa (SEQ | | hygromycin |
| pyoluteorin- | T-dif | rhz 415Kb | | ID NO: 254) | | |
| rhizoxin-2L | | | | | | |
| (415 Kb) | | | | | | |
| P. protegens | E. coli DH10B | E. coli DH10B | TS3 | agctgcaacaatgttgaaaa | cc | kanamycin, |
| pf-5-1R- | hygroCDFCAS | Δdif::lux TS3-P. | | tgccagccaact (SEQ | | hygromycin |
| pyrrolnitrin- | Δdif::lux | pro pyn 60Kb | | ID NO: 249) | | |
| 2L (60 Kb) | T-TS3 | | | | | |
| P. protegens | E. coli DH10B | E. coli DH10B | TS2 | ttagcggaatgctggtacgg | cc | kanamycin, |
| pf-5-1R- | hygroCDFCAS | Δdif::lux TS2-P. | | gctgataaagaa (SEQ | | hygromycin |
| pyoluteorin- | Δdif::lux | pro pyl 66Kb | | ID NO: 248) | | |
| 2L (66 Kb) | T-TS2 | | | | | |
| P. protegens | E. coli DH10B | E. coli DH10B | TS3 | agctgcaacaatgttgaaaa | cc | kanamycin, |
| pf-5-1R- | hygroCDFCAS | Δdif::lux TS3-P. | | tgccagccaact (SEQ | | hygromycin |
| pyoluteorin- | Δdif::lux | pro pyl 66Kb | | ID NO: 249) | | |
| 2L (66 Kb) | T-TS3 | | | | | |
| P. protegens | E. coli DH10B | E. coli DH10B | TS3 | agctgcaacaatgttgaaaa | cc | kanamycin, |
| pf-5-1R- | hygroCDFCAS | TS3-P. pro pyv | | tgccagccaact (SEQ | | hygromycin |
| pyoverdine- | T-TS3 | 246Kb | | ID NO: 249) | | |
| 2L (246 Kb) | | | | | | |
| P. protegens | E. coli DH10B | E. coli DH10B | TS5 | gaaaacacctgatatgaaa | Icc | kanamycin, |
| pf-5-1R- | hygroCDFCAS | TS5-P. pro pyv | | ggcaatgccacca (SEQ | | hygromycin |
| pyoverdine- | T-TS5 | 246Kb | | ID NO: 264) | | |
| 2L (246 Kb) | | | | | | |
| P. protegens | E. coli DH10B | E. coli DH10B | TS3 | agctgcaacaatgttgaaaa | cc | kanamycin, |
| pf-5-1R- | hygroCDFCAS | TS3-P. pro cit- | | tgccagccaact (SEQ | | hygromycin |
| citronellol- | T-TS3 | pyv 373Kb | | ID NO: 249) | | |
| pyoverdine- | | | | | | |
| 2L (373 Kb) | | | | | | |
| P. protegens | E. coli DH10B | E. coli DH10B | TS3 | agctgcaacaatgttgaaaa | cc | kanamycin, |
| pf-5-1R- | hygroCDFCAS | TS3-P. pro pyv- | | tgccagccaact (SEQ | | hygromycin |
| pyoverdine- | T-TS3 | cit 395Kb | | ID NO: 249) | | |
| citronellol- | | | | | | |
| 2L (395 Kb) | | | | | | |
| P. protegens | E. coli DH10B | E. coli DH10B | TS4 | tggtggcggtggtgggagc | cc | kanamycin, |
| pf-5-1R- | hygroCDFCAS | TS4-P. pro pyv- | | tattctcgttctg (SEQ ID | | hygromycin |
| pyoverdine- | T-TS4 | cit 395Kb | | NO: 263) | | |
| citronellol- | | | | | | |
| 2L (395 Kb) | | | | | | |
| P. protegens | E. coli DH10B | E. coli DH10B | dif | ttgttaatgagcatgacaatc | ca | kanamycin, |
| pf-5-1R- | hygroCDFCAS | Δdif::P. pro | | atgaccgccaa (SEQ | | hygromycin |
| pyoverdine- | IT-dif | pyv-cit 395Kb | | ID NO: 254) | | |
| citronellol- | | | | | | |
| 2L (395 Kb) | | | | | | |
| P. protegens | E. coli DH10B | E. coli DH10B | TS3 | agctgcaacaatgttgaaaa | cc | kanamycin, |
| pf-5-1R- | hygroCDFCAS | TS3-P. pro cit | | tgccagccaact (SEQ | | hygromycin |
| citronellol- | T-TS3 | 522Kb | | ID NO: 249) | | |
| 2L (522 Kb) | | | | | | |
| P. protegens | E. coli DH10B | E. coli DH10B | TS4 | tggtggcggtggtgggagc | cc | kanamycin, |
| pf-5-1R- | hygroCDFCAS | TS4-P. pro cit | | tattctcgttctg (SEQ ID | | hygromycin |
| citronellol- | T-TS4 | 522Kb | | NO: 263) | | |
| 2L (522 Kb) | | | | | | |
| P. protegens | E. coli DH10B | E. coli DH10B | dif | ttgttaatgagcatgacaatc | ca | kanamycin, |
| pf-5-1R- | hygroCDFCAS | Δdif::P. pro cit | | atgaccgccaa (SEQ | | hygromycin |
| citronellol- | T-dif | 522Kb | | ID NO: 254) | | |
| 2L (522 Kb) | | | | | | |

TABLE 8
Chimeric Genomes*

| | Genome Size | | TE Insert |
|---|---|---|---|
| Chimeric Strain | (bp) | Stability | orientation |
| E. coli DH10B Δdif::lux TS1-Syn100-10Kb | 4707021 | Stable | RL |
| E. coli DH10B Δdif::lux TS2-Syn100-10Kb | 4707021 | Stable | RL |
| E. coli DH10B Δdif::lux TS3-Syn100-10Kb | 4707021 | Stable | RL |
| E. coli DH10B Δdif::lux TS1-Syn100-50Kb | 4747023 | Stable | RL |
| E. coli DH10B Δdif::lux TS2-Syn100-50Kb | 4747023 | Stable | RL |
| E. coli DH10B Δdif::lux TS3-Syn100-50Kb | 4747023 | Stable | RL |
| E. coli DH10B Δdif::lux TS1-Syn100-100Kb | 4801035 | Stable | RL |
| E. coli DH10B Δdif::lux TS2-Syn100-100Kb | 4801035 | Stable | RL |
| E. coli DH10B Δdif::lux TS3-Syn100-100Kb | 4801035 | Stable | RL |
| E. coli DH10B Δdif::lux luxA*-Syn100-100Kb | 4801035 | Stable | RL |
| E. coli DH10B Δdif::lux luxB1-Syn100-100Kb | 4801035 | Stable | RL |
| E. coli DH10B Δdif::lux luxB2-Syn100-100Kb | 4801035 | Stable | RL |
| E. coli DH10B TS1-MDS42-lacZ-200Kb | 4900215 | Stable | RL |
| E. coli DH10B Δdif::lux TS1-Syn61-100Kbdif | 4803858 | Stable | LR |
| E. coli DH10B Δdif::lux TS2-Syn61-100Kbdif | 4803858 | Stable | RL |
| E. coli DH10B Δdif::lux TS3-Syn61-100Kbdif | 4803858 | Stable | RL |
| E. coli DH10B Δdif::lux TS1-Syn61-600Kbdif | 5289866 | Unstable | RL |
| E. coli DH10B Δdif::lux TS2-Syn61-600Kbdif | 5289866 | Stable | RL |
| E. coli DH10B Δdif::lux TS3-Syn61-600Kbdif | 5289866 | Unstable | RL |
| E. coli DH10B Δdif::lux TS1-Syn61-1Mbdif | 5630492 | Unstable | RL |
| E. coli DH10B Δdif::lux TS2-Syn61-1Mbdif | 5630492 | Stable | RL |
| E. coli DH10B Δdif::lux TS3-Syn61-1Mbdif | 5630492 | Unstable | RL |
| E. coli DH10B Δdif::lux TS1-Syn61-2Mbdif Syn Seg. 1 | 6609266 | Unstable | RL |
| E. coli DH10B Δdif::lux TS2-Syn61-2Mbdif Syn Seg. 1 | 6609266 | Stable | RL |
| E. coli DH10B Δdif::lux TS3-Syn61-2Mbdif Syn Seg. 1 | 6609266 | Unstable | RL |
| E. coli DH10B Δdif::lux TS1-Syn61-1Mb Syn Seg. 2 | 5756079 | Stable | RL |
| E. coli DH10B Δdif::lux TS2-Syn61-1Mb Syn Seg. 2 | 5756079 | Stable | RL |
| E. coli DH10B Δdif::lux TS3-Syn61-1Mb Syn Seg. 2 | 5756079 | Stable | LR |
| E. coli DH10B Δdif::lux TS3-Syn61-1MboriC Syn Seg. 3 | 5675263 | Stable | RL |
| E. coli DH10B Δdif::lux TS1-Syn61-500Kb Syn Seg. 4 | 5255474 | Stable | RL |
| E. coli DH10B Δdif::lux TS2-Syn61-500Kb Syn Seg. 4 | 5255474 | Stable | RL |
| E. coli DH10B Δdif::lux TS3-Syn61-500Kb Syn Seg. 4 | 5255474 | Stable | RL |
| E. coli DH10B Δdif::lux TS1-Syn61-500Kb Syn Seg. 5 | 5232612 | Stable | RL |
| E. coli DH10B Δdif::lux TS2-Syn61-500Kb Syn Seg. 5 | 5232612 | Stable | RL |
| E. coli DH10B Δdif::lux TS3-Syn61-500Kb Syn Seg. 5 | 5232612 | Stable | RL |
| E. coli DH10B Δdif::S. flexneri-1Mbdif | 5570081 | Stable | RL |
| E. coli MDS42 Δdif::S. flexneri-1Mbdif | 4860139 | Stable | RL |
| P. putida KT2440 ΔglmS::Syn100-100Kb | 6292437 | Stable | RL |
| V. natriegens DSM759 ΔglmS::MDS42-lacZ-200Kb | 3462101 | Stable* | RL |
| V. natriegens DSM759 Δdifch1::MDS42-1Mbdif | 4117792 | Stable* | RL |
| V. natriegens DSM759 Δdifch2::MDS42-1Mbdif | 2796899 | Stable* | RL |
| E. coli DH10B Δdif::Syn. Seg 1 | 6601793 | Stable | RL |
| E. coli DH10B Δdif::Syn. Seg 1-S. flexneri-1Mbdif | 7485737 | Stable | RL |

*Stability reported via sequencing, but no growth obtained following inoculation of cultures from glycerol stocks. Synthetic versions of strains were denoted as the taxonomy ID of the preceding WT strain, followed by an S#, where # is an arbitrary number assigned to a given synthetic strain.

TABLE 9
Exemplary Sequences

| Identity | SEQ ID NO: |
|---|---|
| R-TE (RE)-ortho 1 | 343 |
| R-TE (RE)-ortho 2 | 344 |
| L-TE (LE)-ortho 1 | 345 |
| L-TE (LE)-ortho 2 | 346 |
| Cas6 protein sequence-ortho 1 | 347 |
| Cas6 protein sequence-ortho2 | 348 |
| Cas7 protein sequence-ortho 1 | 349 |
| Cas7 protein sequence-ortho2 | 350 |
| Cas8 protein sequence-ortho1 | 351 |
| Cas8 protein sequence-ortho2 | 352 |
| TniQ protein sequence-ortho 1 | 353 |
| TniQ protein sequence-ortho2 | 354 |
| TnsA protein sequence-ortho 1 | 355 |
| TnsA protein sequence-ortho 2 | 356 |
| TnsB protein sequence-ortho 1 | 357 |
| TnsB protein sequence-ortho 2 | 358 |
| TnsC protein sequence-ortho 1 | 359 |
| TnsC protein sequence-ortho 2 | 360 |
| R-TE (RE) ACE Tn6677 | 361 |
| L-TE (LE) ACE Tn6677 | 362 |
| Cas6 protein sequence | 363 |
| Cas7 protein sequence | 364 |
| Cas8 protein sequence | 365 |
| TniQ protein sequence | 366 |
| TnsA protein sequence | 367 |
| TnsB protein sequence | 368 |
| InsC protein sequence | 369 |
| Cassette^{START} | 370 |
| Cassette^{STOP} | 371 |
| Cassette^{START} OASIS | 372, 373 |
| Cassette^{STOP} OASIS | 374, 375 |
| P^{ara}-Trans conj. machinery nt sequence | 376 |
| p^{Ara} -CAST machinery nt sequence | 377 |
| Ortho-Cast1 machinery nt sequence | 378 |
| pOrtho-Cast2 machinery nt sequence | 379 |
| P^{Ara}-Trans Plasmid | 380 |
| P^{Ara} _CAST Plasmid | 381 |
| pOrthoCAST1 vector | 382 |
| pOrthoCAST2 vector | 383 |
| origin of Transfer (oriT) | 384 |
| CAST crRNA repeat-scaffold-repeat | 385 (DNA), 388 (RNA) |
| orthoCAST1 crRNA repeat-scaffold-repeat | 386 (DNA), 389 (RNA) |
| orthoCAST2 crRNA repeat-scaffold-repeat | 387 (DNA), 390 (RNA) |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system for producing a chimeric genome, comprising:
a) a donor cell comprising:
(i) a first start cassette (Cassette^{START}) comprising, from 5′ to 3′, at least one cis-acting component of a first delivery module and a first integration sequence, and
(ii) a first stop cassette (Cassette^{STOP}) comprising, from 5′ to 3′, a second integration sequence and at least one cis-acting component of the first delivery module, wherein the first Cassette^{START} is integrated at a first site in the genome of the donor cell and the first Cassette^{STOP} is integrated at a second site in the genome of the donor cell, wherein intervening sequence between the first Cassette^{START} and the first Cassette^{STOP} defines a first genomic transfer window; and
(iii) one or more trans-acting components of the first delivery module or one or more first helper polynucleotides each comprising a sequence encoding a trans-acting component of the first delivery module, wherein the first delivery module comprises the one or more trans-acting components; and
b) a recipient cell comprising one or more components of an integrator module or one or more second helper polynucleotides each comprising a sequence encoding a component of an integrator module, wherein the integrator module comprises the one or more components.

2. The system of claim 1, wherein:
(1) a sequence defined by the first genomic transfer window is capable of being transferred from the donor cell to the recipient cell upon expression of the one or more trans-acting components of the first delivery module in the donor cell, thereby generating a first donor genomic segment in the recipient cell flanked by the first integration sequence and the second integration sequence of the first Cassette^{START} and the first Cassette^{STOP}; and
(2) the first donor genomic segment is capable of being inserted into a first double-stranded target sequence in the genome of the recipient cell upon expression of the one or more components of the integrator module, thereby generating a chimeric genome in the recipient cell.

3. (canceled)

4. (canceled)

5. The system of claim 1, wherein the first and/or second genomic transfer window is about 10 kb to at least 2 MB in length and/or wherein the chimeric genome is at least about 4 MB in length.

6.-8. (canceled)

9. The system of claim 1, wherein the donor cell, the recipient cell, or any combination thereof is a eukaryotic cell or a prokaryotic cell.

10.-12. (canceled)

13. The system of claim 1, wherein the genera and/or species of the donor cell and the genera and/or the species of the recipient cell are the same or different.

14. A nucleic acid composition for producing a chimeric genome, comprising:
i) a first start cassette (Cassette^{START}) comprising, from 5′ to 3′: at least one cis-acting component of a first delivery module and a first integration sequence;
ii) a first stop cassette (Cassette^{STOP}) comprising, from 5′ to 3′: a second integration sequence and at least one cis-acting component of the first delivery module;
iii) one or more first helper polynucleotides each comprising a sequence encoding a trans-acting component of the first delivery module, wherein the first delivery module comprises one or more trans-acting components.

15.-27. (canceled)

28. The system of claim 1, wherein the first delivery module comprises or is derived from a bacterial conjugation system.

29. The system of claim 28, wherein the bacterial conjugation system is an RP4 (IncP) plasmid system, and wherein:
the at least one cis-acting component comprises an RP4 origin of transfer (oriT) sequence, and wherein the oriT sequence comprises a sequence of SEQ ID NO: 384 or a sequence that is at least 80%, 85%, 90%, 95%, 98%, 99%, or 100% identical to SEQ ID NO: 384; and
the one or more trans-acting components comprise or are derived from gene products of the RP4 plasmid.

30.-33. (canceled)

34. The system of claim 1, wherein the integrator module is derived from a transposase system or a site-directed DNA recombinase system.

35. The system of claim 1, wherein the integrator module is derived from a Type I-B, Type I-D, Type I-F, or Type V-K CRISPR-associated transposase system of a bacteria.

36. (canceled)

37. (canceled)

38. The system of claim 1, wherein:
the one or more components of the integrator module comprise:
i) an RNA-guided DNA binding complex, wherein the RNA-guided DNA binding complex comprises one or more Cas proteins, a transposase, one or more crRNAs, or any combination thereof, and
ii) a transposition complex comprising one or more transposases; and
the first integration sequence of the first Cassette^{START} comprises an R-TE (RE) and the second integration sequence of the first Cassette^{STOP} comprises an L-TE (LE).

39. (canceled)

40. The system of claim 38, wherein:
the one or more Cas proteins comprise a Cas6 protein, a Cas7 protein, and a Cas8 protein;
the transposase of the RNA-guided DNA binding complex comprises a TniQ protein; and/or
the one or more transposases of the transposition complex comprise a TnsA protein, a TnsB protein, and a TnsC protein.

41.-46. (canceled)

47. The system or of claim 38, wherein at least one of the one or more crRNAs comprise a spacer that is complementary to a search target sequence on a first strand of the first double stranded target sequence.

48.-58. (canceled)

59. The system of claim 1, wherein the first Cassette^{START}, the first Cassette^{STOP} or any combination thereof comprise: one or more marker polynucleotides each comprising a sequence encoding a positive or negative screening and/or selection marker.

60. The system of claim 59, wherein:
the positive screening and/or selection marker is selected from the group comprising: a fluorescent protein, an antibiotic resistance cassette, an enzyme, or any combination thereof, and/or
the negative screening and/or selection marker is selected from the group comprising: a fluorescent protein, an enzyme, or a combination thereof.

61.-64. (canceled)

65. The system of claim 1, wherein:
one or more of the one or more first helper polynucleotides comprise a first promoter operably linked to the sequence encoding a trans-acting component of the first delivery module; and/or
one or more of the one or more second helper polynucleotides comprise a second promoter operably linked to the sequence encoding a component of an integrator module.

66.-76. (canceled)

77. The system of claim 1, wherein:
two or more of the one or more first helper polynucleotides are situated on the same nucleic acid, and/or
two or more of the one or more second helper polynucleotides are situated on the same nucleic acid,
and wherein the two or more first and/or second helper polynucleotides situated on the same nucleic acid are comprised within an operon or operably linked via a tandem expression element.

78.-80. (canceled)

81. The system of claim 1, wherein:
i) the first Cassette^{START} comprises the sequence of SEQ ID NO: 370 or a sequence that is at least 80%, 85%, 90%, 95%, 98%, 99%, or 100% identical to SEQ ID NO: 370; and/or
ii) the first Cassette^{STOP} comprises the sequence of SEQ ID NO: 371 or a sequence that is at least 80%, 85%, 90%, 95%, 98%, 99%, or 100% identical to SEQ ID NO: 371.

82. The system of claim 78, wherein:
the two or more first helper polynucleotides comprised within an operon or operably linked via a tandem expression element comprise the sequence of SEQ ID NO: 376 or a sequence that is at least 80%, 85%, 90%, 95%, 98%, 99%, or 100% identical to any one of SEQ ID NO: 376; and/or
the two more second helper polynucleotides comprised within an operon or operably linked via a tandem expression element comprise the sequence of SEQ ID NO: 377 or a sequence that is at least 80%, 85%, 90%, 95%, 98%, 99%, or 100% identical to any one of SEQ ID NO: 377.

83. (canceled)

84. (canceled)

85. A method for producing a chimeric genome, comprising:
contacting the recipient cell of claim 1 with the donor cell of claim 1, and:
expressing the one or more trans-acting components of the first delivery module in the donor cell, thereby the sequence defined by the genomic transfer window is transferred from the donor cell to the recipient cell and thereby the first donor genomic segment comprising the first donor sequence flanked by the first integration sequence and the second integration sequence of the first Cassette^{START} and the first Cassette^{STOP} is generated in the recipient cell; and
expressing the one or more components of the integrator module in the recipient cell, thereby the first donor genomic segment is inserted into the first double-stranded target sequence in the genome of the recipient cell,
thereby generating the chimeric genome in the recipient cell.

86.-93. (canceled)
